SEQUENCE LISTING (384 sequences)

SEQ ID NO: 1 - protein, 142 aa, Homo sapiens
  REGION 1..142: note = Description of sequence: full-length wildtype FKBP13 sequence
MRLSWFRVLTVLSICLSAVATATGAEGKRKLQIGVKKRVDHCPIKSRKGDVLHMHYTGKLEDGTEFDSSLPQNQPFVFSLGTGQVIKGWDQGLLGMCEGEKRKLVIPSELGYGERGAPPKIPGGATLVFEVELLKIERRTEL

SEQ ID NO: 2 - protein, 98 aa, synthetic construct
  REGION 1..98: note = Description of sequence: FKBP13-AcGFP-mCherry Fusion; Construct Name: FKBP-039; FKBP13 DRD in Construct : FKBP13 (WT)
HCPIKSRKGDVLHMHYTGKLEDGTEFDSSLPQNQPFVFSLGTGQVIKGWDQGLLGMCEGEKRKLVIPSELGYGERGAPPKIPGGATLVFEVELLKIER

SEQ ID NO: 3 - protein, 98 aa, synthetic construct
  REGION 1..98: note = Description of sequence: FKBP13-AcGFP-mCherry Fusion; Construct Name: FKBP-040; FKBP13 DRD in Construct : FKBP13 (DRD - D90G)
HCPIKSRKGDVLHMHYTGKLEDGTEFDSSLPQNQPFVFSLGTGQVIKGWGQGLLGMCEGEKRKLVIPSELGYGERGAPPKIPGGATLVFEVELLKIER

SEQ ID NO: 4 - protein, 98 aa, synthetic construct
  REGION 1..98: note = Description of sequence: FKBP13-AcGFP-mCherry Fusion; Construct Name: FKBP-041; FKBP13 DRD in Construct : FKBP13 (DRD - M54A)
HCPIKSRKGDVLHAHYTGKLEDGTEFDSSLPQNQPFVFSLGTGQVIKGWDQGLLGMCEGEKRKLVIPSELGYGERGAPPKIPGGATLVFEVELLKIER

SEQ ID NO: 5 - protein, 98 aa, synthetic construct
  REGION 1..98: note = Description of sequence: FKBP13-AcGFP-mCherry Fusion; Construct Name: FKBP-042; FKBP13 DRD in Construct : FKBP13 (DRD - M96V)
HCPIKSRKGDVLHMHYTGKLEDGTEFDSSLPQNQPFVFSLGTGQVIKGWDQGLLGVCEGEKRKLVIPSELGYGERGAPPKIPGGATLVFEVELLKIER

SEQ ID NO: 6 - protein, 98 aa, synthetic construct
  REGION 1..98: note = Description of sequence: FKBP13-AcGFP-mCherry Fusion; Construct Name: FKBP-043; FKBP13 DRD in Construct : FKBP13 (mutated to match FKBP12 active site - Q72R Q84E A117H) (DRD - D90G)
HCPIKSRKGDVLHMHYTGKLEDGTEFDSSLPRNQPFVFSLGTGEVIKGWGQGLLGMCEGEKRKLVIPSELGYGERGHPPKIPGGATLVFEVELLKIER

SEQ ID NO: 7 - protein, 98 aa, synthetic construct
  REGION 1..98: note = Description of sequence: FKBP13-AcGFP-mCherry Fusion; Construct Name: FKBP-044; FKBP13 DRD in Construct : FKBP13 (mutated to match FKBP12 active site - Q72R Q84E A117H) (DRD - M54A)
HCPIKSRKGDVLHAHYTGKLEDGTEFDSSLPRNQPFVFSLGTGEVIKGWDQGLLGMCEGEKRKLVIPSELGYGERGHPPKIPGGATLVFEVELLKIER

SEQ ID NO: 8 - protein, 98 aa, synthetic construct
  REGION 1..98: note = Description of sequence: FKBP13-AcGFP-mCherry Fusion; Construct Name: FKBP-045; FKBP13 DRD in Construct : FKBP13 (mutated to match FKBP12 active site - Q72R Q84E A117H) (DRD - M96V)
HCPIKSRKGDVLHMHYTGKLEDGTEFDSSLPRNQPFVFSLGTGEVIKGWDQGLLGVCEGEKRKLVIPSELGYGERGHPPKIPGGATLVFEVELLKIER

SEQ ID NO: 9 - protein, 98 aa, synthetic construct
  REGION 1..98: note = Description of sequence: FKBP13-AcGFP-mCherry Fusion; Construct Name: FKBP-053; FKBP13 DRD in Construct : FKBP13 (mutated to match FKBP12 active site - Q72R Q84E A117H) (DRD - D90G F66H)
HCPIKSRKGDVLHMHYTGKLEDGTEHDSSLPRNQPFVFSLGTGEVIKGWGQGLLGMCEGEKRKLVIPSELGYGERGHPPKIPGGATLVFEVELLKIER

SEQ ID NO: 10 - protein, 98 aa, synthetic construct
  REGION 1..98: note = Description of sequence: FKBP13-AcGFP-mCherry Fusion; Construct Name: FKBP-054; FKBP13 DRD in Construct : FKBP13 (mutated to match FKBP12 active site - Q72R Q84E A117H) (DRD - D90G L127K)
HCPIKSRKGDVLHMHYTGKLEDGTEFDSSLPRNQPFVFSLGTGEVIKGWGQGLLGMCEGEKRKLVIPSELGYGERGHPPKIPGGATKVFEVELLKIER

SEQ ID NO: 11 - protein, 98 aa, synthetic construct
  REGION 1..98: note = Description of sequence: FKBP13-AcGFP-mCherry Fusion; Construct Name: FKBP-055; FKBP13 DRD in Construct : FKBP13 mutated to match FKBP12 active site - (Q72R Q84E A117H) (DRD - F78V)
HCPIKSRKGDVLHMHYTGKLEDGTEFDSSLPRNQPFVVSLGTGEVIKGWDQGLLGMCEGEKRKLVIPSELGYGERGHPPKIPGGATLVFEVELLKIER

SEQ ID NO: 12 - protein, 98 aa, synthetic construct
  REGION 1..98: note = Description of sequence: FKBP13-AcGFP-mCherry Fusion; Construct Name: FKBP-056; FKBP13 DRD in Construct : FKBP13 (mutated to match FKBP12 active site - Q72R Q84E A117H) (DRD - F129K)
HCPIKSRKGDVLHMHYTGKLEDGTEFDSSLPRNQPFVFSLGTGEVIKGWDQGLLGMCEGEKRKLVIPSELGYGERGHPPKIPGGATLVKEVELLKIER

SEQ ID NO: 13 - protein, 98 aa, synthetic construct
  REGION 1..98: note = Description of sequence: FKBP13-AcGFP-mCherry Fusion; Construct Name: FKBP-057; FKBP13 DRD in Construct : FKBP13 (mutated to match FKBP12 active site - Q72R Q84E A117H) (DRD - I106A)
HCPIKSRKGDVLHMHYTGKLEDGTEFDSSLPRNQPFVFSLGTGEVIKGWDQGLLGMCEGEKRKLVAPSELGYGERGHPPKIPGGATLVFEVELLKIER

SEQ ID NO: 14 - protein, 98 aa, synthetic construct
  REGION 1..98: note = Description of sequence: FKBP13-AcGFP-mCherry Fusion; Construct Name: FKBP-058; FKBP13 DRD in Construct : FKBP13 (mutated to match FKBP12 active site - Q72R Q84E A117H) (DRD - L52A)
HCPIKSRKGDVAHMHYTGKLEDGTEFDSSLPRNQPFVFSLGTGEVIKGWDQGLLGMCEGEKRKLVIPSELGYGERGHPPKIPGGATLVFEVELLKIER

SEQ ID NO: 15 - protein, 98 aa, synthetic construct
  REGION 1..98: note = Description of sequence: FKBP13-AcGFP-mCherry Fusion; Construct Name: FKBP-059; FKBP13 DRD in Construct : FKBP13 (mutated to match FKBP12 active site - Q72R Q84E A117H) (DRD - L93A)
HCPIKSRKGDVLHMHYTGKLEDGTEFDSSLPRNQPFVFSLGTGEVIKGWDQGALGMCEGEKRKLVIPSELGYGERGHPPKIPGGATLVFEVELLKIER

SEQ ID NO: 16 - protein, 98 aa, synthetic construct
  REGION 1..98: note = Description of sequence: FKBP13-AcGFP-mCherry Fusion; Construct Name: FKBP-060; FKBP13 DRD in Construct : FKBP13 (mutated to match FKBP12 active site - Q72R Q84E A117H) (DRD - L104A)
HCPIKSRKGDVLHMHYTGKLEDGTEFDSSLPRNQPFVFSLGTGEVIKGWDQGLLGMCEGEKRKAVIPSELGYGERGHPPKIPGGATLVFEVELLKIER

SEQ ID NO: 17 - protein, 98 aa, synthetic construct
  REGION 1..98: note = Description of sequence: FKBP13-AcGFP-mCherry Fusion; Construct Name: FKBP-061; FKBP13 DRD in Construct : FKBP13 (mutated to match FKBP12 active site - Q72R Q84E A117H) (DRD - L133A)
HCPIKSRKGDVLHMHYTGKLEDGTEFDSSLPRNQPFVFSLGTGEVIKGWDQGLLGMCEGEKRKLVIPSELGYGERGHPPKIPGGATLVFEVEALKIER

SEQ ID NO: 18 - protein, 98 aa, synthetic construct
  REGION 1..98: note = Description of sequence: FKBP13-AcGFP-mCherry Fusion; Construct Name: FKBP-062; FKBP13 DRD in Construct : FKBP13 (mutated to match FKBP12 active site - Q72R Q84E A117H) (DRD - M54A [-]disulfide)
HSPIKSRKGDVLHAHYTGKLEDGTEFDSSLPRNQPFVFSLGTGEVIKGWDQGLLGMSEGEKRKLVIPSELGYGERGHPPKIPGGATLVFEVELLKIER

SEQ ID NO: 19 - protein, 98 aa, synthetic construct
  REGION 1..98: note = Description of sequence: FKBP13-AcGFP-mCherry Fusion; Construct Name: FKBP-063; FKBP13 DRD in Construct : FKBP13 (mutated to match FKBP12 active site - Q72R Q84E A117H) (DRD - M54A C42S )
HSPIKSRKGDVLHAHYTGKLEDGTEFDSSLPRNQPFVFSLGTGEVIKGWDQGLLGMCEGEKRKLVIPSELGYGERGHPPKIPGGATLVFEVELLKIER

SEQ ID NO: 20 - protein, 98 aa, synthetic construct
  REGION 1..98: note = Description of sequence: FKBP13-AcGFP-mCherry Fusion; Construct Name: FKBP-064; FKBP13 DRD in Construct : FKBP13 (mutated to match FKBP12 active site - Q72R Q84E A117H) (DRD - R102L)
HCPIKSRKGDVLHMHYTGKLEDGTEFDSSLPRNQPFVFSLGTGEVIKGWDQGLLGMCEGEKLKLVIPSELGYGERGHPPKIPGGATLVFEVELLKIER

SEQ ID NO: 21 - protein, 98 aa, synthetic construct
  REGION 1..98: note = Description of sequence: FKBP13-AcGFP-mCherry Fusion; Construct Name: FKBP-065; FKBP13 DRD in Construct : FKBP13 (mutated to match FKBP12 active site - Q72R Q84E A117H)
HCPIKSRKGDVLHMHYTGKLEDGTEFDSSLPRNQPFVFSLGTGEVIKGWDQGLLGMCEGEKRKLVIPSELGYGERGHPPKIPGGATLVFEVELLKIER

SEQ ID NO: 22 - protein, 98 aa, synthetic construct
  REGION 1..98: note = Description of sequence: FKBP13-AcGFP-mCherry Fusion; Construct Name: FKBP-066; FKBP13 DRD in Construct : FKBP13 (mutated to match FKBP12 active site - Q72R Q84E A117H) (DRD - M54K)
HCPIKSRKGDVLHKHYTGKLEDGTEFDSSLPRNQPFVFSLGTGEVIKGWDQGLLGMCEGEKRKLVIPSELGYGERGHPPKIPGGATLVFEVELLKIER

SEQ ID NO: 23 - protein, 98 aa, synthetic construct
  REGION 1..98: note = Description of sequence: FKBP13-AcGFP-mCherry Fusion; Construct Name: FKBP-067; FKBP13 DRD in Construct : FKBP13 (mutated to match FKBP12 active site - Q72R Q84E A117H) (DRD - M54H)
HCPIKSRKGDVLHHHYTGKLEDGTEFDSSLPRNQPFVFSLGTGEVIKGWDQGLLGMCEGEKRKLVIPSELGYGERGHPPKIPGGATLVFEVELLKIER

SEQ ID NO: 24 - protein, 98 aa, synthetic construct
  REGION 1..98: note = Description of sequence: FKBP13-AcGFP-mCherry Fusion; Construct Name: FKBP-068; FKBP13 DRD in Construct : FKBP13 (mutated to match FKBP12 active site - Q72R Q84E A117H) (DRD - M54S)
HCPIKSRKGDVLHSHYTGKLEDGTEFDSSLPRNQPFVFSLGTGEVIKGWDQGLLGMCEGEKRKLVIPSELGYGERGHPPKIPGGATLVFEVELLKIER

SEQ ID NO: 25 - protein, 98 aa, synthetic construct
  REGION 1..98: note = Description of sequence: FKBP13-AcGFP-mCherry Fusion; Construct Name: FKBP-069; FKBP13 DRD in Construct : FKBP13 (mutated to match FKBP12 active site - Q72R Q84E A117H) (DRD - M54G)
HCPIKSRKGDVLHGHYTGKLEDGTEFDSSLPRNQPFVFSLGTGEVIKGWDQGLLGMCEGEKRKLVIPSELGYGERGHPPKIPGGATLVFEVELLKIER

SEQ ID NO: 26 - protein, 98 aa, synthetic construct
  REGION 1..98: note = Description of sequence: FKBP13-AcGFP-mCherry Fusion; Construct Name: FKBP-070; FKBP13 DRD in Construct : FKBP13 (mutated to match FKBP12 active site - Q72R Q84E A117H) (DRD - M54D)
HCPIKSRKGDVLHDHYTGKLEDGTEFDSSLPRNQPFVFSLGTGEVIKGWDQGLLGMCEGEKRKLVIPSELGYGERGHPPKIPGGATLVFEVELLKIER

SEQ ID NO: 27 - protein, 98 aa, synthetic construct
  REGION 1..98: note = Description of sequence: FKBP13-AcGFP-mCherry Fusion; Construct Name: FKBP-071; FKBP13 DRD in Construct : FKBP13 (mutated to match FKBP12 active site - Q72R Q84E A117H) (DRD - M54V)-AcGFP-P2A-mCherry
HCPIKSRKGDVLHVHYTGKLEDGTEFDSSLPRNQPFVFSLGTGEVIKGWDQGLLGMCEGEKRKLVIPSELGYGERGHPPKIPGGATLVFEVELLKIER

SEQ ID NO: 28 - protein, 98 aa, synthetic construct
  REGION 1..98: note = Description of sequence: FKBP13-AcGFP-mCherry Fusion; Construct Name: FKBP-072; FKBP13 DRD in Construct : pELNS-FKBP13 (mutated to match FKBP12 active site - Q72R Q84E A117H) (DRD - L93K)
HCPIKSRKGDVLHMHYTGKLEDGTEFDSSLPRNQPFVFSLGTGEVIKGWDQGKLGMCEGEKRKLVIPSELGYGERGHPPKIPGGATLVFEVELLKIER

SEQ ID NO: 29 - protein, 98 aa, synthetic construct
  REGION 1..98: note = Description of sequence: FKBP13-AcGFP-mCherry Fusion; Construct Name: FKBP-073; FKBP13 DRD in Construct : FKBP13 (mutated to match FKBP12 active site - Q72R Q84E A117H) (DRD - L93H)
HCPIKSRKGDVLHMHYTGKLEDGTEFDSSLPRNQPFVFSLGTGEVIKGWDQGHLGMCEGEKRKLVIPSELGYGERGHPPKIPGGATLVFEVELLKIER

SEQ ID NO: 30 - protein, 98 aa, synthetic construct
  REGION 1..98: note = Description of sequence: FKBP13-AcGFP-mCherry Fusion; Construct Name: FKBP-074; FKBP13 DRD in Construct : FKBP13 (mutated to match FKBP12 active site - Q72R Q84E A117H) (DRD - L93S)
HCPIKSRKGDVLHMHYTGKLEDGTEFDSSLPRNQPFVFSLGTGEVIKGWDQGSLGMCEGEKRKLVIPSELGYGERGHPPKIPGGATLVFEVELLKIER

SEQ ID NO: 31 - protein, 98 aa, synthetic construct
  REGION 1..98: note = Description of sequence: FKBP13-AcGFP-mCherry Fusion; Construct Name: FKBP-075; FKBP13 DRD in Construct : FKBP13 (mutated to match FKBP12 active site - Q72R Q84E A117H) (DRD - L93G)
HCPIKSRKGDVLHMHYTGKLEDGTEFDSSLPRNQPFVFSLGTGEVIKGWDQGGLGMCEGEKRKLVIPSELGYGERGHPPKIPGGATLVFEVELLKIER

SEQ ID NO: 32 - protein, 98 aa, synthetic construct
  REGION 1..98: note = Description of sequence: FKBP13-AcGFP-mCherry Fusion; Construct Name: FKBP-076; FKBP13 DRD in Construct : FKBP13 (mutated to match FKBP12 active site - Q72R Q84E A117H) (DRD - L93D)
HCPIKSRKGDVLHMHYTGKLEDGTEFDSSLPRNQPFVFSLGTGEVIKGWDQGDLGMCEGEKRKLVIPSELGYGERGHPPKIPGGATLVFEVELLKIER

SEQ ID NO: 33 - protein, 97 aa, synthetic construct
  REGION 1..97: note = Description of sequence: FKBP13-AcGFP-mCherry Fusion; Construct Name: FKBP-083; FKBP13 DRD in Construct : FKBP13 trunc1 (mutated to match FKBP12 active site - Q72R Q84E A117H) (DRD - M54A)
CPIKSRKGDVLHAHYTGKLEDGTEFDSSLPRNQPFVFSLGTGEVIKGWDQGLLGMCEGEKRKLVIPSELGYGERGHPPKIPGGATLVFEVELLKIER

SEQ ID NO: 34 - protein, 96 aa, synthetic construct
  REGION 1..96: note = Description of sequence: FKBP13-AcGFP-mCherry Fusion; Construct Name: FKBP-084; FKBP13 DRD in Construct : FKBP13 trunc2 (mutated to match FKBP12 active site Q72R Q84E A117H) (DRD - M54A)
PIKSRKGDVLHAHYTGKLEDGTEFDSSLPRNQPFVFSLGTGEVIKGWDQGLLGMCEGEKRKLVIPSELGYGERGHPPKIPGGATLVFEVELLKIER

SEQ ID NO: 35 - protein, 97 aa, synthetic construct
  REGION 1..97: note = Description of sequence: FKBP13-AcGFP-mCherry Fusion; Construct Name: FKBP-085; FKBP13 DRD in Construct : FKBP13 trunc1WT (mutated to match FKBP12 active site Q72R Q84E A117H) (DRD - WT)
CPIKSRKGDVLHMHYTGKLEDGTEFDSSLPRNQPFVFSLGTGEVIKGWDQGLLGMCEGEKRKLVIPSELGYGERGHPPKIPGGATLVFEVELLKIER

SEQ ID NO: 36 - protein, 96 aa, synthetic construct
  REGION 1..96: note = Description of sequence: FKBP13-AcGFP-mCherry Fusion; Construct Name: FKBP-086; FKBP13 DRD in Construct : FKBP13 trunc2WT (mutated to match FKBP12 active site Q72R Q84E A117H) (DRD - WT)
PIKSRKGDVLHMHYTGKLEDGTEFDSSLPRNQPFVFSLGTGEVIKGWDQGLLGMCEGEKRKLVIPSELGYGERGHPPKIPGGATLVFEVELLKIER

SEQ ID NO: 37 - protein, 98 aa, synthetic construct
  REGION 1..98: note = Description of sequence: FKBP13-AcGFP-mCherry Fusion; Construct Name: FKBP-087; FKBP13 DRD in Construct : FKBP13 (mutated to match FKBP12 active site Q72R Q84E A117H; and I121H) (DRD - M54A)
HCPIKSRKGDVLHAHYTGKLEDGTEFDSSLPRNQPFVFSLGTGEVIKGWDQGLLGMCEGEKRKLVIPSELGYGERGHPPKHPGGATLVFEVELLKIER

SEQ ID NO: 38 - protein, 98 aa, synthetic construct
  REGION 1..98: note = Description of sequence: FKBP13-AcGFP-mCherry Fusion; Construct Name: FKBP-088; FKBP13 DRD in Construct : FKBP13 (mutated to match FKBP12 active site Q72R Q84E A117H; and F129Y) (DRD - M54A)
HCPIKSRKGDVLHAHYTGKLEDGTEFDSSLPRNQPFVFSLGTGEVIKGWDQGLLGMCEGEKRKLVIPSELGYGERGHPPKIPGGATLVYEVELLKIER

SEQ ID NO: 39 - protein, 98 aa, synthetic construct
  REGION 1..98: note = Description of sequence: FKBP13-AcGFP-mCherry Fusion; Construct Name: FKBP-089; FKBP13 DRD in Construct : FKBP13 (mutated to match FKBP12 active site Q72R V85D A117H) (DRD - M54A)
HCPIKSRKGDVLHAHYTGKLEDGTEFDSSLPRNQPFVFSLGTGQDIKGWDQGLLGMCEGEKRKLVIPSELGYGERGHPPKIPGGATLVFEVELLKIER

SEQ ID NO: 40 - protein, 98 aa, synthetic construct
  REGION 1..98: note = Description of sequence: FKBP13-AcGFP-mCherry Fusion; Construct Name: FKBP-090; FKBP13 DRD in Construct : FKBP13 (mutated to match FKBP12 active site Q72R I86D A117H) (DRD - M54A)
HCPIKSRKGDVLHAHYTGKLEDGTEFDSSLPRNQPFVFSLGTGQVDKGWDQGLLGMCEGEKRKLVIPSELGYGERGHPPKIPGGATLVFEVELLKIER

SEQ ID NO: 41 - protein, 98 aa, synthetic construct
  REGION 1..98: note = Description of sequence: FKBP13-AcGFP-mCherry Fusion; Construct Name: FKBP-091; FKBP13 DRD in Construct : FKBP13 (mutated to match FKBP12 active site Q72R Q84E A117W) (DRD - M54A)
HCPIKSRKGDVLHAHYTGKLEDGTEFDSSLPRNQPFVFSLGTGEVIKGWDQGLLGMCEGEKRKLVIPSELGYGERGWPPKIPGGATLVFEVELLKIER

SEQ ID NO: 42 - protein, 98 aa, synthetic construct
  REGION 1..98: note = Description of sequence: FKBP13-AcGFP-mCherry Fusion; Construct Name: FKBP-092; FKBP13 DRD in Construct : FKBP13 (mutated to match FKBP12 active site Q72R Q84E A117H) (DRD - L127A)
HCPIKSRKGDVLHMHYTGKLEDGTEFDSSLPRNQPFVFSLGTGEVIKGWDQGLLGMCEGEKRKLVIPSELGYGERGHPPKIPGGATAVFEVELLKIER

SEQ ID NO: 43 - protein, 98 aa, synthetic construct
  REGION 1..98: note = Description of sequence: FKBP13-AcGFP-mCherry Fusion; Construct Name: FKBP-093; FKBP13 DRD in Construct : FKBP13 (mutated to match FKBP12 active site Q72R Q84E A117H) (DRD - L80A)
HCPIKSRKGDVLHMHYTGKLEDGTEFDSSLPRNQPFVFSAGTGEVIKGWDQGLLGMCEGEKRKLVIPSELGYGERGHPPKIPGGATLVFEVELLKIER

SEQ ID NO: 44 - protein, 98 aa, synthetic construct
  REGION 1..98: note = Description of sequence: FKBP13-AcGFP-mCherry Fusion; Construct Name: FKBP-094; FKBP13 DRD in Construct : FKBP13 (mutated to match FKBP12 active site Q72R Q84E A117H) (DRD - P107S)
HCPIKSRKGDVLHMHYTGKLEDGTEFDSSLPRNQPFVFSLGTGEVIKGWDQGLLGMCEGEKRKLVISSELGYGERGHPPKIPGGATLVFEVELLKIER

SEQ ID NO: 45 - protein, 98 aa, synthetic construct
  REGION 1..98: note = Description of sequence: FKBP13-AcGFP-mCherry Fusion; Construct Name: FKBP-095; FKBP13 DRD in Construct : FKBP13 (mutated to match FKBP12 active site Q72R Q84E A117H) (DRD - P122S)
HCPIKSRKGDVLHMHYTGKLEDGTEFDSSLPRNQPFVFSLGTGEVIKGWDQGLLGMCEGEKRKLVIPSELGYGERGHPPKISGGATLVFEVELLKIER

SEQ ID NO: 46 - protein, 98 aa, synthetic construct
  REGION 1..98: note = Description of sequence: FKBP13-AcGFP-mCherry Fusion; Construct Name: FKBP-096; FKBP13 DRD in Construct : FKBP13 (mutated to match FKBP12 active site Q72R Q84E A117H) (DRD - E100L)
HCPIKSRKGDVLHMHYTGKLEDGTEFDSSLPRNQPFVFSLGTGEVIKGWDQGLLGMCEGLKRKLVIPSELGYGERGHPPKIPGGATLVFEVELLKIER

SEQ ID NO: 47 - protein, 98 aa, synthetic construct
  REGION 1..98: note = Description of sequence: FKBP13-AcGFP-mCherry Fusion; Construct Name: FKBP-097; FKBP13 DRD in Construct : FKBP13 (mutated to match FKBP12 active site Q72R Q84E A117H) (DRD - E100S)
HCPIKSRKGDVLHMHYTGKLEDGTEFDSSLPRNQPFVFSLGTGEVIKGWDQGLLGMCEGSKRKLVIPSELGYGERGHPPKIPGGATLVFEVELLKIER

SEQ ID NO: 48 - protein, 98 aa, synthetic construct
  REGION 1..98: note = Description of sequence: FKBP13-AcGFP-mCherry Fusion; Construct Name: FKBP-098; FKBP13 DRD in Construct : FKBP13 (mutated to match FKBP12 active site Q72R Q84E A117W) (DRD - M54S)
HCPIKSRKGDVLHSHYTGKLEDGTEFDSSLPRNQPFVFSLGTGEVIKGWDQGLLGMCEGEKRKLVIPSELGYGERGWPPKIPGGATLVFEVELLKIER

SEQ ID NO: 49 - protein, 97 aa, synthetic construct
  REGION 1..97: note = Description of sequence: FKBP13-AcGFP-mCherry Fusion; Construct Name: FKBP-099; FKBP13 DRD in Construct : FKBP13 (mutated to match FKBP12 active site Q72R Q84E A117W) (DRD - M54A del c-term R)
HCPIKSRKGDVLHAHYTGKLEDGTEFDSSLPRNQPFVFSLGTGEVIKGWDQGLLGMCEGEKRKLVIPSELGYGERGWPPKIPGGATLVFEVELLKIE

SEQ ID NO: 50 - protein, 98 aa, synthetic construct
  REGION 1..98: note = Description of sequence: FKBP13-AcGFP-mCherry Fusion; Construct Name: FKBP-100; FKBP13 DRD in Construct : FKBP13 (mutated to match FKBP12 active site Q72R Q84E A117W) (DRD - M54A M96V)
HCPIKSRKGDVLHAHYTGKLEDGTEFDSSLPRNQPFVFSLGTGEVIKGWDQGLLGVCEGEKRKLVIPSELGYGERGWPPKIPGGATLVFEVELLKIER

SEQ ID NO: 51 - protein, 96 aa, synthetic construct
  REGION 1..96: note = Description of sequence: FKBP13-AcGFP-mCherry Fusion; Construct Name: FKBP-101; FKBP13 DRD in Construct : FKBP13 (mutated to match FKBP12 active site Q72R Q84E A117W) (DRD - M54A Nterm- 2aa trunc)
PIKSRKGDVLHAHYTGKLEDGTEFDSSLPRNQPFVFSLGTGEVIKGWDQGLLGMCEGEKRKLVIPSELGYGERGWPPKIPGGATLVFEVELLKIER

SEQ ID NO: 52 - protein, 98 aa, synthetic construct
  REGION 1..98: note = Description of sequence: FKBP13-AcGFP-mCherry Fusion; Construct Name: FKBP-102; FKBP13 DRD in Construct : FKBP13 (mutated to match FKBP12 active site Q72R Q84E A117W) (DRD - M54A P118V)
HCPIKSRKGDVLHAHYTGKLEDGTEFDSSLPRNQPFVFSLGTGEVIKGWDQGLLGMCEGEKRKLVIPSELGYGERGWVPKIPGGATLVFEVELLKIER

SEQ ID NO: 53 - protein, 98 aa, synthetic construct
  REGION 1..98: note = Description of sequence: FKBP13-AcGFP-mCherry Fusion; Construct Name: FKBP-103; FKBP13 DRD in Construct : FKBP13 (mutated to match FKBP12 active site Q72R Q84E A117W) (DRD - M54A P119V)
HCPIKSRKGDVLHAHYTGKLEDGTEFDSSLPRNQPFVFSLGTGEVIKGWDQGLLGMCEGEKRKLVIPSELGYGERGWPVKIPGGATLVFEVELLKIER

SEQ ID NO: 54 - protein, 98 aa, synthetic construct
  REGION 1..98: note = Description of sequence: FKBP13-AcGFP-mCherry Fusion; Construct Name: FKBP-104; FKBP13 DRD in Construct : FKBP13 (mutated to match FKBP12 active site Q72R Q84E A117W) K120I (DRD - M54A)
HCPIKSRKGDVLHAHYTGKLEDGTEFDSSLPRNQPFVFSLGTGEVIKGWDQGLLGMCEGEKRKLVIPSELGYGERGWPPIIPGGATLVFEVELLKIER

SEQ ID NO: 55 - protein, 98 aa, synthetic construct
  REGION 1..98: note = Description of sequence: FKBP13-AcGFP-mCherry Fusion; Construct Name: FKBP-105; FKBP13 DRD in Construct : FKBP13 (mutated to match FKBP12 active site Q72R Q84E A117W) K120R (DRD - M54A)
HCPIKSRKGDVLHAHYTGKLEDGTEFDSSLPRNQPFVFSLGTGEVIKGWDQGLLGMCEGEKRKLVIPSELGYGERGWPPRIPGGATLVFEVELLKIER

SEQ ID NO: 56 - protein, 98 aa, synthetic construct
  REGION 1..98: note = Description of sequence: FKBP13-AcGFP-mCherry Fusion; Construct Name: FKBP-106; FKBP13 DRD in Construct : FKBP13 (mutated to match FKBP12 active site Q72R Q84E A117Y (DRD - M54A)
HCPIKSRKGDVLHAHYTGKLEDGTEFDSSLPRNQPFVFSLGTGEVIKGWDQGLLGMCEGEKRKLVIPSELGYGERGYPPKIPGGATLVFEVELLKIER

SEQ ID NO: 57 - protein, 98 aa, synthetic construct
  REGION 1..98: note = Description of sequence: FKBP13-AcGFP-mCherry Fusion; Construct Name: FKBP-107; FKBP13 DRD in Construct : FKBP13 (mutated to match FKBP12 active site Q72R Q84E A117W) (DRD - I106V)
HCPIKSRKGDVLHMHYTGKLEDGTEFDSSLPRNQPFVFSLGTGEVIKGWDQGLLGMCEGEKRKLVVPSELGYGERGWPPKIPGGATLVFEVELLKIER

SEQ ID NO: 58 - protein, 98 aa, synthetic construct
  REGION 1..98: note = Description of sequence: FKBP13-AcGFP-mCherry Fusion; Construct Name: FKBP-108; FKBP13 DRD in Construct : FKBP13 (mutated to match FKBP12 active site Q72R Q84E A117W) (DRD - M54A I106V)
HCPIKSRKGDVLHAHYTGKLEDGTEFDSSLPRNQPFVFSLGTGEVIKGWDQGLLGMCEGEKRKLVVPSELGYGERGWPPKIPGGATLVFEVELLKIER

SEQ ID NO: 59 - protein, 98 aa, synthetic construct
  REGION 1..98: note = Description of sequence: FKBP13-AcGFP-mCherry Fusion; Construct Name: FKBP-109; FKBP13 DRD in Construct : FKBP13 (mutated to match FKBP12 active site Q72R Q84E A117W) (DRD - L104V)
HCPIKSRKGDVLHMHYTGKLEDGTEFDSSLPRNQPFVFSLGTGEVIKGWDQGLLGMCEGEKRKVVIPSELGYGERGWPPKIPGGATLVFEVELLKIER

SEQ ID NO: 60 - protein, 99 aa, synthetic construct
  REGION 1..99: note = Description of sequence: FKBP13-AcGFP-mCherry Fusion; Construct Name: FKBP-110; FKBP13 DRD in Construct : FKBP13 (mutated to match FKBP12 active site Q72R Q84E A117W) (DRD - L110A)
HCPIKSRKGDVLHMHYTGKLEDGTEFDSSLPRNQPFVFSLGTGEVIKGWDQGLLGMCEGEKRKLVIPSEAGYGERGWPPKIPGGATLVFEVELLKIERG

SEQ ID NO: 61 - protein, 98 aa, synthetic construct
  REGION 1..98: note = Description of sequence: FKBP13-AcGFP-mCherry Fusion; Construct Name: FKBP-111; FKBP13 DRD in Construct : FKBP13 (mutated to match FKBP12 active site Q72R Q84E A117W) (DRD - D90A)
HCPIKSRKGDVLHMHYTGKLEDGTEFDSSLPRNQPFVFSLGTGEVIKGWAQGLLGMCEGEKRKLVIPSELGYGERGWPPKIPGGATLVFEVELLKIER

SEQ ID NO: 62 - protein, 98 aa, synthetic construct
  REGION 1..98: note = Description of sequence: FKBP13-AcGFP-mCherry Fusion; Construct Name: FKBP-112; FKBP13 DRD in Construct : FKBP13 (mutated to match FKBP12 active site Q72R Q84E A117W) (DRD - L93V)
HCPIKSRKGDVLHMHYTGKLEDGTEFDSSLPRNQPFVFSLGTGEVIKGWDQGVLGMCEGEKRKLVIPSELGYGERGWPPKIPGGATLVFEVELLKIER

SEQ ID NO: 63 - protein, 98 aa, synthetic construct; identical to SEQ ID NO: 48
  REGION 1..98: note = Description of sequence: FKBP13-AcGFP-mCherry Fusion; Construct Name: FKBP-113; FKBP13 DRD in Construct : FKBP13 (mutated to match FKBP12 active site Q72R Q84E A117W) (DRD - M54S)

SEQ ID NO: 64 - protein, 97 aa, synthetic construct
  REGION 1..97: note = Description of sequence: FKBP13-AcGFP-mCherry Fusion; Construct Name: FKBP-114; FKBP13 DRD in Construct : FKBP13 (mutated to match FKBP12 active site Q72R Q84E A117W) (DRD - M54A Trunc1 )
CPIKSRKGDVLHAHYTGKLEDGTEFDSSLPRNQPFVFSLGTGEVIKGWDQGLLGMCEGEKRKLVIPSELGYGERGWPPKIPGGATLVFEVELLKIER

SEQ ID NO: 65 - protein, 96 aa, synthetic construct
  REGION 1..96: note = Description of sequence: FKBP13-AcGFP-mCherry Fusion; Construct Name: FKBP-115; FKBP13 DRD in Construct : FKBP13 (mutated to match FKBP12 active site Q72R Q84E A117W) (DRD - none Trunc2 )
PIKSRKGDVLHMHYTGKLEDGTEFDSSLPRNQPFVFSLGTGEVIKGWDQGLLGMCEGEKRKLVIPSELGYGERGWPPKIPGGATLVFEVELLKIER

SEQ ID NO: 66 - protein, 95 aa, synthetic construct
  REGION 1..95: note = Description of sequence: FKBP13-AcGFP-mCherry Fusion; Construct Name: FKBP-116; FKBP13 DRD in Construct : FKBP13 (mutated to match FKBP12 active site Q72R Q84E A117W) (DRD - none Trunc3 )
IKSRKGDVLHMHYTGKLEDGTEFDSSLPRNQPFVFSLGTGEVIKGWDQGLLGMCEGEKRKLVIPSELGYGERGWPPKIPGGATLVFEVELLKIER

SEQ ID NO: 67 - protein, 98 aa, synthetic construct
  REGION 1..98: note = Description of sequence: FKBP13-AcGFP-mCherry Fusion; Construct Name: FKBP-117; FKBP13 DRD in Construct : FKBP13 (mutated to match FKBP12 active site Q72R Q84E A117W) (DRD V85R)
HCPIKSRKGDVLHMHYTGKLEDGTEFDSSLPRNQPFVFSLGTGERIKGWDQGLLGMCEGEKRKLVIPSELGYGERGWPPKIPGGATLVFEVELLKIER

SEQ ID NO: 68 - protein, 98 aa, synthetic construct
  REGION 1..98: note = Description of sequence: FKBP13-AcGFP-mCherry Fusion; Construct Name: FKBP-118; FKBP13 DRD in Construct : FKBP13 (mutated to match FKBP12 active site Q72R Q84E A117H) (DRD V85R)
HCPIKSRKGDVLHMHYTGKLEDGTEFDSSLPRNQPFVFSLGTGERIKGWDQGLLGMCEGEKRKLVIPSELGYGERGHPPKIPGGATLVFEVELLKIER

SEQ ID NO: 69 - protein, 98 aa, synthetic construct
  REGION 1..98: note = Description of sequence: FKBP13-AcGFP-mCherry Fusion; Construct Name: FKBP-119; FKBP13 DRD in Construct : FKBP13 (mutated to match FKBP12 active site Q72R Q84E A117W) (DRD K101W )
HCPIKSRKGDVLHMHYTGKLEDGTEFDSSLPRNQPFVFSLGTGEVIKGWDQGLLGMCEGEWRKLVIPSELGYGERGWPPKIPGGATLVFEVELLKIER

SEQ ID NO: 70 - protein, 98 aa, synthetic construct
  REGION 1..98: note = Description of sequence: FKBP13-AcGFP-mCherry Fusion; Construct Name: FKBP-120; FKBP13 DRD in Construct : FKBP13 (mutated to match FKBP12 active site Q72R Q84E A117H) (DRD K101W)
HCPIKSRKGDVLHMHYTGKLEDGTEFDSSLPRNQPFVFSLGTGEVIKGWDQGLLGMCEGEWRKLVIPSELGYGERGHPPKIPGGATLVFEVELLKIER

SEQ ID NO: 71 - protein, 98 aa, synthetic construct
  REGION 1..98: note = Description of sequence: FKBP13-AcGFP-mCherry Fusion; Construct Name: FKBP-121; FKBP13 DRD in Construct : FKBP13 (mutated to match FKBP12 active site Q72R Q84E A117W) (DRD I136V)
HCPIKSRKGDVLHMHYTGKLEDGTEFDSSLPRNQPFVFSLGTGEVIKGWDQGLLGMCEGEKRKLVIPSELGYGERGWPPKIPGGATLVFEVELLKVER

SEQ ID NO: 72 - protein, 98 aa, synthetic construct
  REGION 1..98: note = Description of sequence: FKBP13-AcGFP-mCherry Fusion; Construct Name: FKBP-122; FKBP13 DRD in Construct : FKBP13 (mutated to match FKBP12 active site Q72R Q84E A117H) (DRD I136V)
HCPIKSRKGDVLHMHYTGKLEDGTEFDSSLPRNQPFVFSLGTGEVIKGWDQGLLGMCEGEKRKLVIPSELGYGERGHPPKIPGGATLVFEVELLKVER

SEQ ID NO: 73 - protein, 98 aa, synthetic construct
  REGION 1..98: note = Description of sequence: FKBP13-AcGFP-mCherry Fusion; Construct Name: FKBP-123; FKBP13 DRD in Construct : FKBP13 (mutated to match FKBP12 active site Q72R Q84E A117W) (DRD G49S)
HCPIKSRKSDVLHMHYTGKLEDGTEFDSSLPRNQPFVFSLGTGEVIKGWDQGLLGMCEGEKRKLVIPSELGYGERGWPPKIPGGATLVFEVELLKIER

SEQ ID NO: 74 - protein, 98 aa, synthetic construct
  REGION 1..98: note = Description of sequence: FKBP13-AcGFP-mCherry Fusion; Construct Name: FKBP-124; FKBP13 DRD in Construct : FKBP13 (mutated to match FKBP12 active site Q72R Q84E A117H) (DRD G49S)
HCPIKSRKSDVLHMHYTGKLEDGTEFDSSLPRNQPFVFSLGTGEVIKGWDQGLLGMCEGEKRKLVIPSELGYGERGHPPKIPGGATLVFEVELLKIER

SEQ ID NO: 75 - protein, 98 aa, synthetic construct
  REGION 1..98: note = Description of sequence: FKBP13-AcGFP-mCherry Fusion; Construct Name: FKBP-125; FKBP13 DRD in Construct : FKBP13 (mutated to match FKBP12 active site Q72R Q84E A117W) (DRD D62S)
HCPIKSRKGDVLHMHYTGKLESGTEFDSSLPRNQPFVFSLGTGEVIKGWDQGLLGMCEGEKRKLVIPSELGYGERGWPPKIPGGATLVFEVELLKIER

SEQ ID NO: 76 - protein, 98 aa, synthetic construct
  REGION 1..98: note = Description of sequence: FKBP13-AcGFP-mCherry Fusion; Construct Name: FKBP-126; FKBP13 DRD in Construct : FKBP13 (mutated to match FKBP12 active site Q72R Q84E A117H) (DRD D62S)
HCPIKSRKGDVLHMHYTGKLESGTEFDSSLPRNQPFVFSLGTGEVIKGWDQGLLGMCEGEKRKLVIPSELGYGERGHPPKIPGGATLVFEVELLKIER

SEQ ID NO: 77 - protein, 98 aa, synthetic construct
  REGION 1..98: note = Description of sequence: FKBP13-AcGFP-mCherry Fusion; Construct Name: FKBP-127; FKBP13 DRD in Construct : FKBP13 (mutated to match FKBP12 active site Q72R Q84E A117W) (DRD F76Q)
HCPIKSRKGDVLHMHYTGKLEDGTEFDSSLPRNQPQVFSLGTGEVIKGWDQGLLGMCEGEKRKLVIPSELGYGERGWPPKIPGGATLVFEVELLKIER

SEQ ID NO: 78 - protein, 98 aa, synthetic construct
  REGION 1..98: note = Description of sequence: FKBP13-AcGFP-mCherry Fusion; Construct Name: FKBP-128; FKBP13 DRD in Construct : FKBP13 (mutated to match FKBP12 active site Q72R Q84E A117W) (DRD H53L)
HCPIKSRKGDVLLMHYTGKLEDGTEFDSSLPRNQPFVFSLGTGEVIKGWDQGLLGMCEGEKRKLVIPSELGYGERGWPPKIPGGATLVFEVELLKIER

SEQ ID NO: 79 - protein, 98 aa, synthetic construct
  REGION 1..98: note = Description of sequence: FKBP13-AcGFP-mCherry Fusion; Construct Name: FKBP-129; FKBP13 DRD in Construct : FKBP13 (mutated to match FKBP12 active site Q72R Q84E A117W) (DRD E98L)
HCPIKSRKGDVLHMHYTGKLEDGTEFDSSLPRNQPFVFSLGTGEVIKGWDQGLLGMCLGEKRKLVIPSELGYGERGWPPKIPGGATLVFEVELLKIER

SEQ ID NO: 80 - protein, 98 aa, synthetic construct
  REGION 1..98: note = Description of sequence: FKBP13-AcGFP-mCherry Fusion; Construct Name: FKBP-130; FKBP13 DRD in Construct : FKBP13 (mutated to match FKBP12 active site Q72R Q84E A117W) (DRD P107K)
HCPIKSRKGDVLHMHYTGKLEDGTEFDSSLPRNQPFVFSLGTGEVIKGWDQGLLGMCEGEKRKLVIKSELGYGERGWPPKIPGGATLVFEVELLKIER

SEQ ID NO: 81 - protein, 98 aa, synthetic construct
  REGION 1..98: note = Description of sequence: FKBP13-AcGFP-mCherry Fusion; Construct Name: FKBP-131; FKBP13 DRD in Construct : FKBP13 (mutated to match FKBP12 active site Q72R Q84E A117W) (DRD T126C)
HCPIKSRKGDVLHMHYTGKLEDGTEFDSSLPRNQPFVFSLGTGEVIKGWDQGLLGMCEGEKRKLVIPSELGYGERGWPPKIPGGACLVFEVELLKIER

SEQ ID NO: 82 - protein, 98 aa, synthetic construct
  REGION 1..98: note = Description of sequence: FKBP13-AcGFP-mCherry Fusion; Construct Name: FKBP-132; FKBP13 DRD in Construct : FKBP13 (mutated to match FKBP12 active site Q72R Q84E A117W) (DRD G92Y)
HCPIKSRKGDVLHMHYTGKLEDGTEFDSSLPRNQPFVFSLGTGEVIKGWDQYLLGMCEGEKRKLVIPSELGYGERGWPPKIPGGATLVFEVELLKIER

SEQ ID NO: 83 - protein, 98 aa, synthetic construct
  REGION 1..98: note = Description of sequence: FKBP13-AcGFP-mCherry Fusion; Construct Name: FKBP-133; FKBP13 DRD in Construct : FKBP13 (mutated to match FKBP12 active site Q72R Q84E A117W) (DRD G83T)
HCPIKSRKGDVLHMHYTGKLEDGTEFDSSLPRNQPFVFSLGTTEVIKGWDQGLLGMCEGEKRKLVIPSELGYGERGWPPKIPGGATLVFEVELLKIER

SEQ ID NO: 84 - protein, 98 aa, synthetic construct
  REGION 1..98: note = Description of sequence: FKBP13-AcGFP-mCherry Fusion; Construct Name: FKBP-134; FKBP13 DRD in Construct : FKBP13 (mutated to match FKBP12 active site Q72R Q84E A117W) (DRD H41R)
RCPIKSRKGDVLHMHYTGKLEDGTEFDSSLPRNQPFVFSLGTGEVIKGWDQGLLGMCEGEKRKLVIPSELGYGERGWPPKIPGGATLVFEVELLKIER

SEQ ID NO: 85 - protein, 98 aa, synthetic construct
  REGION 1..98: note = Description of sequence: FKBP13-AcGFP-mCherry Fusion; Construct Name: FKBP-135; FKBP13 DRD in Construct : FKBP13 (mutated to match FKBP12 active site Q72R Q84E A117W) (DRD E61Y)
HCPIKSRKGDVLHMHYTGKLYDGTEFDSSLPRNQPFVFSLGTGEVIKGWDQGLLGMCEGEKRKLVIPSELGYGERGWPPKIPGGATLVFEVELLKIER

SEQ ID NO: 86 - protein, 98 aa, synthetic construct
  REGION 1..98: note = Description of sequence: FKBP13-AcGFP-mCherry Fusion; Construct Name: FKBP-136; FKBP13 DRD in Construct : FKBP13 (mutated to match FKBP12 active site Q72R Q84E A117W) (DRD S108M)
HCPIKSRKGDVLHMHYTGKLEDGTEFDSSLPRNQPFVFSLGTGEVIKGWDQGLLGMCEGEKRKLVIPMELGYGERGWPPKIPGGATLVFEVELLKIER

SEQ ID NO: 87 - protein, 98 aa, synthetic construct
  REGION 1..98: note = Description of sequence: FKBP13-AcGFP-mCherry Fusion; Construct Name: FKBP-137; FKBP13 DRD in Construct : FKBP13 (mutated to match FKBP12 active site Q72R Q84E A117W) (DRD P75L)
HCPIKSRKGDVLHMHYTGKLEDGTEFDSSLPRNQLFVFSLGTGEVIKGWDQGLLGMCEGEKRKLVIPSELGYGERGWPPKIPGGATLVFEVELLKIER

SEQ ID NO: 88 - protein, 98 aa, synthetic construct
  REGION 1..98: note = Description of sequence: FKBP13-AcGFP-mCherry Fusion; Construct Name: FKBP-138; FKBP13 DRD in Construct : FKBP13 (mutated to match FKBP12 active site Q72R Q84E A117W) (DRD G124R)
HCPIKSRKGDVLHMHYTGKLEDGTEFDSSLPRNQPFVFSLGTGEVIKGWDQGLLGMCEGEKRKLVIPSELGYGERGWPPKIPGRATLVFEVELLKIER

SEQ ID NO: 89 - protein, 98 aa, synthetic construct
  REGION 1..98: note = Description of sequence: FKBP13-AcGFP-mCherry Fusion; Construct Name: FKBP-139; FKBP13 DRD in Construct : FKBP13 (mutated to match FKBP12 active site Q72R Q84E A117W) (DRD E130R)
HCPIKSRKGDVLHMHYTGKLEDGTEFDSSLPRNQPFVFSLGTGEVIKGWDQGLLGMCEGEKRKLVIPSELGYGERGWPPKIPGGATLVFRVELLKIER

SEQ ID NO: 90 - protein, 98 aa, synthetic construct
  REGION 1..98: note = Description of sequence: FKBP13-AcGFP-mCherry Fusion; Construct Name: FKBP-140; FKBP13 DRD in Construct : FKBP13 (mutated to match FKBP12 active site Q72R Q84E A117W) (DRD K135Y)
HCPIKSRKGDVLHMHYTGKLEDGTEFDSSLPRNQPFVFSLGTGEVIKGWDQGLLGMCEGEKRKLVIPSELGYGERGWPPKIPGGATLVFEVELLYIER

SEQ ID NO: 91 - protein, 98 aa, synthetic construct
  REGION 1..98: note = Description of sequence: FKBP13-AcGFP-mCherry Fusion; Construct Name: FKBP-141; FKBP13 DRD in Construct : FKBP13 (mutated to match FKBP12 active site Q72R Q84E A117R (DRD L52R)
HCPIKSRKGDVRHMHYTGKLEDGTEFDSSLPRNQPFVFSLGTGEVIKGWDQGLLGMCEGEKRKLVIPSELGYGERGRPPKIPGGATLVFEVELLKIER

SEQ ID NO: 92 - protein, 98 aa, synthetic construct
  REGION 1..98: note = Description of sequence: FKBP13-AcGFP-mCherry Fusion; Construct Name: FKBP-142; FKBP13 DRD in Construct : FKBP13 (mutated to match FKBP12 active site Q72R Q84E A117W) (DRD T64G)
HCPIKSRKGDVLHMHYTGKLEDGGEFDSSLPRNQPFVFSLGTGEVIKGWDQGLLGMCEGEKRKLVIPSELGYGERGWPPKIPGGATLVFEVELLKIER

SEQ ID NO: 93 - protein, 98 aa, synthetic construct
  REGION 1..98: note = Description of sequence: FKBP13-AcGFP-mCherry Fusion; Construct Name: FKBP-143; FKBP13 DRD in Construct : FKBP13 (mutated to match FKBP12 active site Q72R Q84G A117H) (DRD P43R)
HCRIKSRKGDVLHMHYTGKLEDGTEFDSSLPRNQPFVFSLGTGGVIKGWDQGLLGMCEGEKRKLVIPSELGYGERGHPPKIPGGATLVFEVELLKIER

SEQ ID NO: 94 - protein, 98 aa, synthetic construct
  REGION 1..98: note = Description of sequence: FKBP13-AcGFP-mCherry Fusion; Construct Name: FKBP-144; FKBP13 DRD in Construct : FKBP13 (mutated to match FKBP12 active site Q72R Q84E A117H) (DRD D50R E98M)
HCPIKSRKGRVLHMHYTGKLEDGTEFDSSLPRNQPFVFSLGTGEVIKGWDQGLLGMCMGEKRKLVIPSELGYGERGHPPKIPGGATLVFEVELLKIER

SEQ ID NO: 95 - protein, 98 aa, synthetic construct
  REGION 1..98: note = Description of sequence: FKBP13-AcGFP-mCherry Fusion; Construct Name: FKBP-145; FKBP13 DRD in Construct : FKBP13 (mutated to match FKBP12 active site Q72R Q84E A117H) (DRD H41S E61D)
SCPIKSRKGDVLHMHYTGKLDDGTEFDSSLPRNQPFVFSLGTGEVIKGWDQGLLGMCEGEKRKLVIPSELGYGERGHPPKIPGGATLVFEVELLKIER

SEQ ID NO: 96 - protein, 98 aa, synthetic construct
  REGION 1..98: note = Description of sequence: FKBP13-AcGFP-mCherry Fusion; Construct Name: FKBP-146; FKBP13 DRD in Construct : FKBP13 (mutated to match FKBP12 active site Q72R Q84E A117H) (DRD L104S I136L)
HCPIKSRKGDVLHMHYTGKLEDGTEFDSSLPRNQPFVFSLGTGEVIKGWDQGLLGMCEGEKRKSVIPSELGYGERGHPPKIPGGATLVFEVELLKLER

SEQ ID NO: 97 - protein, 98 aa, synthetic construct
  REGION 1..98: note = Description of sequence: FKBP13-AcGFP-mCherry Fusion; Construct Name: FKBP-147; FKBP13 DRD in Construct : FKBP13 (mutated to match FKBP12 active site Q72R Q84E A117H) (DRD T64R S108G E137F)
HCPIKSRKGDVLHMHYTGKLEDGREFDSSLPRNQPFVFSLGTGEVIKGWDQGLLGMCEGEKRKLVIPGELGYGERGHPPKIPGGATLVFEVELLKIFR

SEQ ID NO: 98 - protein, 98 aa, synthetic construct
  REGION 1..98: note = Description of sequence: FKBP13-AcGFP-mCherry Fusion; Construct Name: FKBP-148; FKBP13 DRD in Construct : FKBP13 (mutated to match FKBP12 active site Q72R Q84E A117H) (DRD D62N D67N D90N M96G E130K)
HCPIKSRKGDVLHMHYTGKLENGTEFNSSLPRNQPFVFSLGTGEVIKGWNQGLLGGCEGEKRKLVIPSELGYGERGHPPKIPGGATLVFKVELLKIER

SEQ ID NO: 99 - protein, 98 aa, synthetic construct
  REGION 1..98: note = Description of sequence: FKBP13-AcGFP-mCherry Fusion; Construct Name: FKBP-149; FKBP13 DRD in Construct : FKBP13 (mutated to match FKBP12 active site Q72R Q84E A117H) (DRD T64Q L94G)
HCPIKSRKGDVLHMHYTGKLEDGQEFDSSLPRNQPFVFSLGTGEVIKGWDQGLGGMCEGEKRKLVIPSELGYGERGHPPKIPGGATLVFEVELLKIER

SEQ ID NO: 100 - protein, 98 aa, synthetic construct
  REGION 1..98: note = Description of sequence: FKBP13-AcGFP-mCherry Fusion; Construct Name: FKBP-150; FKBP13 DRD in Construct : FKBP13 (mutated to match FKBP12 active site Q72R Q84E A117H) (DRD E61G G124P)
HCPIKSRKGDVLHMHYTGKLGDGTEFDSSLPRNQPFVFSLGTGEVIKGWDQGLLGMCEGEKRKLVIPSELGYGERGHPPKIPGPATLVFEVELLKIER

SEQ ID NO: 101 - protein, 98 aa, synthetic construct
  REGION 1..98: note = Description of sequence: FKBP13-AcGFP-mCherry Fusion; Construct Name: FKBP-151; FKBP13 DRD in Construct : FKBP13 (mutated to match FKBP12 active site Q72R Q84R A117W) (DRD M54A)
HCPIKSRKGDVLHAHYTGKLEDGTEFDSSLPRNQPFVFSLGTGRVIKGWDQGLLGMCEGEKRKLVIPSELGYGERGWPPKIPGGATLVFEVELLKIER

SEQ ID NO: 102 - protein, 98 aa, synthetic construct
  REGION 1..98: note = Description of sequence: FKBP13-AcGFP-mCherry Fusion; Construct Name: FKBP-152; FKBP13 DRD in Construct : FKBP13 (mutated to match FKBP12 active site Q72R Q84W A117W) (DRD M54A)
HCPIKSRKGDVLHAHYTGKLEDGTEFDSSLPRNQPFVFSLGTGWVIKGWDQGLLGMCEGEKRKLVIPSELGYGERGWPPKIPGGATLVFEVELLKIER

SEQ ID NO: 103 - protein, 98 aa, synthetic construct
  REGION 1..98: note = Description of sequence: FKBP13-AcGFP-mCherry Fusion; Construct Name: FKBP-153; FKBP13 DRD in Construct : FKBP13 (mutated to match FKBP12 active site Q72RS79W Q84E A117W) (DRD M54A)
HCPIKSRKGDVLHAHYTGKLEDGTEFDSSLPRNQPFVFWLGTGEVIKGWDQGLLGMCEGEKRKLVIPSELGYGERGWPPKIPGGATLVFEVELLKIER

SEQ ID NO: 104 - protein, 98 aa, synthetic construct; identical to SEQ ID NO: 41
  REGION 1..98: note = Description of sequence: FKBP13-AcGFP-mCherry Fusion; Construct Name: FKBP-154; FKBP13 DRD in Construct : FKBP13 (mutated to match FKBP12 active site Q72R S79W Q84W A117W) (DRD M54A)

SEQ ID NO: 105 - protein, 98 aa, synthetic construct
  REGION 1..98: note = Description of sequence: FKBP13-AcGFP-mCherry Fusion; Construct Name: FKBP-155; FKBP13 DRD in Construct : FKBP13 (mutated to match FKBP12 active site Q72R S79W Q84R A117W) (DRD M54A)
HCPIKSRKGDVLHAHYTGKLEDGTEFDSSLPRNQPFVFWLGTGRVIKGWDQGLLGMCEGEKRKLVIPSELGYGERGWPPKIPGGATLVFEVELLKIER

SEQ ID NO: 106 - protein, 98 aa, synthetic construct
  REGION 1..98: note = Description of sequence: FKBP13-AcGFP-mCherry Fusion; Construct Name: FKBP-156; FKBP13 DRD in Construct : FKBP13 (mutated to match FKBP12 active site Q72R T82W Q84W A117W) (DRD M54A)
HCPIKSRKGDVLHAHYTGKLEDGTEFDSSLPRNQPFVFSLGWGWVIKGWDQGLLGMCEGEKRKLVIPSELGYGERGWPPKIPGGATLVFEVELLKIER

SEQ ID NO: 107 - protein, 98 aa, synthetic construct
  REGION 1..98: note = Description of sequence: FKBP13-AcGFP-mCherry Fusion; Construct Name: FKBP-157; FKBP13 DRD in Construct : FKBP13 (mutated to match FKBP12 active site Q72R T82W Q84E A117W) (DRD M54A)
HCPIKSRKGDVLHAHYTGKLEDGTEFDSSLPRNQPFVFSLGWGRVIKGWDQGLLGMCEGEKRKLVIPSELGYGERGWPPKIPGGATLVFEVELLKIER

SEQ ID NO: 108 - protein, 98 aa, synthetic construct
  REGION 1..98: note = Description of sequence: FKBP13-AcGFP-mCherry Fusion; Construct Name: FKBP-158; FKBP13 DRD in Construct : FKBP13 (mutated to match FKBP12 active site Q72R Q84E A117W) (DRD M54A G111E)
HCPIKSRKGDVLHAHYTGKLEDGTEFDSSLPRNQPFVFSLGTGEVIKGWDQGLLGMCEGEKRKLVIPSELEYGERGWPPKIPGGATLVFEVELLKIER

SEQ ID NO: 109 - protein, 98 aa, synthetic construct
  REGION 1..98: note = Description of sequence: FKBP13-AcGFP-mCherry Fusion; Construct Name: FKBP-159; FKBP13 DRD in Construct : FKBP13 (mutated to match FKBP12 active site Q72R Q84E A117W) (DRD M54A G111Y)
HCPIKSRKGDVLHAHYTGKLEDGTEFDSSLPRNQPFVFSLGTGEVIKGWDQGLLGMCEGEKRKLVIPSELYYGERGWPPKIPGGATLVFEVELLKIER

SEQ ID NO: 110 - protein, 597 aa, synthetic construct
  REGION 1..597: note = Description of sequence: FKBP13 DRD-AcGFP-mCherry AA sequence : FKBP-039
HCPIKSRKGDVLHMHYTGKLEDGTEFDSSLPQNQPFVFSLGTGQVIKGWDQGLLGMCEGEKRKLVIPSELGYGERGAPPKIPGGATLVFEVELLKIERGGSMVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYKGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 111 - protein, 597 aa, synthetic construct
  REGION 1..597: note = Description of sequence: FKBP13 DRD -AcGFP-mCherry AA sequence : FKBP13-040
HCPIKSRKGDVLHMHYTGKLEDGTEFDSSLPQNQPFVFSLGTGQVIKGWGQGLLGMCEGEKRKLVIPSELGYGERGAPPKIPGGATLVFEVELLKIERGGSMVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYKGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 112 - protein, 597 aa, synthetic construct
  REGION 1..597: note = Description of sequence: FKBP13 DRD-AcGFP-mCherry AA sequence : FKBP-041
HCPIKSRKGDVLHAHYTGKLEDGTEFDSSLPQNQPFVFSLGTGQVIKGWDQGLLGMCEGEKRKLVIPSELGYGERGAPPKIPGGATLVFEVELLKIERGGSMVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYKGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 113 - protein, 597 aa, synthetic construct
  REGION 1..597: note = Description of sequence: FKBP13 DRD-AcGFP-mCherry AA sequence : FKBP-042
HCPIKSRKGDVLHMHYTGKLEDGTEFDSSLPQNQPFVFSLGTGQVIKGWDQGLLGVCEGEKRKLVIPSELGYGERGAPPKIPGGATLVFEVELLKIERGGSMVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYKGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 114 - protein, 597 aa, synthetic construct
  REGION 1..597: note = Description of sequence: FKBP13 DRD-AcGFP-mCherry AA sequence : FKBP-043
HCPIKSRKGDVLHMHYTGKLEDGTEFDSSLPRNQPFVFSLGTGEVIKGWGQGLLGMCEGEKRKLVIPSELGYGERGHPPKIPGGATLVFEVELLKIERGGSMVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYKGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 115 - protein, 597 aa, synthetic construct
  REGION 1..597: note = Description of sequence: FKBP13 DRD-AcGFP-mCherry AA sequence : FKBP-044
HCPIKSRKGDVLHAHYTGKLEDGTEFDSSLPRNQPFVFSLGTGEVIKGWDQGLLGMCEGEKRKLVIPSELGYGERGHPPKIPGGATLVFEVELLKIERGGSMVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYKGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 116 - protein, 597 aa, synthetic construct
  REGION 1..597: note = Description of sequence: FKBP13 DRD-AcGFP-mCherry AA sequence : FKBP-045
HCPIKSRKGDVLHMHYTGKLEDGTEFDSSLPRNQPFVFSLGTGEVIKGWDQGLLGVCEGEKRKLVIPSELGYGERGHPPKIPGGATLVFEVELLKIERGGSMVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYKGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 117 - protein, 239 aa, synthetic construct
  REGION 1..239: note = Description of sequence: AcGFP amino acid sequence
MVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYK

SEQ ID NO: 118 - protein, 254 aa, synthetic construct
  REGION 1..254: note = Description of sequence: mCherry amino acid sequence
ATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 119 - DNA, 294 bp, synthetic construct
  misc_feature 1..294: note = Description of sequence: Construct Name: FKBP-160; Mutation(s): Q72R Q84E A117W V85R DRD M54A
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 120 - protein, 98 aa, synthetic construct
  REGION 1..98: note = Description of sequence: Construct Name: FKBP-160; Mutation(s): Q72R Q84E A117W V85R DRD M54A
HCPIKSRKGDVLHAHYTGKLEDGTEFDSSLPRNQPFVFSLGTGERIKGWDQGLLGMCEGEKRKLVIPSELGYGERGWPPKIPGGATLVFEVELLKIER

SEQ ID NO: 121 - DNA, 294 bp, synthetic construct
  misc_feature 1..294: note = Description of sequence: Construct Name: FKBP-161; Mutation(s): Q72R Q84E A117W DRD M54S V85R
  source 1..294: mol_type = other DNA
cactgcccaatcaagagcagaaaaggcgacgtgctgcacagtcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 122 - protein, 98 aa, synthetic construct
  REGION 1..98: note = Description of sequence: Construct Name: FKBP-161; Mutation(s): Q72R Q84E A117W DRD M54S V85R
HCPIKSRKGDVLHSHYTGKLEDGTEFDSSLPRNQPFVFSLGTGERIKGWDQGLLGMCEGEKRKLVIPSELGYGERGWPPKIPGGATLVFEVELLKIER

SEQ ID NO: 123 - DNA, 291 bp, synthetic construct
  misc_feature 1..291: note = Description of sequence: Construct Name: FKBP-162; Mutation(s): Q72R Q84E A117W DRD M54A del c-term R V85R
  source 1..291: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgag

SEQ ID NO: 124 - protein, 97 aa, synthetic construct
  REGION 1..97: note = Description of sequence: Construct Name: FKBP-162; Mutation(s): Q72R Q84E A117W DRD M54A del c-term R V85R
HCPIKSRKGDVLHAHYTGKLEDGTEFDSSLPRNQPFVFSLGTGERIKGWDQGLLGMCEGEKRKLVIPSELGYGERGWPPKIPGGATLVFEVELLKIE

SEQ ID NO: 125 - DNA, 294 bp, synthetic construct
  misc_feature 1..294: note = Description of sequence: Construct Name: FKBP-163; Mutation(s): Q72R Q84E A117W DRD M54A M96V V85R
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggagtttgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 126 - protein, 98 aa, synthetic construct
  REGION 1..98: note = Description of sequence: Construct Name: FKBP-163; Mutation(s): Q72R Q84E A117W DRD M54A M96V V85R
HCPIKSRKGDVLHAHYTGKLEDGTEFDSSLPRNQPFVFSLGTGERIKGWDQGLLGVCEGEKRKLVIPSELGYGERGWPPKIPGGATLVFEVELLKIER

SEQ ID NO: 127 - DNA, 294 bp, synthetic construct
  misc_feature 1..294: note = Description of sequence: Construct Name: FKBP-164; Mutation(s): Q72R Q84E A117W DRD M54A P118V V85R
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctgggttccaaagattcctggcggagccacactggtgttcgaagtggaactgctgaagatcgagcga

SEQ ID NO: 128 - protein, 98 aa, synthetic construct
  REGION 1..98: note = Description of sequence: Construct Name: FKBP-164; Mutation(s): Q72R Q84E A117W DRD M54A P118V V85R
HCPIKSRKGDVLHAHYTGKLEDGTEFDSSLPRNQPFVFSLGTGERIKGWDQGLLGMCEGEKRKLVIPSELGYGERGWVPKIPGGATLVFEVELLKIER

SEQ ID NO: 129 - DNA, 294 bp, synthetic construct
  misc_feature 1..294: note = Description of sequence: Construct Name: FKBP-165; Mutation(s): Q72R Q84E A117W DRD M54A P119V V85R
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctgttaagattcctggcggagccacactggtgttcgaagtggaactgctgaagatcgagcga

SEQ ID NO: 130 - protein, 98 aa, synthetic construct
  REGION 1..98: note = Description of sequence: Construct Name: FKBP-165; Mutation(s): Q72R Q84E A117W DRD M54A P119V V85R
HCPIKSRKGDVLHAHYTGKLEDGTEFDSSLPRNQPFVFSLGTGERIKGWDQGLLGMCEGEKRKLVIPSELGYGERGWPVKIPGGATLVFEVELLKIER

SEQ ID NO: 131 - DNA, 294 bp, synthetic construct
  misc_feature 1..294: note = Description of sequence: Construct Name: FKBP-166; Mutation(s): Q72R S79W Q84E A117W DRD M54A V85R
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgttttggctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 132 - protein, 98 aa, synthetic construct
  REGION 1..98: note = Description of sequence: Construct Name: FKBP-166; Mutation(s): Q72R S79W Q84E A117W DRD M54A V85R
HCPIKSRKGDVLHAHYTGKLEDGTEFDSSLPRNQPFVFWLGTGERIKGWDQGLLGMCEGEKRKLVIPSELGYGERGWPPKIPGGATLVFEVELLKIER

SEQ ID NO: 133 - DNA, 294 bp, synthetic construct
  misc_feature 1..294: note = Description of sequence: Construct Name: FKBP-167; Mutation(s): Q72R S79W Q84R A117W DRD M54A V85R
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgttttggctcggcaccggcagacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 134 - protein, 98 aa, synthetic construct
  REGION 1..98: note = Description of sequence: Construct Name: FKBP-167; Mutation(s): Q72R S79W Q84R A117W DRD M54A V85R
HCPIKSRKGDVLHAHYTGKLEDGTEFDSSLPRNQPFVFWLGTGRRIKGWDQGLLGMCEGEKRKLVIPSELGYGERGWPPKIPGGATLVFEVELLKIER

SEQ ID NO: 135 - DNA, 294 bp, synthetic construct
  misc_feature 1..294: note = Description of sequence: Construct Name: FKBP-168; Mutation(s): Q72R T82W Q84R A117W DRD M54A V85R
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggctggggcagacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 136 - protein, 98 aa, synthetic construct
  REGION 1..98: note = Description of sequence: Construct Name: FKBP-168; Mutation(s): Q72R T82W Q84R A117W DRD M54A V85R
HCPIKSRKGDVLHAHYTGKLEDGTEFDSSLPRNQPFVFSLGWGRRIKGWDQGLLGMCEGEKRKLVIPSELGYGERGWPPKIPGGATLVFEVELLKIER

SEQ ID NO: 137 - DNA, 294 bp, synthetic construct
  misc_feature 1..294: note = Description of sequence: Construct Name: FKBP-169; Mutation(s): Q72R T82W Q84E A117W DRD M54A
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggctggggcgaagtgatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 138 - protein, 98 aa, synthetic construct
  REGION 1..98: note = Description of sequence: Construct Name: FKBP-169; Mutation(s): Q72R T82W Q84E A117W DRD M54A
HCPIKSRKGDVLHAHYTGKLEDGTEFDSSLPRNQPFVFSLGWGEVIKGWDQGLLGMCEGEKRKLVIPSELGYGERGWPPKIPGGATLVFEVELLKIER

SEQ ID NO: 139 - DNA, 294 bp, synthetic construct
  misc_feature 1..294: note = Description of sequence: Construct Name: FKBP-170; Mutation(s): Q72R T82W Q84E A117W DRD M54A V85R
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggctggggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 140 - protein, 98 aa, synthetic construct
  REGION 1..98: note = Description of sequence: Construct Name: FKBP-170; Mutation(s): Q72R T82W Q84E A117W DRD M54A V85R
HCPIKSRKGDVLHAHYTGKLEDGTEFDSSLPRNQPFVFSLGWGERIKGWDQGLLGMCEGEKRKLVIPSELGYGERGWPPKIPGGATLVFEVELLKIER

SEQ ID NO: 141 - DNA, 294 bp, synthetic construct
  misc_feature 1..294: note = Description of sequence: Construct Name: FKBP-171; Mutation(s): Q72R Q84E A117W DRD M54A D50L
  source 1..294: mol_type = other DNA
cactgcccaatcaagagcagaaaaggccttgtgctgcacgctcactacaccggcaagctggaagatggcaccgagttcgatagcagcctgcctagaaaccagcctttcgtgtttagcctcggcaccggcgaagtgatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 142 - protein, 98 aa, synthetic construct
  REGION 1..98: note = Description of sequence: Construct Name: FKBP-171; Mutation(s): Q72R Q84E A117W DRD M54A D50L
HCPIKSRKGLVLHAHYTGKLEDGTEFDSSLPRNQPFVFSLGTGEVIKGWDQGLLGMCEGEKRKLVIPSELGYGERGWPPKIPGGATLVFEVELLKIER

SEQ ID NO: 143 - DNA, 294 bp, synthetic construct
  misc_feature 1..294: note = Description of sequence: Construct Name: FKBP-172; Mutation(s): Q72R Q84E A117W DRD M54A M96W
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaagtgatcaaaggctgggatcaaggcctgctgggatggtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 144 - protein, 98 aa, synthetic construct
  REGION 1..98: note = Description of sequence: Construct Name: FKBP-172; Mutation(s): Q72R Q84E A117W DRD M54A M96W
HCPIKSRKGDVLHAHYTGKLEDGTEFDSSLPRNQPFVFSLGTGEVIKGWDQGLLGWCEGEKRKLVIPSELGYGERGWPPKIPGGATLVFEVELLKIER

SEQ ID NO: 145 - DNA, 294 bp, synthetic construct
  misc_feature 1..294: note = Description of sequence: Construct Name: FKBP-173; Mutation(s): Q72R Q84E A117W DRD M54A L133G
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaagtgatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaagggctgaagatcgagcga

SEQ ID NO: 146 - protein, 98 aa, synthetic construct
  REGION 1..98: note = Description of sequence: Construct Name: FKBP-173; Mutation(s): Q72R Q84E A117W DRD M54A L133G
HCPIKSRKGDVLHAHYTGKLEDGTEFDSSLPRNQPFVFSLGTGEVIKGWDQGLLGMCEGEKRKLVIPSELGYGERGWPPKIPGGATLVFEVEGLKIER

SEQ ID NO: 147 - DNA, 294 bp, synthetic construct
  misc_feature 1..294: note = Description of sequence: Construct Name: FKBP-174; Mutation(s): Q72R Q84E A117W DRD M54A S36G V131G
  source 1..294: mol_type = other DNA
cactgcccaatcaaggggagaaaaggcgacgtgctgcacgctcactacaccggcaagctggaagatggcaccgagttcgatagcagcctgcctagaaaccagcctttcgtgtttagcctcggcaccggcgaagtgatcaaaggctgggatcaaggcctgctgggaatgtgcgaaggcgagaagagaaagctggtcatccctagcgaactcggctacggcgaaagaggctggcctccaaagattcctggcggagccacactggtgttcgaaggggaactgctgaagatcgagcga

SEQ ID NO: 148 - protein, 98 aa, synthetic construct
  REGION 1..98: note = Description of sequence: Construct Name: FKBP-174; Mutation(s): Q72R Q84E A117W DRD M54A S36G V131G
HCPIKGRKGDVLHAHYTGKLEDGTEFDSSLPRNQPFVFSLGTGEVIKGWDQGLLGMCEGEKRKLVIPSELGYGERGWPPKIPGGATLVFEGELLKIER

SEQ ID NO: 149 - DNA, 294 bp, synthetic construct
  misc_feature 1..294: note = Description of sequence: Construct Name: FKBP-175; Mutation(s): Q72R Q84E A117W DRD M54A D50L T57I I136L
  source 1..294: mol_type = other DNA
cactgcccaatcaagagcagaaaaggcgcggtgctgcacgctcactacatcggcaagctggaagatggcaccgagttcgatagcagcctgcctagaaaccagcctttcgtgtttagcctcggcaccggcgaagtgatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagctggagcga

SEQ ID NO: 150 - protein, 98 aa, synthetic construct
  REGION 1..98: note = Description of sequence: Construct Name: FKBP-175; Mutation(s): Q72R Q84E A117W DRD M54A D50L T57I I136L
HCPIKSRKGAVLHAHYIGKLEDGTEFDSSLPRNQPFVFSLGTGEVIKGWDQGLLGMCEGEKRKLVIPSELGYGERGWPPKIPGGATLVFEVELLKLER

SEQ ID NO: 151 - DNA, 294 bp, synthetic construct
  misc_feature 1..294: note = Description of sequence: Construct Name: FKBP-176; Mutation(s): Q72R Q84E A117W DRD M54A G116E
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaagtgatcaaaggctgggatcaaggcctgctgggaatgtgcgaaggcgagaagagaaagctggtcatccctagcgaactcggctacggcgaaagagagtggcctccaaagattcctggcggagccacactggtgttcgaagtggaactgctgaagatcgagcga

SEQ ID NO: 152 - protein, 98 aa, synthetic construct
  REGION 1..98: note = Description of sequence: Construct Name: FKBP-176; Mutation(s): Q72R Q84E A117W DRD M54A G116E
HCPIKSRKGDVLHAHYTGKLEDGTEFDSSLPRNQPFVFSLGTGEVIKGWDQGLLGMCEGEKRKLVIPSELGYGEREWPPKIPGGATLVFEVELLKIER

SEQ ID NO: 153 - DNA, 294 bp, synthetic construct
  misc_feature 1..294: note = Description of sequence: Construct Name: FKBP-177; Mutation(s): Q72R Q84E A117W DRD M54AL80G
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcgggggcaccggcgaagtgatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 154 - protein, 98 aa, synthetic construct
  REGION 1..98: note = Description of sequence: Construct Name: FKBP-177; Mutation(s): Q72R Q84E A117W DRD M54AL80G
HCPIKSRKGDVLHAHYTGKLEDGTEFDSSLPRNQPFVFSGGTGEVIKGWDQGLLGMCEGEKRKLVIPSELGYGERGWPPKIPGGATLVFEVELLKIER

SEQ ID NO: 155 - DNA, 294 bp, synthetic construct
  misc_feature 1..294: note = Description of sequence: Construct Name: FKBP-178; Mutation(s): Q72R Q84E A117W DRD M54AF78S
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtcgagcctcggcaccggcgaagtgatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 156 - protein, 98 aa, synthetic construct
  REGION 1..98: note = Description of sequence: Construct Name: FKBP-178; Mutation(s): Q72R Q84E A117W DRD M54AF78S
HCPIKSRKGDVLHAHYTGKLEDGTEFDSSLPRNQPFVSSLGTGEVIKGWDQGLLGMCEGEKRKLVIPSELGYGERGWPPKIPGGATLVFEVELLKIER

SEQ ID NO: 157 - DNA, 294 bp, synthetic construct
  misc_feature 1..294: note = Description of sequence: Construct Name: FKBP-179; Mutation(s): Q72R Q84E A117W DRD M54A H53G R72L
  source 1..294: mol_type = other DNA
cactgcccaatcaagagcagaaaaggcgacgtgctgggggctcactacaccggcaagctggaagatggcaccgagttcgatagcagcctgcctcttaaccagcctttcgtgtttagcctcggcaccggcgaagtgatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 158 - protein, 98 aa, synthetic construct
  REGION 1..98: note = Description of sequence: Construct Name: FKBP-179; Mutation(s): Q72R Q84E A117W DRD M54A H53G R72L
HCPIKSRKGDVLGAHYTGKLEDGTEFDSSLPLNQPFVFSLGTGEVIKGWDQGLLGMCEGEKRKLVIPSELGYGERGWPPKIPGGATLVFEVELLKIER

SEQ ID NO: 159 - DNA, 294 bp, synthetic construct
  misc_feature 1..294: note = Description of sequence: Construct Name: FKBP-180; Mutation(s): Q72R Q84E A117W DRD M54A F129T
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaagtgatcaaaggctgggatcaaggcctgctgggaatgtgcgaaggcgagaagagaaagctggtcatccctagcgaactcggctacggcgaaagaggctggcctccaaagattcctggcggagccacactggtgacggaagtggaactgctgaagatcgagcga

SEQ ID NO: 160 - protein, 98 aa, synthetic construct
  REGION 1..98: note = Description of sequence: Construct Name: FKBP-180; Mutation(s): Q72R Q84E A117W DRD M54A F129T
HCPIKSRKGDVLHAHYTGKLEDGTEFDSSLPRNQPFVFSLGTGEVIKGWDQGLLGMCEGEKRKLVIPSELGYGERGWPPKIPGGATLVTEVELLKIER

SEQ ID NO: 161 - DNA, 294 bp, synthetic construct
  misc_feature 1..294: note = Description of sequence: Construct Name: FKBP-181; Mutation(s): Q72R Q84E A117W V85R DRD M54A D50L
  source 1..294: mol_type = other DNA
cactgcccaatcaagagcagaaaaggccttgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 162 - protein, 98 aa, synthetic construct
  REGION 1..98: note = Description of sequence: Construct Name: FKBP-181; Mutation(s): Q72R Q84E A117W V85R DRD M54A D50L
HCPIKSRKGLVLHAHYTGKLEDGTEFDSSLPRNQPFVFSLGTGERIKGWDQGLLGMCEGEKRKLVIPSELGYGERGWPPKIPGGATLVFEVELLKIER

SEQ ID NO: 163 - DNA, 294 bp, synthetic construct
  misc_feature 1..294: note = Description of sequence: Construct Name: FKBP-182; Mutation(s): Q72R Q84E A117W V85R DRD M54A M96W
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggatggtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 164 - protein, 98 aa, synthetic construct
  REGION 1..98: note = Description of sequence: Construct Name: FKBP-182; Mutation(s): Q72R Q84E A117W V85R DRD M54A M96W
HCPIKSRKGDVLHAHYTGKLEDGTEFDSSLPRNQPFVFSLGTGERIKGWDQGLLGWCEGEKRKLVIPSELGYGERGWPPKIPGGATLVFEVELLKIER

SEQ ID NO: 165 - DNA, 294 bp, synthetic construct
  misc_feature 1..294: note = Description of sequence: Construct Name: FKBP-183; Mutation(s): Q72R Q84E A117W V85R DRD M54A L133G
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaagggctgaagatcgagcga

SEQ ID NO: 166 - protein, 98 aa, synthetic construct
  REGION 1..98: note = Description of sequence: Construct Name: FKBP-183; Mutation(s): Q72R Q84E A117W V85R DRD M54A L133G
HCPIKSRKGDVLHAHYTGKLEDGTEFDSSLPRNQPFVFSLGTGERIKGWDQGLLGMCEGEKRKLVIPSELGYGERGWPPKIPGGATLVFEVEGLKIER

SEQ ID NO: 167 - DNA, 294 bp, synthetic construct
  misc_feature 1..294: note = Description of sequence: Construct Name: FKBP-184; Mutation(s): Q72R Q84E A117W V85R DRD M54A S36G V131G
  source 1..294: mol_type = other DNA
cactgcccaatcaaggggagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaaggggaactgctgaagatcgagcga

SEQ ID NO: 168 - protein, 98 aa, synthetic construct
  REGION 1..98: note = Description of sequence: Construct Name: FKBP-184; Mutation(s): Q72R Q84E A117W V85R DRD M54A S36G V131G
HCPIKGRKGDVLHAHYTGKLEDGTEFDSSLPRNQPFVFSLGTGERIKGWDQGLLGMCEGEKRKLVIPSELGYGERGWPPKIPGGATLVFEGELLKIER

SEQ ID NO: 169 - DNA, 294 bp, synthetic construct
  misc_feature 1..294: note = Description of sequence: Construct Name: FKBP-185; Mutation(s): Q72R Q84E A117W V85R DRD M54A D50L T57I I136L
  source 1..294: mol_type = other DNA
cactgcccaatcaagagcagaaaaggcgcggtgctgcacgctcactacatcggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagctggagcga

SEQ ID NO: 170 - protein, 98 aa, synthetic construct
  REGION 1..98: note = Description of sequence: Construct Name: FKBP-185; Mutation(s): Q72R Q84E A117W V85R DRD M54A D50L T57I I136L
HCPIKSRKGAVLHAHYIGKLEDGTEFDSSLPRNQPFVFSLGTGERIKGWDQGLLGMCEGEKRKLVIPSELGYGERGWPPKIPGGATLVFEVELLKLER

SEQ ID NO: 171 - DNA, 294 bp, synthetic construct
  misc_feature 1..294: note = Description of sequence: Construct Name: FKBP-186; Mutation(s): Q72R Q84E A117W V85R DRD M54A G116E
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagagagtggcctccaaagattcctggcggagccacactggtgttcgaagtggaactgctgaagatcgagcga

SEQ ID NO: 172 - protein, 98 aa, synthetic construct
  REGION 1..98: note = Description of sequence: Construct Name: FKBP-186; Mutation(s): Q72R Q84E A117W V85R DRD M54A G116E
HCPIKSRKGDVLHAHYTGKLEDGTEFDSSLPRNQPFVFSLGTGERIKGWDQGLLGMCEGEKRKLVIPSELGYGEREWPPKIPGGATLVFEVELLKIER

SEQ ID NO: 173 - DNA, 294 bp, synthetic construct
  misc_feature 1..294: note = Description of sequence: Construct Name: FKBP-187; Mutation(s): Q72R Q84E A117W V85R DRD M54A L80G
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcgggggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 174 - protein, 98 aa, synthetic construct
  REGION 1..98: note = Description of sequence: Construct Name: FKBP-187; Mutation(s): Q72R Q84E A117W V85R DRD M54A L80G
HCPIKSRKGDVLHAHYTGKLEDGTEFDSSLPRNQPFVFSGGTGERIKGWDQGLLGMCEGEKRKLVIPSELGYGERGWPPKIPGGATLVFEVELLKIER

SEQ ID NO: 175 - DNA, 294 bp, synthetic construct
  misc_feature 1..294: note = Description of sequence: Construct Name: FKBP-188; Mutation(s): Q72R Q84E A117W V85R DRD M54A F78S
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtcgagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 176 - protein, 98 aa, synthetic construct
  REGION 1..98: note = Description of sequence: Construct Name: FKBP-188; Mutation(s): Q72R Q84E A117W V85R DRD M54A F78S
HCPIKSRKGDVLHAHYTGKLEDGTEFDSSLPRNQPFVSSLGTGERIKGWDQGLLGMCEGEKRKLVIPSELGYGERGWPPKIPGGATLVFEVELLKIER

SEQ ID NO: 177 - DNA, 294 bp, synthetic construct
  misc_feature 1..294: note = Description of sequence: Construct Name: FKBP-189; Mutation(s): Q72R Q84E A117W V85R DRD M54A H53G R72L
  source 1..294: mol_type = other DNA
cactgcccaatcaagagcagaaaaggcgacgtgctgggggctcactacaccggcaagctggaagatggcaccgagttcgatagcagcctgcctcttaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 178 - protein, 98 aa, synthetic construct
  REGION 1..98: note = Description of sequence: Construct Name: FKBP-189; Mutation(s): Q72R Q84E A117W V85R DRD M54A H53G R72L
HCPIKSRKGDVLGAHYTGKLEDGTEFDSSLPLNQPFVFSLGTGERIKGWDQGLLGMCEGEKRKLVIPSELGYGERGWPPKIPGGATLVFEVELLKIER

SEQ ID NO: 179 - DNA, 294 bp, synthetic construct
  misc_feature 1..294: note = Description of sequence: Construct Name: FKBP-190; Mutation(s): Q72R Q84E A117W V85R DRD M54A F129T
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgacggaagtggaactgctgaagatcgagcga

SEQ ID NO: 180 - protein, 98 aa, synthetic construct
  REGION 1..98: note = Description of sequence: Construct Name: FKBP-190; Mutation(s): Q72R Q84E A117W V85R DRD M54A F129T
HCPIKSRKGDVLHAHYTGKLEDGTEFDSSLPRNQPFVFSLGTGERIKGWDQGLLGMCEGEKRKLVIPSELGYGERGWPPKIPGGATLVTEVELLKIER

SEQ ID NO: 181 - DNA, 294 bp, synthetic construct
  misc_feature 1..294: note = Description of sequence: Construct Name: FKBP-191; Mutation(s): Q72R Q84E A117W V85R DRD G49S
  source 1..294: mol_type = other DNA
cactgcccaatcaagagcagaaaatccgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 182 - protein, 98 aa, synthetic construct
  REGION 1..98: note = Description of sequence: Construct Name: FKBP-191; Mutation(s): Q72R Q84E A117W V85R DRD G49S
HCPIKSRKSDVLHMHYTGKLEDGTEFDSSLPRNQPFVFSLGTGERIKGWDQGLLGMCEGEKRKLVIPSELGYGERGWPPKIPGGATLVFEVELLKIER

SEQ ID NO: 183 - DNA, 294 bp, synthetic construct
  misc_feature 1..294: note = Description of sequence: Construct Name: FKBP-192; Mutation(s): Q72R Q84E A117W V85R DRD P75L
  source 1..294: mol_type = other DNA
cactgcccaatcaagagcagaaaaggcgacgtgctgcacatgcactacaccggcaagctggaagatggcaccgagttcgatagcagcctgcctagaaaccagctgttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 184 - protein, 98 aa, synthetic construct
  REGION 1..98: note = Description of sequence: Construct Name: FKBP-192; Mutation(s): Q72R Q84E A117W V85R DRD P75L
HCPIKSRKGDVLHMHYTGKLEDGTEFDSSLPRNQLFVFSLGTGERIKGWDQGLLGMCEGEKRKLVIPSELGYGERGWPPKIPGGATLVFEVELLKIER

SEQ ID NO: 185 - DNA, 294 bp, synthetic construct
  misc_feature 1..294: note = Description of sequence: Construct Name: FKBP-193; Mutation(s): Q72R Q84E A117W V85R DRD D90A
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggctcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 186 - protein, 98 aa, synthetic construct
  REGION 1..98: note = Description of sequence: Construct Name: FKBP-193; Mutation(s): Q72R Q84E A117W V85R DRD D90A
HCPIKSRKGDVLHMHYTGKLEDGTEFDSSLPRNQPFVFSLGTGERIKGWAQGLLGMCEGEKRKLVIPSELGYGERGWPPKIPGGATLVFEVELLKIER

SEQ ID NO: 187 - DNA, 294 bp, synthetic construct
  misc_feature 1..294: note = Description of sequence: Construct Name: FKBP-194; Mutation(s): Q72R Q84E A117W V85R DRD L104V
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaaggttgtcatccctagcgaactcggctacggcgaaagaggctggcctccaaagattcctggcggagccacactggtgttcgaagtggaactgctgaagatcgagcga

SEQ ID NO: 188 - protein, 98 aa, synthetic construct
  REGION 1..98: note = Description of sequence: Construct Name: FKBP-194; Mutation(s): Q72R Q84E A117W V85R DRD L104V
HCPIKSRKGDVLHMHYTGKLEDGTEFDSSLPRNQPFVFSLGTGERIKGWDQGLLGMCEGEKRKVVIPSELGYGERGWPPKIPGGATLVFEVELLKIER

SEQ ID NO: 189 - DNA, 294 bp, synthetic construct
  misc_feature 1..294: note = Description of sequence: Construct Name: FKBP-195; Mutation(s): Q72R Q84E A117W V85R DRD K135Y
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgtacatcgagcga

SEQ ID NO: 190 - protein, 98 aa, synthetic construct
  REGION 1..98: note = Description of sequence: Construct Name: FKBP-195; Mutation(s): Q72R Q84E A117W V85R DRD K135Y
HCPIKSRKGDVLHMHYTGKLEDGTEFDSSLPRNQPFVFSLGTGERIKGWDQGLLGMCEGEKRKLVIPSELGYGERGWPPKIPGGATLVFEVELLYIER

SEQ ID NO: 191 - DNA, 294 bp, synthetic construct
  misc_feature 1..294: note = Description of sequence: Construct Name: FKBP-196; Mutation(s): Q72R Q84E A117W V85R DRD I136V
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaaggtggagcga

SEQ ID NO: 192 - protein, 98 aa, synthetic construct
  REGION 1..98: note = Description of sequence: Construct Name: FKBP-196; Mutation(s): Q72R Q84E A117W V85R DRD I136V
HCPIKSRKGDVLHMHYTGKLEDGTEFDSSLPRNQPFVFSLGTGERIKGWDQGLLGMCEGEKRKLVIPSELGYGERGWPPKIPGGATLVFEVELLKVER

SEQ ID NO: 193 - DNA, 294 bp, synthetic construct
  misc_feature 1..294: note = Description of sequence: Construct Name: FKBP-197; Mutation(s): Q72R Q84E A117W V85R DRD E130R
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcagagtggaactgctgaagatcgagcga

SEQ ID NO: 194 - protein, 98 aa, synthetic construct
  REGION 1..98: note = Description of sequence: Construct Name: FKBP-197; Mutation(s): Q72R Q84E A117W V85R DRD E130R
HCPIKSRKGDVLHMHYTGKLEDGTEFDSSLPRNQPFVFSLGTGERIKGWDQGLLGMCEGEKRKLVIPSELGYGERGWPPKIPGGATLVFRVELLKIER

SEQ ID NO: 195 - DNA, 294 bp, synthetic construct
  misc_feature 1..294: note = Description of sequence: Construct Name: FKBP-198; Mutation(s): Q72R Q84E A117W V85R DRD K101W
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagtggagaaagctggtcatccctagcgaactcggctacggcgaaagaggctggcctccaaagattcctggcggagccacactggtgttcgaagtggaactgctgaagatcgagcga

SEQ ID NO: 196 - protein, 98 aa, synthetic construct
  REGION 1..98: note = Description of sequence: Construct Name: FKBP-198; Mutation(s): Q72R Q84E A117W V85R DRD K101W
HCPIKSRKGDVLHMHYTGKLEDGTEFDSSLPRNQPFVFSLGTGERIKGWDQGLLGMCEGEWRKLVIPSELGYGERGWPPKIPGGATLVFEVELLKIER

SEQ ID NO: 197 - DNA, 294 bp, synthetic construct
  misc_feature 1..294: note = Description of sequence: Construct Name: FKBP-199; Mutation(s): Q72R Q84E A117W V85R DRD E98L
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcttgggcgagaagagaaagctggtcatccctagcgaactcggctacggcgaaagaggctggcctccaaagattcctggcggagccacactggtgttcgaagtggaactgctgaagatcgagcga

SEQ ID NO: 198 - protein, 98 aa, synthetic construct
  REGION 1..98: note = Description of sequence: Construct Name: FKBP-199; Mutation(s): Q72R Q84E A117W V85R DRD E98L
HCPIKSRKGDVLHMHYTGKLEDGTEFDSSLPRNQPFVFSLGTGERIKGWDQGLLGMCLGEKRKLVIPSELGYGERGWPPKIPGGATLVFEVELLKIER

SEQ ID NO: 199 - DNA, 294 bp, synthetic construct
  misc_feature 1..294: note = Description of sequence: Construct Name: FKBP-200; Mutation(s): Q72R Q84E A117W V85R DRD D62S
  source 1..294: mol_type = other DNA
cactgcccaatcaagagcagaaaaggcgacgtgctgcacatgcactacaccggcaagctggaatctggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 200 - protein, 98 aa, synthetic construct
  REGION 1..98: note = Description of sequence: Construct Name: FKBP-200; Mutation(s): Q72R Q84E A117W V85R DRD D62S
HCPIKSRKGDVLHMHYTGKLESGTEFDSSLPRNQPFVFSLGTGERIKGWDQGLLGMCEGEKRKLVIPSELGYGERGWPPKIPGGATLVFEVELLKIER

SEQ ID NO: 201 - DNA, 294 bp, synthetic construct
  misc_feature 1..294: note = Description of sequence: Construct Name: FKBP-201; Mutation(s): Q72R Q84E A117W V85R DRD H53L
  source 1..294: mol_type = other DNA
cactgcccaatcaagagcagaaaaggcgacgtgctgttgatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 202 - protein, 98 aa, synthetic construct
  REGION 1..98: note = Description of sequence: Construct Name: FKBP-201; Mutation(s): Q72R Q84E A117W V85R DRD H53L
HCPIKSRKGDVLLMHYTGKLEDGTEFDSSLPRNQPFVFSLGTGERIKGWDQGLLGMCEGEKRKLVIPSELGYGERGWPPKIPGGATLVFEVELLKIER

SEQ ID NO: 203 - DNA, 294 bp, synthetic construct
  misc_feature 1..294: note = Description of sequence: Construct Name: FKBP-202; Mutation(s): Q72R Q84E A117W V85R DRD M54A G49S
  source 1..294: mol_type = other DNA
cactgcccaatcaagagcagaaaatccgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 204 - protein, 98 aa, synthetic construct
  REGION 1..98: note = Description of sequence: Construct Name: FKBP-202; Mutation(s): Q72R Q84E A117W V85R DRD M54A G49S
HCPIKSRKSDVLHAHYTGKLEDGTEFDSSLPRNQPFVFSLGTGERIKGWDQGLLGMCEGEKRKLVIPSELGYGERGWPPKIPGGATLVFEVELLKIER

SEQ ID NO: 205 - DNA, 294 bp, synthetic construct
  misc_feature 1..294: note = Description of sequence: Construct Name: FKBP-203; Mutation(s): Q72R Q84E A117W V85R DRD M54A P75L
  source 1..294: mol_type = other DNA
cactgcccaatcaagagcagaaaaggcgacgtgctgcacgctcactacaccggcaagctggaagatggcaccgagttcgatagcagcctgcctagaaaccagctgttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 206 - protein, 98 aa, synthetic construct
  REGION 1..98: note = Description of sequence: Construct Name: FKBP-203; Mutation(s): Q72R Q84E A117W V85R DRD M54A P75L
HCPIKSRKGDVLHAHYTGKLEDGTEFDSSLPRNQLFVFSLGTGERIKGWDQGLLGMCEGEKRKLVIPSELGYGERGWPPKIPGGATLVFEVELLKIER

SEQ ID NO: 207 - DNA, 294 bp, synthetic construct
  misc_feature 1..294: note = Description of sequence: Construct Name: FKBP-204; Mutation(s): Q72R Q84E A117W V85R DRD M54A D90A
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggctcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 208 - protein, 98 aa, synthetic construct
  REGION 1..98: note = Description of sequence: Construct Name: FKBP-204; Mutation(s): Q72R Q84E A117W V85R DRD M54A D90A
HCPIKSRKGDVLHAHYTGKLEDGTEFDSSLPRNQPFVFSLGTGERIKGWAQGLLGMCEGEKRKLVIPSELGYGERGWPPKIPGGATLVFEVELLKIER

SEQ ID NO: 209 - DNA, 294 bp, synthetic construct
  misc_feature 1..294: note = Description of sequence: Construct Name: FKBP-205; Mutation(s): Q72R Q84E A117W V85R DRD M54A L104V
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaaggttgtcatccctagcgaactcggctacggcgaaagaggctggcctccaaagattcctggcggagccacactggtgttcgaagtggaactgctgaagatcgagcga

SEQ ID NO: 210 - protein, 98 aa, synthetic construct
  REGION 1..98: note = Description of sequence: Construct Name: FKBP-205; Mutation(s): Q72R Q84E A117W V85R DRD M54A L104V
HCPIKSRKGDVLHAHYTGKLEDGTEFDSSLPRNQPFVFSLGTGERIKGWDQGLLGMCEGEKRKVVIPSELGYGERGWPPKIPGGATLVFEVELLKIER

SEQ ID NO: 211 - DNA, 294 bp, synthetic construct
  misc_feature 1..294: note = Description of sequence: Construct Name: FKBP-206; Mutation(s): Q72R Q84E A117W V85R DRD M54A K135Y
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgtacatcgagcga

SEQ ID NO: 212 - protein, 98 aa, synthetic construct
  REGION 1..98: note = Description of sequence: Construct Name: FKBP-206; Mutation(s): Q72R Q84E A117W V85R DRD M54A K135Y
HCPIKSRKGDVLHAHYTGKLEDGTEFDSSLPRNQPFVFSLGTGERIKGWDQGLLGMCEGEKRKLVIPSELGYGERGWPPKIPGGATLVFEVELLYIER

SEQ ID NO: 213 - DNA, 294 bp, synthetic construct
  misc_feature 1..294: note = Description of sequence: Construct Name: FKBP-207; Mutation(s): Q72R Q84E A117W V85R DRD M54A I136V
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaaggtggagcga

SEQ ID NO: 214 - protein, 98 aa, synthetic construct
  REGION 1..98: note = Description of sequence: Construct Name: FKBP-207; Mutation(s): Q72R Q84E A117W V85R DRD M54A I136V
HCPIKSRKGDVLHAHYTGKLEDGTEFDSSLPRNQPFVFSLGTGERIKGWDQGLLGMCEGEKRKLVIPSELGYGERGWPPKIPGGATLVFEVELLKVER

SEQ ID NO: 215 - DNA, 294 bp, synthetic construct
  misc_feature 1..294: note = Description of sequence: Construct Name: FKBP-208; Mutation(s): Q72R Q84E A117W V85R DRD M54A E130R
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcagagtggaactgctgaagatcgagcga

SEQ ID NO: 216 - protein, 98 aa, synthetic construct
  REGION 1..98: note = Description of sequence: Construct Name: FKBP-208; Mutation(s): Q72R Q84E A117W V85R DRD M54A E130R
HCPIKSRKGDVLHAHYTGKLEDGTEFDSSLPRNQPFVFSLGTGERIKGWDQGLLGMCEGEKRKLVIPSELGYGERGWPPKIPGGATLVFRVELLKIER

SEQ ID NO: 217 - DNA, 294 bp, synthetic construct
  misc_feature 1..294: note = Description of sequence: Construct Name: FKBP-209; Mutation(s): Q72R Q84E A117W V85R DRD M54A K101W
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagtggagaaagctggtcatccctagcgaactcggctacggcgaaagaggctggcctccaaagattcctggcggagccacactggtgttcgaagtggaactgctgaagatcgagcga

SEQ ID NO: 218 - protein, 98 aa, synthetic construct
  REGION 1..98: note = Description of sequence: Construct Name: FKBP-209; Mutation(s): Q72R Q84E A117W V85R DRD M54A K101W
HCPIKSRKGDVLHAHYTGKLEDGTEFDSSLPRNQPFVFSLGTGERIKGWDQGLLGMCEGEWRKLVIPSELGYGERGWPPKIPGGATLVFEVELLKIER

SEQ ID NO: 219 - DNA, 294 bp, synthetic construct
  misc_feature 1..294: note = Description of sequence: Construct Name: FKBP-210; Mutation(s): Q72R Q84E A117W V85R DRD M54A E98L
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcttgggcgagaagagaaagctggtcatccctagcgaactcggctacggcgaaagaggctggcctccaaagattcctggcggagccacactggtgttcgaagtggaactgctgaagatcgagcga

SEQ ID NO: 220 - protein, 98 aa, synthetic construct
  REGION 1..98: note = Description of sequence: Construct Name: FKBP-210; Mutation(s): Q72R Q84E A117W V85R DRD M54A E98L
HCPIKSRKGDVLHAHYTGKLEDGTEFDSSLPRNQPFVFSLGTGERIKGWDQGLLGMCLGEKRKLVIPSELGYGERGWPPKIPGGATLVFEVELLKIER

SEQ ID NO: 221 - DNA, 294 bp, synthetic construct
  misc_feature 1..294: note = Description of sequence: Construct Name: FKBP-211; Mutation(s): Q72R Q84E A117W V85R DRD M54A D62S
  source 1..294: mol_type = other DNA
cactgcccaatcaagagcagaaaaggcgacgtgctgcacgctcactacaccggcaagctggaatctggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 222 - protein, 98 aa, synthetic construct
  REGION 1..98: note = Description of sequence: Construct Name: FKBP-211; Mutation(s): Q72R Q84E A117W V85R DRD M54A D62S
HCPIKSRKGDVLHAHYTGKLESGTEFDSSLPRNQPFVFSLGTGERIKGWDQGLLGMCEGEKRKLVIPSELGYGERGWPPKIPGGATLVFEVELLKIER

SEQ ID NO: 223 - DNA, 294 bp, synthetic construct
  misc_feature 1..294: note = Description of sequence: Construct Name: FKBP-212; Mutation(s): Q72R Q84E A117W V85R DRD M54A H53L
  source 1..294: mol_type = other DNA
cactgcccaatcaagagcagaaaaggcgacgtgctgttggctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 224 - protein, 98 aa, synthetic construct
  REGION 1..98: note = Description of sequence: Construct Name: FKBP-212; Mutation(s): Q72R Q84E A117W V85R DRD M54A H53L
HCPIKSRKGDVLLAHYTGKLEDGTEFDSSLPRNQPFVFSLGTGERIKGWDQGLLGMCEGEKRKLVIPSELGYGERGWPPKIPGGATLVFEVELLKIER

SEQ ID NO: 225 - DNA, 294 bp, synthetic construct
  misc_feature 1..294: note = Description of sequence: Construct Name: FKBP-213; Mutation(s): Q72R Q84G A117W DRD P43R M54A
  source 1..294: mol_type = other DNA
cactgcagaatcaagagcagaaaaggcgacgtgctgcacgctcactacaccggcaagctggaagatggcaccgagttcgatagcagcctgcctagaaaccagcctttcgtgtttagcctcggcaccggcggagtgatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 226 - protein, 98 aa, synthetic construct
  REGION 1..98: note = Description of sequence: Construct Name: FKBP-213; Mutation(s): Q72R Q84G A117W DRD P43R M54A
HCRIKSRKGDVLHAHYTGKLEDGTEFDSSLPRNQPFVFSLGTGGVIKGWDQGLLGMCEGEKRKLVIPSELGYGERGWPPKIPGGATLVFEVELLKIER

SEQ ID NO: 227 - DNA, 294 bp, synthetic construct
  misc_feature 1..294: note = Description of sequence: Construct Name: FKBP-214; Mutation(s): Q72R Q84G A117W V85R DRD P43R M54A
  source 1..294: mol_type = other DNA
cactgcagaatcaagagcagaaaaggcgacgtgctgcacgctcactacaccggcaagctggaagatggcaccgagttcgatagcagcctgcctagaaaccagcctttcgtgtttagcctcggcaccggcggacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 228 - protein, 98 aa, synthetic construct
  REGION 1..98: note = Description of sequence: Construct Name: FKBP-214; Mutation(s): Q72R Q84G A117W V85R DRD P43R M54A
HCRIKSRKGDVLHAHYTGKLEDGTEFDSSLPRNQPFVFSLGTGGRIKGWDQGLLGMCEGEKRKLVIPSELGYGERGWPPKIPGGATLVFEVELLKIER

SEQ ID NO: 229 - DNA, 294 bp, synthetic construct
  misc_feature 1..294: note = Description of sequence: Construct Name: FKBP-215; Mutation(s): Q72R Q84G A117W V85R DRD P43R M54A K135Y
  source 1..294: mol_type = other DNA
cactgcagaatcaagagcagaaaaggcgacgtgctgcacgctcactacaccggcaagctggaagatggcaccgagttcgatagcagcctgcctagaaaccagcctttcgtgtttagcctcggcaccggcggacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgtacatcgagcga

SEQ ID NO: 230 - protein, 98 aa, synthetic construct
  REGION 1..98: note = Description of sequence: Construct Name: FKBP-215; Mutation(s): Q72R Q84G A117W V85R DRD P43R M54A K135Y
HCRIKSRKGDVLHAHYTGKLEDGTEFDSSLPRNQPFVFSLGTGGRIKGWDQGLLGMCEGEKRKLVIPSELGYGERGWPPKIPGGATLVFEVELLYIER

SEQ ID NO: 231 - DNA, 294 bp, synthetic construct
  misc_feature 1..294: note = Description of sequence: Construct Name: FKBP-216; Mutation(s): Q72R Q84E A117W DRD M54A E61Y K101W K135Y
  source 1..294: mol_type = other DNA
cactgcccaatcaagagcagaaaaggcgacgtgctgcacgctcactacaccggcaagctgtatgatggcaccgagttcgatagcagcctgcctagaaaccagcctttcgtgtttagcctcggcaccggcgaagtgatcaaaggctgggatcaaggcctgctgggaatgtgcgaaggcgagtggagaaagctggtcatccctagcgaactcggctacggcgaaagaggctggcctccaaagattcctggcggagccacactggtgttcgaagtggaactgctgtacatcgagcga

SEQ ID NO: 232 - protein, 98 aa, synthetic construct
  REGION 1..98: note = Description of sequence: Construct Name: FKBP-216; Mutation(s): Q72R Q84E A117W DRD M54A E61Y K101W K135Y
HCPIKSRKGDVLHAHYTGKLYDGTEFDSSLPRNQPFVFSLGTGEVIKGWDQGLLGMCEGEWRKLVIPSELGYGERGWPPKIPGGATLVFEVELLYIER

SEQ ID NO: 233 - DNA, 294 bp, synthetic construct
  misc_feature 1..294: note = Description of sequence: Construct Name: FKBP-217; Mutation(s): Q72R Q84E A117W V85R DRD M54A E61Y K101W K135Y
  source 1..294: mol_type = other DNA
cactgcccaatcaagagcagaaaaggcgacgtgctgcacgctcactacaccggcaagctgtatgatggcaccgagttcgatagcagcctgcctagaaaccagcctttcgtgtttagcctcggcaccggcgaacggatcaaaggctgggatcaaggcctgctgggaatgtgcgaaggcgagtggagaaagctggtcatccctagcgaactcggctacggcgaaagaggctggcctccaaagattcctggcggagccacactggtgttcgaagtggaactgctgtacatcgagcga

SEQ ID NO: 234 - protein, 98 aa, synthetic construct
  REGION 1..98: note = Description of sequence: Construct Name: FKBP-217; Mutation(s): Q72R Q84E A117W V85R DRD M54A E61Y K101W K135Y
HCPIKSRKGDVLHAHYTGKLYDGTEFDSSLPRNQPFVFSLGTGERIKGWDQGLLGMCEGEWRKLVIPSELGYGERGWPPKIPGGATLVFEVELLYIER

SEQ ID NO: 235 - DNA, 294 bp, synthetic construct
  misc_feature 1..294: note = Description of sequence: Construct Name: FKBP-218; Mutation(s): Q72R Q84E A117W DRD M54A E61G K101G K135E
  source 1..294: mol_type = other DNA
cactgcccaatcaagagcagaaaaggcgacgtgctgcacgctcactacaccggcaagctgggcgatggcaccgagttcgatagcagcctgcctagaaaccagcctttcgtgtttagcctcggcaccggcgaagtgatcaaaggctgggatcaaggcctgctgggaatgtgcgaaggcgagggcagaaagctggtcatccctagcgaactcggctacggcgaaagaggctggcctccaaagattcctggcggagccacactggtgttcgaagtggaactgctggagatcgagcga

SEQ ID NO: 236 - protein, 98 aa, synthetic construct
  REGION 1..98: note = Description of sequence: Construct Name: FKBP-218; Mutation(s): Q72R Q84E A117W DRD M54A E61G K101G K135E
HCPIKSRKGDVLHAHYTGKLGDGTEFDSSLPRNQPFVFSLGTGEVIKGWDQGLLGMCEGEGRKLVIPSELGYGERGWPPKIPGGATLVFEVELLEIER

SEQ ID NO: 237 - DNA, 294 bp, synthetic construct
  misc_feature 1..294: note = Description of sequence: Construct Name: FKBP-219; Mutation(s): Q72R Q84E A117W V85R DRD M54A E61G K101G K135E
  source 1..294: mol_type = other DNA
cactgcccaatcaagagcagaaaaggcgacgtgctgcacgctcactacaccggcaagctgggcgatggcaccgagttcgatagcagcctgcctagaaaccagcctttcgtgtttagcctcggcaccggcgaacggatcaaaggctgggatcaaggcctgctgggaatgtgcgaaggcgagggcagaaagctggtcatccctagcgaactcggctacggcgaaagaggctggcctccaaagattcctggcggagccacactggtgttcgaagtggaactgctggagatcgagcga

SEQ ID NO: 238 - protein, 98 aa, synthetic construct
  REGION 1..98: note = Description of sequence: Construct Name: FKBP-219; Mutation(s): Q72R Q84E A117W V85R DRD M54A E61G K101G K135E
HCPIKSRKGDVLHAHYTGKLGDGTEFDSSLPRNQPFVFSLGTGERIKGWDQGLLGMCEGEGRKLVIPSELGYGERGWPPKIPGGATLVFEVELLEIER

SEQ ID NO: 239 - DNA, 294 bp, synthetic construct
  misc_feature 1..294: note = Description of sequence: Construct Name: FKBP-220; Mutation(s): Q72R Q84E A117W V85R DRD T25G
  source 1..294: mol_type = other DNA
cactgcccaatcaagagcagaaaaggcgacgtgctgcacatgcactacaccggcaagctggaagatggcggggagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 240 - protein, 98 aa, synthetic construct
  REGION 1..98: note = Description of sequence: Construct Name: FKBP-220; Mutation(s): Q72R Q84E A117W V85R DRD T25G
HCPIKSRKGDVLHMHYTGKLEDGGEFDSSLPRNQPFVFSLGTGERIKGWDQGLLGMCEGEKRKLVIPSELGYGERGWPPKIPGGATLVFEVELLKIER

SEQ ID NO: 241 - DNA, 294 bp, synthetic construct
  misc_feature 1..294: note = Description of sequence: Construct Name: FKBP-221; Mutation(s): Q72R Q84E A117W V85R DRD M54A T25G
  source 1..294: mol_type = other DNA
cactgcccaatcaagagcagaaaaggcgacgtgctgcacgctcactacaccggcaagctggaagatggcggggagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 242 - protein, 98 aa, synthetic construct
  REGION 1..98: note = Description of sequence: Construct Name: FKBP-221; Mutation(s): Q72R Q84E A117W V85R DRD M54A T25G
HCPIKSRKGDVLHAHYTGKLEDGGEFDSSLPRNQPFVFSLGTGERIKGWDQGLLGMCEGEKRKLVIPSELGYGERGWPPKIPGGATLVFEVELLKIER

SEQ ID NO: 243 - DNA, 294 bp, synthetic construct
  misc_feature 1..294: note = Description of sequence: Construct Name: FKBP-222; Mutation(s): Q72R Q84E A117W DRD P4W T25R
  source 1..294: mol_type = other DNA
cactgctggatcaagagcagaaaaggcgacgtgctgcacatgcactacaccggcaagctggaagatggccgggagttcgatagcagcctgcctagaaaccagcctttcgtgtttagcctcggcaccggcgaagtgatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 244 - protein, 98 aa, synthetic construct
  REGION 1..98: note = Description of sequence: Construct Name: FKBP-222; Mutation(s): Q72R Q84E A117W DRD P4W T25R
HCWIKSRKGDVLHMHYTGKLEDGREFDSSLPRNQPFVFSLGTGEVIKGWDQGLLGMCEGEKRKLVIPSELGYGERGWPPKIPGGATLVFEVELLKIER

SEQ ID NO: 245 - DNA, 294 bp, synthetic construct
  misc_feature 1..294: note = Description of sequence: Construct Name: FKBP-223; Mutation(s): Q72R Q84E A117W V85R DRD M54A P4W T25R
  source 1..294: mol_type = other DNA
cactgctggatcaagagcagaaaaggcgacgtgctgcacgctcactacaccggcaagctggaagatggccgggagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 246 - protein, 98 aa, synthetic construct
  REGION 1..98: note = Description of sequence: Construct Name: FKBP-223; Mutation(s): Q72R Q84E A117W V85R DRD M54A P4W T25R
HCWIKSRKGDVLHAHYTGKLEDGREFDSSLPRNQPFVFSLGTGERIKGWDQGLLGMCEGEKRKLVIPSELGYGERGWPPKIPGGATLVFEVELLKIER

SEQ ID NO: 247 - DNA, 294 bp, synthetic construct
  misc_feature 1..294: note = Description of sequence: Construct Name: FKBP-224; Mutation(s): Q72R Q84E A117W DRD P43R
  source 1..294: mol_type = other DNA
cactgcagaatcaagagcagaaaaggcgacgtgctgcacatgcactacaccggcaagctggaagatggcaccgagttcgatagcagcctgcctagaaaccagcctttcgtgtttagcctcggcaccggcgaagtgatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 248 - protein, 98 aa, synthetic construct
  REGION 1..98: note = Description of sequence: Construct Name: FKBP-224; Mutation(s): Q72R Q84E A117W DRD P43R
HCRIKSRKGDVLHMHYTGKLEDGTEFDSSLPRNQPFVFSLGTGEVIKGWDQGLLGMCEGEKRKLVIPSELGYGERGWPPKIPGGATLVFEVELLKIER

SEQ ID NO: 249 - DNA, 294 bp, synthetic construct
  misc_feature 1..294: note = Description of sequence: Construct Name: FKBP-225; Mutation(s): Q72R Q84E A117W V85R DRD P43R
  source 1..294: mol_type = other DNA
cactgcagaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 250 - protein, 98 aa, synthetic construct
  REGION 1..98: note = Description of sequence: Construct Name: FKBP-225; Mutation(s): Q72R Q84E A117W V85R DRD P43R
HCRIKSRKGDVLHMHYTGKLEDGTEFDSSLPRNQPFVFSLGTGERIKGWDQGLLGMCEGEKRKLVIPSELGYGERGWPPKIPGGATLVFEVELLKIER

SEQ ID NO: 251 - DNA, 294 bp, synthetic construct
  misc_feature 1..294: note = Description of sequence: Construct Name: FKBP-226; Mutation(s): Q72R Q84E A117W V85R DRD P43R M54A
  source 1..294: mol_type = other DNA
cactgcagaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 252 - protein, 98 aa, synthetic construct
  REGION 1..98: note = Description of sequence: Construct Name: FKBP-226; Mutation(s): Q72R Q84E A117W V85R DRD P43R M54A
HCRIKSRKGDVLHAHYTGKLEDGTEFDSSLPRNQPFVFSLGTGERIKGWDQGLLGMCEGEKRKLVIPSELGYGERGWPPKIPGGATLVFEVELLKIER

SEQ ID NO: 253 - DNA, 294 bp, synthetic construct
  misc_feature 1..294: note = Description of sequence: Construct Name: FKBP-227; Mutation(s): Q72R Q84E A117W DRD E137R
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaagtgatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcaggcga

SEQ ID NO: 254 - protein, 98 aa, synthetic construct
  REGION 1..98: note = Description of sequence: Construct Name: FKBP-227; Mutation(s): Q72R Q84E A117W DRD E137R
HCPIKSRKGDVLHMHYTGKLEDGTEFDSSLPRNQPFVFSLGTGEVIKGWDQGLLGMCEGEKRKLVIPSELGYGERGWPPKIPGGATLVFEVELLKIRR

SEQ ID NO: 255 - DNA, 294 bp, synthetic construct
  misc_feature 1..294: note = Description of sequence: Construct Name: FKBP-228; Mutation(s): Q72R Q84E A117W V85R DRD E137R
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcaggcga

SEQ ID NO: 256 - protein, 98 aa, synthetic construct
  REGION 1..98: note = Description of sequence: Construct Name: FKBP-228; Mutation(s): Q72R Q84E A117W V85R DRD E137R
HCPIKSRKGDVLHMHYTGKLEDGTEFDSSLPRNQPFVFSLGTGERIKGWDQGLLGMCEGEKRKLVIPSELGYGERGWPPKIPGGATLVFEVELLKIRR

SEQ ID NO: 257 - DNA, 294 bp, synthetic construct
  misc_feature 1..294: note = Description of sequence: Construct Name: FKBP-229; Mutation(s): Q72R Q84E A117W V85R DRD E137R M54A
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcaggcga

SEQ ID NO: 258 - protein, 98 aa, synthetic construct
  REGION 1..98: note = Description of sequence: Construct Name: FKBP-229; Mutation(s): Q72R Q84E A117W V85R DRD E137R M54A
HCPIKSRKGDVLHAHYTGKLEDGTEFDSSLPRNQPFVFSLGTGERIKGWDQGLLGMCEGEKRKLVIPSELGYGERGWPPKIPGGATLVFEVELLKIRR

SEQ ID NO: 259 - DNA, 294 bp, synthetic construct
  misc_feature 1..294: note = Description of sequence: Construct Name: FKBP-230; Mutation(s): Q72R Q84E A117W DRD G63M
  source 1..294: mol_type = other DNA
cactgcccaatcaagagcagaaaaggcgacgtgctgcacatgcactacaccggcaagctggaagatatgaccgagttcgatagcagcctgcctagaaaccagcctttcgtgtttagcctcggcaccggcgaagtgatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 260 - protein, 98 aa, synthetic construct
  REGION 1..98: note = Description of sequence: Construct Name: FKBP-230; Mutation(s): Q72R Q84E A117W DRD G63M
HCPIKSRKGDVLHMHYTGKLEDMTEFDSSLPRNQPFVFSLGTGEVIKGWDQGLLGMCEGEKRKLVIPSELGYGERGWPPKIPGGATLVFEVELLKIER

SEQ ID NO: 261 - DNA, 294 bp, synthetic construct
  misc_feature 1..294: note = Description of sequence: Construct Name: FKBP-231; Mutation(s): Q72R Q84E A117W V85R DRD G63M
  source 1..294: mol_type = other DNA
cactgcccaatcaagagcagaaaaggcgacgtgctgcacatgcactacaccggcaagctggaagatatgaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 262 - protein, 98 aa, synthetic construct
  REGION 1..98: note = Description of sequence: Construct Name: FKBP-231; Mutation(s): Q72R Q84E A117W V85R DRD G63M
HCPIKSRKGDVLHMHYTGKLEDMTEFDSSLPRNQPFVFSLGTGERIKGWDQGLLGMCEGEKRKLVIPSELGYGERGWPPKIPGGATLVFEVELLKIER

SEQ ID NO: 263 - DNA, 294 bp, synthetic construct
  misc_feature 1..294: note = Description of sequence: Construct Name: FKBP-232; Mutation(s): Q72R Q84E A117W V85R DRD G63M M54A
  source 1..294: mol_type = other DNA
cactgcccaatcaagagcagaaaaggcgacgtgctgcacgctcactacaccggcaagctggaagatatgaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 264 - protein, 98 aa, synthetic construct
  REGION 1..98: note = Description of sequence: Construct Name: FKBP-232; Mutation(s): Q72R Q84E A117W V85R DRD G63M M54A
HCPIKSRKGDVLHAHYTGKLEDMTEFDSSLPRNQPFVFSLGTGERIKGWDQGLLGMCEGEKRKLVIPSELGYGERGWPPKIPGGATLVFEVELLKIER

SEQ ID NO: 265 - DNA, 294 bp, synthetic construct
  misc_feature 1..294: note = Description of sequence: Construct Name: FKBP-233; Mutation(s): Q72R Q84E A117W DRD D90T
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaagtgatcaaaggctggacgcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 266 - protein, 98 aa, synthetic construct
  REGION 1..98: note = Description of sequence: Construct Name: FKBP-233; Mutation(s): Q72R Q84E A117W DRD D90T
HCPIKSRKGDVLHMHYTGKLEDGTEFDSSLPRNQPFVFSLGTGEVIKGWTQGLLGMCEGEKRKLVIPSELGYGERGWPPKIPGGATLVFEVELLKIER

SEQ ID NO: 267 - DNA, 294 bp, synthetic construct
  misc_feature 1..294: note = Description of sequence: Construct Name: FKBP-234; Mutation(s): Q72R Q84E A117W V85R DRD D90T
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctggacgcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 268 - protein, 98 aa, synthetic construct
  REGION 1..98: note = Description of sequence: Construct Name: FKBP-234; Mutation(s): Q72R Q84E A117W V85R DRD D90T
HCPIKSRKGDVLHMHYTGKLEDGTEFDSSLPRNQPFVFSLGTGERIKGWTQGLLGMCEGEKRKLVIPSELGYGERGWPPKIPGGATLVFEVELLKIER

SEQ ID NO: 269 - DNA, 294 bp, synthetic construct
  misc_feature 1..294: note = Description of sequence: Construct Name: FKBP-235; Mutation(s): Q72R Q84E A117W V85R DRD D90T M54A
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctggacgcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 270 - protein, 98 aa, synthetic construct
  REGION 1..98: note = Description of sequence: Construct Name: FKBP-235; Mutation(s): Q72R Q84E A117W V85R DRD D90T M54A
HCPIKSRKGDVLHAHYTGKLEDGTEFDSSLPRNQPFVFSLGTGERIKGWTQGLLGMCEGEKRKLVIPSELGYGERGWPPKIPGGATLVFEVELLKIER

SEQ ID NO: 271 - DNA, 294 bp, synthetic construct
  misc_feature 1..294: note = Description of sequence: Construct Name: FKBP-236; Mutation(s): Q72R Q84E A117W DRD G113N
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaagtgatcaaaggctgggatcaaggcctgctgggaatgtgcgaaggcgagaagagaaagctggtcatccctagcgaactcggctacaatgaaagaggctggcctccaaagattcctggcggagccacactggtgttcgaagtggaactgctgaagatcgagcga

SEQ ID NO: 272 - protein, 98 aa, synthetic construct
  REGION 1..98: note = Description of sequence: Construct Name: FKBP-236; Mutation(s): Q72R Q84E A117W DRD G113N
HCPIKSRKGDVLHMHYTGKLEDGTEFDSSLPRNQPFVFSLGTGEVIKGWDQGLLGMCEGEKRKLVIPSELGYNERGWPPKIPGGATLVFEVELLKIER

SEQ ID NO: 273 - DNA, 294 bp, synthetic construct
  misc_feature 1..294: note = Description of sequence: Construct Name: FKBP-237; Mutation(s): Q72R Q84E A117W V85R DRD G113N
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacaatgaaagaggctggcctccaaagattcctggcggagccacactggtgttcgaagtggaactgctgaagatcgagcga

SEQ ID NO: 274 - protein, 98 aa, synthetic construct
  REGION 1..98: note = Description of sequence: Construct Name: FKBP-237; Mutation(s): Q72R Q84E A117W V85R DRD G113N
HCPIKSRKGDVLHMHYTGKLEDGTEFDSSLPRNQPFVFSLGTGERIKGWDQGLLGMCEGEKRKLVIPSELGYNERGWPPKIPGGATLVFEVELLKIER

SEQ ID NO: 275 - DNA, 294 bp, synthetic construct
  misc_feature 1..294: note = Description of sequence: Construct Name: FKBP-238; Mutation(s): Q72R Q84E A117W V85R DRD G113N M54A
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacaatgaaagaggctggcctccaaagattcctggcggagccacactggtgttcgaagtggaactgctgaagatcgagcga

SEQ ID NO: 276 - protein, 98 aa, synthetic construct
  REGION 1..98: note = Description of sequence: Construct Name: FKBP-238; Mutation(s): Q72R Q84E A117W V85R DRD G113N M54A
HCPIKSRKGDVLHAHYTGKLEDGTEFDSSLPRNQPFVFSLGTGERIKGWDQGLLGMCEGEKRKLVIPSELGYNERGWPPKIPGGATLVFEVELLKIER

SEQ ID NO: 277 - DNA, 294 bp, synthetic construct
  misc_feature 1..294: note = Description of sequence: Construct Name: FKBP-239; Mutation(s): Q72R Q84E A117W DRD E61Y
  source 1..294: mol_type = other DNA
cactgcccaatcaagagcagaaaaggcgacgtgctgcacatgcactacaccggcaagctgtatgatggcaccgagttcgatagcagcctgcctagaaaccagcctttcgtgtttagcctcggcaccggcgaagtgatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 278 - protein, 98 aa, synthetic construct; identical to SEQ ID NO: 85
  REGION 1..98: note = Description of sequence: Construct Name: FKBP-239; Mutation(s): Q72R Q84E A117W DRD E61Y

SEQ ID NO: 279 - DNA, 294 bp, synthetic construct
  misc_feature 1..294: note = Description of sequence: Construct Name: FKBP-240; Mutation(s): Q72R Q84E A117W V85R DRD E61Y
  source 1..294: mol_type = other DNA
cactgcccaatcaagagcagaaaaggcgacgtgctgcacatgcactacaccggcaagctgtatgatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 280 - protein, 98 aa, synthetic construct
  REGION 1..98: note = Description of sequence: Construct Name: FKBP-240; Mutation(s): Q72R Q84E A117W V85R DRD E61Y
HCPIKSRKGDVLHMHYTGKLYDGTEFDSSLPRNQPFVFSLGTGERIKGWDQGLLGMCEGEKRKLVIPSELGYGERGWPPKIPGGATLVFEVELLKIER

SEQ ID NO: 281 - DNA, 294 bp, synthetic construct
  misc_feature 1..294: note = Description of sequence: Construct Name: FKBP-241; Mutation(s): Q72R Q84E A117W V85R DRD E61Y M54A
  source 1..294: mol_type = other DNA
cactgcccaatcaagagcagaaaaggcgacgtgctgcacgctcactacaccggcaagctgtatgatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 282 - protein, 98 aa, synthetic construct
  REGION 1..98: note = Description of sequence: Construct Name: FKBP-241; Mutation(s): Q72R Q84E A117W V85R DRD E61Y M54A
HCPIKSRKGDVLHAHYTGKLYDGTEFDSSLPRNQPFVFSLGTGERIKGWDQGLLGMCEGEKRKLVIPSELGYGERGWPPKIPGGATLVFEVELLKIER

SEQ ID NO: 283 - DNA, 294 bp, synthetic construct
  misc_feature 1..294: note = Description of sequence: Construct Name: FKBP-242; Mutation(s): Q72R Q84E A117W DRD P118S
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaagtgatcaaaggctgggatcaaggcctgctgggaatgtgcgaaggcgagaagagaaagctggtcatccctagcgaactcggctacggcgaaagaggctggtcgccaaagattcctggcggagccacactggtgttcgaagtggaactgctgaagatcgagcga

SEQ ID NO: 284 - protein, 98 aa, synthetic construct
  REGION 1..98: note = Description of sequence: Construct Name: FKBP-242; Mutation(s): Q72R Q84E A117W DRD P118S
HCPIKSRKGDVLHMHYTGKLEDGTEFDSSLPRNQPFVFSLGTGEVIKGWDQGLLGMCEGEKRKLVIPSELGYGERGWSPKIPGGATLVFEVELLKIER

SEQ ID NO: 285 - DNA, 294 bp, synthetic construct
  misc_feature 1..294: note = Description of sequence: Construct Name: FKBP-243; Mutation(s): Q72R Q84E A117W V85R DRD P118S
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggtcgccaaagattcctggcggagccacactggtgttcgaagtggaactgctgaagatcgagcga

SEQ ID NO: 286 - protein, 98 aa, synthetic construct
  REGION 1..98: note = Description of sequence: Construct Name: FKBP-243; Mutation(s): Q72R Q84E A117W V85R DRD P118S
HCPIKSRKGDVLHMHYTGKLEDGTEFDSSLPRNQPFVFSLGTGERIKGWDQGLLGMCEGEKRKLVIPSELGYGERGWSPKIPGGATLVFEVELLKIER

SEQ ID NO: 287 - DNA, 294 bp, synthetic construct
  misc_feature 1..294: note = Description of sequence: Construct Name: FKBP-244; Mutation(s): Q72R Q84E A117W V85R DRD P118S M54A
  source 1..294: mol_type = other DNA
cactgcccaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggtcgccaaagattcctggcggagccacactggtgttcgaagtggaactgctgaagatcgagcga

SEQ ID NO: 288 - protein, 98 aa, synthetic construct
  REGION 1..98: note = Description of sequence: Construct Name: FKBP-244; Mutation(s): Q72R Q84E A117W V85R DRD P118S M54A
HCPIKSRKGDVLHAHYTGKLEDGTEFDSSLPRNQPFVFSLGTGERIKGWDQGLLGMCEGEKRKLVIPSELGYGERGWSPKIPGGATLVFEVELLKIER

SEQ ID NO: 289 - DNA, 294 bp, synthetic construct
  misc_feature 1..294: note = Description of sequence: Construct Name: FKBP-245; Mutation(s): Q72R Q84E A117W DRD S69H
  source 1..294: mol_type = other DNA
cactgcccaatcaagagcagaaaaggcgacgtgctgcacatgcactacaccggcaagctggaagatggcaccgagttcgatagccatctgcctagaaaccagcctttcgtgtttagcctcggcaccggcgaagtgatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 290 - protein, 98 aa, synthetic construct
  REGION 1..98: note = Description of sequence: Construct Name: FKBP-245; Mutation(s): Q72R Q84E A117W DRD S69H
HCPIKSRKGDVLHMHYTGKLEDGTEFDSHLPRNQPFVFSLGTGEVIKGWDQGLLGMCEGEKRKLVIPSELGYGERGWPPKIPGGATLVFEVELLKIER

SEQ ID NO: 291 - DNA, 294 bp, synthetic construct
  misc_feature 1..294: note = Description of sequence: Construct Name: FKBP-246; Mutation(s): Q72R Q84E A117W V85R DRD S69H
  source 1..294: mol_type = other DNA
cactgcccaatcaagagcagaaaaggcgacgtgctgcacatgcactacaccggcaagctggaagatggcaccgagttcgatagccatctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 292 - protein, 98 aa, synthetic construct
  REGION 1..98: note = Description of sequence: Construct Name: FKBP-246; Mutation(s): Q72R Q84E A117W V85R DRD S69H
HCPIKSRKGDVLHMHYTGKLEDGTEFDSHLPRNQPFVFSLGTGERIKGWDQGLLGMCEGEKRKLVIPSELGYGERGWPPKIPGGATLVFEVELLKIER

SEQ ID NO: 293 - DNA, 294 bp, synthetic construct
  misc_feature 1..294: note = Description of sequence: Construct Name: FKBP-247; Mutation(s): Q72R Q84E A117W V85R DRD S69H M54A
  source 1..294: mol_type = other DNA
cactgcccaatcaagagcagaaaaggcgacgtgctgcacgctcactacaccggcaagctggaagatggcaccgagttcgatagccatctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 294 - protein, 98 aa, synthetic construct
  REGION 1..98: note = Description of sequence: Construct Name: FKBP-247; Mutation(s): Q72R Q84E A117W V85R DRD S69H M54A
HCPIKSRKGDVLHAHYTGKLEDGTEFDSHLPRNQPFVFSLGTGERIKGWDQGLLGMCEGEKRKLVIPSELGYGERGWPPKIPGGATLVFEVELLKIER

SEQ ID NO: 295 - DNA, 294 bp, synthetic construct
  misc_feature 1..294: note = Description of sequence: Construct Name: FKBP-248; Mutation(s): Q72R Q84E A117W DRD S68D
  source 1..294: mol_type = other DNA
cactgcccaatcaagagcagaaaaggcgacgtgctgcacatgcactacaccggcaagctggaagatggcaccgagttcgatgacagcctgcctagaaaccagcctttcgtgtttagcctcggcaccggcgaagtgatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 296 - protein, 98 aa, synthetic construct
  REGION 1..98: note = Description of sequence: Construct Name: FKBP-248; Mutation(s): Q72R Q84E A117W DRD S68D
HCPIKSRKGDVLHMHYTGKLEDGTEFDDSLPRNQPFVFSLGTGEVIKGWDQGLLGMCEGEKRKLVIPSELGYGERGWPPKIPGGATLVFEVELLKIER

SEQ ID NO: 297 - DNA, 294 bp, synthetic construct
  misc_feature 1..294: note = Description of sequence: Construct Name: FKBP-249; Mutation(s): Q72R Q84E A117W V85R DRD S68D
  source 1..294: mol_type = other DNA
cactgcccaatcaagagcagaaaaggcgacgtgctgcacatgcactacaccggcaagctggaagatggcaccgagttcgatgacagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 298 - protein, 98 aa, synthetic construct
  REGION 1..98: note = Description of sequence: Construct Name: FKBP-249; Mutation(s): Q72R Q84E A117W V85R DRD S68D
HCPIKSRKGDVLHMHYTGKLEDGTEFDDSLPRNQPFVFSLGTGERIKGWDQGLLGMCEGEKRKLVIPSELGYGERGWPPKIPGGATLVFEVELLKIER

SEQ ID NO: 299 - DNA, 294 bp, synthetic construct
  misc_feature 1..294: note = Description of sequence: Construct Name: FKBP-250; Mutation(s): Q72R Q84E A117W DRD E130F
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaagtgatcaaaggctgggatcaaggcctgctgggaatgtgcgaaggcgagaagagaaagctggtcatccctagcgaactcggctacggcgaaagaggctggcctccaaagattcctggcggagccacactggtgttctttgtggaactgctgaagatcgagcga

SEQ ID NO: 300 - protein, 98 aa, synthetic construct
  REGION 1..98: note = Description of sequence: Construct Name: FKBP-250; Mutation(s): Q72R Q84E A117W DRD E130F
HCPIKSRKGDVLHMHYTGKLEDGTEFDSSLPRNQPFVFSLGTGEVIKGWDQGLLGMCEGEKRKLVIPSELGYGERGWPPKIPGGATLVFFVELLKIER

SEQ ID NO: 301 - DNA, 294 bp, synthetic construct
  misc_feature 1..294: note = Description of sequence: Construct Name: FKBP-251; Mutation(s): Q72R Q84E A117W V85R DRD E130F
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttctttgtggaactgctgaagatcgagcga

SEQ ID NO: 302 - protein, 98 aa, synthetic construct
  REGION 1..98: note = Description of sequence: Construct Name: FKBP-251; Mutation(s): Q72R Q84E A117W V85R DRD E130F
HCPIKSRKGDVLHMHYTGKLEDGTEFDSSLPRNQPFVFSLGTGERIKGWDQGLLGMCEGEKRKLVIPSELGYGERGWPPKIPGGATLVFFVELLKIER

SEQ ID NO: 303 - DNA, 294 bp, synthetic construct
  misc_feature 1..294: note = Description of sequence: Construct Name: FKBP-252; Mutation(s): Q72R Q84E A117W DRD I136P
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaagtgatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagccagagcga

SEQ ID NO: 304 - protein, 98 aa, synthetic construct
  REGION 1..98: note = Description of sequence: Construct Name: FKBP-252; Mutation(s): Q72R Q84E A117W DRD I136P
HCPIKSRKGDVLHMHYTGKLEDGTEFDSSLPRNQPFVFSLGTGEVIKGWDQGLLGMCEGEKRKLVIPSELGYGERGWPPKIPGGATLVFEVELLKPER

SEQ ID NO: 305 - DNA, 294 bp, synthetic construct
  misc_feature 1..294: note = Description of sequence: Construct Name: FKBP-253; Mutation(s): Q72R Q84E A117W V85R DRD I136P
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagccagagcga

SEQ ID NO: 306 - protein, 98 aa, synthetic construct
  REGION 1..98: note = Description of sequence: Construct Name: FKBP-253; Mutation(s): Q72R Q84E A117W V85R DRD I136P
HCPIKSRKGDVLHMHYTGKLEDGTEFDSSLPRNQPFVFSLGTGERIKGWDQGLLGMCEGEKRKLVIPSELGYGERGWPPKIPGGATLVFEVELLKPER

SEQ ID NO: 307 - DNA, 294 bp, synthetic construct
  misc_feature 1..294: note = Description of sequence: Construct Name: FKBP-254; Mutation(s): Q72R Q84E A117W DRD G99R
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaagtgatcaaaggctgggatcaaggcctgctgggaatgtgcgaacgggagaagagaaagctggtcatccctagcgaactcggctacggcgaaagaggctggcctccaaagattcctggcggagccacactggtgttcgaagtggaactgctgaagatcgagcga

SEQ ID NO: 308 - protein, 98 aa, synthetic construct
  REGION 1..98: note = Description of sequence: Construct Name: FKBP-254; Mutation(s): Q72R Q84E A117W DRD G99R
HCPIKSRKGDVLHMHYTGKLEDGTEFDSSLPRNQPFVFSLGTGEVIKGWDQGLLGMCEREKRKLVIPSELGYGERGWPPKIPGGATLVFEVELLKIER

SEQ ID NO: 309 - DNA, 294 bp, synthetic construct
  misc_feature 1..294: note = Description of sequence: Construct Name: FKBP-255; Mutation(s): Q72R Q84E A117W V85R DRD G99R
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaacgggagaagagaaagctggtcatccctagcgaactcggctacggcgaaagaggctggcctccaaagattcctggcggagccacactggtgttcgaagtggaactgctgaagatcgagcga

SEQ ID NO: 310 - protein, 98 aa, synthetic construct
  REGION 1..98: note = Description of sequence: Construct Name: FKBP-255; Mutation(s): Q72R Q84E A117W V85R DRD G99R
HCPIKSRKGDVLHMHYTGKLEDGTEFDSSLPRNQPFVFSLGTGERIKGWDQGLLGMCEREKRKLVIPSELGYGERGWPPKIPGGATLVFEVELLKIER

SEQ ID NO: 311 - DNA, 294 bp, synthetic construct
  misc_feature 1..294: note = Description of sequence: Construct Name: FKBP-256; Mutation(s): Q72R Q84E A117W V85R DRD E137M
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcatgcga

SEQ ID NO: 312 - protein, 98 aa, synthetic construct
  REGION 1..98: note = Description of sequence: Construct Name: FKBP-256; Mutation(s): Q72R Q84E A117W V85R DRD E137M
HCPIKSRKGDVLHMHYTGKLEDGTEFDSSLPRNQPFVFSLGTGERIKGWDQGLLGMCEGEKRKLVIPSELGYGERGWPPKIPGGATLVFEVELLKIMR

SEQ ID NO: 313 - DNA, 294 bp, synthetic construct
  misc_feature 1..294: note = Description of sequence: Construct Name: FKBP-257; Mutation(s): Q72R Q84E A117W V85R DRD E137F
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatctttcga

SEQ ID NO: 314 - protein, 98 aa, synthetic construct
  REGION 1..98: note = Description of sequence: Construct Name: FKBP-257; Mutation(s): Q72R Q84E A117W V85R DRD E137F
HCPIKSRKGDVLHMHYTGKLEDGTEFDSSLPRNQPFVFSLGTGERIKGWDQGLLGMCEGEKRKLVIPSELGYGERGWPPKIPGGATLVFEVELLKIFR

SEQ ID NO: 315 - DNA, 294 bp, synthetic construct; identical to SEQ ID NO: 301
  misc_feature 1..294: note = Description of sequence: Construct Name: FKBP-258; Mutation(s): Q72R Q84E A117W V85R DRD E130N
  source 1..294: mol_type = other DNA

SEQ ID NO: 316 - protein, 98 aa, synthetic construct; identical to SEQ ID NO: 302
  REGION 1..98: note = Description of sequence: Construct Name: FKBP-258; Mutation(s): Q72R Q84E A117W V85R DRD E130N

SEQ ID NO: 317 - DNA, 294 bp, synthetic construct
  misc_feature 1..294: note = Description of sequence: Construct Name: FKBP-259; Mutation(s): Q72R Q84E A117W V85R DRD E130K
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcaaagtggaactgctgaagatcgagcga

SEQ ID NO: 318 - protein, 98 aa, synthetic construct
  REGION 1..98: note = Description of sequence: Construct Name: FKBP-259; Mutation(s): Q72R Q84E A117W V85R DRD E130K
HCPIKSRKGDVLHMHYTGKLEDGTEFDSSLPRNQPFVFSLGTGERIKGWDQGLLGMCEGEKRKLVIPSELGYGERGWPPKIPGGATLVFKVELLKIER

SEQ ID NO: 319 - DNA, 294 bp, synthetic construct
  misc_feature 1..294: note = Description of sequence: Construct Name: FKBP-260; Mutation(s): Q72R Q84E A117W V85R DRD K101N
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaatagaaagctggtcatccctagcgaactcggctacggcgaaagaggctggcctccaaagattcctggcggagccacactggtgttcgaagtggaactgctgaagatcgagcga

SEQ ID NO: 320 - protein, 98 aa, synthetic construct
  REGION 1..98: note = Description of sequence: Construct Name: FKBP-260; Mutation(s): Q72R Q84E A117W V85R DRD K101N
HCPIKSRKGDVLHMHYTGKLEDGTEFDSSLPRNQPFVFSLGTGERIKGWDQGLLGMCEGENRKLVIPSELGYGERGWPPKIPGGATLVFEVELLKIER

SEQ ID NO: 321 - DNA, 294 bp, synthetic construct
  misc_feature 1..294: note = Description of sequence: Construct Name: FKBP-261; Mutation(s): Q72R Q84E A117W V85R DRD P43R M54A K135Y
  source 1..294: mol_type = other DNA
cactgcagaatcaagagcagaaaaggcgacgtgctgcacgctcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgtacatcgagcga

SEQ ID NO: 322 - protein, 98 aa, synthetic construct
  REGION 1..98: note = Description of sequence: Construct Name: FKBP-261; Mutation(s): Q72R Q84E A117W V85R DRD P43R M54A K135Y
HCRIKSRKGDVLHAHYTGKLEDGTEFDSSLPRNQPFVFSLGTGERIKGWDQGLLGMCEGEKRKLVIPSELGYGERGWPPKIPGGATLVFEVELLYIER

SEQ ID NO: 323 - DNA, 294 bp, synthetic construct
  misc_feature 1..294: note = Description of sequence: Construct Name: FKBP-262; Mutation(s): Q72R Q84E A117W V85R DRD P43R K135Y
  source 1..294: mol_type = other DNA
cactgcagaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgtacatcgagcga

SEQ ID NO: 324 - protein, 98 aa, synthetic construct
  REGION 1..98: note = Description of sequence: Construct Name: FKBP-262; Mutation(s): Q72R Q84E A117W V85R DRD P43R K135Y
HCRIKSRKGDVLHMHYTGKLEDGTEFDSSLPRNQPFVFSLGTGERIKGWDQGLLGMCEGEKRKLVIPSELGYGERGWPPKIPGGATLVFEVELLYIER

SEQ ID NO: 325 - DNA, 294 bp, synthetic construct
  misc_feature 1..294: note = Description of sequence: Construct Name: FKBP-263; Mutation(s): Q72R Q84G A117W V85R DRD P43R K135Y
  source 1..294: mol_type = other DNA
cactgcagaatcaagagcagaaaaggcgacgtgctgcacatgcactacaccggcaagctggaagatggcaccgagttcgatagcagcctgcctagaaaccagcctttcgtgtttagcctcggcaccggcggacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgtacatcgagcga

SEQ ID NO: 326 - protein, 98 aa, synthetic construct
  REGION 1..98: note = Description of sequence: Construct Name: FKBP-263; Mutation(s): Q72R Q84G A117W V85R DRD P43R K135Y
HCRIKSRKGDVLHMHYTGKLEDGTEFDSSLPRNQPFVFSLGTGGRIKGWDQGLLGMCEGEKRKLVIPSELGYGERGWPPKIPGGATLVFEVELLYIER

SEQ ID NO: 327 - DNA, 294 bp, synthetic construct
  misc_feature 1..294: note = Description of sequence: Construct Name: FKBP-264; Mutation(s): Q72R S79W Q84R A117W DRD V85R
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgttttggctcggcaccggcagacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 328 - protein, 98 aa, synthetic construct
  REGION 1..98: note = Description of sequence: Construct Name: FKBP-264; Mutation(s): Q72R S79W Q84R A117W DRD V85R
HCPIKSRKGDVLHMHYTGKLEDGTEFDSSLPRNQPFVFWLGTGRRIKGWDQGLLGMCEGEKRKLVIPSELGYGERGWPPKIPGGATLVFEVELLKIER

SEQ ID NO: 329 - DNA, 294 bp, synthetic construct
  misc_feature 1..294: note = Description of sequence: Construct Name: FKBP-265; Mutation(s): Q72R T82W Q84R A117W DRD V85R
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggctggggcagacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 330 - protein, 98 aa, synthetic construct
  REGION 1..98: note = Description of sequence: Construct Name: FKBP-265; Mutation(s): Q72R T82W Q84R A117W DRD V85R
HCPIKSRKGDVLHMHYTGKLEDGTEFDSSLPRNQPFVFSLGWGRRIKGWDQGLLGMCEGEKRKLVIPSELGYGERGWPPKIPGGATLVFEVELLKIER

SEQ ID NO: 331 - DNA, 294 bp, synthetic construct
  misc_feature 1..294: note = Description of sequence: Construct Name: FKBP-266; Mutation(s): Q72R Q84E A117W V85R DRD D50L
  source 1..294: mol_type = other DNA
cactgcccaatcaagagcagaaaaggccttgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 332 - protein, 98 aa, synthetic construct
  REGION 1..98: note = Description of sequence: Construct Name: FKBP-266; Mutation(s): Q72R Q84E A117W V85R DRD D50L
HCPIKSRKGLVLHMHYTGKLEDGTEFDSSLPRNQPFVFSLGTGERIKGWDQGLLGMCEGEKRKLVIPSELGYGERGWPPKIPGGATLVFEVELLKIER

SEQ ID NO: 333 - DNA, 294 bp, synthetic construct
  misc_feature 1..294: note = Description of sequence: Construct Name: FKBP-267; Mutation(s): Q72R Q84E A117W V85R DRD M96W
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggatggtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 334 - protein, 98 aa, synthetic construct
  REGION 1..98: note = Description of sequence: Construct Name: FKBP-267; Mutation(s): Q72R Q84E A117W V85R DRD M96W
HCPIKSRKGDVLHMHYTGKLEDGTEFDSSLPRNQPFVFSLGTGERIKGWDQGLLGWCEGEKRKLVIPSELGYGERGWPPKIPGGATLVFEVELLKIER

SEQ ID NO: 335 - DNA, 294 bp, synthetic construct
  misc_feature 1..294: note = Description of sequence: Construct Name: FKBP-268; Mutation(s): Q72R Q84E A117W V85R DRD C42R
  source 1..294: mol_type = other DNA
caccgtccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 336 - protein, 98 aa, synthetic construct
  REGION 1..98: note = Description of sequence: Construct Name: FKBP-268; Mutation(s): Q72R Q84E A117W V85R DRD C42R
HRPIKSRKGDVLHMHYTGKLEDGTEFDSSLPRNQPFVFSLGTGERIKGWDQGLLGMCEGEKRKLVIPSELGYGERGWPPKIPGGATLVFEVELLKIER

SEQ ID NO: 337 - DNA, 294 bp, synthetic construct
  misc_feature 1..294: note = Description of sequence: Construct Name: FKBP-269; Mutation(s): Q72R Q84E A117W V85R DRD H53Q
  source 1..294: mol_type = other DNA
cactgcccaatcaagagcagaaaaggcgacgtgctgcagatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 338 - protein, 98 aa, synthetic construct
  REGION 1..98: note = Description of sequence: Construct Name: FKBP-269; Mutation(s): Q72R Q84E A117W V85R DRD H53Q
HCPIKSRKGDVLQMHYTGKLEDGTEFDSSLPRNQPFVFSLGTGERIKGWDQGLLGMCEGEKRKLVIPSELGYGERGWPPKIPGGATLVFEVELLKIER

SEQ ID NO: 339 - DNA, 294 bp, synthetic construct
  misc_feature 1..294: note = Description of sequence: Construct Name: FKBP-270; Mutation(s): Q72R Q84E A117W V85R DRD L52M
  source 1..294: mol_type = other DNA
cactgcccaatcaagagcagaaaaggcgacgtgatgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 340 - protein, 98 aa, synthetic construct
  REGION 1..98: note = Description of sequence: Construct Name: FKBP-270; Mutation(s): Q72R Q84E A117W V85R DRD L52M
HCPIKSRKGDVMHMHYTGKLEDGTEFDSSLPRNQPFVFSLGTGERIKGWDQGLLGMCEGEKRKLVIPSELGYGERGWPPKIPGGATLVFEVELLKIER

SEQ ID NO: 341 - DNA, 294 bp, synthetic construct
  misc_feature 1..294: note = Description of sequence: Construct Name: FKBP-271; Mutation(s): Q72R Q84E A117W V85R DRD K103R
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagacgtctggtcatccctagcgaactcggctacggcgaaagaggctggcctccaaagattcctggcggagccacactggtgttcgaagtggaactgctgaagatcgagcga

SEQ ID NO: 342 - protein, 98 aa, synthetic construct
  REGION 1..98: note = Description of sequence: Construct Name: FKBP-271; Mutation(s): Q72R Q84E A117W V85R DRD K103R
HCPIKSRKGDVLHMHYTGKLEDGTEFDSSLPRNQPFVFSLGTGERIKGWDQGLLGMCEGEKRRLVIPSELGYGERGWPPKIPGGATLVFEVELLKIER

SEQ ID NO: 343 - DNA, 294 bp, synthetic construct
  misc_feature 1..294: note = Description of sequence: Construct Name: FKBP-272; Mutation(s): Q72R Q84E A117W V85R DRD K135L
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgttgatcgagcga

SEQ ID NO: 344 - protein, 98 aa, synthetic construct
  REGION 1..98: note = Description of sequence: Construct Name: FKBP-272; Mutation(s): Q72R Q84E A117W V85R DRD K135L
HCPIKSRKGDVLHMHYTGKLEDGTEFDSSLPRNQPFVFSLGTGERIKGWDQGLLGMCEGEKRKLVIPSELGYGERGWPPKIPGGATLVFEVELLLIER

SEQ ID NO: 345 - DNA, 294 bp, synthetic construct
  misc_feature 1..294: note = Description of sequence: Construct Name: FKBP-273; Mutation(s): Q72R Q84E A117W V85R DRD E114G
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcggtagaggctggcctccaaagattcctggcggagccacactggtgttcgaagtggaactgctgaagatcgagcga

SEQ ID NO: 346 - protein, 98 aa, synthetic construct
  REGION 1..98: note = Description of sequence: Construct Name: FKBP-273; Mutation(s): Q72R Q84E A117W V85R DRD E114G
HCPIKSRKGDVLHMHYTGKLEDGTEFDSSLPRNQPFVFSLGTGERIKGWDQGLLGMCEGEKRKLVIPSELGYGGRGWPPKIPGGATLVFEVELLKIER

SEQ ID NO: 347 - DNA, 294 bp, synthetic construct
  misc_feature 1..294: note = Description of sequence: Construct Name: FKBP-274; Mutation(s): Q72R Q84E A117W V85R DRD E109K
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcaaactcggctacggcgaaagaggctggcctccaaagattcctggcggagccacactggtgttcgaagtggaactgctgaagatcgagcga

SEQ ID NO: 348 - protein, 98 aa, synthetic construct
  REGION 1..98: note = Description of sequence: Construct Name: FKBP-274; Mutation(s): Q72R Q84E A117W V85R DRD E109K
HCPIKSRKGDVLHMHYTGKLEDGTEFDSSLPRNQPFVFSLGTGERIKGWDQGLLGMCEGEKRKLVIPSKLGYGERGWPPKIPGGATLVFEVELLKIER

SEQ ID NO: 349 - DNA, 294 bp, synthetic construct
  misc_feature 1..294: note = Description of sequence: Construct Name: FKBP-275; Mutation(s): Q72R Q84E A117W V85R DRD G49D
  source 1..294: mol_type = other DNA
cactgcccaatcaagagcagaaaagacgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 350 - protein, 98 aa, synthetic construct
  REGION 1..98: note = Description of sequence: Construct Name: FKBP-275; Mutation(s): Q72R Q84E A117W V85R DRD G49D
HCPIKSRKDDVLHMHYTGKLEDGTEFDSSLPRNQPFVFSLGTGERIKGWDQGLLGMCEGEKRKLVIPSELGYGERGWPPKIPGGATLVFEVELLKIER

SEQ ID NO: 351 - DNA, 294 bp, synthetic construct
  misc_feature 1..294: note = Description of sequence: Construct Name: FKBP-276; Mutation(s): Q72R Q74R Q84E A117W V85R
  source 1..294: mol_type = other DNA
cactgcccaatcaagagcagaaaaggcgacgtgctgcacatgcactacaccggcaagctggaagatggcaccgagttcgatagcagcctgcctagaaacagacctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 352 - protein, 98 aa, synthetic construct
  REGION 1..98: note = Description of sequence: Construct Name: FKBP-276; Mutation(s): Q72R Q74R Q84E A117W V85R
HCPIKSRKGDVLHMHYTGKLEDGTEFDSSLPRNRPFVFSLGTGERIKGWDQGLLGMCEGEKRKLVIPSELGYGERGWPPKIPGGATLVFEVELLKIER

SEQ ID NO: 353 - DNA, 294 bp, synthetic construct
  misc_feature 1..294: note = Description of sequence: Construct Name: FKBP-277; Mutation(s): Q72R Q74R Q84E A117W V85R K135Y
  source 1..294: mol_type = other DNA
cactgcccaatcaagagcagaaaaggcgacgtgctgcacatgcactacaccggcaagctggaagatggcaccgagttcgatagcagcctgcctagaaacagacctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgtacatcgagcga

SEQ ID NO: 354 - protein, 98 aa, synthetic construct
  REGION 1..98: note = Description of sequence: Construct Name: FKBP-277; Mutation(s): Q72R Q74R Q84E A117W V85R K135Y
HCPIKSRKGDVLHMHYTGKLEDGTEFDSSLPRNRPFVFSLGTGERIKGWDQGLLGMCEGEKRKLVIPSELGYGERGWPPKIPGGATLVFEVELLYIER

SEQ ID NO: 355 - DNA, 294 bp, synthetic construct
  misc_feature 1..294: note = Description of sequence: Construct Name: FKBP-278; Mutation(s): Q72R Q74R Q84E A117W V85R E130F
  source 1..294: mol_type = other DNA
cactgcccaatcaagagcagaaaaggcgacgtgctgcacatgcactacaccggcaagctggaagatggcaccgagttcgatagcagcctgcctagaaacagacctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttctttgtggaactgctgaagatcgagcga

SEQ ID NO: 356 - protein, 98 aa, synthetic construct
  REGION 1..98: note = Description of sequence: Construct Name: FKBP-278; Mutation(s): Q72R Q74R Q84E A117W V85R E130F
HCPIKSRKGDVLHMHYTGKLEDGTEFDSSLPRNRPFVFSLGTGERIKGWDQGLLGMCEGEKRKLVIPSELGYGERGWPPKIPGGATLVFFVELLKIER

SEQ ID NO: 357 - DNA, 294 bp, synthetic construct
  misc_feature 1..294: note = Description of sequence: Construct Name: FKBP-279; Mutation(s): Q72R Q74R Q84E A117W V85R K120I
  source 1..294: mol_type = other DNA
cactgcccaatcaagagcagaaaaggcgacgtgctgcacatgcactacaccggcaagctggaagatggcaccgagttcgatagcagcctgcctagaaacagacctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaatcattcctggcggagccacactggtgttcgaagtggaactgctgaagatcgagcga

SEQ ID NO: 358 - protein, 98 aa, synthetic construct
  REGION 1..98: note = Description of sequence: Construct Name: FKBP-279; Mutation(s): Q72R Q74R Q84E A117W V85R K120I
HCPIKSRKGDVLHMHYTGKLEDGTEFDSSLPRNRPFVFSLGTGERIKGWDQGLLGMCEGEKRKLVIPSELGYGERGWPPIIPGGATLVFEVELLKIER

SEQ ID NO: 359 - DNA, 294 bp, synthetic construct
  misc_feature 1..294: note = Description of sequence: Construct Name: FKBP-280; Mutation(s): Q72R Q74R Q84E A117W V85R K135Y K120I
  source 1..294: mol_type = other DNA
cactgcccaatcaagagcagaaaaggcgacgtgctgcacatgcactacaccggcaagctggaagatggcaccgagttcgatagcagcctgcctagaaacagacctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaatcattcctggcggagccacactggtgttcgaagtggaactgctgtacatcgagcga

SEQ ID NO: 360 - protein, 98 aa, synthetic construct
  REGION 1..98: note = Description of sequence: Construct Name: FKBP-280; Mutation(s): Q72R Q74R Q84E A117W V85R K135Y K120I
HCPIKSRKGDVLHMHYTGKLEDGTEFDSSLPRNRPFVFSLGTGERIKGWDQGLLGMCEGEKRKLVIPSELGYGERGWPPIIPGGATLVFEVELLYIER

SEQ ID NO: 361 - DNA, 294 bp, synthetic construct
  misc_feature 1..294: note = Description of sequence: Construct Name: FKBP-281; Mutation(s): Q72R Q74R Q84E A117W V85R E130F K120I
  source 1..294: mol_type = other DNA
cactgcccaatcaagagcagaaaaggcgacgtgctgcacatgcactacaccggcaagctggaagatggcaccgagttcgatagcagcctgcctagaaacagacctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaatcattcctggcggagccacactggtgttctttgtggaactgctgaagatcgagcga

SEQ ID NO: 362 - protein, 98 aa, synthetic construct
  REGION 1..98: note = Description of sequence: Construct Name: FKBP-281; Mutation(s): Q72R Q74R Q84E A117W V85R E130F K120I
HCPIKSRKGDVLHMHYTGKLEDGTEFDSSLPRNRPFVFSLGTGERIKGWDQGLLGMCEGEKRKLVIPSELGYGERGWPPIIPGGATLVFFVELLKIER

SEQ ID NO: 363 - DNA, 294 bp, synthetic construct
  misc_feature 1..294: note = Description of sequence: Construct Name: FKBP-282; Mutation(s): Q72R Q74W Q84E A117W V85R
  source 1..294: mol_type = other DNA
cactgcccaatcaagagcagaaaaggcgacgtgctgcacatgcactacaccggcaagctggaagatggcaccgagttcgatagcagcctgcctagaaactggcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgaagatcgagcga

SEQ ID NO: 364 - protein, 98 aa, synthetic construct
  REGION 1..98: note = Description of sequence: Construct Name: FKBP-282; Mutation(s): Q72R Q74W Q84E A117W V85R
HCPIKSRKGDVLHMHYTGKLEDGTEFDSSLPRNWPFVFSLGTGERIKGWDQGLLGMCEGEKRKLVIPSELGYGERGWPPKIPGGATLVFEVELLKIER

SEQ ID NO: 365 - DNA, 294 bp, synthetic construct
  misc_feature 1..294: note = Description of sequence: Construct Name: FKBP-283; Mutation(s): Q72R Q74W Q84E A117W V85R K135Y
  source 1..294: mol_type = other DNA
cactgcccaatcaagagcagaaaaggcgacgtgctgcacatgcactacaccggcaagctggaagatggcaccgagttcgatagcagcctgcctagaaactggcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgtacatcgagcga

SEQ ID NO: 366 - protein, 98 aa, synthetic construct
  REGION 1..98: note = Description of sequence: Construct Name: FKBP-283; Mutation(s): Q72R Q74W Q84E A117W V85R K135Y
HCPIKSRKGDVLHMHYTGKLEDGTEFDSSLPRNWPFVFSLGTGERIKGWDQGLLGMCEGEKRKLVIPSELGYGERGWPPKIPGGATLVFEVELLYIER

SEQ ID NO: 367 - DNA, 294 bp, synthetic construct
  misc_feature 1..294: note = Description of sequence: Construct Name: FKBP-284; Mutation(s): Q72R Q74W Q84E A117W V85R E130F
  source 1..294: mol_type = other DNA
cactgcccaatcaagagcagaaaaggcgacgtgctgcacatgcactacaccggcaagctggaagatggcaccgagttcgatagcagcctgcctagaaactggcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttctttgtggaactgctgaagatcgagcga

SEQ ID NO: 368 - protein, 98 aa, synthetic construct
  REGION 1..98: note = Description of sequence: Construct Name: FKBP-284; Mutation(s): Q72R Q74W Q84E A117W V85R E130F
HCPIKSRKGDVLHMHYTGKLEDGTEFDSSLPRNWPFVFSLGTGERIKGWDQGLLGMCEGEKRKLVIPSELGYGERGWPPKIPGGATLVFFVELLKIER

SEQ ID NO: 369 - DNA, 294 bp, synthetic construct
  misc_feature 1..294: note = Description of sequence: Construct Name: FKBP-285; Mutation(s): Q72R Q74W Q84E A117W V85R K120I
  source 1..294: mol_type = other DNA
cactgcccaatcaagagcagaaaaggcgacgtgctgcacatgcactacaccggcaagctggaagatggcaccgagttcgatagcagcctgcctagaaactggcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaatcattcctggcggagccacactggtgttcgaagtggaactgctgaagatcgagcga

SEQ ID NO: 370 - protein, 98 aa, synthetic construct
  REGION 1..98: note = Description of sequence: Construct Name: FKBP-285; Mutation(s): Q72R Q74W Q84E A117W V85R K120I
HCPIKSRKGDVLHMHYTGKLEDGTEFDSSLPRNWPFVFSLGTGERIKGWDQGLLGMCEGEKRKLVIPSELGYGERGWPPIIPGGATLVFEVELLKIER

SEQ ID NO: 371 - DNA, 294 bp, synthetic construct
  misc_feature 1..294: note = Description of sequence: Construct Name: FKBP-286; Mutation(s): Q72R Q74W Q84E A117W V85R K135Y K120I
  source 1..294: mol_type = other DNA
cactgcccaatcaagagcagaaaaggcgacgtgctgcacatgcactacaccggcaagctggaagatggcaccgagttcgatagcagcctgcctagaaactggcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaatcattcctggcggagccacactggtgttcgaagtggaactgctgtacatcgagcga

SEQ ID NO: 372 - protein, 98 aa, synthetic construct
  REGION 1..98: note = Description of sequence: Construct Name: FKBP-286; Mutation(s): Q72R Q74W Q84E A117W V85R K135Y K120I
HCPIKSRKGDVLHMHYTGKLEDGTEFDSSLPRNWPFVFSLGTGERIKGWDQGLLGMCEGEKRKLVIPSELGYGERGWPPIIPGGATLVFEVELLYIER

SEQ ID NO: 373 - DNA, 294 bp, synthetic construct
  misc_feature 1..294: note = Description of sequence: Construct Name: FKBP-287; Mutation(s): Q72R Q74W Q84E A117W V85R E130F K120I
  source 1..294: mol_type = other DNA
cactgcccaatcaagagcagaaaaggcgacgtgctgcacatgcactacaccggcaagctggaagatggcaccgagttcgatagcagcctgcctagaaactggcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaatcattcctggcggagccacactggtgttctttgtggaactgctgaagatcgagcga

SEQ ID NO: 374 - protein, 98 aa, synthetic construct
  REGION 1..98: note = Description of sequence: Construct Name: FKBP-287; Mutation(s): Q72R Q74W Q84E A117W V85R E130F K120I
HCPIKSRKGDVLHMHYTGKLEDGTEFDSSLPRNWPFVFSLGTGERIKGWDQGLLGMCEGEKRKLVIPSELGYGERGWPPIIPGGATLVFFVELLKIER

SEQ ID NO: 375 - DNA, 294 bp, synthetic construct
  misc_feature 1..294: note = Description of sequence: Construct Name: FKBP-288; Mutation(s): Q72R Q84E A117W V85R K120I
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaatcattcctggcggagccacactggtgttcgaagtggaactgctgaagatcgagcga

SEQ ID NO: 376 - protein, 98 aa, synthetic construct
  REGION 1..98: note = Description of sequence: Construct Name: FKBP-288; Mutation(s): Q72R Q84E A117W V85R K120I
HCPIKSRKGDVLHMHYTGKLEDGTEFDSSLPRNQPFVFSLGTGERIKGWDQGLLGMCEGEKRKLVIPSELGYGERGWPPIIPGGATLVFEVELLKIER

SEQ ID NO: 377 - DNA, 294 bp, synthetic construct
  misc_feature 1..294: note = Description of sequence: Construct Name: FKBP-289; Mutation(s): Q72R Q84E A117W V85R K120I K135Y
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaatcattcctggcggagccacactggtgttcgaagtggaactgctgtacatcgagcga

SEQ ID NO: 378 - protein, 98 aa, synthetic construct
  REGION 1..98: note = Description of sequence: Construct Name: FKBP-289; Mutation(s): Q72R Q84E A117W V85R K120I K135Y
HCPIKSRKGDVLHMHYTGKLEDGTEFDSSLPRNQPFVFSLGTGERIKGWDQGLLGMCEGEKRKLVIPSELGYGERGWPPIIPGGATLVFEVELLYIER

SEQ ID NO: 379 - DNA, 294 bp, synthetic construct
  misc_feature 1..294: note = Description of sequence: Construct Name: FKBP-290; Mutation(s): Q72R Q84E A117W V85R K120I E130F
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaatcattcctggcggagccacactggtgttctttgtggaactgctgaagatcgagcga

SEQ ID NO: 380 - protein, 98 aa, synthetic construct
  REGION 1..98: note = Description of sequence: Construct Name: FKBP-290; Mutation(s): Q72R Q84E A117W V85R K120I E130F
HCPIKSRKGDVLHMHYTGKLEDGTEFDSSLPRNQPFVFSLGTGERIKGWDQGLLGMCEGEKRKLVIPSELGYGERGWPPIIPGGATLVFFVELLKIER

SEQ ID NO: 381 - DNA, 294 bp, synthetic construct
  misc_feature 1..294: note = Description of sequence: Construct Name: FKBP-291; Mutation(s): Q72R Q84E A117W V85R E130N
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcaatgtggaactgctgaagatcgagcga

SEQ ID NO: 382 - protein, 98 aa, synthetic construct
  REGION 1..98: note = Description of sequence: Construct Name: FKBP-291; Mutation(s): Q72R Q84E A117W V85R E130N
HCPIKSRKGDVLHMHYTGKLEDGTEFDSSLPRNQPFVFSLGTGERIKGWDQGLLGMCEGEKRKLVIPSELGYGERGWPPKIPGGATLVFNVELLKIER

SEQ ID NO: 383 - DNA, 294 bp, synthetic construct
  misc_feature 1..294: note = Description of sequence: Construct Name: FKBP-292; Mutation(s): Q72R Q84E A117W V85R K135Q
  source 1..294: mol_type = other DNA
cactgcccaatcaagagcagaaaaggcgacgtgctgcacatgcactacaccggcaagctggaagatggcaccgagttcgatagcagcctgcctagaaaccagcctttcgtgtttagcctcggcaccggcgaacggatcaaaggctgggatcaaggcctgctgggaatgtgcgaaggcgagaagagaaagctggtcatccctagcgaactcggctacggcgaaagaggctggcctccaaagattcctggcggagccacactggtgttcgaagtggaactgctgcagatcgagcga

SEQ ID NO: 384 - protein, 98 aa, synthetic construct
  REGION 1..98: note = Description of sequence: Construct Name: FKBP-292; Mutation(s): Q72R Q84E A117W V85R K135Q
HCPIKSRKGDVLHMHYTGKLEDGTEFDSSLPRNQPFVFSLGTGERIKGWDQGLLGMCEGEKRKLVIPSELGYGERGWPPKIPGGATLVFEVELLQIER

(19) United States
(12) Patent Application Publication
(10) Pub. No.: US 2025/0186614 A1
(43) Pub. Date: Jun. 12, 2025

(54) COMPACT DRUG RESPONSIVE DOMAINS FOR REGULATION OF FUNCTION/ABUNDANCE AND DELIVERY OF POLYPEPTIDE PAYLOADS

(71) Applicant: OBSIDIAN THERAPEUTICS, INC., Cambridge, MA (US)

(72) Inventors: Dhruv Kam Sethi, Westwood, MA (US); Jan ter Meulen, Cambridge, MA (US); Dexue Sun, Cambridge, MA (US); Kerri-Lynn Sheahan, Cambridge, MA (US); Andres Alvarado, Cambridge, MA (US)

(73) Assignee: OBSIDIAN THERAPEUTICS, INC., Cambridge, MA (US)

(21) Appl. No.: 18/843,910

(22) PCT Filed: Mar. 17, 2023
(86) PCT No.: PCT/US2023/064612

§ 371 Date: Sep. 4, 2024

Related U.S. Application Data
(60) Provisional application No. 63/269,582, filed on Mar. 18, 2022.

(51) Int. Cl. A61K 48/00; C12N 9/22; C12N 9/90; C12N 15/11; C12N 15/86

(52) U.S. Cl. CPC A61K 48/005; C12N 9/22; C12N 9/90; C12N 15/111; C12N 15/86; C12N 2310/20; C12N 2750/14143; C12Y 502/01008

(57) ABSTRACT

Provided herein are engineered, regulatable polypeptides comprising a payload and a drug responsive domains (DRD), wherein the DRD is operably linked to the payload and wherein the DRD is responsive to a ligand. The DRD is optionally FKBP13, either wildtype or a variant thereof.

### CROSS REFERENCE TO RELATED APPLICATION

This application claims priority to U.S. Provisional Application No. 63/269,582, filed Mar. 18, 2022, which is incorporated by reference herein in its entirety.

### SEQUENCE LISTING

A Sequence Listing conforming to the rules of WIPO Standard ST.26 is hereby incorporated by reference. Said Sequence Listing has been filed as an electronic document via Patent Center in ASCII format encoded as XML. The electronic document, created on Jun. 16, 2023, is entitled “108407-1365434-002WO1_ST26”, and is 573 kilobytes in size.

### BACKGROUND

Administration of biological agents by providing genetically modified cells to a subject or by the use of gene editing methods, such as CRISPR/Cas9 methods, shows great promise but present substantial challenges. Means of regulating the genetically modified cells or gene editing agents are needed in order to optimize the therapeutic benefits and to reduce off-target effects. Regulation of payloads in genetically modified cells can be achieved by coupling the payloads to drug responsive domains (DRDs) that bind a ligand such that administration of the ligand regulates the activity of the payload in a dose dependent fashion. The size of the payload and other components within a nucleic acid construct encoding the regulatable payload operably linked to a DRD, however, may preclude use of known DRDs because of the limited capacity within a selected viral or non-viral vector. Thus, regulation of larger payloads, such as gene-editing agents, or transduction using smaller vectors, such as adeno-associated virus (AAV) vectors, present substantial challenges in regulation.

### SUMMARY

This disclosure relates to compositions and systems capable of delivering a regulatable payload using a DRD, as well as methods of making and using the compositions and systems. By way of example, gene-editing functions can be regulated by modifying and/or modulating, among other things, abundance and/or activity of the payload. Abundance and/or activity of a gene-editing payload can be regulated by operably linking the payload to a drug-responsive domain (DRD) responsive to a ligand and providing the ligand to the DRD operably linked to the gene-editing payload. In some instances, the DRD must be tailored such that the nucleic acid encoding the DRD can be accommodated in an expression system, such as a viral vector, along with other nucleic acids encoding additional elements, including components of a gene-editing system. Accordingly, provided herein is a compact DRD and a nucleic acid encoding it.

The compact DRD is optionally an FKBP13 or a variant thereof that is responsive to a ligand such as FK506 (tacrolimus) and/or rapamycin (sirolimus). In the presence of the ligand, the payload abundance or activity increases. Thus, when the payload comprises gene-editing components, the FKBP13 in the presence of the ligand, increases the abundance of the gene-editing components and/or increases the gene-editing activity. In the absence of the ligand, the payload is down-regulated or turned off and gene editing is reduced or eliminated.

Provided herein is a nucleic acid that encodes a payload and an FKBP13 or variant thereof that binds a ligand, wherein, upon expression, the FKBP13 or variant thereof is operably linked to the payload. Optionally the FKBP13 variant includes one or more mutations in a ligand binding site and can further include one or more mutations in a non-ligand binding region. For example, the FKBP13 can be a fragment of FKBP13 (e.g., a C-terminal fragment of less than 100 amino acids). The fragment optionally includes one or more mutations in the ligand binding site and optionally further includes one or more mutations outside the ligand binding site. Optionally, the one or more mutations differentially affect binding of FK5065 and rapamycin. For example, the encoded FKBP13 variant can be designed to modulate the biological activity of the payload in response to FK506 more than in response to rapamycin. Optionally, this differential effect between responses to rapamycin and FK506 is achieved by mutations that reduce the binding affinity of the FKBP13 variant for rapamycin.

The nucleic acid encoding the FKBP13 or variant thereof and the payload can further include or encode additional components, such as one or more promoters or linkers.

The encoded payload can be one or more biological molecules having the desired biological activity. The desired biological activity can be, for example, gene editing. Accordingly, the payload optionally comprises an RNA-guided endonuclease (e.g., a Cas9 endonuclease). The payload optionally further comprises at least one guide RNA. By way of example, provided herein is a nucleic acid encoding FKBP13 or a variant thereof responsive to a ligand, wherein the nucleic acid is configured for packaging in an AAV vector as a single nucleotide sequence that includes a promoter sequence, a guide RNA, and a sequence that encodes a Cas9 endonuclease payload, wherein the promoter sequence, the guide RNA, and the sequence encoding the Cas9 endonuclease comprise at least 3000 base pairs.

Also provided herein is a genetically modified FKBP13, for example, an FKBP13 fragment (e.g., a C-terminal fragment) with one or more mutations in the ligand binding site and/or one or more mutations in a non-ligand binding site. The genetically modified FKBP13 fragment optionally has a lower binding affinity for rapamycin and/or a higher binding affinity for FK506 than a control FKBP13 fragment without mutations.

Provided herein is a recombinant polypeptide comprising a DRD and a payload, wherein the payload is operably linked to the FKBP13 or variant thereof and wherein activity of the payload in a biological system (e.g., mammalian blood) is regulatable by the concentration of the ligand contacting the FKBP13. Optionally, the EC₅₀ of FK506 for the FKBP13 or variant thereof in the biological system is about 2-fold to about 20-fold lower than the Cₘₐₓ of FK506 in the biological system.

Further provided are vectors comprising the expressible nucleic acids and cells comprising the vectors or expressible nucleic acids as described herein. By way of example, the vector or cell can include a nucleotide sequence that encodes an RNA-guided endonuclease, one or more guide RNA sequences comprising a first nucleotide sequence that hybridizes to a target DNA in the genome of a cell and a second nucleotide sequence configured to interact with the RNA-guided endonuclease. Optionally the vector is a viral vector (e.g., an AAV vector).

Also provided are methods of producing a recombinant cell by introducing the vector into a target cell. Further provided are methods of regulating a payload in the cell by contacting the target cell comprising the vector with a ligand. When the payload comprises the components for gene editing, the method or regulation provides regulation of gene editing by contacting the target cell comprising the vector with a ligand to modify the DNA expressed in the cell.

For example, the method of modifying target DNA in a cell includes introducing into the cell a vector comprising (1) an RNA-guided endonuclease-encoding nucleic acid comprising a sequence that encodes an FKBP13 or variant thereof responsive to a ligand, a promoter sequence, and a sequence that encodes an RNA-guided endonuclease, and (2) one or more guide RNAs comprising a nucleotide sequence that hybridizes to a target DNA in the genome of a cell and a nucleotide sequence configured to interact with the RNA-guided endonuclease. The method further comprises contacting the vector-containing cell with an effective amount of ligand to induce or increase activity of the RNA-guided endonuclease, wherein the RNA-guided endonuclease interacts with the guide RNA and wherein the RNA-guided endonuclease specifically binds and cleaves the target DNA in the cell. Also provided are methods of treating a disease or disorder responsive to genetic modification in a subject in need thereof by introducing into one or more cells of the subject the vector and contacting the vector-containing one or more cells with an effective amount of ligand to induce or increase activity of the RNA-guided endonuclease, wherein the RNA-guided endonuclease interacts with the guide RNA and wherein the RNA-guided endonuclease specifically binds and cleaves the target DNA in one or more cells of the subject. Also provided is a method of treating a disease or disorder responsive to genetic modification in a subject in need thereof by administering to the subject one or more cells comprising the vector and administering to the subject an effective amount of ligand to induce activity of the RNA-guided endonuclease, wherein the RNA-guided endonuclease interacts with the guide RNA and wherein the RNA-guided endonuclease specifically modifies the genome of one or more cells.

Disclosed herein are methods of controlling the dose or duration of administration of a payload to a subject, comprising administering to the subject one or more nucleic acid constructs, vectors, or cells as described herein. The method optionally further includes administering to the subject a selected amount of a ligand to deliver a selected activity of the payload to the subject thereby controlling the activity level of the payload administered by way of the one or more nucleic acid constructs, vectors or cells.

Also disclosed herein are methods of regulating expression of a downstream target protein or peptide of a gene editing process. Such methods can comprise engineering a cell to express an engineered, regulatable oligomer or polypeptide comprising a payload such as a Cas9 protein or transcription factor protein involved in controlling a gene editor, wherein the payload is operably linked to a DRD. In such methods, a nucleic acid construct or vector described herein is administered to a cell (in vivo or in vitro). Optionally, a cell comprising the nucleic acid or vector is administered to a subject to regulate expression of the target protein or peptide by the cell.

The identified embodiments are exemplary only and are therefore non-limiting. The details of one or more non-limiting embodiments of the invention are set forth in the accompanying drawing and the description below. Other embodiments of the invention should be apparent to those of ordinary skill in the art after consideration of the present disclosure.

### BRIEF DESCRIPTION OF THE DRAWINGS

FIG. 1A is a schematic showing the general organization of an exemplary nucleic acid construct comprising (i) a nucleic acid sequence encoding a potential FKBP DRD sequence; (ii) a nucleic acid sequence encoding Aequorea coerulescens GFP (AcGFP); (iii) a nucleic acid sequence encoding an exemplary self-cleaving peptide (porcine teschovirus-1 2A (P2A)); and (iv) a nucleic acid sequence encoding mCherry.

FIG. 1B provides fluorescence activated cell sorting (FACS) data for tacrolimus dose response studies in transiently transfected HEK293 cells. Several FKBP13 mutations (D90G, M54A and M96V) were effective in destabilizing AcGFP and reducing AcGFP fluorescence.

FIG. 2 is a graph showing tacrolimus dose response curves for FKBP13 constructs designated FKBP-039, FKBP-040, FKBP-041, FKBP-042, FKBP-043, FKBP-044, and FKBP-045 stably transduced into Jurkat cells.

FIG. 3 is a superimposition of rapamycin and tacrolimus represented as sticks.

FIG. 4 is a graph showing tacrolimus dose response fluorescence-activated cell sorting (FACS) data for Jurkat cells stably transduced with FKBP13 constructs designated FKBP-044, FKBP-052, FKBP-053, FKBP-054, FKBP-055, FKBP-056, FKBP-057, FKBP-058, FKBP-059, FKBP-060, FKBP-061, FKBP-062, FKBP-063, or FKBP-064.

FIG. 5 is a graph showing tacrolimus dose response curves for FKBP13 constructs designated FKBP-044, FKBP-066, FKBP-067, FKBP-068, FKBP-069, FKBP-070, FKBP-071, and FKBP-072 transiently transfected into HEK293 cells and EC₅₀ values for each construct.

FIG. 6 is a graph showing tacrolimus dose response curves for FKBP13 constructs designated FKBP-044, FKBP-073, FKBP-074, FKBP-075, FKBP-076, and FKBP-077, transiently transfected into HEK293 cells and EC₅₀ values for each construct.

FIG. 7 is a graph showing tacrolimus dose response curves for FKBP13 constructs designated FKBP-044, FKBP-083, FKBP-084, FKBP-085, and FKBP-086 transiently transfected into HEK293 cells and EC₅₀ values for each construct.

FIG. 8 is a graph showing tacrolimus dose response curves for FKBP13 constructs designated FKBP-044, FKBP-087, FKBP-088, FKBP-089, FKBP-090, and FKBP-091 transiently transfected into HEK293 cells and EC₅₀ values for each construct.

FIG. 9 is a graph showing tacrolimus dose response curves for FKBP13 constructs designated FKBP-044, FKBP-092, FKBP-093, FKBP-094, FKBP-095, FKBP-096 and FKBP-097 transiently transfected into HEK293 cells and EC₅₀ values for each construct.

FIG. 10 is a graph showing tacrolimus dose response FACS data (GFP mean fluorescent intensity (MFI)) for Jurkat cells stably transduced with FKBP13 constructs designated FKBP-044, FKBP-059, FKBP-068, FKBP-077, FKBP-084, FKBP-085, FKBP-091, FKBP-094, FKBP-095, and FKBP-097.

FIG. 11 is a graph showing tacrolimus dose response curves for FKBP13 constructs designated FKBP-091, FKBP-098, FKBP-099, FKBP-100, and FKBP-101 transiently transfected into HEK293 cells and EC₅₀ values for each construct.

FIG. 12 is a graph showing tacrolimus dose response curves for FKBP13 constructs designated FKBP-091, FKBP-102, FKBP-103, FKBP-104, FKBP-105 and FKBP-106 transiently transfected into HEK293 cells and EC₅₀ values for each construct.

FIG. 13 is a graph showing tacrolimus dose response curves for FKBP13 constructs designated FKBP-091, FKBP-107, FKBP-108, FKBP-109, FKBP-110, and FKBP-111 transiently transfected into HEK293 cells and EC₅₀ values for each construct.

FIG. 14 is a graph showing tacrolimus dose response curves for FKBP13 constructs designated FKBP-091, FKBP-112, FKBP-113, FKBP-114, FKBP-115 and FKBP-116 transiently transfected into HEK293 cells and EC₅₀ values for each construct.

FIG. 15A is a graph showing tacrolimus dose response curves FACS data for FKBP13 constructs designated FKBP-091, FKBP-117, FKBP-118, FKBP-119, FKBP-120, FKBP-121, FKBP-122, and FKBP-123 in stably transduced Jurkat cells as measured using FACS and MFI for GFP.

FIG. 15B is another representation of the tacrolimus dose response curves for FKBP13 constructs designated FKBP-091, FKBP-117, FKBP-118, FKBP-119, FKBP-120, FKBP-121, FKBP-122, and FKBP-123, the data is presented as the ratio of GFP MFI to mCherry MFI.

FIG. 15C is a graph showing tacrolimus dose response curves for FKBP13 constructs designated FKBP-091, FKBP-124, FKBP-125, FKBP-126, FKBP-127, FKBP-128, FKBP-129, and FKBP-130 as measured using FACS and MFI for GFP.

FIG. 15D is another representation of the tacrolimus dose response curves for FKBP13 constructs designated FKBP-091, FKBP-124, FKBP-125, FKBP-126, FKBP-127, FKBP-128, FKBP-129, and FKBP-130, the data is presented as the ratio of GFP MFI to mCherry MFI.

FIG. 15E is a graph showing tacrolimus dose response curves for FKBP13 constructs designated FKBP-091, FKBP-131, FKBP-132, FKBP-133, FKBP-134, FKBP-135, FKBP-136, and FKBP-137 as measured using FACS based MFI for GFP.

FIG. 15F is another representation of the tacrolimus dose response curves for FKBP13 constructs designated FKBP-091, FKBP-131, FKBP-132, FKBP-133, FKBP-134, FKBP-135, FKBP-136, and FKBP-137, the data is presented as the ratio of GFP MFI to mCherry MFI.

FIG. 15G is a graph showing tacrolimus dose response curves for FKBP13 constructs designated FKBP-091, FKBP-138, FKBP-139, FKBP-140, FKBP-141, FKBP-142, FKBP-143, and FKBP-144 as measured using FACS based MFI for GFP.

FIG. 15H is another representation of the tacrolimus dose response curves for FKBP13 constructs designated FKBP-091, FKBP-138, FKBP-139, FKBP-140, FKBP-141, FKBP-142, FKBP-143, and FKBP-144, the data is presented as the ratio of GFP MFI to mCherry MFI.

FIG. 15I is a graph showing tacrolimus dose response curves for FKBP13 constructs designated FKBP-091, FKBP-145, FKBP-146, FKBP-147, FKBP-148, FKBP-149, and FKBP-150 as measured using FACS based MFI for GFP.

FIG. 15J is another representation of the tacrolimus dose response curves for FKBP13 constructs designated FKBP-091, FKBP-145, FKBP-146, FKBP-147, FKBP-148, FKBP-149, and FKBP-150, the data is presented as the ratio of GFP MFI to mCherry MFI.

FIG. 16A is a graph showing the tacrolimus (T) vs. rapamycin(S) response dose curves for FKBP13 constructs designated FKBP-151, FKBP-152, and FKBP-153 as measured using FACS based MFI for GFP.

FIG. 16B is another representation of the tacrolimus (T) vs. rapamycin(S) response dose curves for FKBP13 constructs designated FKBP-151, FKBP-152, and FKBP-153, the data is presented as the ratio of GFP MFI to mCherry MFI.

FIG. 17A is a graph showing the tacrolimus (T) vs. rapamycin(S) response dose curves for FKBP13 constructs designated FKBP-153, FKBP-155, and FKBP-157 as measured using FACS based MFI for GFP.

FIG. 17B is another representation of the tacrolimus (T) vs. rapamycin(S) response dose curves for FKBP13 constructs designated FKBP-153, FKBP-155, and FKBP-157, the data is presented as the ratio of GFP MFI to mCherry MFI.

FIG. 18A is a graph showing the tacrolimus (T) vs. rapamycin(S) response dose curves for FKBP13 constructs designated FKBP-154, FKBP-155, and FKBP-156 as measured using FACS based MFI for GFP.

FIG. 18B is another representation of the tacrolimus (T) vs. rapamycin(S) response dose curves for FKBP13 constructs designated FKBP-154, FKBP-155, and FKBP-156, the data is presented as the ratio of GFP MFI to mCherry MFI.

FIG. 19A is a graph showing the tacrolimus (T) vs. rapamycin(S) response dose curves for FKBP13 constructs designated FKBP-157, FKBP-158, and FKBP-159 as measured using FACS based MFI for GFP.

FIG. 19B is another representation of the tacrolimus (T) vs. rapamycin(S) response dose curves for FKBP13 constructs designated FKBP-157, FKBP-158, and FKBP-159, the data is presented as the ratio of GFP MFI to mCherry MFI.

FIG. 20A is a graph showing the tacrolimus (T) vs. rapamycin(S) response dose curves for FKBP13 constructs designated FKBP-044, and FKBP-117 as measured using FACS based MFI for GFP.

FIG. 20B is another representation of the tacrolimus (T) vs. rapamycin(S) response dose curves for FKBP13 constructs designated FKBP-044 and FKBP-117, the data is presented as the ratio of GFP MFI to mCherry MFI.

FIG. 21 is a graph showing the tacrolimus dose response curves for FKBP13 constructs designated FKBP-044, FKBP-059, FKBP-091 and AcGFP-001 as measured using FACS based MFI for GFP.

FIG. 22 is a graph showing the tacrolimus dose response curves for FKBP13 constructs designated FKBP-191, FKBP-199, FKBP-197, FKBP-200, FKBP-91 and FKBP-117 as measured using FACS based MFI for GFP.

FIG. 23 is a graph showing the tacrolimus dose response curves for FKBP13 constructs designated FKBP-201, FKBP-243, FKBP-244, FKBP-251, FKBP-91 and FKBP-117 as measured using FACS based MFI for GFP.

FIG. 24 is a graph showing the tacrolimus dose response curves for FKBP13 constructs designated FKBP-195, FKBP-198, FKBP-209, FKBP-91 and FKBP-117 as measured using FACS based MFI for GFP.

FIG. 25 is a graph showing the tacrolimus dose response curves for FKBP13 constructs designated FKBP-214, FKBP-215, FKBP-220, FKBP-91 and FKBP-117 as measured using FACS based MFI for GFP.

FIG. 26 is a graph showing the tacrolimus dose response curves for FKBP13 constructs designated FKBP-223, FKBP-226, FKBP-240, FKBP-91 and FKBP-117 as measured using FACS based MFI for GFP.

FIG. 27 is a graph showing the tacrolimus dose response curves for FKBP13 constructs designated FKBP-192, FKBP-203, FKBP-228, FKBP-229, FKBP-91 and FKBP-117 as measured using FACS based MFI for GFP.

FIG. 28 is a graph showing the tacrolimus dose response curves for FKBP13 constructs designated FKBP-230, FKBP-233, FKBP-255, FKBP-91 and FKBP-117 as measured using FACS based MFI for GFP.

FIG. 29 is a graph showing the tacrolimus dose response curves for FKBP13 constructs designated FKBP-256, FKBP-257, FKBP-258, FKBP-259, and FKBP-195 as measured using FACS based MFI for GFP.

FIG. 30 is a graph showing the tacrolimus dose response curves for FKBP13 constructs designated FKBP-230, FKBP-233, FKBP-255, FKBP-91 and FKBP-117, the data is presented as the ratio of GFP MFI to mCherry MFI.

FIG. 31 is a graph showing the tacrolimus dose response curves for FKBP13 constructs designated FKBP-260, FKBP-262, FKBP-263, FKBP-265, FKBP-268 and FKBP-195 as measured using FACS based MFI for GFP.

FIG. 32 is a graph showing the tacrolimus dose response curves for FKBP13 constructs designated FKBP-260, FKBP-262, FKBP-263, FKBP-265, FKBP-268 and FKBP-195, the data is presented as the ratio of GFP MFI to mCherry MFI.

FIG. 33 is a graph showing the tacrolimus dose response curves for FKBP13 constructs designated FKBP-270, FKBP-271, FKBP-273, FKBP-274, FKBP-275 and FKBP-195 as measured using FACS based MFI for GFP.

FIG. 34 is a graph showing the tacrolimus dose response curves for FKBP13 constructs designated FKBP-270, FKBP-271, FKBP-273, FKBP-274, FKBP-275 and FKBP-195, the data is presented as the ratio of GFP MFI to mCherry MFI.

FIG. 35 is a graph showing the tacrolimus dose response curves for FKBP13 constructs designated FKBP-276, FKBP-277, FKBP-278, FKBP-279, FKBP-117, FKBP-195 and FKBP-291 as measured using FACS based MFI for GFP.

FIG. 36 is a graph showing the tacrolimus dose response curves for FKBP13 constructs designated FKBP-280, FKBP-281, FKBP-282, FKBP-283, FKBP-117, FKBP-195 and FKBP-291 as measured using FACS based MFI for GFP.

FIG. 37 is a graph showing the tacrolimus dose response curves for FKBP13 constructs designated FKBP-284, FKBP-285, FKBP-286, FKBP-287, FKBP-117, FKBP-195 and FKBP-251 as measured using FACS based MFI for GFP.

FIG. 38 is a graph showing the tacrolimus dose response curves for FKBP13 constructs designated FKBP-288, FKBP-289, FKBP-290, FKBP-291, FKBP-292, FKBP-195, FKBP-251 and FKBP-117 as measured using FACS based MFI for GFP.

FIG. 39 is a graph showing the drug dose response curves for FKBP13 constructs designated FKBP-272 and FKBP-195 as measured using FACS based MFI for GFP.

FIG. 40 is a graph showing the drug dose response curves for FKBP13 constructs designated FKBP-258, FKBP-251, and FKBP-259 as measured using FACS based MFI for GFP.

FIG. 41 is a graph showing the drug dose response curves for FKBP13 constructs designated FKBP-256, FKBP-257, and FKBP-228 as measured using FACS based MFI for GFP.

FIG. 42 is a graph showing the tacrolimus dose response curves for FKBP13 construct designated FKBP-228 as measured using FACS based MFI for GFP. BR1 and BR2 refer to repeated experiment 1 and repeated experiment 2.

FIG. 43 is a graph showing the tacrolimus dose response curves for FKBP13 constructs designated FKBP-256 and FKBP-257 as measured using FACS based MFI for GFP. BR1 and BR2 refer to repeated experiment 1 and repeated experiment 2.

FIG. 44 is a graph showing the tacrolimus dose response curves for FKBP13 constructs designated FKBP-167 and FKBP-168 as measured using FACS based MFI for GFP.

FIG. 45 is a graph showing the tacrolimus dose response curves for FKBP13 constructs designated FKBP-265 and FKBP-264 as measured using FACS based MFI for GFP. BR1 and BR2 refer to repeated experiment 1 and repeated experiment 2.

FIG. 46 is a graph showing the tacrolimus dose response curves for FKBP13 constructs designated FKBP-197 and FKBP-251 as measured using FACS based MFI for GFP.

FIG. 47 is a graph showing the tacrolimus dose response curves for FKBP13 constructs designated FKBP-258 and FKBP-259 as measured using FACS based MFI for GFP. BR1 and BR2 refer to repeated experiment 1 and repeated experiment 2.

FIG. 48 is a graph showing the tacrolimus dose response curves for FKBP13 constructs designated FKBP-215 as measured using FACS based MFI for GFP. BR1 and BR2 refer to repeated experiment 1 and repeated experiment 2.

FIG. 49 is a graph showing the tacrolimus dose response curves for FKBP13 constructs designated FKBP-262 and FKBP-263 as measured using FACS based MFI for GFP. BR1 and BR2 refer to repeated experiment 1 and repeated experiment 2.

FIG. 50 is a graph showing the tacrolimus dose response curves for FKBP13 constructs designated FKBP-195 and FKBP-272 as measured using FACS-based MFI for GFP.

FIG. 51 is a graph showing the tacrolimus dose response curves for FKBP13 construct designated FKBP-195 under different serum concentrations as measured using FACS based MFI for GFP.

FIG. 52 is a graph showing the tacrolimus dose response curves for FKBP13 construct designated FKBP-251 under different serum concentrations as measured using FACS based MFI for GFP.

FIG. 53 is a graph showing the tacrolimus dose response curves for FKBP13 construct designated FKBP-195 under different serum concentrations as measured using FACS based MFI for GFP.

FIG. 54 is a graph showing the tacrolimus dose response curves for FKBP13 construct designated FKBP-251 under different serum concentrations as measured using FACS based MFI for GFP.

### DETAILED DESCRIPTION

The present disclosure provides compositions and systems for regulatory control of a payload. Abundance and/or activity of the payload can be regulated by operably linking the payload to a drug-responsive domain (DRD) responsive to a ligand and providing the ligand to the DRD operably linked to the payload such that abundance and/or activity of the payload is increased in the presence of the ligand in a dose-dependent fashion. The DRD is optionally an FKBP13 or a variant thereof that is responsive to a ligand such as FK506 (tacrolimus) and/or rapamycin (sirolimus). By way of example, when the payload comprises gene-editing components, the FKBP13 in the presence of the ligand, increases the abundance of the gene-editing components and/or increases the gene-editing activity. In the absence of the ligand, the payload abundance is down-regulated and gene editing is consequently reduced or eliminated. Thus, provided herein are regulatable polypeptides comprising a payload, a DRD, and optionally one or more additional components (e.g., linkers, hinges and transmembrane domains, tails, tags); nucleic acids encoding the regulatable polypeptides; vectors comprising the nucleic acids that encode the regulatable polypeptides; cells comprising the nucleic acids or vectors described herein; and methods of making and using the regulatable polypeptides (e.g., methods of gene editing and methods of treating subjects with the regulated payload).

### Compositions

### Engineered, Regulatable Polypeptides

Provided herein are engineered, regulatable polypeptides having at least one payload and at least one FKBP13 drug responsive domain (DRD), wherein the FKBP13 DRD is operably linked to a payload, and wherein the FKBP13 DRD is responsive to a ligand. The activity level of the payload ranges from a basal activity level in the absence of the ligand to a maximum activity level in the presence of a saturating amount of the ligand. Saturating amount of the ligand refers to any amount of ligand at or above the amount that results in the maximum abundance and/or activity of the payload. By way of example, the activity level of the at least one payload ranges from a basal activity level in the absence of the ligand to a maximum activity level in the presence of a saturating amount of the ligand.

Without meaning to be limited by theory, DRDs are thought to be unstable polypeptides that degrade in the absence of their corresponding stabilizing ligand (also referred to as the paired ligand or ligand), but whose stability is rescued by binding to the stabilizing ligand. Because binding of the ligand to the DRD is reversible, later removal of the ligand could result in the DRD unfolding, becoming unstable, and ultimately being tagged for degradation by the ubiquitin-proteasome system (“UPS”). Accordingly, it is believed that when a DRD, for example, an FKBP13 DRD, is operably linked to a payload, the entire construct (i.e., DRD plus payload) is rendered unstable and is degraded by the UPS. However, in the presence of the paired ligand, the construct is stabilized, and the payload remains available for use. Further, it is believed that the conditional nature of DRD stability allows a rapid and non-perturbing switch from stable polypeptide to unstable UPS substrate, which may facilitate regulation or modulation of a payload's activity level.

Because the abundance and availability of a payload are related to the activity of a payload, for purposes of this disclosure, the terms abundance, availability, activity, and the phrase abundance and/or activity (and similarly level of abundance, level of availability, level of activity, and level of abundance and/or activity) are used interchangeably throughout this disclosure and are generally referred to as activity, unless explicitly stated otherwise or nonsensical in context. Further, measurements of abundance or availability are used as a proxy for activity level and may be used herein to reflect the activity level. Consequently, changes in the abundance or availability of a payload in the presence of an effective amount of ligand as compared to in the absence of ligand optionally serves as a proxy for measuring changes in activity level.

Example components (building blocks) of the regulatable polypeptides described herein are referenced throughout this disclosure and provided below. Any of the polypeptides described herein can include at least a payload, for example, a gene editing payload, and a DRD. Optionally, the polypeptide can include one or more additional components such as linkers, hinges (e.g., sheddable and non-sheddable hinges), tails (e.g., cytoplasmic tails), and transmembrane domains. As described below in the section related to methods of making, one of skill in the art may select from the various components using guideposts provided below to achieve a desired outcome (e.g., location of the payload or DRD relative to the cell in which it is expressed and the desired activity of the payload).

### DRDs

DRDs interact with a ligand such that, when the DRD is operatively linked to a payload, it confers ligand-dependent reversible regulation of a characteristic of the payload (for example, activity). Although referred to as drug responsive domains, the ligand to which a DRD is responsive need not be a drug. Suitable FKBP13 DRDs (and their paired ligands), which may be referred to as destabilizing domains or ligand binding domains, are provided throughout the specification.

The FKBP13 DRD, by way of example, can be an amino acid sequence comprising a wildtype FKBP13 DRD or a variant (e.g., genetically modified) FKBP13 DRD that is responsive to a ligand. Optionally, the FKBP13 DRD is a full-length wild-type FKBP13 polypeptide (SEQ ID NO: 1) or a fragment (i.e., a DRD functional fragment that binds the ligand) thereof. By way of example, the FKBP13 DRD can be a fragment of FKBP13, for example, a C-terminal fragment of SEQ ID NO: 1, such as SEQ ID NO: 2. In some embodiments, the C-terminal fragment comprises less than 100 amino acids. In some embodiments, the fragment comprises SEQ ID NO: 2, or an amino acid sequence having at least 85%, 86%, 87%, 88%, 89%, 90%, 91%, 92%, 93%, 94%, 95%, 96%, 97%, 98%, 99% or 100% identity to SEQ ID NO: 2.

The genetically modified FKBP13 optionally comprises one or more mutations relative to a wildtype FKBP13 DRD (SEQ ID NO: 1). Optionally, the genetically modified FKBP13 is a C-terminal fragment of SEQ ID NO: 1 of less than 100 amino acids comprising an amino acid sequence selected from the group consisting of SEQ ID NOs: 3-109, or an amino acid sequence having at least 85%, 86%, 87%, 88%, 89%, 90%, 91%, 92%, 93%, 94%, 95%, 96%, 97%, 98%, 99% or 100% identity to an amino acid sequence selected from the group consisting of SEQ ID NOs: 3-109. One or more genetic modifications, for example, mutations (including truncations, substitutions, and deletions) in the amino acid sequence of FKBP13 can be advantageous, as outlined in the Examples.

The term identity or substantial identity, as used in the context of a polypeptide or polynucleotide sequence described herein, refers to a sequence that has at least 80% sequence identity to a reference sequence. Alternatively, percent identity can be any integer from 80% to 100%. Exemplary embodiments include at least 80%, 85%, 90%, 91%, 92%, 93%, 94%, 95%, 96%, 97%, 98%, or 99%, as compared to a reference sequence using programs known to those of skill in the art, for example, BLAST, using standard parameters, as described below.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window, as used herein, includes reference to a segment of any one of the number of contiguous positions selected from the group consisting of from 20 to 600, about 20 to 50, about 20 to 100, about 50 to about 200 or about 100 to about 150, in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10-20.

In some embodiments, the genetically modified FKBP13 comprises one or more mutations in a binding site for FK506. In some embodiments, the genetically modified FKBP13 fragment has a lower binding affinity for rapamycin than a control FKBP13 fragment, for example, a control FKBP13 fragment lacking certain mutations in the binding site or outside the binding site such that three-dimensional structure of the binding site is altered. In some embodiments, the genetically modified FKBP13 fragment has a higher binding affinity for FK506 than a control FKBP13 fragment without mutations in the ligand binding site. In some embodiments, the genetically modified FKBP13 fragment has a higher binding affinity for FK506 than a control FKBP13 fragment and, optionally, has lower binding affinity for rapamycin than a control FKBP13 fragment.

Numerous FKBP13 DRDs are described herein, but one of skill in the art can identify additional FKBP13 DRDs suitable for use in regulatable compositions according to this disclosure. By way of example, FKBP13 DRDs can be identified using library screening and structure-guided engineering to select the optimal FKBP13 DRD variant with sufficient instability in the absence of the ligand and sufficient stability in the presence of the ligand. A variant library can be generated using random mutagenesis screening by transducing cells (e.g., Jurkat cells) with mutant FKBP13 DRD candidates. To produce an enriched library, cells with the desired characteristics (low basal activity/expression and high dynamic range) are selected by testing polypeptide abundance across a range of concentrations of ligand. Single cell clones are then produced and characterized to identify candidate FKBP13 DRDs. The FKBP13 DRDs described herein are responsive to a paired ligand (also referred to as a stabilizing ligand or simply as a ligand.

Optionally, the EC₅₀ of FK506 for the FKBP13 or variant thereof in the biological system is about 2-fold to about 20-fold lower than the Cₘₐₓ of FK506 in the biological system. Biological systems include, but are not limited to, tissues, cells, or blood (e.g., mammalian blood).

### Payloads

By way of example, the payload can be any polypeptide or polypeptides having a desired biological function. Such payloads can be modified polypeptides, such as glycosylated polypeptides or lipopeptides, or any active portion thereof. For example, a payload can be an RNA-guided endonuclease, for example, a Cas9 polypeptide or an active portion thereof.

In some embodiments, payloads of the present disclosure may be one or more components of a gene editing system. In such embodiments, the engineered, regulatable polypeptide regulates activity of the gene editing system and consequently expression of a downstream target protein. A target protein, as used herein, refers to a protein selected for gene editing, including, for example, a protein having a genetic mutation that results in a deleterious effect in a subject or in a cell. Any of the polypeptides described herein comprising one or more components of a gene editing system can be used to modify the genome of a cell, in vitro or in vitro, to alter expression and/or activity of a target protein in the cell.

When the payloads include an RNA-guided endonuclease, any one of several endonucleases can be selected. For example, the selected RNA-guided endonuclease can be a Cas protein (CRISPR-associated protein) such as Cas9 and Cas12. The CRISPR/Cas system refers to a widespread class of bacterial systems for defense against foreign nucleic acids. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

As used herein, the term Cas9 refers to an RNA-mediated endonuclease (e.g., of bacterial or archeal origin, or derived/modified therefrom). Exemplary RNA-mediated nucleases in the payload include the Cas9 proteins and homologs thereof. Other RNA-mediated nucleases include Cpf1 (Cas12a) (See, e.g., Zetsche et al., Cell, Volume 163, Issue 3, p759-771, 22 Oct. 2015) and homologs thereof. It is understood that in any of the embodiments described herein, a Cas9 nuclease can be subsitututed with a Cpf1 nuclease or any other guided nuclease.

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi et al., RNA Biol. 2013 May 1; 10 (5): 726-737; Makarova et al., Nat. Rev. Microbiol. 2011 June; 9 (6): 467-477; Hou et al., Proc Natl Acad Sci USA. 2013 Sep. 24; 110 (39):15644-9; Sampson et al., Nature. 2013 May 9; 497 (7448): 254-7; and Jinek et al., Science. 2012 Aug. 17; 337 (6096): 816-21. Variants of any of the Cas9 nucleases provided herein can be optimized for efficient activity or enhanced stability in the host cell. Thus, engineered Cas9 nucleases are also contemplated. See, for example, Slaymaker et al., Science 351 (6268): 84-88 (2016)).

In some cases, the Cas9 protein is a nickase, such that when bound to target nucleic acid as part of a complex with a guide RNA, a single strand break or nick is introduced into the target nucleic acid. A pair of Cas9 nickases, each bound to a structurally different guide RNA, can be targeted to two proximal sites of a target genomic region and thus introduce a pair of proximal single stranded breaks into the target genomic region. Exemplary Cas9 nickases include Cas9 nucleases having a D10A or H840A mutation (See, for example, Ran et al., Cell 154 (6): 1380-1389 (2013)).

The RNA-guided endonuclease, for example, a Cas9 endonuclease, can also be an inactive Cas9, for example, dCas9. As used herein, a dCas9 polypeptide is a deactivated or nuclease-dead Cas9 that has been modified to inactivate Cas9 nuclease activity. Modifications include, but are not limited to, altering one or more amino acids to inactivate the nuclease activity or the nuclease domain. Other modifications include removing all or a portion of the nuclease domain of Cas9, such that the sequences exhibiting nuclease activity are absent from Cas9. Accordingly, a dCas9 may include polypeptide sequences modified to inactivate nuclease activity or removal of a polypeptide sequence or sequences to inactivate nuclease activity. The dCas9 retains the ability to bind to DNA even though the nuclease activity has been inactivated. Accordingly, dCas9 includes the polypeptide sequence or sequences required for DNA binding but includes modified nuclease sequences or lacks nuclease sequences responsible for nuclease activity. It is understood that similar modifications can be made to inactivate nuclease activity in other site-directed nucleases, for example, in Cpf1.

Optionally, the polypeptide comprising an RNA-guided endonuclease, for example, a Cas9 endonuclease linked to an FKBP13 DRD described herein, is complexed with one or more guide RNAs in vitro, and the resulting complex is introduced into the cell. The complex can optionally include a nucleotide sequence, for example, a DNA template, for insertion at a specific site in the genomic sequence of the cell. As used herein, the phrase introducing in the context of introducing a nucleic acid, a polypeptide, or a complex described herein, refers to the translocation of the nucleic acid sequence, polypeptide, or complex from outside a cell to inside the cell. In some cases, introducing refers to translocation of the nucleic acid, polypeptide or complex from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

As used herein, the phrase modifying in the context of modifying a genome of a cell refers to inducing a structural change in the sequence of the genome at a target genomic region. For example, the modifying can take the form of inserting a nucleotide sequence into the genome of the cell. For example, a nucleotide sequence encoding one or more polypeptides can be inserted into the genomic sequence, at a specific locus, of the cell. Such modifying can be performed, for example, by inducing a double stranded break within a target genomic region, or a pair of single stranded nicks on opposite strands and flanking the target genomic region. Methods for inducing single or double stranded breaks at or within a target genomic region include the use of a Cas9 nuclease domain, or a derivative thereof, and a guide RNA, or pair of guide RNAs, directed to the target genomic region.

Other gene editing payloads include nucleases (e.g., Zinc finger nuclease, TALEN (Transcription activator-like effector-based nucleases), or meganucleases) and/or recombinases, such as a Cre recombinase. As used herein, the term “TALEN” means a protein comprising a Transcription Activator-like (TAL) effector binding domain and a nuclease domain and includes monomeric TALENs that are functional per se as well as others that require dimerization with another monomeric TALEN. The dimerization can result in a homodimeric TALEN when both monomeric TALEN are identical or can result in a heterodimeric TALEN when monomeric TALEN are different. TALENs are often used in pairs but monomeric TALENs are known. A genetic modification made by TALENs or other tools may be, for example, chosen from the list consisting of an insertion, a deletion, insertion of an exogenous nucleic acid fragment, and a substitution. In general, a target DNA site is identified, and a TALEN-pair is created that will specifically bind to the site. The TALEN is delivered to the cell or embryo, e.g., as a protein, mRNA or by a vector that encodes the TALEN. The TALEN cleaves the DNA to make a double-strand break that is then repaired, often resulting in the creation of an indel, or incorporating sequences or polymorphisms contained in an accompanying exogenous nucleic acid that is either inserted into the chromosome or serves as a template for repair of the break with a modified sequence.

Zinc-finger nucleases (ZFNs) are artificial restriction enzymes generated by fusing a zinc finger DNA-binding domain to a DNA-cleavage domain. Zinc finger domains can be engineered to target desired DNA sequences, and this enables zinc-finger nucleases to target unique sequences within complex genomes. By taking advantage of endogenous DNA repair machinery, these reagents can be used to alter the genomes of higher organisms. ZFNs may be used in method of inactivating genes. Materials and methods for using zinc fingers and zinc finger nucleases for making genetically modified animals are disclosed in, e.g., U.S. Pat. No. 8,106,255; U.S. 2012/0192298; U.S. 2011/0023159; and U.S. 2011/0281306.

### Linkers

Numerous linker sequences (linkers) are known in the art. Linkers include, for example, GS linkers, GSG linkers, and GGSG linkers. These linkers are repeats of the subunit one or more times. Thus, a GS linker is a GSn linker where n is a numerical number being 1, 2, 3, 4, 5, 6, 7, 8, 9, 10 or more. Similarly, a GSG linker is a GSGn linker where n is a numerical number being 1, 2, 3, 4, 5, 6, 7, 8, 9, 10, or more. A GGSG linker is a GGSGn linker where n is a numerical number being 1, 2, 3, 4, 5, 6, 7, 8, 9, 10 or more.

### Hinges and Transmembrane Domains

A hinge sequence is a short sequence of amino acids that facilitates flexibility between connected components. The hinge sequence can be any suitable sequence derived or obtained from any suitable molecule. The hinge sequence may be derived from all or part of an immunoglobulin (e.g., IgG1, IgG2, IgG3, IgG4) hinge region, i.e., the sequence that falls between the CH1 and CH2 domains of an immunoglobulin (e.g., an IgG4 Fc hinge), or the extracellular regions of type 1 membrane proteins such as CD8α CD4, CD28 and CD7, which may be a wild-type sequence or a derivative thereof. Some hinge regions include an immunoglobulin CH3 domain or both a CH3 domain and a CH2 domain. In some embodiments, the hinge is derived from a transmembrane domain.

Transmembrane domains, useful in the engineered regulatable polypeptide constructs of the present disclosure can include, for example, a MHC1 transmembrane domain, a CD8α transmembrane domain, a B7-1 transmembrane domain, a CD4 transmembrane domain, a CD28 transmembrane domain, a CTLA-4 transmembrane domain, a PD-1 transmembrane domain, or a human IgG4 Fc region.

### Intracellular/Cytoplasmic or Transmembrane Tails

Optionally, the herein provided polypeptide constructs comprise an intracellular/cytoplasmic or transmembrane tail. Optionally, the intracellular/cytoplasmic or transmembrane tail is a CD8, CD40L, LIGHT, NKG2C, or B7.1 intracellular tail. The absence of a transmembrane region in a construct can be, for example, designed for a secreted payload or a payload with intracellular or intranuclear activity.

### Tags

Optionally, the polypeptides described herein include a tag. Such a tag allows for isolation or detection of the polypeptide or for isolation or detection of the payload. Such tags optionally include fluorescent proteins (e.g., green fluorescent protein), His-tag, HA-tag, Myc tag, FLAG tag, mCherry, CD20, CD34, nerve growth factor receptor (NGFR), truncated NGFR (tNGFR), epidermal growth factor (EGFR), or a truncated EGFR (tEGFR).

### Ligands

Ligands may be any agent that binds to the FKBP13 DRDs of the engineered, regulatable polypeptides described herein, an effective amount of which results in a measurable change in a characteristic (e.g., abundance, availability, activity) of a payload operably linked to the FKBP13 DRD. In some embodiments, ligands may be synthetic molecules. In some embodiments, stabilizing ligands of the present disclosure may be small molecule compounds. Stabilizing ligands are optionally small molecule therapeutic drugs previously approved by a regulatory agency, such as the U.S. Food and Drug Administration (FDA). Optionally, the FKBP13 DRDs are responsive to a paired ligand that is a small molecule drug, such as an FDA-approved small molecule, for example, tacrolimus (FK506) and/or sirolimus (rapamycin). As described herein, the DRD can be modified to affect the binding affinity of the ligand to the DRD. Optionally, the DRD can be modified to differentially affect binding of a first ligand (e.g., tacrolimus) as compared to a second ligand (e.g., sirolimus). For example, the modified DRD may show increased binding affinity for a first ligand and reduced binding affinity for a second ligand as compared to a control unmodified DRD.

### Nucleic Acids

Provided herein are expressible nucleic acid constructs encoding one or more engineered, regulatable polypeptides as described herein. For example, provided herein is a nucleic acid encoding an FKBP13 or a variant (e.g., genetically modified FKBP13) thereof responsive to a ligand and a payload having a biological activity, wherein, upon expression of the nucleic acid, the FKBP13 is operably linked to the payload. Upon expression of the nucleic acid, the FKBP13 is capable of interacting with an effective amount of the ligand to modulate the biological activity of the payload. In some embodiments, the nucleic acid constructs encode a payload (e.g., a gene editing payload) and a DRD, and optionally encode additional components, such as hinges, linkers, transmembrane domains, tags, and intracellular/cytoplasmic and transmembrane tails as described herein. The nucleic acid constructs optionally also encode additional components such as signal sequences and cleavage sites including shedding domains. The constructs optionally further comprise a promoter sequence and other regulatory elements (enhancers, translational control elements (e.g., IRES), and elements that control half-life).

As used throughout, the term nucleic acid refers to deoxyribonucleic acids (DNA) or ribonucleic acids (RNA) and polymers thereof in either single- or double-stranded form. It is understood that, when a DNA sequence is described, its corresponding RNA is also described, wherein thymidine is represented as uridine. Unless otherwise indicated, a particular nucleic acid sequence also implicitly encompasses modified variants thereof, alleles, orthologs, SNPs, and complementary sequences as well as the sequence explicitly indicated.

Optionally the nucleic acid encodes an FKBP13 fragment responsive to the ligand. Optionally, the FKBP13 fragment, for example, a C-terminal fragment, is less than 100 amino acids. Optionally, the encoded FKBP13 fragment is less than 99, 98, 97, 96, 95, 94, 93, 92, 91, or 90 amino acids. Optionally, the encoded FKBP13 fragment is 90-99 amino acids.

In some embodiments, the nucleic acid encodes SEQ ID NO: 1 (i.e., a wildtype FKBP13) or a fragment thereof. Optionally, the nucleic acid encodes a variant of SEQ ID NO: 1 or a fragment thereof. The encoded FKBP13 fragment is optionally SEQ ID NO: 2. In some embodiments, the encoded FKBP13 is a variant of SEQ ID NO: 2 comprising one or more mutations relative to SEQ ID NO: 2, for example, an FKPB13 comprising an amino acid sequence having at least 80%, 85%, 90%, 95%, 99% or 100% identify with any one of SEQ ID NOs: 3-109. In some embodiments, the encoded FKBP13 variant comprises one or more mutations in a ligand binding site. Residues of FKBP13 that are within 4 angstroms of tacrolimus were deemed to be part of the binding site for FKBP13. The binding site of FKBP13 comprises residues Y56, F66, D67, F76, Q84, V85, I86, W89, Y112, R115, K120, I121 and F129

The FKBP13 ligand is optionally FK506 (tacrolimus), rapamycin (sirolimus), or both FK506 (tacrolimus) and rapamycin (sirolimus), such that the encoded FKBP13 binds one or both ligands. In some embodiments, the encoded FKBP13 variant modulates the biological activity of the payload in response to FK506 more than in response to rapamycin by a factor of 5-10×, for example about 7×.

Some nucleic acid sequences described herein encode a payload comprising an RNA-guided endonuclease, for example, a CRISPR/Cas-associated endonuclease. Optionally, the CRISPR/Cas-associated endonuclease is a Cas9 endonuclease or a Cas12 endonuclease. Optionally, the Cas9 endonuclease is a Staphylococcus aureus Cas9 (SaCas9) or Staphylococcus lugdunensis Cas9 (SluCas9). Optionally, the nucleic acid further encodes at least one guide RNA sequence. As used throughout, a guide RNA (gRNA) is a sequence that interacts with a site-specific or targeted nuclease, for example, an RNA-guided endonuclease, and specifically binds to or hybridizes to a target nucleic acid within the genome of a cell, such that the gRNA and the targeted nuclease co-localize to the target nucleic acid in the genome of the cell. Each gRNA includes a DNA targeting sequence or protospacer sequence of about 10 to 50 nucleotides in length that specifically binds to or hybridizes to a target DNA sequence in the genome. For example, the DNA targeting sequence is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the gRNA comprises a crRNA sequence and a transactivating crRNA (tracrRNA) sequence. In some embodiments, the gRNA does not comprise a tracrRNA sequence.

In some embodiments, the RNA-guided endonuclease, for example, a Cas9 protein, can be in an active endonuclease form, such that, when bound to a target nucleic acid as part of a complex with a guide RNA, a double strand break is introduced into the target nucleic acid.

### Vectors and Cells

Also provided herein are vectors for expressing one or more of the nucleic acids. Such a vector can be chosen from viral vectors and non-viral vectors, plasmids, cosmids, and artificial chromosomes. By way of example, the vector can be a viral vector, such as an adeno-associated viral (AAV) vector, a lentiviral vector, a retroviral vector, or an adenoviral vector. The vector optionally comprises nucleic acid sequences that encode transposases and/or nucleases. Optionally, the vector comprises one or more inverted terminal repeats (ITRs).

In some cases, the vector comprises any of the nucleic acid sequences described herein, wherein the encoded payload is an RNA-guided endonuclease and the vector further comprises one or more guide RNA sequences comprising a first nucleotide sequence that hybridizes to a target DNA in the genome of a cell and a second nucleotide sequence configured to interact with the RNA-guided endonuclease.

In some embodiments, the nucleic acid is configured for packaging as a single nucleotide sequence in vector, for example, a viral vector, with a promoter sequence, a guide RNA, and a sequence that encodes a Cas9 endonuclease payload. Optionally, the nucleic acid is configured for packaging as a single nucleotide sequence in an AAV vector with a promoter sequence, a guide RNA, and a sequence that encodes a Cas9 endonuclease payload, and wherein the promoter sequence, the guide RNA, and the sequence encoding the Cas9 comprise at least 3000 base pairs. Such a vector construct, given the limited capacity of the vector and the size of the payload, requires a compact DRD. Nucleic acids encoding a FKBP13 DRD or a variant thereof, such as a fragment of FKBP13 of 100 or fewer amino acids, are particularly well-suited to such a vector construct.

Cells containing one or more nucleic acid constructs or vectors as described herein are provided. Optionally, the cells are immune cells, stem cells, muscle cells, etc. Optionally, the immune cells are primary human T cells, such as T cells derived from human peripheral blood mononuclear cells (PBMCs), PBMCs collected after stimulation with G-CSF, bone marrow, or umbilical cord blood. In some embodiments, the immune cells are tumor infiltrating lymphocytes (TILs), for example collected from a tumor. The immune effector cells may also be NK cells, αβ T cells, iNKT cells, γδ T cells, macrophages, B cells, dendritic cells, myeloid derived progenitor cells, eosinophils, basophils, neutrophils, or Tregs. Optionally, the stem cells are hematopoietic stem cells, human embryonic stem cells, or induced pluripotent stem cells (iPSCs). The cells provided herein are optionally mammalian cells, or, more specifically, human cells.

As used herein, the phrase hematopoietic stem cell refers to a type of stem cell that can give rise to a blood cell. Hematopoietic stem cells can give rise to cells of the myeloid or lymphoid lineages or a combination thereof. Hematopoietic stem cells are predominantly found in bone marrow, although they can be isolated from peripheral blood. Various cell surface markers can be used to identify, sort, or purify hematopoietic stem cells. In some cases, hematopoietic stem cells are identified as c-kit⁺ and lin⁻. In some cases, human hematopoietic stem cells are identified as CD34⁺, CD59⁺, Thy1/CD90⁺, CD38^{lo/-}, C-kit/CD117⁺, lin⁻. In some cases, human hematopoietic stem cells are identified as CD34⁻, CD59⁺, Thy1/CD90⁺, CD38^{lo/-}, C-kit/CD117⁺, lin⁻. In some cases, human hematopoietic stem cells are identified as CD133⁺, CD59⁺, Thy1/CD90⁺, CD38^{lo/-}, C-kit/CD117⁺, lin⁻. In some cases, the hematopoietic stem cells are CD150⁺CD48⁻CD244⁻.

As used herein, the phrase hematopoietic cell refers to a cell derived from a hematopoietic stem cell. The hematopoietic cell may be obtained or provided by isolation from an organism, system, organ, or tissue (e.g., blood or a fraction thereof). Alternatively, a hematopoietic stem cell can be isolated, and the hematopoietic cell obtained or provided by differentiating the stem cell. Hematopoietic cells include cells with limited potential to differentiate into further cell types. Such hematopoietic cells include, but are not limited to, multipotent progenitor cells, lineage-restricted progenitor cells, common myeloid progenitor cells, granulocyte-macrophage progenitor cells, or megakaryocyte-erythroid progenitor cells. Hematopoietic cells include cells of the lymphoid and myeloid lineages, such as lymphocytes, erythrocytes, granulocytes, monocytes, and thrombocytes. In some embodiments, the hematopoietic cell is an immune cell, such as a T cell, B cell, macrophage, a natural killer (NK) cell or dendritic cell. In some embodiments the cell is an innate immune cell.

As used herein, the phrase T cell refers to a lymphoid cell that expresses a T cell receptor molecule. T cells include human alpha beta (aß) T cells and human gamma delta (78) T cells. T cells include, but are not limited to, naïve T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. T cells can be CD4⁺, CD8⁺, or both CD4⁺ and CD8⁺. T cells can also be CD4⁻, CD8⁻, or both CD4⁻ and CD8⁻. T cells can be helper cells, for example, helper cells of type T_{H}1, T_{H}2, T_{H}3, T_{H}9, T_{H}17, or T_{FH}. T cells can be cytotoxic T cells. Regulatory T cells can be FOXP3⁺ or FOXP3⁻. In some cases, the T cell is a CD4⁺CD25^{hi}CD127^{lo} regulatory T cell. In some cases, the T cell is a regulatory T cell selected from the group consisting of type 1 regulatory (Tr1), T_{H}3, CD8+CD28−, Treg17, and Qa-1 restricted T cells, or a combination or sub-population thereof. In some cases, the T cell is a FOXP3⁺ T cell. In some cases, the T cell is a CD4⁺CD25^{lo}CD127^{hi} effector T cell. In some cases, the T cell is a CD4⁺CD25^{lo}CD127^{hi}CD45RA^{hi}CD45RO⁻ naïve T cell. A T cell can be a recombinant T cell that has been genetically manipulated.

As used herein, the phrase primary in the context of a primary cell is a cell that has not been transformed or immortalized. Such primary cells can be cultured, sub-cultured, or passaged a limited number of times (e.g., passaged 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized directly without culturing or sub-culturing. In some cases, the primary cells are stimulated, activated, or differentiated. For example, primary T cells can be activated by contact with (e.g., culturing in the presence of) CD3, CD28 agonists, IL-2, IFN-γ, or a combination thereof.

This disclosure also includes any of the engineered or genetically modified cells, as well as populations of engineered or genetically modified cells produced by any of the methods described herein.

### Methods of Making

The present disclosure provides methods of making nucleic acid constructs and vectors encoding polypeptides of this disclosure, methods of making the polypeptides of this disclosure by expression of nucleic acid constructs, and methods of making cells comprising the nucleic acid constructs or vectors described herein. Generally, a DRD/ligand pair, for example, FKBP13/FK506, is chosen for regulation of the desired payload. Optionally, the ligand is an FDA-approved small molecule drug. In some embodiments, the DRD/ligand pair is chosen such that it is clinically tractable. For example, the pair is selected such that the ligand is capable of regulating the abundance of or regulating the activity of the payload within an acceptable range of the ligand. Such a range is optionally the approved FDA dose range of the ligand. Thereafter, the nucleic acid or vector is designed to encode the building blocks/components of the regulatable polypeptide described herein, which components include in the case of polypeptides at least a payload and an FKBP13 DRD. The nucleic acid or vector may be further designed to encode additional building block/components such as transmembrane domains, linkers, hinges, tags, or other components mentioned herein. One of skill in the art, using this disclosure, can select the appropriate components and order them within the construct to achieve the desired outcome. For example, the order of building blocks influences whether the DRD regulates the payload at the N- or C-terminus.

As another example, linker and linker length may influence constitutive activity level (i.e., basal activity in the absence of ligand) and in certain embodiments, the specific linker and length is chosen to maximize the on state (e.g., maximum activity level in the presence of ligand) while maintaining low basal activity level in the absence of ligand. As yet another example, the specific hinge may allow for conformational changes and thereby influence ligand responsiveness, and the hinge is thus chosen to result in a sufficient dynamic range to obtain a desired range of payload abundance and biologic activity (i.e., an acceptable payload activity range that corresponds to variation in ligand from zero or minimal activity to maximum saturation activity).

In some cases, the nucleic acid constructs are optionally configured to encode payloads associated with an intracellular or transmembrane domain such that the payload is optionally tethered to the cell membrane.

Vehicles/vectors are chosen and designed to deliver the nucleic acid constructs into the desired cell. For example, the vehicles may be chosen from those previously described including viral vectors (such as adeno-associated vectors, lentiviral vectors, retroviral vectors, and), plasmids, cosmids, and artificial chromosomes. Such vectors can be designed to encode transposases, nucleases, and elements that control translation (e.g., IRES). The choice of vector may also influence the choice of various building block components. For example, vectors which demand smaller constructs may require using smaller DRDs. Appropriate components such as promoters, enhancers, multicistronic expression, translation control and half-life control elements are selected to achieve the desired control of payload abundance or activity.

Additionally, nucleic acid constructs are designed for cistronic or multicistronic expression as required for the desired expression of various engineered components. In some embodiments, for multi-cistronic expression, two or more nucleic acids encoding two or more polypeptides, for example, an FKBP13 and payload fusion and a second payload, can be separated by nucleic acid sequences encoding self-cleaving peptides. Examples of self-cleaving peptides include, but are not limited to, self-cleaving viral 2A peptides, for example, a porcine teschovirus-1 (P2A) peptide, a Thosea asigna virus (T2A) peptide, an equine rhinitis A virus (E2A) peptide, or a foot-and-mouth disease virus (F2A) peptide. Self-cleaving 2A peptides allow expression of multiple gene products from a single construct. (See, for example, Chng et al., MAbs 7 (2): 403-412 (2015)). In some embodiments, the nucleic acid construct comprises two or more self-cleaving peptides. In some embodiments, the two or more self-cleaving peptides are all the same. In other embodiments, at least one of the two or more self-cleaving peptides is different.

The cell to which the nucleic acid is delivered is selected based, at least in part, on the ability of the cell to allow expression of the regulatable polypeptides disclosed herein and to allow payload activity in a sufficient dynamic range. Optionally, the cell expresses little or none of the payload in the absence of the provided nucleic acid or vector. In certain embodiments, one of skill in the art would select a cell in need of an increase in payload activity or abundance in a cell that naturally expresses the payload. In certain embodiments, one of skill in the art would select a cell in need of gene editing. In certain embodiments the cell is selected as an effector cell, for example, an immune effector cell.

A person of ordinary skill in the art applying knowledge from this disclosure can build a variety of regulatable polypeptides, as well as nucleic acids and vectors encoding and cells engineered to express the regulatable polypeptides, within the scope of this disclosure beyond those explicitly exemplified herein.

### Methods of Use

Disclosed herein are methods of regulating a payload, for example, methods of regulating abundance, availability and/or activity of a gene editing payload. In some embodiments, the method of regulation is a method of modifying the activity of the payload by engineering a cell to express a payload operably linked to an FKBP13 DRD responsive to a ligand. The activity of the payload (e.g., corresponding to the abundance and/or availability of the payload) is reduced as compared to the activity of a payload in a control cell, for example, a cell engineered to express the payload independent of a DRD.

For example, provided herein is a method of regulating a payload in a cell, comprising introducing into the cell a vector comprising a nucleic acid encoding an FKBP13 or variant thereof responsive to a ligand, wherein the nucleic acid further comprises a promoter sequence and a sequence that encodes the payload. The method further comprises contacting the cell with an effective amount of the ligand such that the desired level of payload activity is achieved.

Optionally, the payload has a biological activity level ranging from a basal activity level in the absence of ligand to a maximum activity in the presence of a saturating amount of ligand, and the method is a method of modulating the activity of a payload comprising contacting a cell engineered to express a payload (e.g., a gene editing payload) operably linked to an FKBP13 DRD with an effective amount of ligand such that the activity of the payload is increased relative to the basal activity level. In some embodiments, the method comprises contacting the cell with a selected amount of ligand, wherein the selected amount of ligand results in a selected activity level of the payload. In certain embodiments, the method comprises alternatively contacting the cell with varying selected amounts of ligand, to achieve varying selected activity levels ranging from the basal level to the maximum level.

The contacting step can occur in vivo or in vitro. The contacting step is optionally performed to achieve a continuous selected activity of the payload (i.e., to achieve a continuous on-state of the payload) or to achieve intermittent activity of the payload (i.e., to provide a pulsed delivery of the payload between an on-state and an off-state). Off-state means the payload activity is the basal activity level. On-state means a selected activity level in the presence of an effective amount of ligand, which is greater than the off-state. Continuous activity of the payload can be achieved by continuous contact of the FKBP13 DRD with an effective amount of ligand or by providing a subsequent contacting step or steps of the FKBP DRD with the ligand, wherein the subsequent contacting step or steps is/are performed before the activity level of the payload from the previous contacting step reaches the basal activity level. Each contacting step can be varied with regard to the amount of ligand such that, when more ligand is used, more payload activity results, and, when less ligand is used, less payload activity results within the dynamic range of the ligand/DRD pair. Thus, the amount of ligand can be varied with subsequent contacting steps to tune up or tune down the amount and/or activity of the payload over time. Each contacting step can also be varied with respect to frequency in order to achieve a desired pattern of activity level.

Also provided is a method of modifying target DNA in a cell by (a) introducing into the cell a vector comprising (1) an RNA-guided endonuclease-encoding nucleic acid comprising a sequence that encodes an FKBP13 or variant thereof responsive to a ligand, a promoter sequence, and a sequence that encodes an RNA-guided endonuclease, and (2) one or more guide RNAs comprising a nucleotide sequence that hybridizes to a target DNA in the genome of a cell and a nucleotide sequence configured to interact with the RNA-guided endonuclease; and (b) contacting the vector-containing cell with an effective amount of ligand to induce activity of the RNA-guided endonuclease, wherein the RNA-guided endonuclease interacts with the guide RNA and wherein the RNA-guided endonuclease specifically binds and cleaves the target DNA in the cell. Any of the methods for modifying a cell described herein can be performed, in vitro or in vivo. Any of the cells described herein can be genetically modified by any of the methods described herein.

### Treatment Methods

Provided herein are methods of delivering a payload operably linked to a DRD to a subject by administering to the subject nucleic acids or vectors as described herein or a cell containing a nucleic acid or vector described herein. Thus, methods of delivering a payload to a subject in need thereof is provided. The payload can be delivered by administering to the subject a nucleic acid construct or a vector as described herein. The payload can be any biologically active payload including, for example, a therapeutically effective gene editing payload. The method results in expression and gene editing in target cells of the subject. Also provided are methods of delivering to a subject one or more cells as that comprises a nucleic acid or vector that encodes a regulatable polypeptide. The one or more cells into which the vector is introduced can be derived from the same subject or a different subject. The one or more cells can be derived from the same or different subject and then expanded in culture prior to and/or after introduction of the vector.

By way of example, provided herein is a method of treating a disease or disorder responsive to genetic modification in a subject in need of genetic modification by (a) introducing into one or more cells of the subject a vector comprising (1) an RNA-guided endonuclease encoding nucleic acid comprising a sequence that encodes an FKBP13 or variant thereof responsive to a ligand, a promoter sequence and a sequence that encodes an RNA-guided endonuclease, and (2) at least one guide RNA comprising a nucleotide sequence that hybridizes to a target DNA in the genome of a cell and a nucleotide sequence configured to interact with the RNA-guided endonuclease; (b) contacting the vector-containing one or more cells with an effective amount of ligand to induce activity of the RNA-guided endonuclease, wherein the RNA-guided endonuclease interacts with the guide RNA and wherein the RNA-guided endonuclease specifically binds and cleaves the target DNA one or more cells of the subject.

Also provided herein is a method of treating a disease or disorder responsive to genetic modification in a subject in need thereof, comprising (a) administering to the subject one or more cells comprising a vector, wherein the vector comprises (1) an RNA-guided endonuclease encoding nucleic acid comprising a sequence that encodes an FKBP13 or variant thereof responsive to a ligand, a promoter sequence and a sequence that encodes an RNA-guided endonuclease, and (2) a guide RNA comprising a nucleotide sequence that hybridizes to a target DNA in the genome of the one or more cells and a nucleotide sequence configured to interact with the RNA-guided endonuclease; (b) administering to the subject an effective amount of ligand to induce activity of the RNA-guided endonuclease, wherein the RNA-guided endonuclease interacts with the guide RNA and wherein the RNA-guided endonuclease specifically modifies the genome of one or more cells.

Any method described herein wherein one or more cells comprising a nucleic acid or vector that encodes a regulatable polypeptide are delivered to the subject can further comprise isolating cells from a subject, transducing the isolated cells with the nucleic acids or vectors encoding the regulatable polypeptides described in this disclosure, expanding the cells in vitro, and providing the cells to the same or different subject. The subject may have a genetic mutation. The cells can be isolated from the same subject (autologous source) that receives the transduced cells or the cells can be isolated from a different subject (e.g., an allogeneic source). In certain embodiments, the cells are administered in an amount from about 1000 cells/injection to up to about 10 billion cells/injection, such as 1×10¹⁰, 1×10⁹, 1×10⁸, 1×10⁷, 5×10⁷, 1×10⁶, 5×10⁶, 1×10⁵, 5×10⁵, 1×10⁴, 5×10⁴, 1×10³, 5×10³ cells per injection, or any ranges between any two of the numbers, end points inclusive. Optionally, from 1×10⁸ to 1×10¹⁰ cells are administered to the subject. Optionally, the cells are administered one, two, three, or four times as needed. In some methods, one or more genetically modified cells described herein or made by a method described herein are administered to the subject.

The methods may further comprise controlling the dose or duration of administration of a payload to a subject by administering to the subject a selected amount of a paired ligand to deliver a selected activity of the payload to the subject. The ligand can be delivered to achieve continuous or intermittent payload activity in the subject. Continuous payload activity may be a substantially consistent level of activity, or the level of activity may be modulated. Intermittent activity, between the off-state and on-state includes modulating activity between the off-state and a substantially consistent on-state, or between the off-state and varying on-state activity levels. A higher dose or longer duration of administration of the ligand is administered when more activity of the payload is desired, and reduction or elimination of the ligand dose is chosen when less activity is desired. The dose and duration of ligand administration and the resulting activity of the payload may be selected to avoid unacceptable or undesired side effects or toxicity in the subject. Dosages of ligand and schedules for administering the dosages of ligand may be determined empirically by one skilled in the art based on the amount of resulting payload, the activity of the payload, or based on one or more signs of the effect of the payload activity. The ranges for administration of the ligand range from any amount above zero to a saturating dose and the resulting payload activity ranges from a basal level to a maximal level, optionally with a sufficient dynamic range that allows for the desired dose-response to the ligand and concomitant activity range for the payload (e.g., for a given ligand and payload, the range of difference in off-state and maximum payload activity would result from at least a 10 fold range of ligand). This sufficient dynamic range allows for fine tuning and a dose response curve that is not unacceptably steep. In embodiments, the dosage or frequency of administration of ligand and resulting abundance and activity of payload is chosen to avoid, mitigate against, or limit unacceptable or undesired adverse side effects and will vary with the age, condition, and/or sex of the subject, and type of condition being treated, the extent of the condition, or, and whether other therapeutic agents are included in the treatment regimen. Guidance can be found in the literature for appropriate dosages for given classes of ligands. Exemplary dosages, for example, clinically approved dosages of tacrolimus for various tissue/organ transplants are 0.1-0.3 mg/kg of body weight. Such dosages can achieve a Cₘₐₓ value of 20-68 ng/ml.

In the treatment methods described herein, the ligand dosage regimen including the selected amount of ligand for administration to the subject and frequency of administration of the selected amount of ligand is chosen to result in regulation of the payload and/or a desired outcome for the subject. The subject is optionally monitored for the outcome. Thus, for example, for treatment of cancer in a subject, the number of malignant cells in a sample, the circulating tumor DNA in a sample, or the size of a solid tumor upon imaging can be detected. If the desired end point is achieved (e.g., showing successful treatment of a genetic mutation), the ligand can be reduced or discontinued so as to reduce or eliminate the gene editing payload, for example to reduce the abundance, availability and/or activity of the payload below a pre-determined threshold to eliminate or mitigate against unwanted or undesired side effects. Similarly, if the subject develops unacceptable off-target effects or other adverse effects from the payload, the ligand can be reduced or discontinued.

Also disclosed herein are methods of regulating expression of a downstream target protein of a gene editing process. In some embodiments, such a method comprises engineering a cell to express an oligomer or engineered, regulatable polypeptide comprising a payload such as a CAS9 protein or transcription factor protein operably linked to a DRD. In certain embodiments, for a subject with a genetic mutation, a nucleic acid construct or vector according to this disclosure is provided to cells of the subject to deliver a payload that provides a nucleic acid editing polypeptide. Target cells in the subject are transduced with the nucleic acid construct or vector to allow the gene editing payload to modify the nucleic acid (e.g., genomic DNA or RNA) of the transduced cell. The activity level of the payload and therefore expression of the target protein can be regulated by administration of ligand to the subject.

Also provided is a method of treating a disease or disorder comprising administering to the subject one or more cells that have been edited using any of the gene editing systems described herein, for example CRISPR/Cas9 editing, to edit the genome of a cell.

Any of the treatment methods described herein can be used to treat a disease or disorder associated with a genetic mutation, for example, but not limited to, Duchenne muscular dystrophy, myotonic dystrophy, cystic fibrosis, sickle cell, beta thalassemia, alpha-1 antitrypsin deficiency, APOL1-mediated kidney disease, or Type 1 diabetes.

### Definitions

Unless defined otherwise, all technical and scientific terms used herein have the same meaning as commonly understood by one of ordinary skill in the art to which this disclosure belongs.

As used in the specification and the appended claims, the singular forms “a,” “an,” and “the” include plural referents unless the context clearly dictates otherwise. For example, reference to “a transcript” or “the transcript” may include a plurality of transcripts.

The use of any and all examples or exemplary language (e.g., “such as”) provided herein, is intended merely to better illustrate the invention and does not pose a limitation on the scope of the invention unless otherwise claimed.

The terms “optional” and “optionally” mean that the subsequently described event, circumstance, or material may or may not occur or be present, and that the description includes instances where the event, circumstance, or material occurs or is present as well as instances where it does not occur or is not present.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of” those certain elements.

As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

The terms “about” and “approximate,” when used to refer to a measurable value such as an amount, concentration, dose, time, temperature, activity, level, number, frequency, percentage, dimension, size, weight, position, length and the like, is meant to account for variations due to experimental error, which could encompass variations of ±15%, ±10%, ±5%, ±1%, ±0.5%, or even ±0.1% of the specified amount, concentration, dose, time, temperature, activity, level, number, frequency, percentage, dimension, size, weight, position, length and the like. All measurements or numbers are implicitly understood to be modified by the word about, even if the measurement or number is not explicitly modified by the word about. In instances in which the terms “about” and “approximate” are used in connection with the location or position of regions within a reference polypeptide, these terms encompass variations of ±up to 20 amino acid residues, ±up to 15 amino acid residues, ±up to 10 amino acid residues, ±up to 5 amino acid residues, ±up to 4 amino acid residues, ±up to 3 amino acid residues, ±up to 2 amino acid residues, or even±1 amino acid residue.

Reference is made herein to a “basal activity level.” Basal level as used herein can be zero, near zero, or any amount in the absence of exogenous ligand. Basal activity may occur because of endogenous levels of the same or different ligand or may occur because of a resting level of payload production in the absence of exogenous ligand.

Reference to “biological activity” is understood to mean under appropriate conditions even if not so stated.

As used herein “contacting” is understood to mean providing an agent (such as a ligand) to a target (such as a DRD) such that the agent and target may come into contact with one another. For example, contacting includes providing a ligand in vitro to a cell (e.g., when the DRD is located extracellularly or on the cell surface). As another example, contacting also includes providing the ligand to a cell, wherein the DRD is located intracellularly, such that the ligand reaches the cytoplasm. Similarly, a cell can be contacted in vivo, by administering a ligand to a subject such that the ligand reaches a cell or DRD.

“DRD” is understood to mean a domain responsive to a ligand, even if not so stated.

The terms “ligand,” “paired ligand,” and “stabilizing ligand” are used interchangeably and mean the same thing when used in reference to a drug responsive domain (“DRD”).

As used herein, “operably linked” means that, in the presence of a paired ligand, the DRD is linked to the payload directly or indirectly so as to alter a measurable characteristic of the payload (e.g., alters the level of activity of the payload as compared to the level of activity in the absence of the paired ligand). In some embodiments, the measured level of amount and/or activity of the payload increases in the presence of an effective amount of ligand as compared to the measured level of expression or activity in the absence of ligand. An effective amount of ligand means the amount of ligand needed to see an increase in the measure of the amount or activity of the payload. In some embodiments, the effective amount is not so great as to produce unacceptable toxicity or off-target effects. Optionally, the measurable characteristic is a therapeutic outcome, an amount of the payload in a sample, or a biological activity level of the payload (for which measuring the amount of payload can serve as a proxy).

The term “payload” refers to the agent whose abundance, activity, availability, expression, function or other characteristic is desired to be regulated by a DRD.

Wherever the phrase “linked” or “bound” or the like is used, the phrase “directly or indirectly” is understood to follow unless explicitly stated otherwise or nonsensical in context.

The details of one or more embodiments of the present disclosure are set forth in the description and accompanying drawings. It is to be understood that other embodiments may be utilized and structural or process changes made without departing from the scope of the disclosure. In other words, illustrative embodiments and aspects are described below. But it will be appreciated that in the development of any such actual embodiment, numerous implementation-specific decisions may be made to achieve the developer's specific goals, such as compliance with system-related and business-related constraints, which may vary from one implementation to another. Moreover, it will be appreciated that such development effort might be complex and time-consuming but would nevertheless be a routine undertaking for those of ordinary skill in the art having the benefit of this disclosure.

Publications cited herein and the material for which they are cited are hereby specifically incorporated by reference in their entireties.

The examples below are intended to further illustrate certain aspects of the methods and compositions described herein and are not intended to limit the scope of the claims.

### EXAMPLES

### Example 1

FKBP13 and fragments thereof were studied to identify DRDs that are responsive to FDA approved drugs. For the purposes of these experiments, tacrolimus was used as an exemplary FDA approved drug. Tacrolimus's binding affinity for FKBP13 is 166 nM, which is substantially lower than the binding affinity for FKBP12, which is 0.2 nM. To increase FKBP13 responsiveness to tacrolimus, the binding site of FKBP13 was targeted with the goal of making the FBKP13 binding site more similar to that of FKBP12. To this end, the tacrolimus bound three dimensional structures of FKBP12 and FKBP13 were compared. Mutations that could increase the drug responsiveness of FKBP13 were designed based on expert visual examination. Library based screening was also conducted. The DRDs corresponding to SEQ ID NOs: 3-109 were derived from library selections. The library was generated as a site-saturation library using the binding site having an A117H variant (Q72R, Q84E, and A117H binding site mutations), where 117H is the residue found in the FKBP12 binding site. While the library was under selection, about five additional binding site mutations were designed, and it was shown that an A117W mutation improved EC₅₀, and likely resulted in a 6-fold increase in binding affinity. When the library output was analyzed, via sequencing, the DRD-genic mutations were made with the A117W version of the binding site. As a control, five of the top FKBP13 mutants were also produced with the A117H sequence that came out of the library. While FKBP-117, 119, 121, 123, 125 were formatted with the improved binding site (A117W; identified in FKBP-091); FKBP-118, 120, 122, 124, 126 are exactly as sequenced from the library and have the A117H mutation in the binding site. The rest of the library output (127-150) was only formatted with the improved binding site mutations (i.e., with Q72R, Q84E, and A117W).

The full-length wildtype FKBP13 sequence, which served as the starting point, is as follows:

(SEQ ID NO: 1)
MRLSWFRVLTVLSICLSAVATATGAEGKRKLQIGVKKRVDHCPIKSRKG
DVLHMHYTGKLEDGTEFDSSLPQNQPFVFSLGTGQVIKGWDQGLLGMCE
GEKRKLVIPSELGYGERGAPPKIPGGATLVFEVELLKIERRTEL.

Table 1 provides exemplary FKBP13 DRD sequences that were used in the constructs described throughout the Examples. A wildtype FKBP13 fragment is designated SEQ ID NO: 2. SEQ ID NOs: 3-109 are examples of genetically modified FKBP13 polypeptides derived from fragment corresponding to SEQ ID NO:2. As used throughout, reference to constructs FKBP-039-FKBP—

TABLE 1-Exem
plary DRD Sequences

| Construct Name | | | |
|---|---|---|---|
| (FKBP13-AcGFP- | FKBP13 DRD in | | |
| mCherry Fusion) | Construct | FKBP13 DRD Sequence | SEQ ID NO: |
| FKBP-039 | FKBP13 (WT) | HCPIKSRKGDVLHMHYTG | 2 |
| | | KLEDGTEFDSSLPQNQPFV | |
| | | FSLGTGQVIKGWDQGLLG | |
| | | MCEGEKRKLVIPSELGYG | |
| | | ERGAPPKIPGGATLVFEVE | |
| | | LLKIER | |
| FKBP-040 | FKBP13 | HCPIKSRKGDVLHMHYTG | 3 |
| | (DRD-D90G) | KLEDGTEFDSSLPQNQPFV | |
| | | FSLGTGQVIKGWGQGLLG | |
| | | MCEGEKRKLVIPSELGYG | |
| | | ERGAPPKIPGGATLVFEVE | |
| | | LLKIER | |
| FKBP-041 | FKBP13 | HCPIKSRKGDVLHAHYTG | 4 |
| | (DRD-M54A) | KLEDGTEFDSSLPQNQPFV | |
| | | FSLGTGQVIKGWDQGLLG | |
| | | MCEGEKRKLVIPSELGYG | |
| | | ERGAPPKIPGGATLVFEVE | |
| | | LLKIER | |
| FKBP-042 | FKBP13 | HCPIKSRKGDVLHMHYTG | 5 |
| | (DRD-M96V) | KLEDGTEFDSSLPQNQPFV | |
| | | FSLGTGQVIKGWDQGLLG | |
| | | VCEGEKRKLVIPSELGYGE | |
| | | RGAPPKIPGGATLVFEVEL | |
| | | LKIER | |
| FKBP-043 | FKBP13 (mutated to | HCPIKSRKGDVLHMHYTG | 6 |
| | match FKBP12 active | KLEDGTEFDSSLPRNQPFV | |
| | site-Q72R, Q84E, | FSLGTGEVIKGWGQGLLG | |
| | A117H) | MCEGEKRKLVIPSELGYG | |
| | (DRD-D90G) | ERGHPPKIPGGATLVFEVE | |
| | | LLKIER | |
| FKBP-044 | FKBP13 (mutated to | HCPIKSRKGDVLHAHYTG | 7 |
| | match FKBP12 active | KLEDGTEFDSSLPRNQPFV | |
| | site-Q72R, Q84E, | FSLGTGEVIKGWDQGLLG | |
| | A117H) | MCEGEKRKLVIPSELGYG | |
| | (DRD-M54A) | ERGHPPKIPGGATLVFEVE | |
| | | LLKIER | |
| FKBP-045 | FKBP13 (mutated to | HCPIKSRKGDVLHMHYTG | 8 |
| | match FKBP12 active | KLEDGTEFDSSLPRNQPFV | |
| | site-Q72R, Q84E, | FSLGTGEVIKGWDQGLLG | |
| | A117H) | VCEGEKRKLVIPSELGYGE | |
| | (DRD-M96V) | RGHPPKIPGGATLVFEVEL | |
| | | LKIER | |
| FKBP-053 | FKBP13 (mutated to | HCPIKSRKGDVLHMHYTG | 9 |
| | match FKBP12 active | KLEDGTEHDSSLPRNQPF | |
| | site-Q72R, Q84E, | VFSLGTGEVIKGWGQGLL | |
| | A117H) | GMCEGEKRKLVIPSELGY | |
| | (DRD-D90G, F66H) | GERGHPPKIPGGATLVFEV | |
| | | ELLKIER | |
| FKBP-054 | FKBP13 (mutated to | HCPIKSRKGDVLHMHYTG | 10 |
| | match FKBP12 active | KLEDGTEFDSSLPRNQPFV | |
| | site-Q72R, Q84E, | FSLGTGEVIKGWGQGLLG | |
| | A117H) | MCEGEKRKLVIPSELGYG | |
| | (DRD-D90G, L127K) | ERGHPPKIPGGATKVFEVE | |
| | | LLKIER | |
| FKBP-055 | FKBP13 mutated to | HCPIKSRKGDVLHMHYTG | 11 |
| | match FKBP12 active | KLEDGTEFDSSLPRNQPFV | |
| | site-(Q72R, Q84E, | VSLGTGEVIKGWDQGLLG | |
| | A117H) | MCEGEKRKLVIPSELGYG | |
| | (DRD-F78V) | ERGHPPKIPGGATLVFEVE | |
| | | LLKIER | |
| FKBP-056 | FKBP13 (mutated to | HCPIKSRKGDVLHMHYTG | 12 |
| | match FKBP12 active | KLEDGTEFDSSLPRNQPFV | |
| | site-Q72R, Q84E, | FSLGTGEVIKGWDQGLLG | |
| | A117H) | MCEGEKRKLVIPSELGYG | |
| | (DRD-F129K) | ERGHPPKIPGGATLVKEVE | |
| | | LLKIER | |
| FKBP-057 | FKBP13 (mutated to | HCPIKSRKGDVLHMHYTG | 13 |
| | match FKBP12 active | KLEDGTEFDSSLPRNQPFV | |
| | site-Q72R, Q84E, | FSLGTGEVIKGWDQGLLG | |
| | A117H) | MCEGEKRKLVAPSELGYG | |
| | (DRD-I106A) | ERGHPPKIPGGATLVFEVE | |
| | | LLKIER | |
| FKBP-058 | FKBP13 (mutated to | HCPIKSRKGDVAHMHYTG | 14 |
| | match FKBP12 active | KLEDGTEFDSSLPRNQPFV | |
| | site-Q72R, Q84E, | FSLGTGEVIKGWDQGLLG | |
| | A117H) | MCEGEKRKLVIPSELGYG | |
| | (DRD-L52A) | ERGHPPKIPGGATLVFEVE | |
| | | LLKIER | |
| FKBP-059 | FKBP13 (mutated to | HCPIKSRKGDVLHMHYTG | 15 |
| | match FKBP12 active | KLEDGTEFDSSLPRNQPFV | |
| | site-Q72R, Q84E, | FSLGTGEVIKGWDQGALG | |
| | A117H) | MCEGEKRKLVIPSELGYG | |
| | (DRD-L93A) | ERGHPPKIPGGATLVFEVE | |
| | | LLKIER | |
| FKBP-060 | FKBP13 (mutated to | HCPIKSRKGDVLHMHYTG | 16 |
| | match FKBP12 active | KLEDGTEFDSSLPRNQPFV | |
| | site-Q72R, Q84E, | FSLGTGEVIKGWDQGLLG | |
| | A117H) | MCEGEKRKAVIPSELGYG | |
| | (DRD-L104A) | ERGHPPKIPGGATLVFEVE | |
| | | LLKIER | |
| FKBP-061 | FKBP13 (mutated to | HCPIKSRKGDVLHMHYTG | 17 |
| | match FKBP12 active | KLEDGTEFDSSLPRNQPFV | |
| | site-Q72R, Q84E, | FSLGTGEVIKGWDQGLLG | |
| | A117H) | MCEGEKRKLVIPSELGYG | |
| | (DRD-L133A) | ERGHPPKIPGGATLVFEVE | |
| | | ALKIER | |
| FKBP-062 | FKBP13 (mutated to | HSPIKSRKGDVLHAHYTG | 18 |
| | match FKBP12 active | KLEDGTEFDSSLPRNQPFV | |
| | site-Q72R, Q84E, | FSLGTGEVIKGWDQGLLG | |
| | A117H) (DRD- | MSEGEKRKLVIPSELGYG | |
| | M54A, [−]disulfide) | ERGHPPKIPGGATLVFEVE | |
| | | LLKIER | |
| FKBP-063 | FKBP13 (mutated to | HSPIKSRKGDVLHAHYTG | 19 |
| | match FKBP12 active | KLEDGTEFDSSLPRNQPFV | |
| | site-Q72R, Q84E, | FSLGTGEVIKGWDQGLLG | |
| | A117H) | MCEGEKRKLVIPSELGYG | |
| | (DRD-M54A, C42S) | ERGHPPKIPGGATLVFEVE | |
| | | LLKIER | |
| FKBP-064 | FKBP13 (mutated to | HCPIKSRKGDVLHMHYTG | 20 |
| | match FKBP12 active | KLEDGTEFDSSLPRNQPFV | |
| | site-Q72R, Q84E, | FSLGTGEVIKGWDQGLLG | |
| | A117H) | MCEGEKLKLVIPSELGYG | |
| | (DRD-R102L) | ERGHPPKIPGGATLVFEVE | |
| | | LLKIER | |
| FKBP-065 | FKBP13 (mutated to | HCPIKSRKGDVLHMHYTG | 21 |
| | match FKBP12 active | KLEDGTEFDSSLPRNQPFV | |
| | site-Q72R, Q84E, | FSLGTGEVIKGWDQGLLG | |
| | A117H) | MCEGEKRKLVIPSELGYG | |
| | | ERGHPPKIPGGATLVFEVE | |
| | | LLKIER | |
| FKBP-066 | FKBP13 (mutated to | HCPIKSRKGDVLHKHYTG | 22 |
| | match FKBP12 active | KLEDGTEFDSSLPRNQPFV | |
| | site-Q72R, Q84E, | FSLGTGEVIKGWDQGLLG | |
| | A117H) | MCEGEKRKLVIPSELGYG | |
| | (DRD-M54K) | ERGHPPKIPGGATLVFEVE | |
| | | LLKIER | |
| FKBP-067 | FKBP13 (mutated to | HCPIKSRKGDVLHHHYTG | 23 |
| | match FKBP12 active | KLEDGTEFDSSLPRNQPFV | |
| | site-Q72R, Q84E, | FSLGTGEVIKGWDQGLLG | |
| | A117H) | MCEGEKRKLVIPSELGYG | |
| | (DRD-M54H) | ERGHPPKIPGGATLVFEVE | |
| | | LLKIER | |
| FKBP-068 | FKBP13 (mutated to | HCPIKSRKGDVLHSHYTG | 24 |
| | match FKBP12 active | KLEDGTEFDSSLPRNQPFV | |
| | site-Q72R, Q84E, | FSLGTGEVIKGWDQGLLG | |
| | A117H) | MCEGEKRKLVIPSELGYG | |
| | (DRD-M54S) | ERGHPPKIPGGATLVFEVE | |
| | | LLKIER | |
| FKBP-069 | FKBP13 (mutated to | HCPIKSRKGDVLHGHYTG | 25 |
| | match FKBP12 active | KLEDGTEFDSSLPRNQPFV | |
| | site-Q72R, Q84E, | FSLGTGEVIKGWDQGLLG | |
| | A117H) | MCEGEKRKLVIPSELGYG | |
| | (DRD-M54G) | ERGHPPKIPGGATLVFEVE | |
| | | LLKIER | |
| FKBP-070 | FKBP13 (mutated to | HCPIKSRKGDVLHDHYTG | 26 |
| | match FKBP12 active | KLEDGTEFDSSLPRNQPFV | |
| | site-Q72R, Q84E, | FSLGTGEVIKGWDQGLLG | |
| | A117H) | MCEGEKRKLVIPSELGYG | |
| | (DRD-M54D) | ERGHPPKIPGGATLVFEVE | |
| | | LLKIER | |
| FKBP-071 | FKBP13 (mutated to | HCPIKSRKGDVLHVHYTG | 27 |
| | match FKBP12 active | KLEDGTEFDSSLPRNQPFV | |
| | site-Q72R, Q84E, | FSLGTGEVIKGWDQGLLG | |
| | A117H) | MCEGEKRKLVIPSELGYG | |
| | (DRD-M54V)-AcGFP- | ERGHPPKIPGGATLVFEVE | |
| | P2A-mCherry | LLKIER | |
| FKBP-072 | pELNS-FKBP13 | HCPIKSRKGDVLHMHYTG | 28 |
| | (mutated to match | KLEDGTEFDSSLPRNQPFV | |
| | FKBP12 active site- | FSLGTGEVIKGWDQGKLG | |
| | Q72R, Q84E, | MCEGEKRKLVIPSELGYG | |
| | A117H) | ERGHPPKIPGGATLVFEVE | |
| | (DRD-L93K) | LLKIER | |
| FKBP-073 | FKBP13 (mutated to | HCPIKSRKGDVLHMHYTG | 29 |
| | match FKBP12 active | KLEDGTEFDSSLPRNQPFV | |
| | site-Q72R, Q84E, | FSLGTGEVIKGWDQGHLG | |
| | A117H) | MCEGEKRKLVIPSELGYG | |
| | (DRD-L93H) | ERGHPPKIPGGATLVFEVE | |
| | | LLKIER | |
| FKBP-074 | FKBP13 (mutated to | HCPIKSRKGDVLHMHYTG | 30 |
| | match FKBP12 active | KLEDGTEFDSSLPRNQPFV | |
| | site-Q72R, Q84E, | FSLGTGEVIKGWDQGSLG | |
| | A117H) | MCEGEKRKLVIPSELGYG | |
| | (DRD-L93S) | ERGHPPKIPGGATLVFEVE | |
| | | LLKIER | |
| FKBP-075 | FKBP13 (mutated to | HCPIKSRKGDVLHMHYTG | 31 |
| | match FKBP12 active | KLEDGTEFDSSLPRNQPFV | |
| | site-Q72R, Q84E, | FSLGTGEVIKGWDQGGLG | |
| | A117H) | MCEGEKRKLVIPSELGYG | |
| | (DRD-L93G) | ERGHPPKIPGGATLVFEVE | |
| | | LLKIER | |
| FKBP-076 | FKBP13 (mutated to | HCPIKSRKGDVLHMHYTG | 32 |
| | match FKBP12 active | KLEDGTEFDSSLPRNQPFV | |
| | site-Q72R, Q84E, | FSLGTGEVIKGWDQGDLG | |
| | A117H) | MCEGEKRKLVIPSELGYG | |
| | (DRD-L93D) | ERGHPPKIPGGATLVFEVE | |
| | | LLKIER | |
| FKBP-083 | FKBP13 trunc1 | CPIKSRKGDVLHAHYTGK | 33 |
| | (mutated to match | LEDGTEFDSSLPRNQPFVF | |
| | FKBP12 active site- | SLGTGEVIKGWDQGLLG | |
| | Q72R, Q84E, | MCEGEKRKLVIPSELGYG | |
| | A117H) | ERGHPPKIPGGATLVFEVE | |
| | (DRD-M54A) | LLKIER | |
| FKBP-084 | FKBP13 trunc2 | PIKSRKGDVLHAHYTGKL | 34 |
| | (mutated to match | EDGTEFDSSLPRNQPFVFS | |
| | FKBP12 active site | LGTGEVIKGWDQGLLGM | |
| | Q72R, Q84E, | CEGEKRKLVIPSELGYGER | |
| | A117H) | GHPPKIPGGATLVFEVELL | |
| | (DRD-M54A) | KIER | |
| FKBP-085 | FKBP13 trunc 1WT | CPIKSRKGDVLHMHYTGK | 35 |
| | (mutated to match | LEDGTEFDSSLPRNQPFVF | |
| | FKBP12 active site | SLGTGEVIKGWDQGLLG | |
| | Q72R, Q84E, | MCEGEKRKLVIPSELGYG | |
| | A117H) (DRD-WT) | ERGHPPKIPGGATLVFEVE | |
| | | LLKIER | |
| FKBP-086 | FKBP13 trunc2WT | PIKSRKGDVLHMHYTGKL | 36 |
| | (mutated to match | EDGTEFDSSLPRNQPFVFS | |
| | FKBP12 active site | LGTGEVIKGWDQGLLGM | |
| | Q72R, Q84E, | CEGEKRKLVIPSELGYGER | |
| | A117H) (DRD-WT) | GHPPKIPGGATLVFEVELL | |
| | | KIER | |
| FKBP-087 | FKBP13 (mutated to | HCPIKSRKGDVLHAHYTG | 37 |
| | match FKBP12 active | KLEDGTEFDSSLPRNQPFV | |
| | site Q72R, Q84E, | FSLGTGEVIKGWDQGLLG | |
| | A117H; and I121H) | MCEGEKRKLVIPSELGYG | |
| | (DRD-M54A) | ERGHPPKHPGGATLVFEV | |
| | | ELLKIER | |
| FKBP-088 | FKBP13 (mutated to | HCPIKSRKGDVLHAHYTG | 38 |
| | match FKBP12 active | KLEDGTEFDSSLPRNQPFV | |
| | site Q72R, Q84E, | FSLGTGEVIKGWDQGLLG | |
| | A117H; and F129Y) | MCEGEKRKLVIPSELGYG | |
| | (DRD-M54A) | ERGHPPKIPGGATLVYEVE | |
| | | LLKIER | |
| FKBP-089 | FKBP13 (mutated to | HCPIKSRKGDVLHAHYTG | 39 |
| | match FKBP12 active | KLEDGTEFDSSLPRNQPFV | |
| | site Q72R, V85D, | FSLGTGQDIKGWDQGLLG | |
| | A117H) | MCEGEKRKLVIPSELGYG | |
| | (DRD-M54A) | ERGHPPKIPGGATLVFEVE | |
| | | LLKIER | |
| FKBP-090 | FKBP13 (mutated to | HCPIKSRKGDVLHAHYTG | 40 |
| | match FKBP12 active | KLEDGTEFDSSLPRNQPFV | |
| | site Q72R, 186D, | FSLGTGQVDKGWDQGLL | |
| | A117H) | GMCEGEKRKLVIPSELGY | |
| | (DRD-M54A) | GERGHPPKIPGGATLVFEV | |
| | | ELLKIER | |
| FKBP-091 | FKBP13 (mutated to | HCPIKSRKGDVLHAHYTG | 41 |
| | match FKBP12 active | KLEDGTEFDSSLPRNQPFV | |
| | site Q72R, Q84E, | FSLGTGEVIKGWDQGLLG | |
| | A117W) | MCEGEKRKLVIPSELGYG | |
| | (DRD-M54A) | ERGWPPKIPGGATLVFEV | |
| | | ELLKIER | |
| FKBP-092 | FKBP13 (mutated to | HCPIKSRKGDVLHMHYTG | 42 |
| | match FKBP12 active | KLEDGTEFDSSLPRNQPFV | |
| | site Q72R, Q84E, | FSLGTGEVIKGWDQGLLG | |
| | A117H) | MCEGEKRKLVIPSELGYG | |
| | (DRD-L127A) | ERGHPPKIPGGATAVFEVE | |
| | | LLKIER | |
| FKBP-093 | FKBP13 (mutated to | HCPIKSRKGDVLHMHYTG | 43 |
| | match FKBP12 active | KLEDGTEFDSSLPRNQPFV | |
| | site Q72R, Q84E, | FSAGTGEVIKGWDQGLLG | |
| | A117H) | MCEGEKRKLVIPSELGYG | |
| | (DRD-L80A) | ERGHPPKIPGGATLVFEVE | |
| | | LLKIER | |
| FKBP-094 | FKBP13 (mutated to | HCPIKSRKGDVLHMHYTG | 44 |
| | match FKBP12 active | KLEDGTEFDSSLPRNQPFV | |
| | site Q72R, Q84E, | FSLGTGEVIKGWDQGLLG | |
| | A117H) | MCEGEKRKLVISSELGYG | |
| | (DRD-P107S) | ERGHPPKIPGGATLVFEVE | |
| | | LLKIER | |
| FKBP-095 | FKBP13 (mutated to | HCPIKSRKGDVLHMHYTG | 45 |
| | match FKBP12 active | KLEDGTEFDSSLPRNQPFV | |
| | site Q72R, Q84E, | FSLGTGEVIKGWDQGLLG | |
| | A117H) | MCEGEKRKLVIPSELGYG | |
| | (DRD-P122S) | ERGHPPKISGGATLVFEVE | |
| | | LLKIER | |
| FKBP-096 | FKBP13 (mutated to | HCPIKSRKGDVLHMHYTG | 46 |
| | match FKBP12 active | KLEDGTEFDSSLPRNQPFV | |
| | site Q72R, Q84E, | FSLGTGEVIKGWDQGLLG | |
| | A117H) | MCEGLKRKLVIPSELGYG | |
| | (DRD-E100L) | ERGHPPKIPGGATLVFEVE | |
| | | LLKIER | |
| FKBP-097 | FKBP13 (mutated to | HCPIKSRKGDVLHMHYTG | 47 |
| | match FKBP12 active | KLEDGTEFDSSLPRNQPFV | |
| | site Q72R, Q84E, | FSLGTGEVIKGWDQGLLG | |
| | A117H) | MCEGSKRKLVIPSELGYG | |
| | (DRD-E100S) | ERGHPPKIPGGATLVFEVE | |
| | | LLKIER | |
| FKBP-098 | FKBP13 (mutated to | HCPIKSRKGDVLHSHYTG | 48 |
| | match FKBP12 active | KLEDGTEFDSSLPRNQPFV | |
| | site Q72R, Q84E, | FSLGTGEVIKGWDQGLLG | |
| | A117W) (DRD- | MCEGEKRKLVIPSELGYG | |
| | M54S) | ERGWPPKIPGGATLVFEV | |
| | | ELLKIER | |
| FKBP-099 | FKBP13 (mutated to | HCPIKSRKGDVLHAHYTG | 49 |
| | match FKBP12 active | KLEDGTEFDSSLPRNQPFV | |
| | site Q72R, Q84E, | FSLGTGEVIKGWDQGLLG | |
| | A117W) (DRD- | MCEGEKRKLVIPSELGYG | |
| | M54A, del c-term R) | ERGWPPKIPGGATLVFEV | |
| | | ELLKIE | |
| FKBP-100 | FKBP13 (mutated to | HCPIKSRKGDVLHAHYTG | 50 |
| | match FKBP12 active | KLEDGTEFDSSLPRNQPFV | |
| | site Q72R, Q84E, | FSLGTGEVIKGWDQGLLG | |
| | A117W) (DRD- | VCEGEKRKL VIPSELGYGE | |
| | M54A, M96V) | RGWPPKIPGGATLVFEVE | |
| | | LLKIER | |
| FKBP-101 | FKBP13 (mutated to | PIKSRKGDVLHAHYTGKL | 51 |
| | match FKBP12 active | EDGTEFDSSLPRNQPFVFS | |
| | site Q72R, Q84E, | LGTGEVIKGWDQGLLGM | |
| | A117W) (DRD- | CEGEKRKLVIPSELGYGER | |
| | M54A, Nterm-2 aa | GWPPKIPGGATLVFEVELL | |
| | trunc) | KIER | |
| FKBP-102 | FKBP13 (mutated to | HCPIKSRKGDVLHAHYTG | 52 |
| | match FKBP12 active | KLEDGTEFDSSLPRNQPFV | |
| | site Q72R, Q84E, | FSLGTGEVIKGWDQGLLG | |
| | A117W) (DRD- | MCEGEKRKLVIPSELGYG | |
| | M54A, P118V) | ERGWVPKIPGGATLVFEV | |
| | | ELLKIER | |
| FKBP-103 | FKBP13 (mutated to | HCPIKSRKGDVLHAHYTG | 53 |
| | match FKBP12 active | KLEDGTEFDSSLPRNQPFV | |
| | site Q72R, Q84E, | FSLGTGEVIKGWDQGLLG | |
| | A117W) (DRD- | MCEGEKRKLVIPSELGYG | |
| | M54A, P119V) | ERGWPVKIPGGATLVFEV | |
| | | ELLKIER | |
| FKBP-104 | FKBP13 (mutated to | HCPIKSRKGDVLHAHYTG | 54 |
| | match FKBP12 active | KLEDGTEFDSSLPRNQPFV | |
| | site Q72R, Q84E, | FSLGTGEVIKGWDQGLLG | |
| | A117W), K120I | MCEGEKRKLVIPSELGYG | |
| | (DRD-M54A) | ERGWPPIIPGGATLVFEVE | |
| | | LLKIER | |
| FKBP-105 | FKBP13 (mutated to | HCPIKSRKGDVLHAHYTG | 55 |
| | match FKBP12 active | KLEDGTEFDSSLPRNQPFV | |
| | site Q72R, Q84E, | FSLGTGEVIKGWDQGLLG | |
| | A117W), K120R | MCEGEKRKLVIPSELGYG | |
| | (DRD-M54A) | ERGWPPRIPGGATLVFEVE | |
| | | LLKIER | |
| FKBP-106 | FKBP13 (mutated to | HCPIKSRKGDVLHAHYTG | 56 |
| | match FKBP12 active | KLEDGTEFDSSLPRNQPFV | |
| | site Q72R, Q84E, | FSLGTGEVIKGWDQGLLG | |
| | A117Y (DRD- | MCEGEKRKLVIPSELGYG | |
| | M54A) | ERGYPPKIPGGATLVFEVE | |
| | | LLKIER | |
| FKBP-107 | FKBP13 (mutated to | HCPIKSRKGDVLHMHYTG | 57 |
| | match FKBP12 active | KLEDGTEFDSSLPRNQPFV | |
| | site Q72R, Q84E, | FSLGTGEVIKGWDQGLLG | |
| | A117W) (DRD- | MCEGEKRKLVVPSELGYG | |
| | I106V) | ERGWPPKIPGGATLVFEV | |
| | | ELLKIER | |
| FKBP-108 | FKBP13 (mutated to | HCPIKSRKGDVLHAHYTG | 58 |
| | match FKBP12 active | KLEDGTEFDSSLPRNQPFV | |
| | site Q72R, Q84E, | FSLGTGEVIKGWDQGLLG | |
| | A117W) (DRD- | MCEGEKRKLVVPSELGYG | |
| | M54A I106V) | ERGWPPKIPGGATLVFEV | |
| | | ELLKIER | |
| FKBP-109 | FKBP13 (mutated to | HCPIKSRKGDVLHMHYTG | 59 |
| | match FKBP12 active | KLEDGTEFDSSLPRNQPFV | |
| | site Q72R, Q84E, | FSLGTGEVIKGWDQGLLG | |
| | A117W) (DRD- | MCEGEKRKVVIPSELGYG | |
| | L104V) | ERGWPPKIPGGATLVFEV | |
| | | ELLKIER | |
| FKBP-110 | FKBP13 (mutated to | HCPIKSRKGDVLHMHYTG | 60 |
| | match FKBP12 active | KLEDGTEFDSSLPRNQPFV | |
| | site Q72R, Q84E, | FSLGTGEVIKGWDQGLLG | |
| | A117W) (DRD- | MCEGEKRKLVIPSEAGYG | |
| | L110A) | ERGWPPKIPGGATLVFEV | |
| | | ELLKIERG | |
| FKBP-111 | FKBP13 (mutated to | HCPIKSRKGDVLHMHYTG | 61 |
| | match FKBP12 active | KLEDGTEFDSSLPRNQPFV | |
| | site Q72R, Q84E, | FSLGTGEVIKGWAQGLLG | |
| | A117W) (DRD- | MCEGEKRKLVIPSELGYG | |
| | D90A) | ERGWPPKIPGGATLVFEV | |
| | | ELLKIER | |
| FKBP-112 | FKBP13 (mutated to | HCPIKSRKGDVLHMHYTG | 62 |
| | match FKBP12 active | KLEDGTEFDSSLPRNQPFV | |
| | site Q72R, Q84E, | FSLGTGEVIKGWDQGVLG | |
| | A117W) (DRD- | MCEGEKRKLVIPSELGYG | |
| | L93V) | ERGWPPKIPGGATLVFEV | |
| | | ELLKIER | |
| FKBP-113 | FKBP13 (mutated to | HCPIKSRKGDVLHSHYTG | 63 |
| | match FKBP12 active | KLEDGTEFDSSLPRNQPFV | |
| | site Q72R, Q84E, | FSLGTGEVIKGWDQGLLG | |
| | A117W) (DRD- | MCEGEKRKLVIPSELGYG | |
| | M54S) | ERGWPPKIPGGATLVFEV | |
| | | ELLKIER | |
| FKBP-114 | FKBP13 (mutated to | CPIKSRKGDVLHAHYTGK | 64 |
| | match FKBP12 active | LEDGTEFDSSLPRNQPFVF | |
| | site Q72R, Q84E, | SLGTGEVIKGWDQGLLG | |
| | A117W) (DRD- | MCEGEKRKLVIPSELGYG | |
| | M54A, Trunc1) | ERGWPPKIPGGATLVFEV | |
| | | ELLKIER | |
| FKBP-115 | FKBP13 (mutated to | PIKSRKGDVLHMHYTGKL | 65 |
| | match FKBP12 active | EDGTEFDSSLPRNQPFVFS | |
| | site Q72R, Q84E, | LGTGEVIKGWDQGLLGM | |
| | A117W) (DRD- | CEGEKRKLVIPSELGYGER | |
| | none, Trunc2) | GWPPKIPGGATLVFEVELL | |
| | | KIER | |
| FKBP-116 | FKBP13 (mutated to | IKSRKGDVLHMHYTGKLE | 66 |
| | match FKBP12 active | DGTEFDSSLPRNQPFVFSL | |
| | site Q72R, Q84E, | GTGEVIKGWDQGLLGMC | |
| | A117W) (DRD- | EGEKRKLVIPSELGYGERG | |
| | none, Trunc3) | WPPKIPGGATLVFEVELLK | |
| | | IER | |
| FKBP-117 | FKBP13 (mutated to | HCPIKSRKGDVLHMHYTG | 67 |
| | match FKBP12 active | KLEDGTEFDSSLPRNQPFV | |
| | site Q72R, Q84E, | FSLGTGERIKGWDQGLLG | |
| | A117W) (DRD | MCEGEKRKLVIPSELGYG | |
| | V85R) | ERGWPPKIPGGATLVFEV | |
| | | ELLKIER | |
| FKBP-118 | FKBP13 (mutated to | HCPIKSRKGDVLHMHYTG | 68 |
| | match FKBP12 active | KLEDGTEFDSSLPRNQPFV | |
| | site Q72R, Q84E, | FSLGTGERIKGWDQGLLG | |
| | A117H) (DRD | MCEGEKRKLVIPSELGYG | |
| | V85R) | ERGHPPKIPGGATLVFEVE | |
| | | LLKIER | |
| FKBP-119 | FKBP13 (mutated to | HCPIKSRKGDVLHMHYTG | 69 |
| | match FKBP12 active | KLEDGTEFDSSLPRNQPFV | |
| | site Q72R, Q84E, | FSLGTGEVIKGWDQGLLG | |
| | A117W) (DRD | MCEGEWRKLVIPSELGYG | |
| | K101W) | ERGWPPKIPGGATLVFEV | |
| | | ELLKIER | |
| FKBP-120 | FKBP13 (mutated to | HCPIKSRKGDVLHMHYTG | 70 |
| | match FKBP12 active | KLEDGTEFDSSLPRNQPFV | |
| | site Q72R, Q84E, | FSLGTGEVIKGWDQGLLG | |
| | A117H) (DRD | MCEGEWRKLVIPSELGYG | |
| | K101W) | ERGHPPKIPGGATLVFEVE | |
| | | LLKIER | |
| FKBP-121 | FKBP13 (mutated to | HCPIKSRKGDVLHMHYTG | 71 |
| | match FKBP12 active | KLEDGTEFDSSLPRNQPFV | |
| | site Q72R, Q84E, | FSLGTGEVIKGWDQGLLG | |
| | A117W) (DRD | MCEGEKRKLVIPSELGYG | |
| | I136V) | ERGWPPKIPGGATLVFEV | |
| | | ELLKVER | |
| FKBP-122 | FKBP13 (mutated to | HCPIKSRKGDVLHMHYTG | 72 |
| | match FKBP12 active | KLEDGTEFDSSLPRNQPFV | |
| | site Q72R, Q84E, | FSLGTGEVIKGWDQGLLG | |
| | A117H) (DRD | MCEGEKRKLVIPSELGYG | |
| | I136V) | ERGHPPKIPGGATLVFEVE | |
| | | LLKVER | |
| FKBP-123 | FKBP13 (mutated to | HCPIKSRKSDVLHMHYTG | 73 |
| | match FKBP12 active | KLEDGTEFDSSLPRNQPFV | |
| | site Q72R, Q84E, | FSLGTGEVIKGWDQGLLG | |
| | A117W) (DRD | MCEGEKRKLVIPSELGYG | |
| | G49S) | ERGWPPKIPGGATLVFEV | |
| | | ELLKIER | |
| FKBP-124 | FKBP13 (mutated to | HCPIKSRKSDVLHMHYTG | 74 |
| | match FKBP12 active | KLEDGTEFDSSLPRNQPFV | |
| | site Q72R, Q84E, | FSLGTGEVIKGWDQGLLG | |
| | A117H) (DRD G49S) | MCEGEKRKLVIPSELGYG | |
| | | ERGHPPKIPGGATLVFEVE | |
| | | LLKIER | |
| FKBP-125 | FKBP13 (mutated to | HCPIKSRKGDVLHMHYTG | 75 |
| | match FKBP12 active | KLESGTEFDSSLPRNQPFV | |
| | site Q72R, Q84E, | FSLGTGEVIKGWDQGLLG | |
| | A117W) (DRD | MCEGEKRKLVIPSELGYG | |
| | D62S) | ERGWPPKIPGGATLVFEV | |
| | | ELLKIER | |
| FKBP-126 | FKBP13 (mutated to | HCPIKSRKGDVLHMHYTG | 76 |
| | match FKBP12 active | KLESGTEFDSSLPRNQPFV | |
| | site Q72R, Q84E, | FSLGTGEVIKGWDQGLLG | |
| | A117H) (DRD D62S) | MCEGEKRKLVIPSELGYG | |
| | | ERGHPPKIPGGATLVFEVE | |
| | | LLKIER | |
| FKBP-127 | FKBP13 (mutated to | HCPIKSRKGDVLHMHYTG | 77 |
| | match FKBP12 active | KLEDGTEFDSSLPRNQPQ | |
| | site Q72R, Q84E, | VFSLGTGEVIKGWDQGLL | |
| | A117W) (DRD | GMCEGEKRKLVIPSELGY | |
| | F76Q) | GERGWPPKIPGGATLVFE | |
| | | VELLKIER | |
| FKBP-128 | FKBP13 (mutated to | HCPIKSRKGDVLLMHYTG | 78 |
| | match FKBP12 active | KLEDGTEFDSSLPRNQPFV | |
| | site Q72R, Q84E, | FSLGTGEVIKGWDQGLLG | |
| | A117W) (DRD | MCEGEKRKLVIPSELGYG | |
| | H53L) | ERGWPPKIPGGATLVFEV | |
| | | ELLKIER | |
| FKBP-129 | FKBP13 (mutated to | HCPIKSRKGDVLHMHYTG | 79 |
| | match FKBP12 active | KLEDGTEFDSSLPRNQPFV | |
| | site Q72R, Q84E, | FSLGTGEVIKGWDQGLLG | |
| | A117W) (DRD | MCLGEKRKLVIPSELGYG | |
| | E98L) | ERGWPPKIPGGATLVFEV | |
| | | ELLKIER | |
| FKBP-130 | FKBP13 (mutated to | HCPIKSRKGDVLHMHYTG | 80 |
| | match FKBP12 active | KLEDGTEFDSSLPRNQPFV | |
| | site Q72R, Q84E, | FSLGTGEVIKGWDQGLLG | |
| | A117W) (DRD | MCEGEKRKLVIKSELGYG | |
| | P107K) | ERGWPPKIPGGATLVFEV | |
| | | ELLKIER | |
| FKBP-131 | FKBP13 (mutated to | HCPIKSRKGDVLHMHYTG | 81 |
| | match FKBP12 active | KLEDGTEFDSSLPRNQPFV | |
| | site Q72R, Q84E, | FSLGTGEVIKGWDQGLLG | |
| | A117W) (DRD | MCEGEKRKLVIPSELGYG | |
| | T126C) | ERGWPPKIPGGACLVFEV | |
| | | ELLKIER | |
| FKBP-132 | FKBP13 (mutated to | HCPIKSRKGDVLHMHYTG | 82 |
| | match FKBP12 active | KLEDGTEFDSSLPRNQPFV | |
| | site Q72R, Q84E, | FSLGTGEVIKGWDQYLLG | |
| | A117W) (DRD | MCEGEKRKLVIPSELGYG | |
| | G92Y) | ERGWPPKIPGGATLVFEV | |
| | | ELLKIER | |
| FKBP-133 | FKBP13 (mutated to | HCPIKSRKGDVLHMHYTG | 83 |
| | match FKBP12 active | KLEDGTEFDSSLPRNQPFV | |
| | site Q72R, Q84E, | FSLGTTEVIKGWDQGLLG | |
| | A117W) (DRD | MCEGEKRKLVIPSELGYG | |
| | G83T) | ERGWPPKIPGGATLVFEV | |
| | | ELLKIER | |
| FKBP-134 | FKBP13 (mutated to | RCPIKSRKGDVLHMHYTG | 84 |
| | match FKBP12 active | KLEDGTEFDSSLPRNQPFV | |
| | site Q72R, Q84E, | FSLGTGEVIKGWDQGLLG | |
| | A117W) (DRD | MCEGEKRKLVIPSELGYG | |
| | H41R) | ERGWPPKIPGGATLVFEV | |
| | | ELLKIER | |
| FKBP-135 | FKBP13 (mutated to | HCPIKSRKGDVLHMHYTG | 85 |
| | match FKBP12 active | KLYDGTEFDSSLPRNQPFV | |
| | site Q72R, Q84E, | FSLGTGEVIKGWDQGLLG | |
| | A117W) (DRD | MCEGEKRKLVIPSELGYG | |
| | E61Y) | ERGWPPKIPGGATLVFEV | |
| | | ELLKIER | |
| FKBP-136 | FKBP13 (mutated to | HCPIKSRKGDVLHMHYTG | 86 |
| | match FKBP12 active | KLEDGTEFDSSLPRNQPFV | |
| | site Q72R, Q84E, | FSLGTGEVIKGWDQGLLG | |
| | A117W) (DRD | MCEGEKRKLVIPMELGYG | |
| | S108M) | ERGWPPKIPGGATLVFEV | |
| | | ELLKIER | |
| FKBP-137 | FKBP13 (mutated to | HCPIKSRKGDVLHMHYTG | 87 |
| | match FKBP12 active | KLEDGTEFDSSLPRNQLFV | |
| | site Q72R, Q84E, | FSLGTGEVIKGWDQGLLG | |
| | A117W) (DRD | MCEGEKRKLVIPSELGYG | |
| | P75L) | ERGWPPKIPGGATLVFEV | |
| | | ELLKIER | |
| FKBP-138 | FKBP13 (mutated to | HCPIKSRKGDVLHMHYTG | 88 |
| | match FKBP12 active | KLEDGTEFDSSLPRNQPFV | |
| | site Q72R, Q84E, | FSLGTGEVIKGWDQGLLG | |
| | A117W) (DRD | MCEGEKRKLVIPSELGYG | |
| | G124R) | ERGWPPKIPGRATLVFEVE | |
| | | LLKIER | |
| FKBP-139 | FKBP13 (mutated to | HCPIKSRKGDVLHMHYTG | 89 |
| | match FKBP12 active | KLEDGTEFDSSLPRNQPFV | |
| | site Q72R, Q84E, | FSLGTGEVIKGWDQGLLG | |
| | A117W) (DRD | MCEGEKRKLVIPSELGYG | |
| | E130R) | ERGWPPKIPGGATLVFRV | |
| | | ELLKIER | |
| FKBP-140 | FKBP13 (mutated to | HCPIKSRKGDVLHMHYTG | 90 |
| | match FKBP12 active | KLEDGTEFDSSLPRNQPFV | |
| | site Q72R, Q84E, | FSLGTGEVIKGWDQGLLG | |
| | A117W) (DRD | MCEGEKRKLVIPSELGYG | |
| | K135Y) | ERGWPPKIPGGATLVFEV | |
| | | ELLYIER | |
| FKBP-141 | FKBP13 (mutated to | HCPIKSRKGDVRHMHYTG | 91 |
| | match FKBP12 active | KLEDGTEFDSSLPRNQPFV | |
| | site Q72R, Q84E, | FSLGTGEVIKGWDQGLLG | |
| | A117R (DRD L52R) | MCEGEKRKLVIPSELGYG | |
| | | ERGRPPKIPGGATLVFEVE | |
| | | LLKIER | |
| FKBP-142 | FKBP13 (mutated to | HCPIKSRKGDVLHMHYTG | 92 |
| | match FKBP12 active | KLEDGGEFDSSLPRNQPFV | |
| | site Q72R, Q84E, | FSLGTGEVIKGWDQGLLG | |
| | A117W) (DRD | MCEGEKRKLVIPSELGYG | |
| | T64G) | ERGWPPKIPGGATLVFEV | |
| | | ELLKIER | |
| FKBP-143 | FKBP13 (mutated to | HCRIKSRKGDVLHMHYTG | 93 |
| | match FKBP12 active | KLEDGTEFDSSLPRNQPFV | |
| | site Q72R, Q84G, | FSLGTGGVIKGWDQGLLG | |
| | A117H) (DRD P43R) | MCEGEKRKLVIPSELGYG | |
| | | ERGHPPKIPGGATLVFEVE | |
| | | LLKIER | |
| FKBP-144 | FKBP13 (mutated to | HCPIKSRKGRVLHMHYTG | 94 |
| | match FKBP12 active | KLEDGTEFDSSLPRNQPFV | |
| | site Q72R, Q84E, | FSLGTGEVIKGWDQGLLG | |
| | A117H) (DRD | MCMGEKRKLVIPSELGYG | |
| | D50R, E98M) | ERGHPPKIPGGATLVFEVE | |
| | | LLKIER | |
| FKBP-145 | FKBP13 (mutated to | SCPIKSRKGDVLHMHYTG | 95 |
| | match FKBP12 active | KLDDGTEFDSSLPRNQPFV | |
| | site Q72R, Q84E, | FSLGTGEVIKGWDQGLLG | |
| | A117H) (DRD H41S, | MCEGEKRKLVIPSELGYG | |
| | E61D) | ERGHPPKIPGGATLVFEVE | |
| | | LLKIER | |
| FKBP-146 | FKBP13 (mutated to | HCPIKSRKGDVLHMHYTG | 96 |
| | match FKBP12 active | KLEDGTEFDSSLPRNQPFV | |
| | site Q72R, Q84E, | FSLGTGEVIKGWDQGLLG | |
| | A117H) (DRD | MCEGEKRKSVIPSELGYG | |
| | L104S, I136L) | ERGHPPKIPGGATLVFEVE | |
| | | LLKLER | |
| FKBP-147 | FKBP13 (mutated to | HCPIKSRKGDVLHMHYTG | 97 |
| | match FKBP12 active | KLEDGREFDSSLPRNQPFV | |
| | site Q72R, Q84E, | FSLGTGEVIKGWDQGLLG | |
| | A117H) (DRD T64R, | MCEGEKRKLVIPGELGYG | |
| | S108G, E137F) | ERGHPPKIPGGATLVFEVE | |
| | | LLKIFR | |
| FKBP-148 | FKBP13 (mutated to | HCPIKSRKGDVLHMHYTG | 98 |
| | match FKBP12 active | KLENGTEFNSSLPRNQPFV | |
| | site Q72R, Q84E, | FSLGTGEVIKGWNQGLLG | |
| | A117H) (DRD | GCEGEKRKLVIPSELGYGE | |
| | D62N, D67N, D90N, | RGHPPKIPGGATLVFKVEL | |
| | M96G, E130K) | LKIER | |
| FKBP-149 | FKBP13 (mutated to | HCPIKSRKGDVLHMHYTG | 99 |
| | match FKBP12 active | KLEDGQEFDSSLPRNQPFV | |
| | site Q72R, Q84E, | FSLGTGEVIKGWDQGLGG | |
| | A117H) (DRD T64Q, | MCEGEKRKLVIPSELGYG | |
| | L94G) | ERGHPPKIPGGATLVFEVE | |
| | | LLKIER | |
| FKBP-150 | FKBP13 (mutated to | HCPIKSRKGDVLHMHYTG | 100 |
| | match FKBP12 active | KLGDGTEFDSSLPRNQPFV | |
| | site Q72R, Q84E, | FSLGTGEVIKGWDQGLLG | |
| | A117H) (DRD E61G, | MCEGEKRKLVIPSELGYG | |
| | G124P) | ERGHPPKIPGPATLVFEVE | |
| | | LLKIER | |
| FKBP-151 | FKBP13 (mutated to | HCPIKSRKGDVLHAHYTG | 101 |
| | match FKBP12 active | KLEDGTEFDSSLPRNQPFV | |
| | site Q72R, Q84R, | FSLGTGRVIKGWDQGLLG | |
| | A117W) (DRD | MCEGEKRKLVIPSELGYG | |
| | M54A) | ERGWPPKIPGGATLVFEV | |
| | | ELLKIER | |
| FKBP-152 | FKBP13 (mutated to | HCPIKSRKGDVLHAHYTG | 102 |
| | match FKBP12 active | KLEDGTEFDSSLPRNQPFV | |
| | site Q72R, Q84W, | FSLGTGWVIKGWDQGLL | |
| | A117W) (DRD | GMCEGEKRKLVIPSELGY | |
| | M54A) | GERGWPPKIPGGATLVFE | |
| | | VELLKIER | |
| FKBP-153 | FKBP13 (mutated to | HCPIKSRKGDVLHAHYTG | 103 |
| | match FKBP12 active | KLEDGTEFDSSLPRNQPFV | |
| | site Q72R, S79W, | FWLGTGEVIKGWDQGLL | |
| | Q84E, A117W) | GMCEGEKRKLVIPSELGY | |
| | (DRD M54A) | GERGWPPKIPGGATLVFE | |
| | | VELLKIER | |
| FKBP-154 | FKBP13 (mutated to | HCPIKSRKGDVLHAHYTG | 104 |
| | match FKBP12 active | KLEDGTEFDSSLPRNQPFV | |
| | site Q72R, S79W, | FSLGTGEVIKGWDQGLLG | |
| | Q84W, A117W) | MCEGEKRKLVIPSELGYG | |
| | (DRD M54A) | | |
| | | ERGWPPKIPGGATLVFEV | |
| | | ELLKIER | |
| FKBP-155 | FKBP13 (mutated to | HCPIKSRKGDVLHAHYTG | 105 |
| | match FKBP12 active | KLEDGTEFDSSLPRNQPFV | |
| | site Q72R, S79W, | FWLGTGRVIKGWDQGLL | |
| | Q84R, A117W) | GMCEGEKRKLVIPSELGY | |
| | (DRD M54A) | GERGWPPKIPGGATLVFE | |
| | | VELLKIER | |
| FKBP-156 | FKBP13 (mutated to | HCPIKSRKGDVLHAHYTG | 106 |
| | match FKBP12 active | KLEDGTEFDSSLPRNQPFV | |
| | site Q72R, T82W, | FSLGWGWVIKGWDQGLL | |
| | Q84W, A117W) | GMCEGEKRKLVIPSELGY | |
| | (DRD M54A) | GERGWPPKIPGGATLVFE | |
| | | VELLKIER | |
| FKBP-157 | FKBP13 (mutated to | HCPIKSRKGDVLHAHYTG | 107 |
| | match FKBP12 active | KLEDGTEFDSSLPRNQPFV | |
| | site Q72R, T82W, | FSLGWGRVIKGWDQGLL | |
| | Q84E, A117W) | GMCEGEKRKLVIPSELGY | |
| | (DRD M54A) | GERGWPPKIPGGATLVFE | |
| | | VELLKIER | |
| FKBP-158 | FKBP13 (mutated to | HCPIKSRKGDVLHAHYTG | 108 |
| | match FKBP12 active | KLEDGTEFDSSLPRNQPFV | |
| | site Q72R, Q84E, | FSLGTGEVIKGWDQGLLG | |
| | A117W) (DRD | MCEGEKRKLVIPSELEYG | |
| | M54A G111E) | ERGWPPKIPGGATLVFEV | |
| | | ELLKIER | |
| FKBP-159 | FKBP13 (mutated to | HCPIKSRKGDVLHAHYTG | 109 |
| | match FKBP12 active | KLEDGTEFDSSLPRNQPFV | |
| | site Q72R, Q84E, | FSLGTGEVIKGWDQGLLG | |
| | A117W) (DRD | MCEGEKRKLVIPSELYYG | |
| | M54A G111Y) | ERGWPPKIPGGATLVFEV | |
| | | ELLKIER | |

Constructs comprising the DRD sequences set forth in Table 1 were made to determine the effect of each DRD on an exemplary payload polypeptide (i.e., Aequorea coerulescens GFP). These constructs are set forth in Table 2.

TABLE 2
Exemplary DRD/AcGFP Constructs

| Name | Description |
|---|---|
| FKBP-044 | pELNS-FKBP13 (matching FKBP12 Q72R, Q84E, A117H DRD M54A)- |
| | AcGFP-P2A-mCherry |
| FKBP-052 | pELNS-FKBP13 (matching FKBP12 Q72R, Q84E, A117H, [−]disulfide)- |
| | AcGFP-P2A-mCherry |
| FKBP-053 | pELNS-FKBP13 (matching FKBP12 Q72R, Q84E, A117H DRD D90G, |
| | F66H)-AcGFP-P2A-mCherry |
| FKBP-054 | pELNS-FKBP13 (matching FKBP12 Q72R, Q84E, A117H DRD D90G, |
| | L127K)-AcGFP-P2A-mCherry |
| FKBP-055 | pELNS-FKBP13 (matching FKBP12 Q72R, Q84E, A117H DRD F78V)- |
| | AcGFP-P2A-mCherry |
| FKBP-056 | pELNS-FKBP13 (matching FKBP12 Q72R, Q84E, A117H DRD |
| | F129K)-AcGFP-P2A-mCherry |
| FKBP-057 | pELNS-FKBP13 (matching FKBP12 Q72R, Q84E, A117H DRD I106A)- |
| | AcGFP-P2A-mCherry |
| FKBP-058 | pELNS-FKBP13 (matching FKBP12 Q72R, Q84E, A117H DRD L52A)- |
| | AcGFP-P2A-mCherry |
| FKBP-059 | pELNS-FKBP13 (matching FKBP12 Q72R, Q84E, A117H DRD L93A)- |
| | AcGFP-P2A-mCherry |
| FKBP-060 | pELNS-FKBP13 (matching FKBP12 Q72R, Q84E, A117H DRD |
| | L104A)-AcGFP-P2A-mCherry |
| FKBP-061 | pELNS-FKBP13 (matching FKBP12 Q72R, Q84E, A117H DRD |
| | L133A)-AcGFP-P2A-mCherry |
| FKBP-062 | pELNS-FKBP13 (matching FKBP12 Q72R, Q84E, A117H DRD M54A, |
| | [−]disulfide)-AcGFP-P2A-mCherry |
| FKBP-063 | pELNS-FKBP13 (matching FKBP12 Q72R, Q84E, A117H DRD M54A, |
| | C42S)-AcGFP-P2A-mCherry |
| FKBP-064 | pELNS-FKBP13 (matching FKBP12 Q72R, Q84E, A117H DRD |
| | R102L)-AcGFP-P2A-mCherry |
| FKBP-066 | pELNS-FKBP13 (matching FKBP12 Q72R, Q84E, A117H DRD M54K)- |
| | AcGFP-P2A-mCherry |
| FKBP-067 | pELNS-FKBP13 (matching FKBP12 Q72R, Q84E, A117H DRD M54H)- |
| | AcGFP-P2A-mCherry |
| FKBP-068 | pELNS-FKBP13 (matching FKBP12 Q72R, Q84E, A117H DRD M54S)- |
| | AcGFP-P2A-mCherry |
| FKBP-069 | pELNS-FKBP13 (matching FKBP12 Q72R, Q84E, A117H DRD M54G)- |
| | AcGFP-P2A-mCherry |
| FKBP-070 | pELNS-FKBP13 (matching FKBP12 Q72R, Q84E, A117H DRD M54D)- |
| | AcGFP-P2A-mCherry |
| FKBP-071 | pELNS-FKBP13 (matching FKBP12 Q72R, Q84E, A117H DRD M54V)- |
| | AcGFP-P2A-mCherry |
| FKBP-072 | pELNS-FKBP13 (matching FKBP12 Q72R, Q84E, A117H DRD L93K)- |
| | AcGFP-P2A-mCherry |
| FKBP-074 | pELNS-FKBP13 (matching FKBP12 Q72R, Q84E, A117H DRD L93S)- |
| | AcGFP-P2A-mCherry |
| FKBP-075 | pELNS-FKBP13 (matching FKBP12 Q72R, Q84E, A117H DRD L93G)- |
| | AcGFP-P2A-mCherry |
| FKBP-076 | pELNS-FKBP13 (matching FKBP12 Q72R, Q84E, A117H DRD L93D)- |
| | AcGFP-P2A-mCherry |
| FKBP-077 | pELNS-FKBP13 (matching FKBP12 Q72R, Q84E, A117H DRD L93V)- |
| | AcGFP-P2A-mCherry |
| FKBP-083 | pELNS-FKBP13 trunc1 (mutated to match FKBP12 active site Q72R, |
| | Q84E, A117H DRD M54A)-AcGFP-P2A-mCherry |
| FKBP-084 | pELNS-FKBP13 trunc2 (mutated to match FKBP12 active site Q72R, |
| | Q84E, A117H DRD M54A)-AcGFP-P2A-mCherry |
| FKBP-085 | pELNS-FKBP13 trunc1WT (mutated to match FKBP12 active site Q72R, |
| | Q84E, A117H DRD WT)-AcGFP-P2A-mCherry |
| FKBP-086 | pELNS-FKBP13 trunc2WT (mutated to match FKBP12 active site Q72R, |
| | Q84E, A117H DRD WT)-AcGFP-P2A-mCherry |
| FKBP-087 | pELNS-FKBP13 (mutated to match FKBP12 active site Q72R, Q84E, |
| | A117H and I121H DRD M54A)-AcGFP-P2A-mCherry |
| FKBP-088 | pELNS-FKBP13 (mutated to match FKBP12 active site Q72R, Q84E, |
| | A117H and F129Y DRD M54A)-AcGFP-P2A-mCherry |
| FKBP-089 | pELNS-FKBP13 (mutated to match FKBP12 active site Q72R, V85D, |
| | A117H DRD M54A)-AcGFP-P2A-mCherry |
| FKBP-090 | pELNS-FKBP13 (mutated to match FKBP12 active site Q72R, I86D, |
| | A117H DRD M54A)-AcGFP-P2A-mCherry |
| FKBP-091 | pELNS-FKBP13 (mutated to match FKBP12 active site Q72R, Q84E, |
| | A117W DRD M54A)-AcGFP-P2A-mCherry |
| FKBP-092 | pELNS-FKBP13 (mutated to match FKBP12 active site Q72R, Q84E, |
| | A117H DRD L127A)-AcGFP-P2A-mCherry |
| FKBP-093 | pELNS-FKBP13 (mutated to match FKBP12 active site Q72R, Q84E, |
| | A117H DRD L80A)-AcGFP-P2A-mCherry |
| FKBP-094 | pELNS-FKBP13 (mutated to match FKBP12 active site Q72R, Q84E, |
| | A117H DRD P107S)-AcGFP-P2A-mCherry |
| FKBP-095 | pELNS-FKBP13 (mutated to match FKBP12 active site Q72R, Q84E, |
| | A117H DRD P122S)-AcGFP-P2A-mCherry |
| FKBP-096 | pELNS-FKBP13 (mutated to match FKBP12 active site Q72R, Q84E, |
| | A117H DRD E100L)-AcGFP-P2A-mCherry |
| FKBP-097 | pELNS-FKBP13 (mutated to match FKBP12 active site Q72R, Q84E, |
| | A117H DRD E100S)-AcGFP-P2A-mCherry |
| FKBP-098 | pELNS-FKBP13 (mutated to match FKBP12 active site Q72R, Q84E, |
| | A117W DRD M54S)-AcGFP-P2A-mCherry |
| FKBP-099 | pELNS-FKBP13 (mutated to match FKBP12 active site Q72R, Q84E, |
| | A117W DRD M54A, del c-term R)-AcGFP-P2A-mCherry |
| FKBP-100 | pELNS-FKBP13 (mutated to match FKBP12 active site Q72R, Q84E, |
| | A117W DRD M54A, M96V)-AcGFP-P2A-mCherry |
| FKBP-101 | pELNS-FKBP13 (mutated to match FKBP12 active site Q72R, Q84E, |
| | A117W DRD M54A, Nterm- 2aa trunc)-AcGFP-P2A-mCherry |
| FKBP-102 | pELNS-FKBP13 (mutated to match FKBP12 active site Q72R, Q84E, |
| | A117W DRD M54A, P118V)-AcGFP-P2A-mCherry |
| FKBP-103 | pELNS-FKBP13 (mutated to match FKBP12 active site Q72R, Q84E, |
| | A117W DRD M54A, P119V)-AcGFP-P2A-mCherry |
| FKBP-104 | pELNS-FKBP13 (mutated to match FKBP12 active site Q72R, Q84E, |
| | A117W, K120I DRD M54A)-AcGFP-P2A-mCherry |
| FKBP-105 | pELNS-FKBP13 (mutated to match FKBP12 active site Q72R, Q84E, |
| | A117W, K120R DRD M54A)-AcGFP-P2A-mCherry |
| FKBP-106 | pELNS-FKBP13 (mutated to match FKBP12 active site Q72R, Q84E, |
| | A117Y DRD M54A)-AcGFP-P2A-mCherry |
| FKBP-107 | pELNS-FKBP13 (mutated to match FKBP12 active site Q72R, Q84E, |
| | A117W DRD I106V)-AcGFP-P2A-mCherry |
| FKBP-108 | pELNS-FKBP13 (mutated to match FKBP12 active site Q72R, Q84E, |
| | A117W DRD M54A I106V)-AcGFP-P2A-mCherry |
| FKBP-109 | pELNS-FKBP13 (mutated to match FKBP12 active site Q72R, Q84E, |
| | A117W DRD L104V)-AcGFP-P2A-mCherry |
| FKBP-110 | pELNS-FKBP13 (mutated to match FKBP12 active site Q72R, Q84E, |
| | A117W DRD L110A)-AcGFP-P2A-mCherry |
| FKBP-111 | pELNS-FKBP13 (mutated to match FKBP12 active site Q72R, Q84E, |
| | A117W DRD D90A)-AcGFP-P2A-mCherry |
| FKBP-112 | pELNS-FKBP13 (mutated to match FKBP12 active site Q72R, Q84E, |
| | A117W DRD L93V)-AcGFP-P2A-mCherry |
| FKBP-113 | pELNS-FKBP13 (mutated to match FKBP12 active site Q72R, Q84E, |
| | A117W DRD M54S)-AcGFP-P2A-mCherry |
| FKBP-114 | pELNS-FKBP13 (mutated to match FKBP12 active site Q72R, Q84E, |
| | A117W DRD M54A, Trunc1)-AcGFP-P2A-mCherry |
| FKBP-115 | pELNS-FKBP13 (mutated to match FKBP12 active site Q72R, Q84E, |
| | A117W DRD none, Trunc2)-AcGFP-P2A-mCherry |
| FKBP-116 | pELNS-FKBP13 (mutated to match FKBP12 active site Q72R, Q84E, |
| | A117W DRD none, Trunc3)-AcGFP-P2A-mCherry |
| FKBP-117 | pELNS-FKBP13 (mutated to match FKBP12 active site Q72R, Q84E, |
| | A117W DRD V85R)-AcGFP-P2A-mCherry |
| FKBP-118 | pELNS-FKBP13 (mutated to match FKBP12 active site Q72R, Q84E, |
| | A117H DRD V85R)-AcGFP-P2A-mCherry |
| FKBP-119 | pELNS-FKBP13 (mutated to match FKBP12 active site Q72R, Q84E, |
| | A117W DRD K101W)-AcGFP-P2A-mCherry |
| FKBP-120 | pELNS-FKBP13 (mutated to match FKBP12 active site Q72R, Q84E, |
| | A117H DRD K101W)-AcGFP-P2A-mCherry |
| FKBP-121 | pELNS-FKBP13 (mutated to match FKBP12 active site Q72R, Q84E, |
| | A117W DRD I136V)-AcGFP-P2A-mCherry |
| FKBP-122 | pELNS-FKBP13 (mutated to match FKBP12 active site Q72R, Q84E, |
| | A117H DRD I136V)-AcGFP-P2A-mCherry |
| FKBP-123 | pELNS-FKBP13 (mutated to match FKBP12 active site Q72R, Q84E, |
| | A117W DRD G49S)-AcGFP-P2A-mCherry |
| FKBP-124 | pELNS-FKBP13 (mutated to match FKBP12 active site Q72R, Q84E, |
| | A117H DRD G49S)-AcGFP-P2A-mCherry |
| FKBP-125 | pELNS-FKBP13 (mutated to match FKBP12 active site Q72R, Q84E, |
| | A117W DRD D62S)-AcGFP-P2A-mCherry |
| FKBP-126 | pELNS-FKBP13 (mutated to match FKBP12 active site Q72R, Q84E, |
| | A117H DRD D62S)-AcGFP-P2A-mCherry |
| FKBP-127 | pELNS-FKBP13 (mutated to match FKBP12 active site Q72R, Q84E, |
| | A117W DRD F76Q)-AcGFP-P2A-mCherry |
| FKBP-128 | pELNS-FKBP13 (mutated to match FKBP12 active site Q72R, Q84E, |
| | A117W DRD H53L)-AcGFP-P2A-mCherry |
| FKBP-129 | pELNS-FKBP13 (mutated to match FKBP12 active site Q72R, Q84E, |
| | A117W DRD E98L)-AcGFP-P2A-mCherry |
| FKBP-130 | pELNS-FKBP13 (mutated to match FKBP12 active site Q72R, Q84E, |
| | A117W DRD P107K)-AcGFP-P2A-mCherry |
| FKBP-131 | pELNS-FKBP13 (mutated to match FKBP12 active site Q72R, Q84E, |
| | A117W DRD T126C)-AcGFP-P2A-mCherry |
| FKBP-132 | pELNS-FKBP13 (mutated to match FKBP12 active site Q72R, Q84E, |
| | A117W DRD G92Y)-AcGFP-P2A-mCherry |
| FKBP-133 | pELNS-FKBP13 (mutated to match FKBP12 active site Q72R, Q84E, |
| | A117W DRD G83T)-AcGFP-P2A-mCherry |
| FKBP-134 | pELNS-FKBP13 (mutated to match FKBP12 active site Q72R, Q84E, |
| | A117W DRD H41R)-AcGFP-P2A-mCherry |
| FKBP-135 | pELNS-FKBP13 (mutated to match FKBP12 active site Q72R, Q84E, |
| | A117W DRD E61Y)-AcGFP-P2A-mCherry |
| FKBP-136 | pELNS-FKBP13 (mutated to match FKBP12 active site Q72R, Q84E, |
| | A117W DRD S108M)-AcGFP-P2A-mCherry |
| FKBP-137 | pELNS-FKBP13 (mutated to match FKBP12 active site Q72R, Q84E, |
| | A117W DRD P75L)-AcGFP-P2A-mCherry |
| FKBP-138 | pELNS-FKBP13 (mutated to match FKBP12 active site Q72R, Q84E, |
| | A117W DRD G124R)-AcGFP-P2A-mCherry |
| FKBP-139 | pELNS-FKBP13 (mutated to match FKBP12 active site Q72R, Q84E, |
| | A117W DRD E130R)-AcGFP-P2A-mCherry |
| FKBP-140 | pELNS-FKBP13 (mutated to match FKBP12 active site Q72R, Q84E, |
| | A117W DRD K135Y)-AcGFP-P2A-mCherry |
| FKBP-141 | pELNS-FKBP13 (mutated to match FKBP12 active site Q72R, Q84E, |
| | A117R DRD L52R)-AcGFP-P2A-mCherry |
| FKBP-142 | pELNS-FKBP13 (mutated to match FKBP12 active site Q72R, Q84E, |
| | A117W DRD T64G)-AcGFP-P2A-mCherry |
| FKBP-143 | pELNS-FKBP13 (mutated to match FKBP12 active site Q72R, Q84G, |
| | A117H DRD P43R)-AcGFP-P2A-mCherry |
| FKBP-144 | pELNS-FKBP13 (mutated to match FKBP12 active site Q72R, Q84E, |
| | A117H DRD D50R, E98M)-AcGFP-P2A-mCherry |
| FKBP-145 | pELNS-FKBP13 (mutated to match FKBP12 active site Q72R, Q84E, |
| | A117H DRD H41S, E61D)-AcGFP-P2A-mCherry |
| FKBP-146 | pELNS-FKBP13 (mutated to match FKBP12 active site Q72R, Q84E, |
| | A117H DRD L104S, I136L)-AcGFP-P2A-mCherry |
| FKBP-147 | pELNS-FKBP13 (mutated to match FKBP12 active site Q72R, Q84E, |
| | A117H DRD T64R, S108G, E137F)-AcGFP-P2A-mCherry |
| FKBP-148 | pELNS-FKBP13 (mutated to match FKBP12 active site Q72R, Q84E, |
| | A117H DRD D62N, D67N, D90N, M96G, E130K)-AcGFP-P2A- |
| | mCherry |
| FKBP-149 | pELNS-FKBP13 (mutated to match FKBP12 active site Q72R, Q84E, |
| | A117H DRD T64Q, L94G)-AcGFP-P2A-mCherry |
| FKBP-150 | pELNS-FKBP13 (mutated to match FKBP12 active site Q72R, Q84E, |
| | A117H DRD E61G, G124P)-AcGFP-P2A-mCherry |
| FKBP-151 | pELNS-FKBP13 (mutated to match FKBP12 active site Q72R, Q84R, |
| | A117W DRD M54A)-AcGFP-P2A-mCherry |
| FKBP-152 | pELNS-FKBP13 (mutated to match FKBP12 active site Q72R, Q84W, |
| | A117W DRD M54A)-AcGFP-P2A-mCherry |
| FKBP-153 | pELNS-FKBP13 (mutated to match FKBP12 active site Q72R, S79W, |
| | Q84E, A117W DRD M54A)-AcGFP-P2A-mCherry |
| FKBP-154 | pELNS-FKBP13 (mutated to match FKBP12 active site Q72R, S79W, |
| | Q84W, A117W DRD M54A)-AcGFP-P2A-mCherry |
| FKBP-155 | pELNS-FKBP13 (mutated to match FKBP12 active site Q72R, S79W, |
| | Q84R, A117W DRD M54A)-AcGFP-P2A-mCherry |
| FKBP-156 | pELNS-FKBP13 (mutated to match FKBP12 active site Q72R, T82W, |
| | Q84W, A117W DRD M54A)-AcGFP-P2A-mCherry |
| FKBP-157 | pELNS-FKBP13 (mutated to match FKBP12 active site Q72R, T82W, |
| | Q84E, A117W DRD M54A)-AcGFP-P2A-mCherry |
| FKBP-158 | pELNS-FKBP13 (mutated to match FKBP12 active site Q72R, Q84E, |
| | A117W DRD M54A G111E)-AcGFP-P2A-mCherry |
| FKBP-159 | pELNS-FKBP13 (mutated to match FKBP12 active site Q72R, Q84E, |
| | A117W DRD M54A G111Y)-AcGFP-P2A-mCherry |

To test the effect of mutations that increase FKBP13 responsiveness to tacrolimus (i.e., DRD-genic mutations) in the presence and absence of a binding site mutation, constructs FKBP13-039, FKBP13-040, FKBP13-041, FKBP13-042, FKBP13-043, FKBP13-044 and FKBP13-045 were made. The sequences for these constructs are set forth below. In constructs FKBP13-039, FKBP13-040, FKBP13-041, FKBP13-042, FKBP13-043, FKBP13-044 and FKBP13-045, the FKBP13 DRD sequence is bolded, the AcGFP amino acid sequence is underlined, and the mCherry sequence is italicized. It is understood that any of the constructs described herein comprising an FBKP13 DRD sequence can also comprise an AcGFP amino acid sequence (SEQ ID NO: 117, underlined in SEQ ID NOs:110-116 below) and/or a mCherry amino acid sequence (SEQ ID NO: 118, in italics SEQ ID NOs:110-116 below) as set forth in any one of constructs FKBP13-039, FKBP13-040, FKBP13-041, FKBP13-042, FKBP13-043, FKBP13-044 and FKBP13-045.

FKBP-039
FKBP13 DRD-AcGFP-mCherry AA sequence (SEQ ID NO: 110)
HCPIKSRKGDVLHMHYTGKLEDGTEFDSSLPQNQPFVFSLGTGQVIKGWDQGL
LGMCEGEKRKLVIPSELGYGERGAPPKIPGGATLVFEVELLKIERGGSMVSKGA
ELFTGIVPILIELNGDVNGHKFSVSGEGEGDATYGKLTLKFICTTGKLPVPWPTLVTTL
SYGVQCFSRYPDHMKQHDFFKSAMPEGYIQERTIFFEDDGNYKSRAEVKFEGDTLV
NRIELTGTDFKEDGNILGNKMEYNYNAHNVYIMTDKAKNGIKVNFKIRHNIEDGSVQ
LADHYQQNTPIGDGPVLLPDNHYLSTQSALSKDPNEKRDHMIYFGFVTAAAITHGM
DELYKGSGATNFSLLKQAGDVEENPGPLSKGEEDNMAIIKEFMRFKVHMEGSVNGHEF
EIEGEGEGRPYEGTQTAKLKVTKGGPLPFAWDILSPQFMYGSKAYVKHPADIPDYLKLSF
PEGFKWERVMNFEDGGVVTVTQDSSLQDGEFIYKVKLRGTNFPSDGPVMQKKTMGWEA
SSERMYPEDGALKGEIKQRLKLKDGGHYDAEVKTTYKAKKPVQLPGAYNVNIKLDITSHN
EDYTIVEQYERAEGRHSTGGMDELYK
FKBP13-040
FKBP13 DRD-AcGFP-mCherry AA sequence (SEQ ID NO: 111)
HCPIKSRKGDVLHMHYTGKLEDGTEFDSSLPQNQPFVFSLGTGQVIKGWGQGL
LGMCEGEKRKLVIPSELGYGERGAPPKIPGGATLVFEVELLKIERGGSMVSKGA
ELFTGIVPILIELNGDVNGHKFSVSGEGEGDATYGKLTLKFICTTGKLPVPWPTLVTTL
SYGVQCFSRYPDHMKQHDFFKSAMPEGYIQERTIFFEDDGNYKSRAEVKFEGDTLV
NRIELTGTDFKEDGNILGNKMEYNYNAHNVYIMTDKAKNGIKVNFKIRHNIEDGSVQ
LADHYQQNTPIGDGPVLLPDNHYLSTQSALSKDPNEKRDHMIYFGFVTAAAITHGM
DELYKGSGATNFSLLKQAGDVEENPGPLSKGEEDNMAIIKEFMRFKVHMEGSVNGHEF
EIEGEGEGRPYEGTQTAKLKVTKGGPLPFAWDILSPQFMYGSKAYVKHPADIPDYLKLSF
PEGFKWERVMNFEDGGVVTVTQDSSLQDGEFIYKVKLRGTNFPSDGPVMQKKTMGWEA
SSERMYPEDGALKGEIKQRLKLKDGGHYDAEVKTTYKAKKPVQLPGAYNVNIKLDITSHN
EDYTIVEQYERAEGRHSTGGMDELYK
FKBP-041
FKBP13 DRD-AcGFP-mCherry AA sequence (SEQ ID NO: 112)
HCPIKSRKGDVLHAHYTGKLEDGTEFDSSLPQNQPFVFSLGTGQVIKGWDQGL
LGMCEGEKRKLVIPSELGYGERGAPPKIPGGATLVFEVELLKIERGGSMVSKGA
ELFTGIVPILIELNGDVNGHKFSVSGEGEGDATYGKLTLKFICTTGKLPVPWPTLVTTL
SYGVQCFSRYPDHMKQHDFFKSAMPEGYIQERTIFFEDDGNYKSRAEVKFEGDTLV
NRIELTGTDFKEDGNILGNKMEYNYNAHNVYIMTDKAKNGIKVNFKIRHNIEDGSVQ
LADHYQQNTPIGDGPVLLPDNHYLSTQSALSKDPNEKRDHMIYFGFVTAAAITHGM
DELYKGSGATNFSLLKQAGDVEENPGPLSKGEEDNMAIIKEFMRFKVHMEGSVNGHEF
EIEGEGEGRPYEGTQTAKLKVTKGGPLPFAWDILSPQFMYGSKAYVKHPADIPDYLKLSF
PEGFKWERVMNFEDGGVVTVTQDSSLQDGEFIYKVKLRGTNFPSDGPVMQKKTMGWEA
SSERMYPEDGALKGEIKQRLKLKDGGHYDAEVKTTYKAKKPVQLPGAYNVNIKLDITSHN
EDYTIVEQYERAEGRHSTGGMDELYK
FKBP-042
FKBP13 DRD-AcGFP-mCherry AA sequence (SEQ ID NO: 113)
HCPIKSRKGDVLHMHYTGKLEDGTEFDSSLPQNQPFVFSLGTGQVIKGWDQGL
LGVCEGEKRKLVIPSELGYGERGAPPKIPGGATLVFEVELLKIERGGSMVSKGAE
LFTGIVPILIELNGDVNGHKFSVSGEGEGDATYGKLTLKFICTTGKLPVPWPTLVTTLS
YGVQCFSRYPDHMKQHDFFKSAMPEGYIQERTIFFEDDGNYKSRAEVKFEGDTLVN
RIELTGTDFKEDGNILGNKMEYNYNAHNVYIMTDKAKNGIKVNFKIRHNIEDGSVQL
ADHYQQNTPIGDGPVLLPDNHYLSTQSALSKDPNEKRDHMIYFGFVTAAAITHGMD
ELYKGSGATNFSLLKQAGDVEENPGPLSKGEEDNMAIIKEFMRFKVHMEGSVNGHEFEI
EGEGEGRPYEGTQTAKLKVTKGGPLPFAWDILSPQFMYGSKAYVKHPADIPDYLKLSFPE
GFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNED
YTIVEQYERAEGRHSTGGMDELYK
FKBP-043
FKBP13 DRD-AcGFP-mCherry AA sequence (SEQ ID NO: 114)
HCPIKSRKGDVLHMHYTGKLEDGTEFDSSLPRNQPFVFSLGTGEVIKGWGQGL
LGMCEGEKRKLVIPSELGYGERGHPPKIPGGATLVFEVELLKIERGGSMVSKGA
ELFTGIVPILIELNGDVNGHKFSVSGEGEGDATYGKLTLKFICTTGKLPVPWPTLVTTL
SYGVQCFSRYPDHMKQHDFFKSAMPEGYIQERTIFFEDDGNYKSRAEVKFEGDTLV
NRIELTGTDFKEDGNILGNKMEYNYNAHNVYIMTDKAKNGIKVNFKIRHNIEDGSVQ
LADHYQQNTPIGDGPVLLPDNHYLSTQSALSKDPNEKRDHMIYFGFVTAAAITHGM
DELYKGSGATNFSLLKQAGDVEENPGPLSKGEEDNMAIIKEFMRFKVHMEGSVNGHEF
EIEGEGEGRPYEGTQTAKLKVTKGGPLPFAWDILSPQFMYGSKAYVKHPADIPDYLKLSF
PEGFKWERVMNFEDGGVVTVTQDSSLQDGEFIYKVKLRGTNFPSDGPVMQKKTMGWEA
SSERMYPEDGALKGEIKQRLKLKDGGHYDAEVKTTYKAKKPVQLPGAYNVNIKLDITSHN
EDYTIVEQYERAEGRHSTGGMDELYK
FKBP-044
FKBP13 DRD-AcGFP-mCherry AA sequence (SEQ ID NO: 115)
HCPIKSRKGDVLHAHYTGKLEDGTEFDSSLPRNQPFVFSLGTGEVIKGWDQGLL
GMCEGEKRKLVIPSELGYGERGHPPKIPGGATLVFEVELLKIERGGSMVSKGAE
LFTGIVPILIELNGDVNGHKFSVSGEGEGDATYGKLTLKFICTTGKLPVPWPTLVTTLS
YGVQCFSRYPDHMKQHDFFKSAMPEGYIQERTIFFEDDGNYKSRAEVKFEGDTLVN
RIELTGTDFKEDGNILGNKMEYNYNAHNVYIMTDKAKNGIKVNFKIRHNIEDGSVQL
ADHYQQNTPIGDGPVLLPDNHYLSTQSALSKDPNEKRDHMIYFGFVTAAAITHGMD
ELYKGSGATNFSLLKQAGDVEENPGPLSKGEEDNMAIIKEFMRFKVHMEGSVNGHEFEI
EGEGEGRPYEGTQTAKLKVTKGGPLPFAWDILSPQFMYGSKAYVKHPADIPDYLKLSFPE
GFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNED
YTIVEQYERAEGRHSTGGMDELYK
FKBP-045
FKBP13 DRD-AcGFP-mCherry AA sequence (SEQ ID NO: 116)
HCPIKSRKGDVLHMHYTGKLEDGTEFDSSLPRNQPFVFSLGTGEVIKGWDQGL
LGVCEGEKRKLVIPSELGYGERGHPPKIPGGATLVFEVELLKIERGGSMVSKGA
ELFTGIVPILIELNGDVNGHKFSVSGEGEGDATYGKLTLKFICTTGKLPVPWPTLVTTL
SYGVQCFSRYPDHMKQHDFFKSAMPEGYIQERTIFFEDDGNYKSRAEVKFEGDTLV
NRIELTGTDFKEDGNILGNKMEYNYNAHNVYIMTDKAKNGIKVNFKIRHNIEDGSVQ
LADHYQQNTPIGDGPVLLPDNHYLSTQSALSKDPNEKRDHMIYFGFVTAAAITHGM
DELYKGSGATNFSLLKQAGDVEENPGPLSKGEEDNMAIIKEFMRFKVHMEGSVNGHEF
EIEGEGEGRPYEGTQTAKLKVTKGGPLPFAWDILSPQFMYGSKAYVKHPADIPDYLKLSF
PEGFKWERVMNFEDGGVVTVTQDSSLQDGEFIYKVKLRGTNFPSDGPVMQKKTMGWEA
SSERMYPEDGALKGEIKQRLKLKDGGHYDAEVKTTYKAKKPVQLPGAYNVNIKLDITSHN
EDYTIVEQYERAEGRHSTGGMDELYK

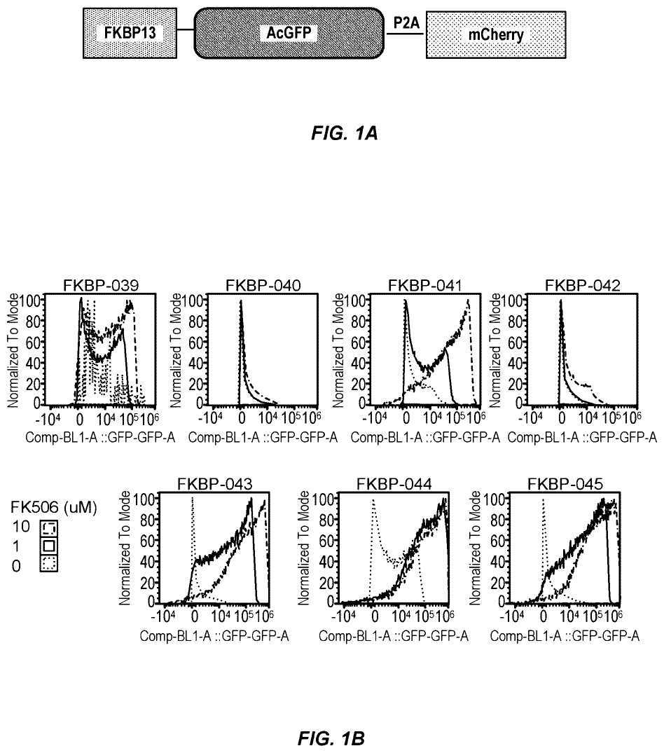

FIG. 1A is a schematic showing the general organization of an exemplary nucleic acid construct comprising (i) a nucleic acid sequence encoding a potential FKBP DRD sequence; (ii) a nucleic acid sequence encoding Aequorea coerulescens GFP (AcGFP); (iii) a nucleic acid sequence encoding an exemplary self-cleaving peptide (porcine teschovirus-1 2A (P2A)); and (iv) a nucleic acid sequence encoding mCherry.

HEK-293T cells were cultured in standard media (Dulbecco's Modified Eagle Medium (DMEM) (Fisher Scientific, Hampton, NH, Cat #11-960-044) with 10% FBS (Fisher Scientific, Cat #10-082-147) and 1% penicillin-streptomycin (Thermo Fisher Scientific, Waltham, MA, Cat #15140122); and transiently transfected with FKBP13-039, FKBP13-040, FKBP13-041, FKBP13-042, FKBP13-043, FKBP13-044 or FKBP13-045 constructs in a six well plate, using Lipofectamine® 2000 (Fisher Scientific, Cat #11-668-027). 24 hours later transfected HEK-293T cells were treated with either 0, 1 or 10 μM FK506 (tacrolimus). After 24 hours of FK506 treatment, cells were harvested by trypsinization, and flow analysis was performed using the Attune NXT flow cytometer (Thermo Fisher Scientific).

The DRD in FKBP13-039 was a wildtype (WT) FKBP13 (i.e., a 98 amino acid fragment of SEQ ID NO: 1). The DRDs in FKBP13-040, FKBP13-041 and FKBP13-042 were designed to comprise DRD-genic mutations D90G, M54A and M96V, respectively, but did not include binding site mutations. The FKBP13-043, FKBP13-044, and FKBP13-045 constructs included DRD-genic mutations D90G, M54A and M96V, respectively, and three binding site mutations (Q72R, Q84E, and A117H).

As shown in FIG. 1B, all DRD-genic mutations were effective in destabilizing AcGFP and reducing AcGFP fluorescence. AcGFP was stabilized and the fluorescence signal was increased more effectively in the presence of the binding site mutations (FKBP13-043, FKBP13-044 or FKBP13-045). This is evidenced by constructs without the binding site mutation having less responsiveness to 1 μM FK506. Interestingly, WT FKBP13 (FKBP13-043) also had DRD activity.

To determine if any of the FKBP13 DRDs had EC₅₀ values below achievable plasma drug concentration in humans, tacrolimus dose response experiments were carried out. The results showed that, overall, the response to tacrolimus was improved in the presence of the binding site mutations (Table 3).

TABLE 3
Tacrolimus response for exemplary FKBP13 DRDs

| | DRD | Binding site | | Fold |
|---|---|---|---|---|
| Construct | mutant | matched | EC₅₀ (μM) | change |
| FKBP13-039 | WT | No | NA | 4.9 |
| FKBP13-040 | D90G | No | NA | 1 |
| FKBP13-041 | M54A | No | NA | 7.8 |
| FKBP13-042 | M96V | No | NA | 1.3 |
| FKBP13-043 | D90G | Yes | NA | 26.5 |
| FKBP13-044 | M54A | Yes | 0.23 | 9.4 |
| FKBP13-045 | M96V | Yes | NA | 23.2 |

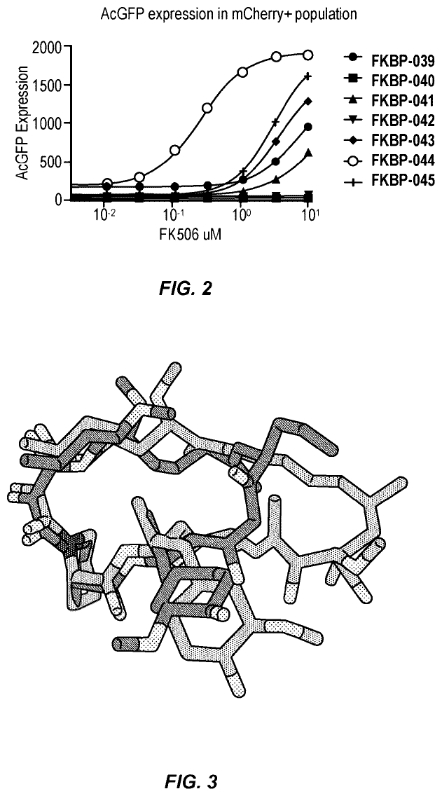

The regulation of AcGFP, from FKBP13-AcGFP-mCherry constructs, in Jurkat cells, was also studied. Jurkat Clone E6-1 cells were cultured in standard media (RPMI+GlutaMAX Supplement (Life Technologies, Carlsbad, CA Cat #61870127), Fetal Bovine Serum (FBS) (Life Technologies Cat #10-082-147)). Jurkat cells were transduced with lentivirus produced with each of FKBP-039, FKBP-040, FKBP-041, FKBP-042, FKBP-043, FKBP-044, or FKBP-045 constructs. Jurkat cells were transduced until they were 10-25% positive, as determined on day 5 post-transduction, by using mCherry as a transduction marker. On day 5 post-transduction, cells were treated with doses of tacrolimus starting with a top concentration of 10 μM or DMSO. After a 24 hr incubation period, GFP expression was measured in cells treated with tacrolimus or DMSO. The Geometric Mean Fluorescent Intensity (MFI) of GFP expression in mCherry+ cells was plotted and dose response curve fits were performed using Prism Software (San Diego, CA). The dose response curves are provided in FIG. 2. The FKBP-044 construct had the best EC₅₀ and fold-change characteristics and provided a guidepost for further FKBP13 DRD development.

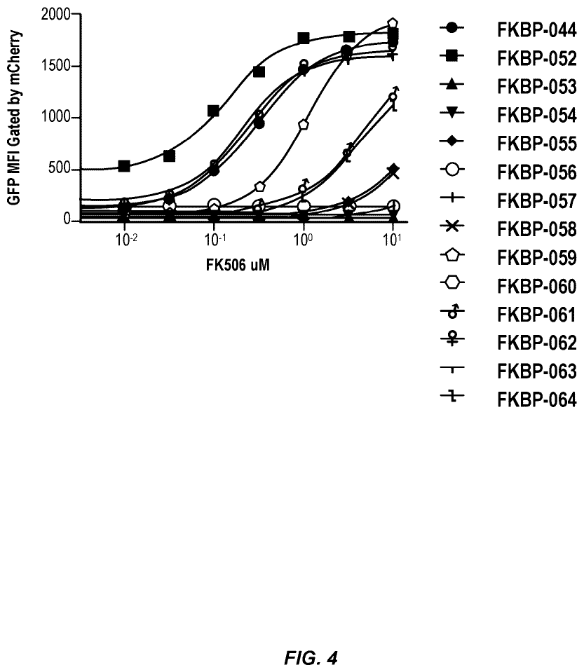

In another experiment, several FKBP13 DRD constructs were transiently transfected into HEK293 cells, as described above, and analyzed using a Celigo Image Cytometer (Nexcelom, Lawrence, MA). FIG. 4 provides tacrolimus dose response FACS data for Jurkat cells stably transduced with FKBP-044, FKBP-052, FKBP-053, FKBP-054, FKBP-055, FKBP-056, FKBP-057, FKBP-058, FKBP-059, FKBP-060, FKBP-061, FKBP-062, FKBP-063, and FKBP-064 constructs.

Two, different Protein Data Bank (PDB) codes (2PBC and 4NNR) exist for crystal structures of FKBP13. Unlike the 98 amino acid sequence in 4NNR, which corresponds to truncated WT (SEQ ID NO: 2), the sequence in PDB ID 2PBC has two mutations, H41G and C42S. Since disulfides are often implicated in protein stabilization, the role of this disulfide bond, in stabilization of FKBP13, was investigated. Constructs FKBP-052, FKBP-062 and FKBP-063 have abrogated disulfides. While the FKBP-052 construct has a wildtype FKBP13 sequence, the FKBP-062 and FKBP-063 constructs have the same DRD-genic mutations as the FKBP-044 construct, with the FKBP-062 construct having two cysteine to serine mutations, while the FKBP-063 construct only has one. Unexpectedly, abrogating the disulfide bonds did not create a significant difference in DRD function, as shown in FIG. 4.

TABLE 4
Changes Associated with Binding Site Mutations

| Con- | DRD | Binding site | Disul- | | Fold |
|---|---|---|---|---|---|
| struct | mutant | Mutations | fide | EC₅₀(μM) | change |
| FKBP-044 | M54A | Q72R, Q84E, | Yes | 0.3 | 12.6 |
| | | A117H | | | |
| FKBP-052 | WT | Q72R, Q84E, | Abro- | 0.14 | 3.5 |
| | | A117H | gated | | |
| FKBP-053 | D90G, | Q72R, Q84E, | Yes | NA | 1.0 |
| | F66H | A117H | | | |
| FKBP-054 | D90G, | Q72R, Q84E, | Yes | NA | 0.9 |
| | L127K | A117H | | | |
| FKBP-055 | F78V | Q72R, Q84E, | Yes | 43 | 9.0 |
| | | A117H | | | |
| FKBP-056 | F129K | Q72R, Q84E, | Yes | NA | 1.0 |
| | | A117H | | | |
| FKBP-057 | I106A | Q72R, Q84E, | Yes | ~200 | 2.7 |
| | | A117H | | | |
| FKBP-058 | L52A | Q72R, Q84E, | Yes | 22 | 8.1 |
| | | A117H | | | |
| FKBP-059 | L93A | Q72R, Q84E, | Yes | 1.1 | 27.3 |
| | | A117H | | | |
| FKBP-060 | L104A | Q72R, Q84E, | Yes | NA | 2.7 |
| | | A117H | | | |
| FKBP-061 | L133A | Q72R, Q84E, | Yes | 5.5 | 16.6 |
| | | A117H | | | |
| FKBP-062 | M54A, | Q72R, Q84E, | Abro- | 0.25 | 10.4 |
| | C42S, C97S | A117H | gated | | |
| FKBP-063 | M54A, | Q72R, Q84E, | Abro- | 0.22 | 7.3 |
| | C42S | A117H | gated | | |
| FKBP-064 | R102L | Q72R, Q84E, | Yes | 4.2 | 21.6 |
| | | A117H | | | |

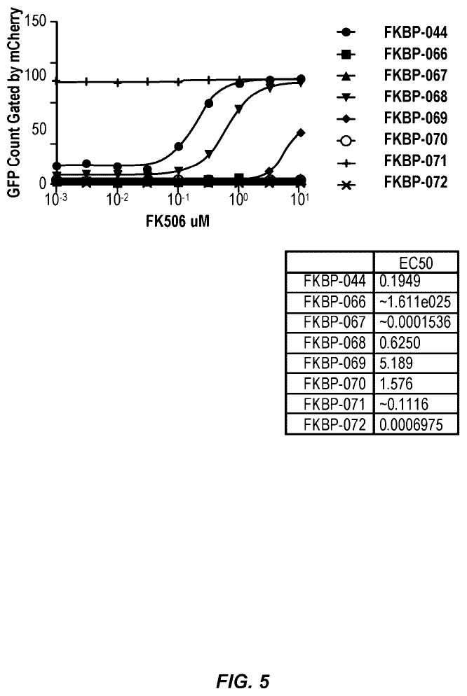

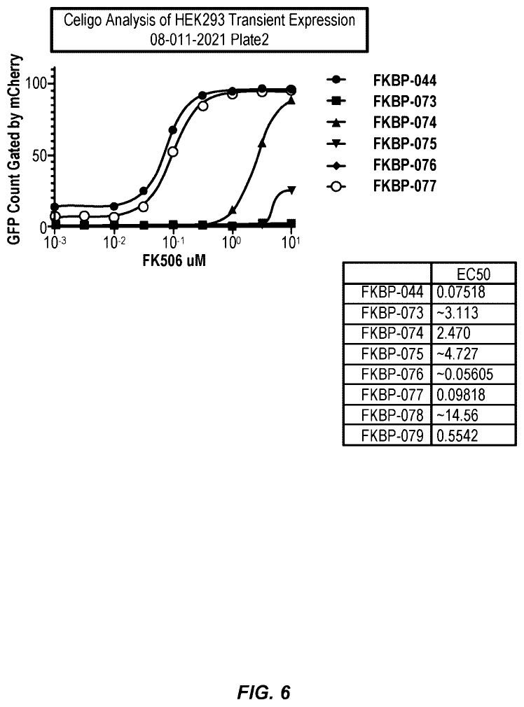

To test the effects of other substitutions instead of methionine at residue number 54 and leucine at position 93 FKBP13 DRD constructs were generated and were transiently transfected into HEK293 cells, as described above, and analyzed using a Celigo Image Cytometer (Nexcelom, Lawrence, MA). Tacrolimus dose response curves for FKBP-044 (M54A), FKBP-066 (M54K), FKBP-067 (M54H), FKBP-068 (M54S), FKBP-069 (M54G), FKBP-070 (M54D) and FKBP-071 (M54V). FKBP-072 (L93K) are shown in FIG. 5. FKBP-068 was the closest in performance to the original M54A substitution. Tacrolimus dose response curves for FKBP-044, FKBP-073 (L93H), FKBP-074 (L93S), FKBP-075 (L93G), FKBP-076 (L93D), FKBP-077 (L93V) are shown in FIG. 6. FKBP-077 performed similarly as FKBP-044.

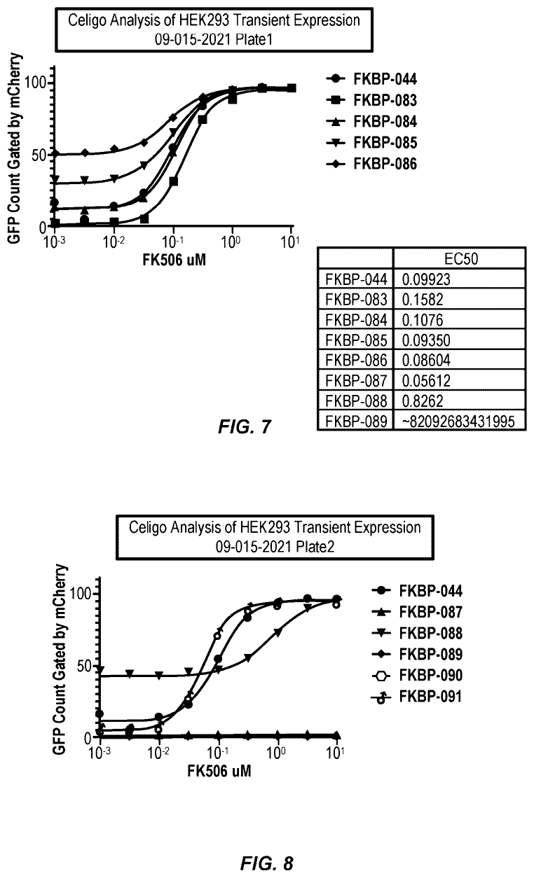

In another experiment, to explore the possibility of further truncating FKBP13, constructs (FKBP-044, FKBP-083, FKBP-084, FKBP-085, and FKBP-086) were transiently transfected into HEK293 cells, as described above, and analyzed using a Celigo Image Cytometer (Nexcelom, Lawrence, MA). Tacrolimus dose response curves are shown in FIG. 7. A combination of DRD-genic M54A with truncation of the N-terminal histidine lowers basal (FKBP-083).

In another experiment, several constructs (FKBP-044, FKBP-087, FKBP-088, FKBP-089, FKBP-090, and FKBP-091), which had alternate binding sites designed to improve sensitivity to tacrolimus, were transiently transfected into HEK293 cells, as described above, and analyzed using a Celigo Image Cytometer (Nexcelom, Lawrence, MA). Tacrolimus dose response curves are shown in FIG. 8. FKBP-091 with the mutation A117W, instead of A117H in FKBP-044 combined with DRD-genic mutation M54A has superior drug responsiveness.

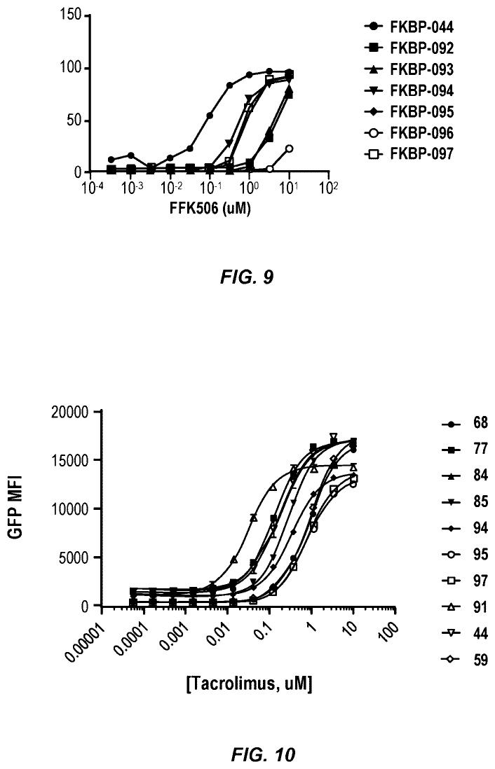

In another experiment, several constructs (FKBP-044, FKBP-092, FKBP-093, FKBP-094, FKBP-095, FKBP-096 and FKBP-097) designed to explore additional DRD-genic mutations were transiently transfected into HEK293 cells, as described above, and analyzed using a Celigo Image Cytometer (Nexcelom, Lawrence, MA). Tacrolimus dose response curves are shown in FIG. 9.

FIG. 10 provides tacrolimus dose response FACS data for Jurkat cells stably transduced with FKBP-044, FKBP-059, FKBP-068, FKBP-077, FKBP-084, FKBP-085, FKBP-091, FKBP-094, FKBP-095, and FKBP-097 constructs. These experiments included a second round of binding site design and some mutants M54S (068), L93V (077), L93A (059) and M54A (044) previously identified as being of interest. The EC₅₀ of FKBP-091, was found to be 30 nM, in this experiment. See Table 5.

TABLE 5
EC 50 Changes Associated with Mutations

| Construct | Mutations | EC₅₀ | Fold Change |
|---|---|---|---|
| FKBP-044 | Q72R, Q84E, A117H, M54A | 0.1645 | 11.805 |
| FKBP-059 | Q72R, Q84E, A117H, L93A | 0.898 | 31.085 |
| FKBP-068 | Q72R, Q84E, A117H, M54S | 0.8948 | 43.62 |
| FKBP-077 | Q72R, Q84E, A117H, L93V | 0.1318 | 10.3 |
| FKBP-084 | Q72R, Q84E, A117H, trunc2, M54A | 0.1597 | 12.42 |
| FKBP-085 | Q72R, Q84E, A117H, trunc 2, WT | 0.277 | 16.09 |
| FKBP-091 | Q72R, Q84E, A117W, M54A | 0.03043 | 12.36 |
| FKBP-094 | Q72R, Q84E, A117H, P107S | 0.3383 | 13.5 |
| FKBP-095 | Q72R, Q84E, A117H, P122S | 0.7926 | 31.4 |
| FKBP-097 | Q72R, Q84E, A117H, E100S | 0.8092 | 36.75 |

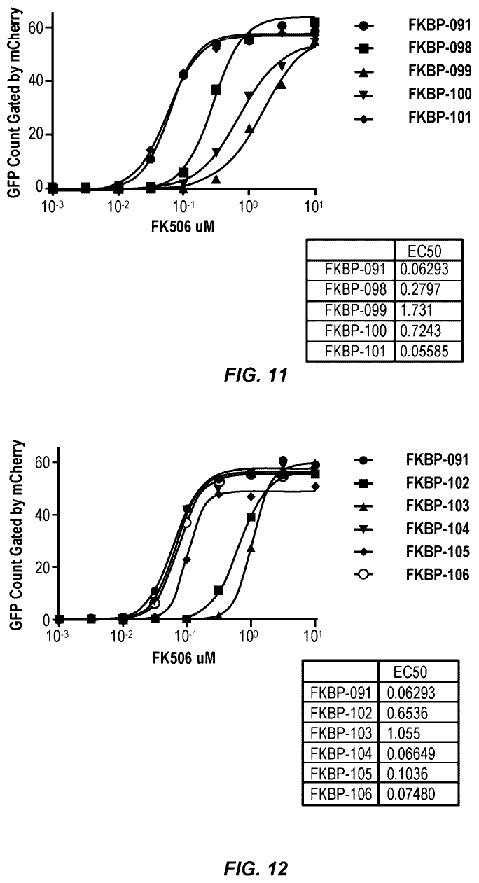

Other constructs (FKBP-091, FKBP-098, FKBP-099, FKBP-100, and FKBP-101) were transiently transfected into HEK293 cells, as described above, and analyzed using a Celigo Image Cytometer (Nexcelom, Lawrence, MA). Tacrolimus dose response curves are shown in FIG. 11.

In another experiment, several constructs (FKBP-091, FKBP-102, FKBP-103, FKBP-104, FKBP-105 and FKBP-106), designed to combine the binding site mutation identified in FKBP-091 with novel DRD-genic mutations, were transiently transfected into HEK293 cells, as described above, and analyzed using a Celigo Image Cytometer (Nexcelom, Lawrence, MA). Tacrolimus dose response curves are shown in FIG. 12. All constructs have a second generation binding site (i.e., the binding site in FKBP-091 (Q72R, Q84E, A117W), except—the FKBP-106 construct, which has a different binding site (Q72R, Q84E, A117Y).

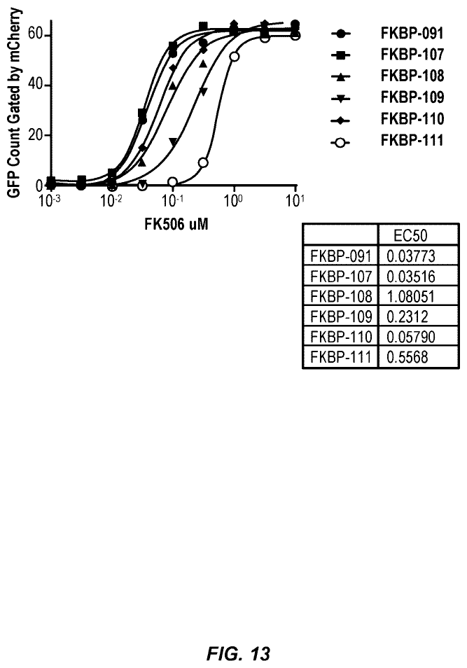

In another experiment, several other constructs (FKBP-091, FKBP-107, FKBP-108, FKBP-109, FKBP-110, and FKBP-111), designed to combine the binding site mutation identified in FKBP-091 with novel DRD-genic mutations, were transiently transfected into HEK293 cells, as described above, and analyzed using a Celigo Image Cytometer (Nexcelom, Lawrence, MA). Tacrolimus dose response curves are shown in FIG. 13. All constructs have a second-generation binding site (i.e., the binding site in FKBP-091 (Q72R, Q84E, A117W)).

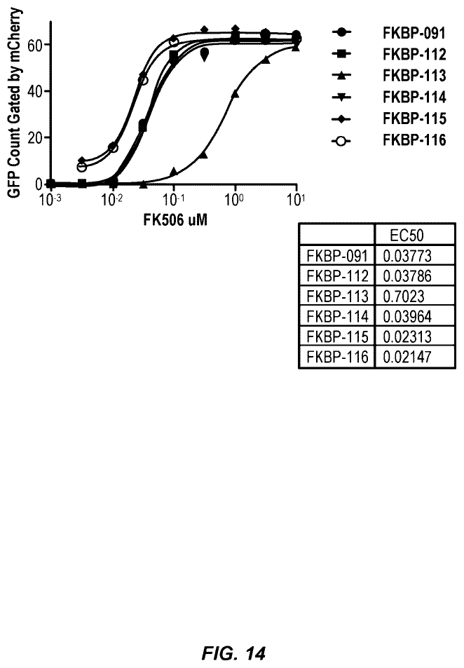

In another experiment, several constructs (FKBP-091, FKBP-112, FKBP-113, FKBP-114, FKBP-115 and FKBP-116), designed to combine the binding site mutation identified in FKBP-091 with novel DRD-genic mutations, were transiently transfected into HEK293 cells, as described above, and analyzed using a Celigo Image Cytometer (Nexcelom, Lawrence, MA). Tacrolimus dose response curves are shown in FIG. 14.

### Example 2

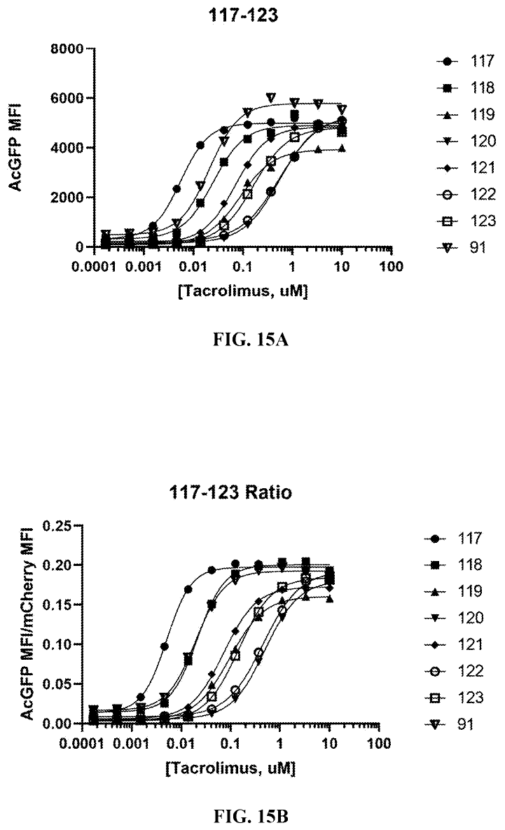

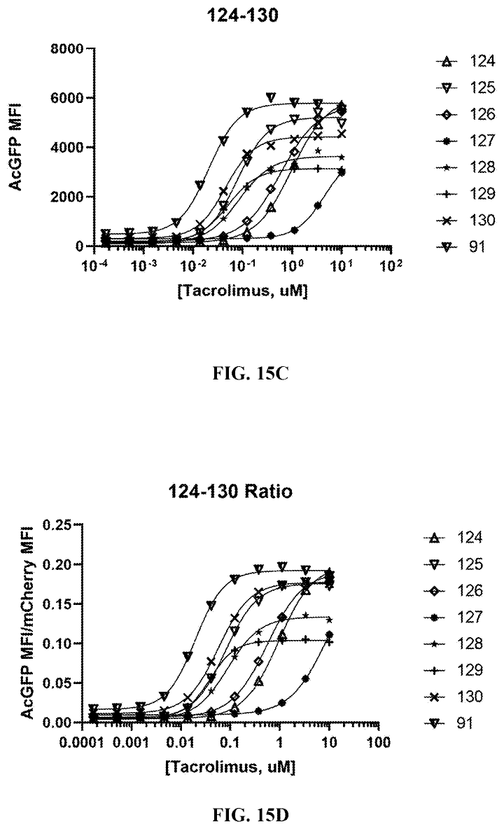

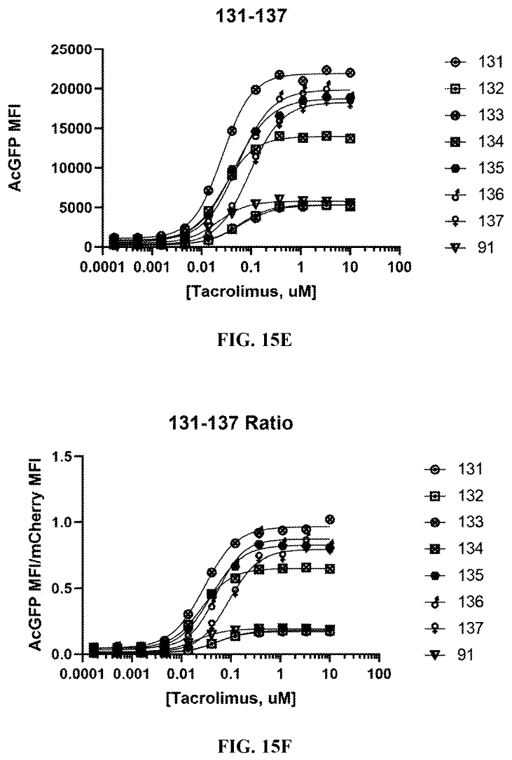

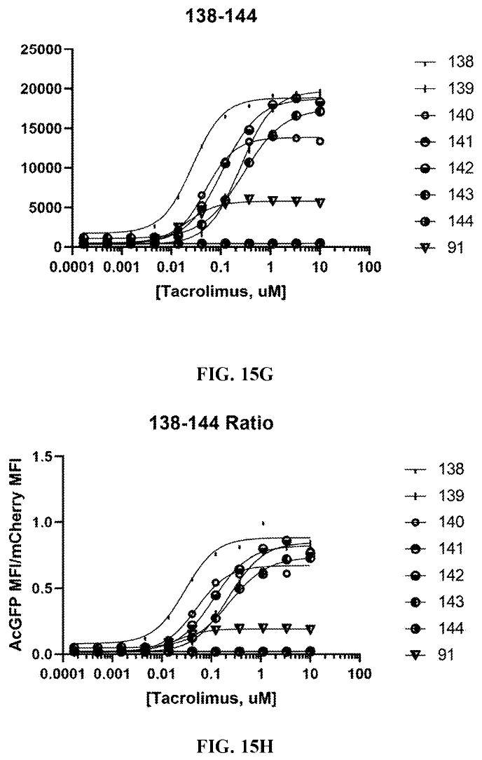

Additional experiments were performed with constructs FKBP-117 through FKBP-150 to determine sensitivity to tacrolimus. Constructs FKBP-117 through FKBP-150 were stably transduced into Jurkat cells, as described above, and their response to tacrolimus was analyzed using flow cytometry. FKBP-091 was used as a comparative construct. The EC₅₀ value for AcGFP, EC₅₀ value for AcGFP/mCherry and Fold Change for each construct are set forth in Table 6. Tacrolimus dose response curves for constructs FKBP-117 through FKBP-150 are set forth in FIGS. 15A-15J.

TABLE 6
EC₅₀ Values for Constructs FKBP-117 through FKBP-150

| Construct Name | Ec₅₀ AcGFP | Ec₅₀ AcGFP/mCherry | Fold Change |
|---|---|---|---|
| FKBP-091 | 0.0208 | 0.0196 | 11.29 |
| FKBP-117 | 0.0055 | 0.005082 | 15.75 |
| FKBP-118 | 0.02488 | 0.02124 | 12.95 |
| FKBP-119 | 0.08649 | 0.09446 | 31.91 |
| FKBP-120 | 0.5578 | 0.4971 | 30.53 |
| FKBP-121 | 0.07026 | 0.07177 | 28.35 |
| FKBP-122 | 0.5581 | 0.4584 | 22.51 |
| FKBP-123 | 0.1616 | 0.1451 | 48.38 |
| FKBP-124 | 0.9797 | 0.9361 | 40.13 |
| FKBP-125 | 0.07903 | 0.08004 | 29.07 |
| FKBP-126 | 0.5836 | 0.5538 | 25.70 |
| FKBP-127 | 4.497 | 9.253 | 10.25 |
| FKBP-128 | 0.08399 | 0.08937 | 31.75 |
| FKBP-129 | 0.05061 | 0.03872 | 17.11 |
| FKBP-130 | 0.04522 | 0.05776 | 14.40 |
| FKBP-131 | 0.05978 | 0.05128 | 21.30 |
| FKBP-132 | 0.05827 | 0.05606 | 16.27 |
| FKBP-133 | 0.02618 | 0.02875 | 19.11 |
| FKBP-134 | 0.02787 | 0.02542 | 15.64 |
| FKBP-135 | 0.04373 | 0.03843 | 25.52 |
| FKBP-136 | 0.05329 | 0.05134 | 22.46 |
| FKBP-137 | 0.09312 | 0.09171 | 45.79 |
| FKBP-138 | 0.02692 | 0.0291 | 10.43 |
| FKBP-139 | 0.2447 | 0.2239 | 52.24 |
| FKBP-140 | 0.04851 | 0.0498 | 28.70 |
| FKBP-141 | 9.262 | 0.00205 | 1.00 |
| FKBP-142 | 0.1067 | 0.1127 | 33.42 |
| FKBP-143 | 0.2829 | 0.2332 | 14.97 |
| FKBP-144 | 15.33 | Unstable | 1.24 |
| FKBP-145 | 0.2016 | 0.1488 | 11.05 |
| FKBP-146 | 5.075 | Unstable | 1.19 |
| FKBP-147 | 9.419 | 7.233 | 26.65 |
| FKBP-148 | Unstable | Unstable | 1.09 |
| FKBP-149 | 0.9762 | 1.032 | 39.28 |
| FKBP-150 | 7.124 | 7.356 | 21.15 |

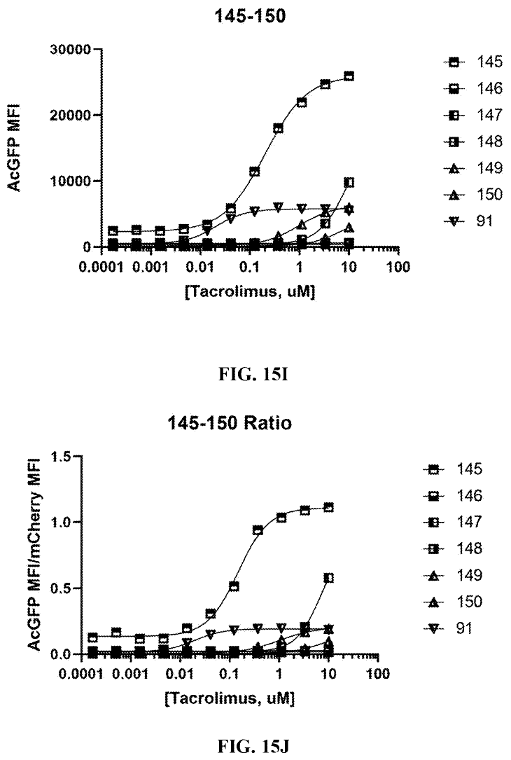

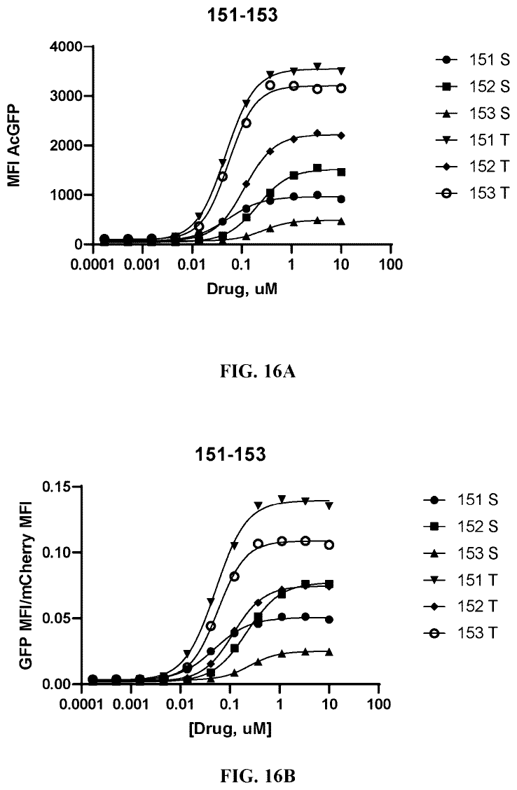

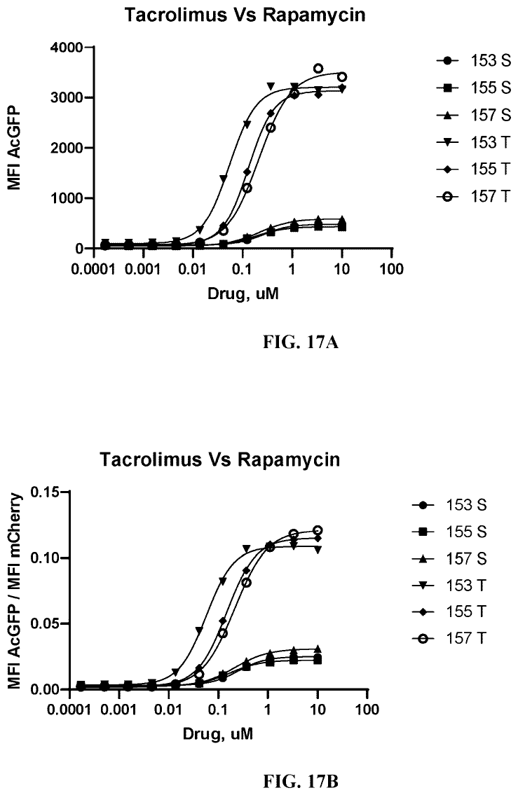

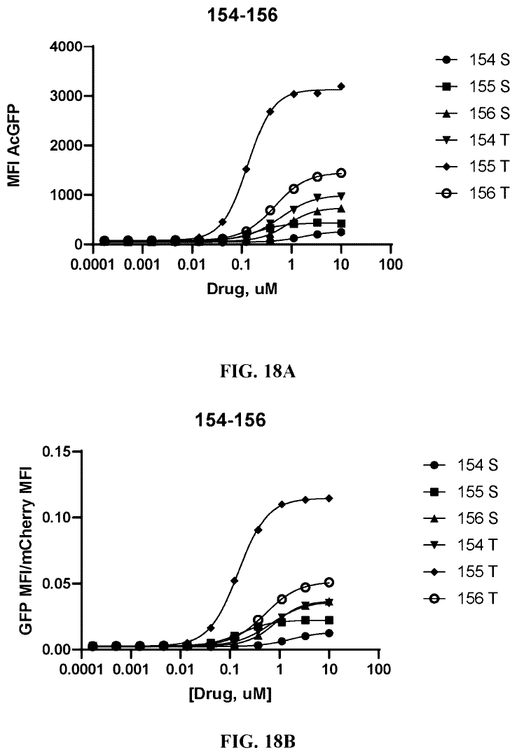

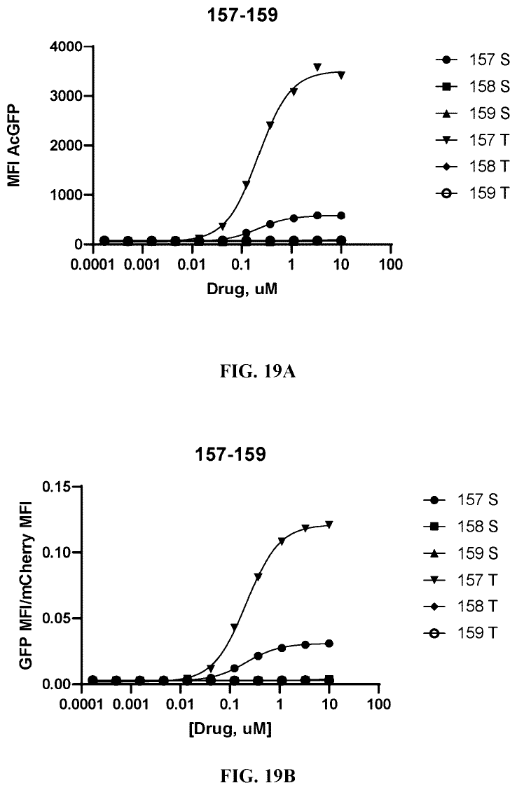

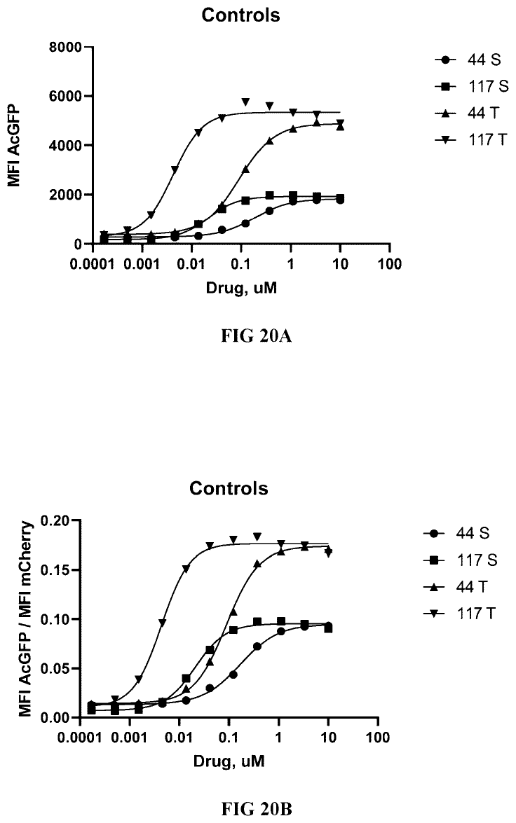

Constructs FKBP-151-FKBP-159 were analyzed for differences in tacrolimus and sirolimus (rapamycin) sensitivity. These constructs have mutations in the periphery of the binding site for FKBP13, which were designed to enable differential sensitivity to tacrolimus and sirolimus. Differences in the shape and size of rapamycin and tacrolimus (FIG. 3) were used to design these mutants. Constructs FKBP-151 through FKBP-159, along with controls FKBP-044 and FKBP-117 were stably transduced into Jurkat cells, as described above, and their response to tacrolimus or sirolimus was analyzed using flow cytometry. The EC₅₀ value, Norm EC₅₀ value and Fold Change for each construct, for tacrolimus and rapamycin, are set forth in Tables 7 and 8, respectively. Normalized EC₅₀ values were calculated from the mCherry normalized GFP MFI values, which accounted for any differences in mCherry intensity between samples. Tacrolimus vs. Rapamycin dose response curves for constructs FKBP-151, FKBP-152 and FKBP-153 are shown in FIGS. 16A and 16B. Tacrolimus vs. Rapamycin dose response curves for constructs FKBP-153, FKBP-155 and FKBP-157 are shown in FIGS. 17A and 17B. Tacrolimus vs. rapamycin dose response curves for constructs Tacrolimus vs. rapamycin dose response curves for constructs FKBP-154, FKBP-155 and FKBP-156 are shown in FIGS. 18A and 18B. Tacrolimus vs. Rapamycin dose response curves for constructs FKBP-157, FKBP-158 and FKBP-159 are shown in FIGS. 19A and 19B. Tacrolimus vs. Rapamycin dose response curves for comparative constructs FKBP-044 and FKBP-117 are shown in FIGS. 20A and 20B. Constructs 153, 155 and 157 show the largest difference between rapamycin and tacrolimus as shown in Table 9.

TABLE 7
Tacrolimus Response Values for Constructs FKBP-151-FKBP-159

| Construct | EC₅₀ | Norm EC₅₀ | Fold Change |
|---|---|---|---|
| FKBP-44 | 0.09165 | 0.0893 | 12.98 |
| FKBP-117 | 0.004257 | 0.004386 | 18.6 |
| FKBP-151 | 0.04744 | 0.05103 | 42.03 |
| FKBP-152 | 0.114 | 0.1169 | 30.34 |
| FKBP-153 | 0.05422 | 0.05714 | 30.07 |
| FKBP-154 | 0.5824 | 0.6682 | 14 |
| FKBP-155 | 0.1315 | 0.1481 | 41.68 |
| FKBP-156 | 0.4549 | 0.5122 | 18.36 |
| FKBP-157 | 0.2135 | 0.214 | 51.33 |
| FKBP-158 | 613.6 | — | 1.25 |
| FKBP-159 | 4.474 | 1.153 | 1.06 |

TABLE 8
Rapamycin Response Values for Constructs FKBP-151-FKBP-159

| Construct | EC₅₀ | Norm EC₅₀ | Fold Change |
|---|---|---|---|
| FKBP-44 | 0.185 | 0.1704 | 4.4 |
| FKBP-117 | 0.02165 | 0.02117 | 6.9 |
| FKBP-151 | 0.052 | 0.0482 | 10.15 |
| FKBP-152 | 0.2076 | 0.2194 | 23.17 |
| FKBP-153 | 0.265 | 0.2512 | 4.8 |
| FKBP-154 | 1.659 | 1.624 | 3.9 |
| FKBP-155 | 0.1832 | 0.1662 | 5.6 |
| FKBP-156 | 0.816 | 0.7944 | 8.77 |
| FKBP-157 | 0.2198 | 0.2132 | 7.92 |
| FKBP-158 | 3.541 | 6.749 | 0.84 |
| FKBP-159 | 3.669 | 4.851 | 0.81 |

TABLE 9
Ratio of EC₅₀ and fold change reported
for Tacrolimus and Rapamycin

| | EC₅₀ (tacrolimus)/ | Fold change (tacrolimus)/ |
|---|---|---|
| | EC₅₀ | Fold change |
| Construct | (rapamycin) | (rapamycin) |
| FKBP-44 | 0.50 | 2.95 |
| FKBP-117 | 0.20 | 2.70 |
| FKBP-151 | 0.91 | 4.14 |
| FKBP-152 | 0.55 | 1.31 |
| FKBP-153 | 0.20 | 6.26 |
| FKBP-154 | 0.35 | 3.59 |
| FKBP-155 | 0.72 | 7.44 |
| FKBP-156 | 0.56 | 2.09 |
| FKBP-157 | 0.97 | 6.48 |
| FKBP-158 | NA | 1.49 |
| FKBP-159 | 1.22 | 1.31 |

### Example 3

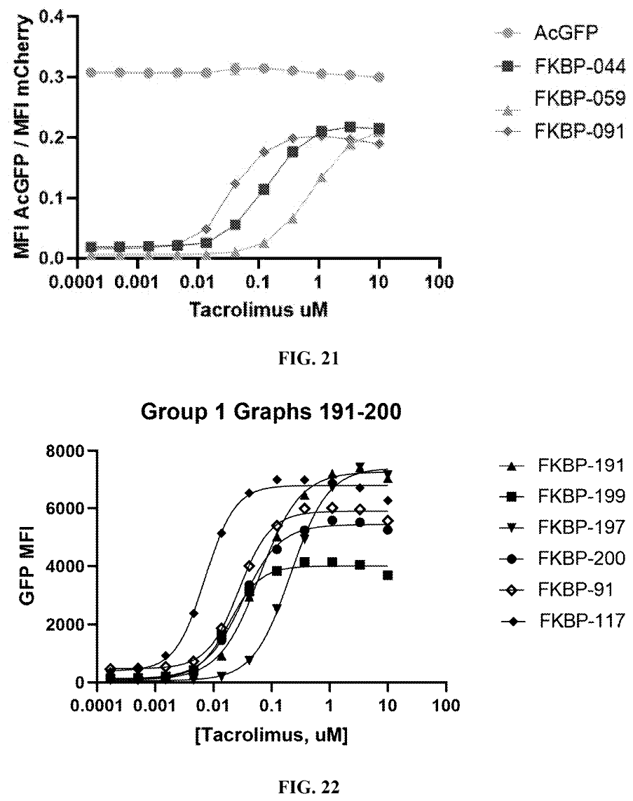

Additional experiments were performed to compare constitutively expressed AcGFP abundance with FKBP13/tacrolimus regulated AcGFP abundance levels. FKBP13 was fused to a protein of interest (i.e., AcGFP) to examine the effects on the maximal abundance of AcGFP. Constructs FKBP-044, FKBP-059 and FKBP-091 and AcGFP-001 were stably transduced into Jurkat cells as previously described and dose response experiments were performed. MFI was read by flow cytometry (FIG. 21). Regulated FKBP13 reached approximately 70% of constitutive AcGFP MFI values.

### Example 4

Additional experiments were performed for constructs FKBP-160-292. The constructs are shown in Table 10 and the top 30 drug responsive domains for Cas9 screening are shown in Table 11.

TABLE 10
FKBP-160-292 Construct Sequences

| Construct | | | |
|---|---|---|---|
| Name | Mutation | Nucleic Acid Sequence | Amino Acid Sequence |
| FKBP-160 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIER |
| | M54A | GCGAGAAGAGAAAGCTGGTCATCCCTAGCGAACTC | (SEQ ID NO: 120) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 119) | |
| FKBP-161 | Q72R, | CACTGCCCAATCAAGAGCAGAAAAGGCGACGTGCT | HCPIKSRKGDVLHSHYTGKL |
| | Q84E, | GCACagtCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | DRD | TTCGTGTTTAGCCTCGGCACCGGCgaacggATCAA | KRKLVIPSELGYGERGWPPK |
| | M54S | AGGCTGGGATCAAGGCCTGCTGGGAATGTGCGAAG | IPGGATLVFEVELLKIER |
| | V85R | GCGAGAAGAGAAAGCTGGTCATCCCTAGCGAACTC | (SEQ ID NO: 122) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 121) | |
| FKBP-162 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | DRD | TTCGTGTTTAGCCTCGGCACCGGCgaacggATCAA | KRKLVIPSELGYGERGWPPK |
| | M54A, | AGGCTGGGATCAAGGCCTGCTGGGAATGTGCGAAG | IPGGATLVFEVELLKIE |
| | del | GCGAGAAGAGAAAGCTGGTCATCCCTAGCGAACTC | (SEQ ID NO: 124) |
| | c-term | GGCTACGGCGAAAGAGGCtggCCTCCAAAGATTCC | |
| | R | TGGCGGAGCCACACTGGTGTTCGAAGTGGAACTGC | |
| | V85R | TGAAGATCGAG (SEQ ID NO: 123) | |
| FKBP-163 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGVCEGE |
| | DRD | TTCGTGTTTAGCCTCGGCACCGGCgaacggATCAA | KRKLVIPSELGYGERGWPPK |
| | M54A, | AGGCTGGGATCAAGGCCTGCTGGGAgttTGCGAAG | IPGGATLVFEVELLKIER |
| | M96V | GCGAGAAGAGAAAGCTGGTCATCCCTAGCGAACTC | (SEQ ID NO: 126) |
| | V85R | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 125) | |
| FKBP-164 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | DRD | TTCGTGTTTAGCCTCGGCACCGGCgaacggATCAA | KRKLVIPSELGYGERGWVPK |
| | M54A, | AGGCTGGGATCAAGGCCTGCTGGGAATGTGCGAAG | IPGGATLVFEVELLKIER |
| | P118V | GCGAGAAGAGAAAGCTGGTCATCCCTAGCGAACTC | (SEQ ID NO: 128) |
| | V85R | GGCTACGGCGAAAGAGGCtgggttCCAAAGATTCC | |
| | | TGGCGGAGCCACACTGGTGTTCGAAGTGGAACTGC | |
| | | TGAAGATCGAGCGA (SEQ ID NO: 127) | |
| FKBP-165 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | DRD | TTCGTGTTTAGCCTCGGCACCGGCgaacggATCAA | KRKLVIPSELGYGERGWPVK |
| | M54A, | AGGCTGGGATCAAGGCCTGCTGGGAATGTGCGAAG | IPGGATLVFEVELLKIER |
| | P119V | GCGAGAAGAGAAAGCTGGTCATCCCTAGCGAACTC | (SEQ ID NO: 130) |
| | V85R | GGCTACGGCGAAAGAGGCtggCCTgttAAGATTCC | |
| | | TGGCGGAGCCACACTGGTGTTCGAAGTGGAACTGC | |
| | | TGAAGATCGAGCGA (SEQ ID NO: 129) | |
| FKBP-166 | Q72R, | CACTGCCCAATCAAGAGCAGAAAAGGCGACGTGCT | HCPIKSRKGDVLHAHYTGKL |
| | S79W, | GCACgctCACTACACCGGCAAGCTGGAAGATGGCA | EDGTEFDSSLPRNQPFVFWL |
| | Q84E, | CCGAGTTCGATAGCAGCCTGCCTagaAACCAGCCT | GTGERIKGWDQGLLGMCEGE |
| | A117W | TTCGTGTTTtggCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIER |
| | M54A | GCGAGAAGAGAAAGCTGGTCATCCCTAGCGAACTC | (SEQ ID NO: 132) |
| | V85R | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 131) | |
| FKBP-167 | Q72R, | CACTGCCCAATCAAGAGCAGAAAAGGCGACGTGCT | HCPIKSRKGDVLHAHYTGKL |
| | S79W, | GCACgctCACTACACCGGCAAGCTGGAAGATGGCA | EDGTEFDSSLPRNQPFVFWL |
| | Q84R, | CCGAGTTCGATAGCAGCCTGCCTagaAACCAGCCT | GTGRRIKGWDQGLLGMCEGE |
| | A117W | TTCGTGTTTtggCTCGGCACCGGCagacggATCAA | KRKLVIPSELGYGERGWPPK |
| | DRD | AGGCTGGGATCAAGGCCTGCTGGGAATGTGCGAAG | IPGGATLVFEVELLKIER |
| | M54A | GCGAGAAGAGAAAGCTGGTCATCCCTAGCGAACTC | (SEQ ID NO: 134) |
| | V85R | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 133) | |
| FKBP-168 | Q72R, | CACTGCCCAATCAAGAGCAGAAAAGGCGACGTGCT | HCPIKSRKGDVLHAHYTGKL |
| | T82W, | GCACgctCACTACACCGGCAAGCTGGAAGATGGCA | EDGTEFDSSLPRNQPFVFSL |
| | Q84R, | CCGAGTTCGATAGCAGCCTGCCTagaAACCAGCCT | GWGRRIKGWDQGLLGMCEGE |
| | A117W | TTCGTGTTTAGCCTCGGCtggGGCagacggATCAA | KRKLVIPSELGYGERGWPPK |
| | DRD | AGGCTGGGATCAAGGCCTGCTGGGAATGTGCGAAG | IPGGATLVFEVELLKIER |
| | M54A | GCGAGAAGAGAAAGCTGGTCATCCCTAGCGAACTC | (SEQ ID NO: 136) |
| | V85R | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 135) | |
| FKBP-169 | Q72R, | CACTGCCCAATCAAGAGCAGAAAAGGCGACGTGCT | HCPIKSRKGDVLHAHYTGKL |
| | T82W, | GCACgctCACTACACCGGCAAGCTGGAAGATGGCA | EDGTEFDSSLPRNQPFVFSL |
| | Q84E, | CCGAGTTCGATAGCAGCCTGCCTagaAACCAGCCT | GWGEVIKGWDQGLLGMCEGE |
| | A117W | TTCGTGTTTAGCCTCGGCtggGGCgaaGTGATCAA | KRKLVIPSELGYGERGWPPK |
| | DRD | AGGCTGGGATCAAGGCCTGCTGGGAATGTGCGAAG | IPGGATLVFEVELLKIER |
| | M54A | GCGAGAAGAGAAAGCTGGTCATCCCTAGCGAACTC | (SEQ ID NO: 138) |
| | | GGCTACGGCGAAAGAGGCtggCCTCCAAAGATTCC | |
| | | GTGGCGGACCACACTGGTGTTCGAAGTGGAACTGC | |
| | | TGAAGATCGAGCGA (SEQ ID NO: 137) | |
| FKBP-170 | Q72R, | CACTGCCCAATCAAGAGCAGAAAAGGCGACGTGCT | HCPIKSRKGDVLHAHYTGKL |
| | T82W, | GCACgctCACTACACCGGCAAGCTGGAAGATGGCA | EDGTEFDSSLPRNQPFVFSL |
| | Q84E, | CCGAGTTCGATAGCAGCCTGCCTagaAACCAGCCT | GWGERIKGWDQGLLGMCEGE |
| | A117W | TTCGTGTTTAGCCTCGGCtggGGCgaacggATCAA | KRKLVIPSELGYGERGWPPK |
| | DRD | AGGCTGGGATCAAGGCCTGCTGGGAATGTGCGAAG | IPGGATLVFEVELLKIER |
| | M54A | GCGAGAAGAGAAAGCTGGTCATCCCTAGCGAACTC | (SEQ ID NO: 140) |
| | V85R | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 139) | |
| FKBP-171 | Q72R, | CACTGCCCAATCAAGAGCAGAAAAGGCcttGTGCT | HCPIKSRKGLVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGEVIKGWDQGLLGMCEGE |
| | DRD | TTCGTGTTTAGCCTCGGCACCGGCgaaGTGATCAA | KRKLVIPSELGYGERGWPPK |
| | M54A | AGGCTGGGATCAAGGCCTGCTGGGAATGTGCGAAG | IPGGATLVFEVELLKIER |
| | D50L | GCGAGAAGAGAAAGCTGGTCATCCCTAGCGAACTC | (SEQ ID NO: 142) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 141) | |
| FKBP-172 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGEVIKGWDQGLLGWCEGE |
| | DRD | TTCGTGTTTAGCCTCGGCACCGGCgaaGTGATCAA | KRKLVIPSELGYGERGWPPK |
| | M54A | AGGCTGGGATCAAGGCCTGCTGGGAtggTGCGAAG | IPGGATLVFEVELLKIER |
| | M96W | GCGAGAAGAGAAAGCTGGTCATCCCTAGCGAACTC | (SEQ ID NO: 144) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 143) | |
| FKBP-173 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGEVIKGWDQGLLGMCEGE |
| | DRD | TTCGTGTTTAGCCTCGGCACCGGCgaaGTGATCAA | KRKLVIPSELGYGERGWPPK |
| | M54A | AGGCTGGGATCAAGGCCTGCTGGGAATGTGCGAAG | IPGGATLVFEVEGLKIER |
| | L133G | GCGAGAAGAGAAAGCTGGTCATCCCTAGCGAACTC | (SEQ ID NO: 146) |
| | | GGCTACGGCGAAAGAGGCtggCCTCCAAAGATTCC | |
| | | TGGCGGAGCCACACTGGTGTTCGAAGTGGAAgggC | |
| | | TGAAGATCGAGCGA (SEQ ID NO: 145) | |
| FKBP-174 | Q72R, | CACTGCCCAATCAAGgggAGAAAAGGCGACGTGCT | HCPIKGRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGEVIKGWDQGLLGMCEGE |
| | DRD | TTCGTGTTTAGCCTCGGCACCGGCgaaGTGATCAA | KRKLVIPSELGYGERGWPPK |
| | M54A | AGGCTGGGATCAAGGCCTGCTGGGAATGTGCGAAG | IPGGATLVFEGELLKIER |
| | S36G, | GCGAGAAGAGAAAGCTGGTCATCCCTAGCGAACTC | (SEQ ID NO: 148) |
| | V131G | GGCTACGGCGAAAGAGGCtggCCTCCAAAGATTCC | |
| | | TGGCGGAGCCACACTGGTGTTCGAAgggGAACTGC | |
| | | TGAAGATCGAGCGA (SEQ ID NO: 147) | |
| FKBP-175 | Q72R, | CACTGCCCAATCAAGAGCAGAAAAGGCgcgGTGCT | HCPIKSRKGAVLHAHYIGKL |
| | Q84E, | GCACgctCACTACatcGGCAAGCTGGAAGATGGCA | EDGTEFDSSLPRNQPFVFSL |
| | A117W | CCGAGTTCGATAGCAGCCTGCCTagaAACCAGCCT | GTGEVIKGWDQGLLGMCEGE |
| | DRD | TTCGTGTTTAGCCTCGGCACCGGCgaaGTGATCAA | KRKLVIPSELGYGERGWPPK |
| | M54A | AGGCTGGGATCAAGGCCTGCTGGGAATGTGCGAAG | IPGGATLVFEVELLKLER |
| | D50L, | GCGAGAAGAGAAAGCTGGTCATCCCTAGCGAACTC | (SEQ ID NO: 150) |
| | T57I, | GGCTACGGCGAAAGAGGCtggCCTCCAAAGATTCC | |
| | I136L | TGGCGGAGCCACACTGGTGTTCGAAGTGGAACTGC | |
| | | TGAAGctgGAGCGA (SEQ ID NO: 149) | |
| FKBP-176 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGEVIKGWDQGLLGMCEGE |
| | DRD | TTCGTGTTTAGCCTCGGCACCGGCgaaGTGATCAA | KRKLVIPSELGYGEREWPPK |
| | M54A | AGGCTGGGATCAAGGCCTGCTGGGAATGTGCGAAG | IPGGATLVFEVELLKIER |
| | G116E | GCGAGAAGAGAAAGCTGGTCATCCCTAGCGAACTC | (SEQ ID NO: 152) |
| | | GGCTACGGCGAAAGAgagtggCCTCCAAAGATTCC | |
| | | TGGCGGAGCCACACTGGTGTTCGAAGTGGAACTGC | |
| | | TGAAGATCGAGCGA (SEQ ID NO: 151) | |
| FKBP-177 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSG |
| | A117W | CCGAGTTCGATAGCAGCCTGCCTagaAACCAGCCT | GTGEVIKGWDQGLLGMCEGE |
| | DRD | TTCGTGTTTAGCgggGGCACCGGCgaaGTGATCAA | KRKLVIPSELGYGERGWPPK |
| | M54A | AGGCTGGGATCAAGGCCTGCTGGGAATGTGCGAAG | IPGGATLVFEVELLKIER |
| | L80G | GCGAGAAGAGAAAGCTGGTCATCCCTAGCGAACTC | (SEQ ID NO: 154) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 153) | |
| FKBP-178 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVSSL |
| | A117W | CCGAGTTCGATAGCAGCCTGCCTagaAACCAGCCT | GTGEVIKGWDQGLLGMCEGE |
| | DRD | TTCGTGtcgAGCCTCGGCACCGGCgaaGTGATCAA | KRKLVIPSELGYGERGWPPK |
| | M54A | AGGCTGGGATCAAGGCCTGCTGGGAATGTGCGAAG | IPGGATLVFEVELLKIER |
| | F78S | GCGAGAAGAGAAAGCTGGTCATCCCTAGCGAACTC | (SEQ ID NO: 156) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 155) | |
| FKBP-179 | Q72R, | CACTGCCCAATCAAGAGCAGAAAAGGCGACGTGCT | HCPIKSRKGDVLGAHYTGKL |
| | Q84E, | GggggctCACTACACCGGCAAGCTGGAAGATGGCA | EDGTEFDSSLPLNQPFVFSL |
| | A117W | CCGAGTTCGATAGCAGCCTGCCTcttAACCAGCCT | GTGEVIKGWDQGLLGMCEGE |
| | DRD | TTCGTGTTTAGCCTCGGCACCGGCgaaGTGATCAA | KRKLVIPSELGYGERGWPPK |
| | M54A | AGGCTGGGATCAAGGCCTGCTGGGAATGTGCGAAG | IPGGATLVFEVELLKIER |
| | H53G, | GCGAGAAGAGAAAGCTGGTCATCCCTAGCGAACTC | (SEQ ID NO: 158) |
| | R72L | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 157) | |
| FKBP-180 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGEVIKGWDQGLLGMCEGE |
| | DRD | TTCGTGTTTAGCCTCGGCACCGGCgaaGTGATCAA | KRKLVIPSELGYGERGWPPK |
| | M54A | AGGCTGGGATCAAGGCCTGCTGGGAATGTGCGAAG | IPGGATLVTEVELLKIER |
| | F129T | GCGAGAAGAGAAAGCTGGTCATCCCTAGCGAACTC | (SEQ ID NO: 160) |
| | | GGCTACGGCGAAAGAGGCtggCCTCCAAAGATTCC | |
| | | TGGCGGAGCCACACTGGTGacgGAAGTGGAACTGC | |
| | | TGAAGATCGAGCGA (SEQ ID NO: 159) | |
| FKBP-181 | Q72R, | CACTGCCCAATCAAGAGCAGAAAAGGCcttGTGCT | HCPIKSRKGLVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIER |
| | M54A | GCGAGAAGAGAAAGCTGGTCATCCCTAGCGAACTC | (SEQ ID NO: 162) |
| | D50L | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 161) | |
| FKBP-182 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGWCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAtggTGCGAAG | IPGGATLVFEVELLKIER |
| | M54A | GCGAGAAGAGAAAGCTGGTCATCCCTAGCGAACTC | (SEQ ID NO: 164) |
| | M96W | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 163) | |
| FKBP-183 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVEGLKIER |
| | M54A | GCGAGAAGAGAAAGCTGGTCATCCCTAGCGAACTC | (SEQ ID NO: 166) |
| | L133G | GGCTACGGCGAAAGAGGCtggCCTCCAAAGATTCC | |
| | | TGGCGGAGCCACACTGGTGTTCGAAGTGGAAgggC | |
| | | TGAAGATCGAGCGA (SEQ ID NO: 165) | |
| FKBP-184 | Q72R, | CACTGCCCAATCAAGgggAGAAAAGGCGACGTGCT | HCPIKGRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEGELLKIER |
| | M54A | GCGAGAAGAGAAAGCTGGTCATCCCTAGCGAACTC | (SEQ ID NO: 168) |
| | S36G, | GGCTACGGCGAAAGAGGCtggCCTCCAAAGATTCC | |
| | V131G | TGGCGGAGCCACACTGGTGTTCGAAgggGAACTGC | |
| | | TGAAGATCGAGCGA (SEQ ID NO: 167) | |
| FKBP-185 | Q72R, | CACTGCCCAATCAAGAGCAGAAAAGGCgcgGTGCT | HCPIKSRKGAVLHAHYIGKL |
| | Q84E, | GCACgctCACTACatcGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKLER |
| | M54A | GCGAGAAGAGAAAGCTGGTCATCCCTAGCGAACTC | (SEQ ID NO: 170) |
| | D50L, | GGCTACGGCGAAAGAGGCtggCCTCCAAAGATTCC | |
| | T57I, | TGGCGGAGCCACACTGGTGTTCGAAGTGGAACTGC | |
| | I136L | TGAAGctgGAGCGA (SEQ ID NO: 169) | |
| FKBP-186 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGEREWPPK |
| | DRD | AGGCTGGGATCAAGGCCTGCTGGGAATGTGCGAAG | IPGGATLVFEVELLKIER |
| | M54A | GCGAGAAGAGAAAGCTGGTCATCCCTAGCGAACTC | (SEQ ID NO: 172) |
| | G116E | GGCTACGGCGAAAGAgagtggCCTCCAAAGATTCC | |
| | | TGGCGGAGCCACACTGGTGTTCGAAGTGGAACTGC | |
| | | TGAAGATCGAGCGA (SEQ ID NO: 171) | |
| FKBP-187 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSG |
| | A117W | CCGAGTTCGATAGCAGCCTGCCTagaAACCAGCCT | GTGERIKGWDQGLLGMCEGE |
| | V85R | TTCGTGTTTAGCgggGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIER |
| | M54A | GCGAGAAGAGAAAGCTGGTCATCCCTAGCGAACTC | (SEQ ID NO: 174) |
| | L80G | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 173) | |
| FKBP-188 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVSSL |
| | A117W | CCGAGTTCGATAGCAGCCTGCCTagaAACCAGCCT | GTGERIKGWDQGLLGMCEGE |
| | V85R | TTCGTGtcgAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIER |
| | M54A | GCGAGAAGAGAAAGCTGGTCATCCCTAGCGAACTC | (SEQ ID NO: 176) |
| | F78S | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 175) | |
| FKBP-189 | Q72R, | CACTGCCCAATCAAGAGCAGAAAAGGCGACGTGCT | HCPIKSRKGDVLGAHYTGKL |
| | Q84E, | GggggctCACTACACCGGCAAGCTGGAAGATGGCA | EDGTEFDSSLPLNQPFVFSL |
| | A117W | CCGAGTTCGATAGCAGCCTGCCTcttAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIER |
| | M54A | GCGAGAAGAGAAAGCTGGTCATCCCTAGCGAACTC | (SEQ ID NO: 178) |
| | H53G, | GGCTACGGCGAAAGAGGCtggCCTCCAAAGATTCC | |
| | R72L | TGGCGGAGCCACACTGGTGTTCGAAGTGGAACTGC | |
| | | TGAAGATCGAGCGA (SEQ ID NO: 177) | |

| | | | |
|---|---|---|---|
| FKBP-190 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVTEVELLKIER |
| | M54A | GCGAGAAGAGAAAGCTGGTCATCCCTAGCGAACTC | (SEQ ID NO: 180) |
| | F129T | GGCTACGGCGAAAGAGGCtggCCTCCAAAGATTCC | |
| | | TGGCGGAGCCACACTGGTGacgGAAGTGGAACTGC | |
| | | TGAAGATCGAGCGA (SEQ ID NO: 179) | |
| FKBP-191 | Q72R, | CACTGCCCAATCAAGAGCAGAAAAtccGACGTGCT | HCPIKSRKSDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGERIKGWDQGLLGMCEGE |
| | V85R | TTCGTGTTTAGCCTCGGCACCGGCGAAcggATCAA | KRKLVIPSELGYGERGWPPK |
| | DRD | AGGCTGGGATCAAGGCCTGCTGGGAATGTGCGAAG | IPGGATLVFEVELLKIER |
| | G49S | GCGAGAAGAGAAAGCTGGTCATCCCTAGCGAACTC | (SEQ ID NO: 182) |
| | | GGCTACGGCGAAAGAGGCTGGCCTCCAAAGATTCC | |
| | | TGGCGGAGCCACACTGGTGTTCGAAGTGGAACTGC | |
| | | TGAAGATCGAGCGA (SEQ ID NO: 181) | |
| FKBP-192 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQLFVFSL |
| | A117W | CCGAGTTCGATAGCAGCCTGCCTAGAAACCAGctg | GTGERIKGWDQGLLGMCEGE |
| | V85R | TTCGTGTTTAGCCTCGGCACCGGCGAAcggATCAA | KRKLVIPSELGYGERGWPPK |
| | DRD | AGGCTGGGATCAAGGCCTGCTGGGAATGTGCGAAG | IPGGATLVFEVELLKIER |
| | P75L | GCGAGAAGAGAAAGCTGGTCATCCCTAGCGAACTC | (SEQ ID NO: 184) |
| | | GGCTACGGCGAAAGAGGCTGGCCTCCAAAGATTCC | |
| | | TGGCGGAGCCACACTGGTGTTCGAAGTGGAACTGC | |
| | | TGAAGATCGAGCGA (SEQ ID NO: 183) | |
| FKBP-193 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWAQGLLGMCEGE |
| | V85R | TTCGTGTTTAGCCTCGGCACCGGCgaacggATCAA | KRKLVIPSELGYGERGWPPK |
| | DRD | AGGCTGGgctCAAGGCCTGCTGGGAATGTGCGAAG | IPGGATLVFEVELLKIER |
| | D90A | GCGAGAAGAGAAAGCTGGTCATCCCTAGCGAACTC | (SEQ ID NO: 186) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 185) | |
| FKBP-194 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKVVIPSELGYGERGWPPK |
| | DRD | AGGCTGGGATCAAGGCCTGCTGGGAATGTGCGAAG | IPGGATLVFEVELLKIER |
| | L104V | GCGAGAAGAGAAAGgttGTCATCCCTAGCGAACTC | (SEQ ID NO: 188) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 187) | |
| FKBP-195 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGERIKGWDQGLLGMCEGE |
| | V85R | TTCGTGTTTAGCCTCGGCACCGGCGAAcggATCAA | KRKLVIPSELGYGERGWPPK |
| | DRD | AGGCTGGGATCAAGGCCTGCTGGGAATGTGCGAAG | IPGGATLVFEVELLYIER |
| | K135Y | GCGAGAAGAGAAAGCTGGTCATCCCTAGCGAACTC | (SEQ ID NO: 190) |
| | | GGCTACGGCGAAAGAGGCTGGCCTCCAAAGATTCC | |
| | | TGGCGGAGCCACACTGGTGTTCGAAGTGGAACTGC | |
| | | TGtacATCGAGCGA (SEQ ID NO: 189) | |
| FKBP-196 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGERIKGWDQGLLGMCEGE |
| | V85R | TTCGTGTTTAGCCTCGGCACCGGCGAAcggATCAA | KRKLVIPSELGYGERGWPPK |
| | DRD | AGGCTGGGATCAAGGCCTGCTGGGAATGTGCGAAG | IPGGATLVFEVELLKVER |
| | I136V | GCGAGAAGAGAAAGCTGGTCATCCCTAGCGAACTC | (SEQ ID NO: 192) |
| | | GGCTACGGCGAAAGAGGCTGGCCTCCAAAGATTCC | |
| | | TGGCGGAGCCACACTGGTGTTCGAAGTGGAACTGC | |
| | | TGAAGgtgGAGCGA (SEQ ID NO: 191) | |
| FKBP-197 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGERIKGWDQGLLGMCEGE |
| | V85R | TTCGTGTTTAGCCTCGGCACCGGCGAAcggATCAA | KRKLVIPSELGYGERGWPPK |
| | DRD | AGGCTGGGATCAAGGCCTGCTGGGAATGTGCGAAG | IPGGATLVFRVELLKIER |
| | E130R | GCGAGAAGAGAAAGCTGGTCATCCCTAGCGAACTC | (SEQ ID NO: 194) |
| | | GGCTACGGCGAAAGAGGCTGGCCTCCAAAGATTCC | |
| | | TGGCGGAGCCACACTGGTGTTCagaGTGGAACTGC | |
| | | TGAAGATCGAGCGA (SEQ ID NO: 193) | |
| FKBP-198 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGERIKGWDQGLLGMCEGE |
| | V85R | TTCGTGTTTAGCCTCGGCACCGGCGAAcggATCAA | WRKLVIPSELGYGERGWPPK |
| | DRD | AGGCTGGGATCAAGGCCTGCTGGGAATGTGCGAAG | IPGGATLVFEVELLKIER |
| | K101W | GCGAGtggAGAAAGCTGGTCATCCCTAGCGAACTC | (SEQ ID NO: 196) |
| | | GGCTACGGCGAAAGAGGCTGGCCTCCAAAGATTCC | |
| | | TGGCGGAGCCACACTGGTGTTCGAAGTGGAACTGC | |
| | | TGAAGATCGAGCGA (SEQ ID NO: 195) | |
| FKBP-199 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGERIKGWDQGLLGMCLGE |
| | V85R | TTCGTGTTTAGCCTCGGCACCGGCGAAcggATCAA | KRKLVIPSELGYGERGWPPK |
| | DRD | AGGCTGGGATCAAGGCCTGCTGGGAATGTGCttgG | IPGGATLVFEVELLKIER |
| | E98L | GCGAGAAGAGAAAGCTGGTCATCCCTAGCGAACTC | (SEQ ID NO: 198) |
| | | GGCTACGGCGAAAGAGGCTGGCCTCCAAAGATTCC | |
| | | TGGCGGAGCCACACTGGTGTTCGAAGTGGAACTGC | |
| | | TGAAGATCGAGCGA (SEQ ID NO: 197) | |
| FKBP-200 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAtctGGCA | ESGTEFDSSLPRNQPFVFSL |
| | A117W | CCGAGTTCGATAGCAGCCTGCCTAGAAACCAGCCT | GTGERIKGWDQGLLGMCEGE |
| | V85R | TTCGTGTTTAGCCTCGGCACCGGCGAAcggATCAA | KRKLVIPSELGYGERGWPPK |
| | DRD | AGGCTGGGATCAAGGCCTGCTGGGAATGTGCGAAG | IPGGATLVFEVELLKIER |
| | D62S | GCGAGAAGAGAAAGCTGGTCATCCCTAGCGAACTC | (SEQ ID NO: 200) |
| | | GGCTACGGCGAAAGAGGCTGGCCTCCAAAGATTCC | |
| | | TGGCGGAGCCACACTGGTGTTCGAAGTGGAACTGC | |
| | | TGAAGATCGAGCGA (SEQ ID NO: 199) | |
| FKBP-201 | Q72R, | CACTGCCCAATCAAGAGCAGAAAAGGCGACGTGCT | HCPIKSRKGDVLLMHYTGKL |
| | Q84E, | GttgATGCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGERIKGWDQGLLGMCEGE |
| | V85R | TTCGTGTTTAGCCTCGGCACCGGCGAAcggATCAA | KRKLVIPSELGYGERGWPPK |
| | DRD | AGGCTGGGATCAAGGCCTGCTGGGAATGTGCGAAG | IPGGATLVFEVELLKIER |
| | H53L | GCGAGAAGAGAAAGCTGGTCATCCCTAGCGAACTC | (SEQ ID NO: 202) |
| | | GGCTACGGCGAAAGAGGCTGGCCTCCAAAGATTCC | |
| | | TGGCGGAGCCACACTGGTGTTCGAAGTGGAACTGC | |
| | | TGAAGATCGAGCGA (SEQ ID NO: 201) | |
| FKBP-202 | Q72R, | CACTGCCCAATCAAGAGCAGAAAAtccGACGTGCT | HCPIKSRKSDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGERIKGWDQGLLGMCEGE |
| | V85R | TTCGTGTTTAGCCTCGGCACCGGCGAAcggATCAA | KRKLVIPSELGYGERGWPPK |
| | DRD | AGGCTGGGATCAAGGCCTGCTGGGAATGTGCGAAG | IPGGATLVFEVELLKIER |
| | M54A | GCGAGAAGAGAAAGCTGGTCATCCCTAGCGAACTC | (SEQ ID NO: 204) |
| | G49S | GGCTACGGCGAAAGAGGCTGGCCTCCAAAGATTCC | |
| | | TGGCGGAGCCACACTGGTGTTCGAAGTGGAACTGC | |
| | | TGAAGATCGAGCGA (SEQ ID NO: 203) | |
| FKBP-203 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQLFVFSL |
| | A117W | CCGAGTTCGATAGCAGCCTGCCTAGAAACCAGctg | GTGERIKGWDQGLLGMCEGE |
| | V85R | TTCGTGTTTAGCCTCGGCACCGGCGAAcggATCAA | KRKLVIPSELGYGERGWPPK |
| | DRD | AGGCTGGGATCAAGGCCTGCTGGGAATGTGCGAAG | IPGGATLVFEVELLKIER |
| | M54A | GCGAGAAGAGAAAGCTGGTCATCCCTAGCGAACTC | (SEQ ID NO: 206) |
| | P75L | GGCTACGGCGAAAGAGGCTGGCCTCCAAAGATTCC | |
| | | TGGCGGAGCCACACTGGTGTTCGAAGTGGAACTGC | |
| | | TGAAGATCGAGCGA (SEQ ID NO: 205) | |
| FKBP-204 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWAQGLLGMCEGE |
| | V85R | TTCGTGTTTAGCCTCGGCACCGGCgaacggATCAA | KRKLVIPSELGYGERGWPPK |
| | DRD | AGGCTGGgctCAAGGCCTGCTGGGAATGTGCGAAG | IPGGATLVFEVELLKIER |
| | M54A | GCGAGAAGAGAAAGCTGGTCATCCCTAGCGAACTC | (SEQ ID NO: 208) |
| | D90A | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 207) | |
| FKBP-205 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKVVIPSELGYGERGWPPK |
| | DRD | AGGCTGGGATCAAGGCCTGCTGGGAATGTGCGAAG | IPGGATLVFEVELLKIER |
| | M54A | GCGAGAAGAGAAAGgttGTCATCCCTAGCGAACTC | (SEQ ID NO: 210) |
| | L104V | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 209) | |
| FKBP-206 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGERIKGWDQGLLGMCEGE |
| | V85R | TTCGTGTTTAGCCTCGGCACCGGCGAAcggATCAA | KRKLVIPSELGYGERGWPPK |
| | DRD | AGGCTGGGATCAAGGCCTGCTGGGAATGTGCGAAG | IPGGATLVFEVELLYIER |
| | M54A | GCGAGAAGAGAAAGCTGGTCATCCCTAGCGAACTC | (SEQ ID NO: 212) |
| | K135Y | GGCTACGGCGAAAGAGGCTGGCCTCCAAAGATTCC | |
| | | TGGCGGAGCCACACTGGTGTTCGAAGTGGAACTGC | |
| | | TGtacATCGAGCGA (SEQ ID NO: 211) | |
| FKBP-207 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGERIKGWDQGLLGMCEGE |
| | V85R | TTCGTGTTTAGCCTCGGCACCGGCGAAcggATCAA | KRKLVIPSELGYGERGWPPK |
| | DRD | AGGCTGGGATCAAGGCCTGCTGGGAATGTGCGAAG | IPGGATLVFEVELLKVER |
| | M54A | GCGAGAAGAGAAAGCTGGTCATCCCTAGCGAACTC | (SEQ ID NO: 214) |
| | I136V | GGCTACGGCGAAAGAGGCTGGCCTCCAAAGATTCC | |
| | | TGGCGGAGCCACACTGGTGTTCGAAGTGGAACTGC | |
| | | TGAAGgtgGAGCGA (SEQ ID NO: 213) | |
| FKBP-208 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGERIKGWDQGLLGMCEGE |
| | V85R | TTCGTGTTTAGCCTCGGCACCGGCGAAcggATCAA | KRKLVIPSELGYGERGWPPK |
| | DRD | AGGCTGGGATCAAGGCCTGCTGGGAATGTGCGAAG | IPGGATLVFRVELLKIER |
| | M54A | GCGAGAAGAGAAAGCTGGTCATCCCTAGCGAACTC | (SEQ ID NO: 216) |
| | E130R | GGCTACGGCGAAAGAGGCTGGCCTCCAAAGATTCC | |
| | | TGGCGGAGCCACACTGGTGTTCagaGTGGAACTGC | |
| | | TGAAGATCGAGCGA (SEQ ID NO: 215) | |
| FKBP-209 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGERIKGWDQGLLGMCEGE |
| | V85R | TTCGTGTTTAGCCTCGGCACCGGCGAAcggATCAA | WRKLVIPSELGYGERGWPPK |
| | DRD | AGGCTGGGATCAAGGCCTGCTGGGAATGTGCGAAG | IPGGATLVFEVELLKIER |
| | M54A | GCGAGtggAGAAAGCTGGTCATCCCTAGCGAACTC | (SEQ ID NO: 218) |
| | K101W | GGCTACGGCGAAAGAGGCTGGCCTCCAAAGATTCC | |
| | | TGGCGGAGCCACACTGGTGTTCGAAGTGGAACTGC | |
| | | TGAAGATCGAGCGA (SEQ ID NO: 217) | |
| FKBP-210 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGERIKGWDQGLLGMCLGE |
| | V85R | TTCGTGTTTAGCCTCGGCACCGGCGAAcggATCAA | KRKLVIPSELGYGERGWPPK |
| | DRD | AGGCTGGGATCAAGGCCTGCTGGGAATGTGCttgG | IPGGATLVFEVELLKIER |
| | M54A | GCGAGAAGAGAAAGCTGGTCATCCCTAGCGAACTC | (SEQ ID NO: 220) |
| | E98L | GGCTACGGCGAAAGAGGCTGGCCTCCAAAGATTCC | |
| | | TGGCGGAGCCACACTGGTGTTCGAAGTGGAACTGC | |
| | | TGAAGATCGAGCGA (SEQ ID NO: 219) | |
| FKBP-211 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAtctGGCA | ESGTEFDSSLPRNQPFVFSL |
| | A117W | CCGAGTTCGATAGCAGCCTGCCTAGAAACCAGCCT | GTGERIKGWDQGLLGMCEGE |
| | V85R | TTCGTGTTTAGCCTCGGCACCGGCGAAcggATCAA | KRKLVIPSELGYGERGWPPK |
| | DRD | AGGCTGGGATCAAGGCCTGCTGGGAATGTGCGAAG | IPGGATLVFEVELLKIER |
| | M54A | GCGAGAAGAGAAAGCTGGTCATCCCTAGCGAACTC | (SEQ ID NO: 222) |
| | D62S | GGCTACGGCGAAAGAGGCTGGCCTCCAAAGATTCC | |
| | | TGGCGGAGCCACACTGGTGTTCGAAGTGGAACTGC | |
| | | TGAAGATCGAGCGA (SEQ ID NO: 221) | |
| FKBP-212 | Q72R, | CACTGCCCAATCAAGAGCAGAAAAGGCGACGTGCT | HCPIKSRKGDVLLAHYTGKL |
| | Q84E, | GttggctCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGERIKGWDQGLLGMCEGE |
| | V85R | TTCGTGTTTAGCCTCGGCACCGGCGAAcggATCAA | KRKLVIPSELGYGERGWPPK |
| | DRD | AGGCTGGGATCAAGGCCTGCTGGGAATGTGCGAAG | IPGGATLVFEVELLKIER |
| | M54A | GCGAGAAGAGAAAGCTGGTCATCCCTAGCGAACTC | (SEQ ID NO: 224) |
| | H53L | GGCTACGGCGAAAGAGGCTGGCCTCCAAAGATTCC | |
| | | TGGCGGAGCCACACTGGTGTTCGAAGTGGAACTGC | |
| | | TGAAGATCGAGCGA (SEQ ID NO: 223) | |
| FKBP-213 | Q72R, | CACTGCagaATCAAGAGCAGAAAAGGCGACGTGCT | HCRIKSRKGDVLHAHYTGKL |
| | Q84G, | GCACgctCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGGVIKGWDQGLLGMCEGE |
| | DRD | TTCGTGTTTAGCCTCGGCACCGGCggaGTGATCAA | KRKLVIPSELGYGERGWPPK |
| | P43R | AGGCTGGGATCAAGGCCTGCTGGGAATGTGCGAAG | IPGGATLVFEVELLKIER |
| | M54A | GCGAGAAGAGAAAGCTGGTCATCCCTAGCGAACTC | (SEQ ID NO: 226) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 225) | |
| FKBP-214 | Q72R, | CACTGCagaATCAAGAGCAGAAAAGGCGACGTGCT | HCRIKSRKGDVLHAHYTGKL |
| | Q84G, | GCACgctCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGGRIKGWDQGLLGMCEGE |
| | V85R | TTCGTGTTTAGCCTCGGCACCGGCggacggATCAA | KRKLVIPSELGYGERGWPPK |
| | DRD | AGGCTGGGATCAAGGCCTGCTGGGAATGTGCGAAG | IPGGATLVFEVELLKIER |
| | P43R | GCGAGAAGAGAAAGCTGGTCATCCCTAGCGAACTC | (SEQ ID NO: 228) |
| | M54A | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 227) | |
| FKBP-215 | Q72R, | CACTGCagaATCAAGAGCAGAAAAGGCGACGTGCT | HCRIKSRKGDVLHAHYTGKL |
| | Q84G, | GCACgctCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGGRIKGWDQGLLGMCEGE |
| | V85R | TTCGTGTTTAGCCTCGGCACCGGCggacggATCAA | KRKLVIPSELGYGERGWPPK |
| | DRD | AGGCTGGGATCAAGGCCTGCTGGGAATGTGCGAAG | IPGGATLVFEVELLYIER |
| | P43R | GCGAGAAGAGAAAGCTGGTCATCCCTAGCGAACTC | (SEQ ID NO: 230) |
| | M54A | GGCTACGGCGAAAGAGGCtggCCTCCAAAGATTCC | |
| | K135Y | TGGCGGAGCCACACTGGTGTTCGAAGTGGAACTGC | |
| | | TGtacATCGAGCGA (SEQ ID NO: 229) | |
| FKBP-216 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGtatGATGGCA | YDGTEFDSSLPRNQPFVFSL |
| | A117W | CCGAGTTCGATAGCAGCCTGCCTagaAACCAGCCT | GTGEVIKGWDQGLLGMCEGE |
| | DRD | TTCGTGTTTAGCCTCGGCACCGGCgaaGTGATCAA | WRKLVIPSELGYGERGWPPK |
| | M54A | AGGCTGGGATCAAGGCCTGCTGGGAATGTGCGAAG | IPGGATLVFEVELLYIER |
| | E61Y, | GCGAGtggAGAAAGCTGGTCATCCCTAGCGAACTC | (SEQ ID NO: 232) |
| | K101W, | GGCTACGGCGAAAGAGGCtggCCTCCAAAGATTCC | |
| | K135Y | TGGCGGAGCCACACTGGTGTTCGAAGTGGAACTGC | |
| | | TGtacATCGAGCGA (SEQ ID NO: 231) | |
| FKBP-217 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGtatGATGGCA | YDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | WRKLVIPSELGYGERGWPPK |
| | DRD | AGGCTGGGATCAAGGCCTGCTGGGAATGTGCGAAG | IPGGATLVFEVELLYIER |
| | M54A | GCGAGtggAGAAAGCTGGTCATCCCTAGCGAACTC | (SEQ ID NO: 234) |
| | E61Y, | GGCTACGGCGAAAGAGGCtggCCTCCAAAGATTCC | |
| | K101W, | TGGCGGAGCCACACTGGTGTTCGAAGTGGAACTGC | |
| | K135Y | TGtacATCGAGCGA (SEQ ID NO: 233) | |
| FKBP-218 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGggcGATGGCA | GDGTEFDSSLPRNQPFVFSL |
| | A117W | CCGAGTTCGATAGCAGCCTGCCTagaAACCAGCCT | GTGEVIKGWDQGLLGMCEGE |
| | DRD | TTCGTGTTTAGCCTCGGCACCGGCgaaGTGATCAA | GRKLVIPSELGYGERGWPPK |
| | M54A | AGGCTGGGATCAAGGCCTGCTGGGAATGTGCGAAG | IPGGATLVFEVELLEIER |
| | E61G, | GCGAGggcAGAAAGCTGGTCATCCCTAGCGAACTC | (SEQ ID NO: 236) |
| | K101G, | GGCTACGGCGAAAGAGGCtggCCTCCAAAGATTCC | |
| | K135E | TGGCGGAGCCACACTGGTGTTCGAAGTGGAACTGC | |
| | | TGgagATCGAGCGA (SEQ ID NO: 235) | |
| FKBP-219 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGggcGATGGCA | GDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | GRKLVIPSELGYGERGWPPK |
| | DRD | AGGCTGGGATCAAGGCCTGCTGGGAATGTGCGAAG | IPGGATLVFEVELLEIER |
| | M54A | GCGAGggcAGAAAGCTGGTCATCCCTAGCGAACTC | (SEQ ID NO: 238) |
| | E61G, | GGCTACGGCGAAAGAGGCtggCCTCCAAAGATTCC | |
| | K101G, | TGGCGGAGCCACACTGGTGTTCGAAGTGGAACTGC | |
| | K135E | TGgagATCGAGCGA (SEQ ID NO: 237) | |
| FKBP-220 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCg | EDGGEFDSSLPRNQPFVFSL |
| | A117W | ggGAGTTCGATAGCAGCCTGCCTAGAAACCAGCCT | GTGERIKGWDQGLLGMCEGE |
| | V85R | TTCGTGTTTAGCCTCGGCACCGGCGAAcggATCAA | KRKLVIPSELGYGERGWPPK |
| | DRD | AGGCTGGGATCAAGGCCTGCTGGGAATGTGCGAAG | IPGGATLVFEVELLKIER |
| | T25G | GCGAGAAGAGAAAGCTGGTCATCCCTAGCGAACTC | (SEQ ID NO: 240) |
| | | GGCTACGGCGAAAGAGGCTGGCCTCCAAAGATTCC | |
| | | TGGCGGAGCCACACTGGTGTTCGAAGTGGAACTGC | |
| | | TGAAGATCGAGCGA (SEQ ID NO: 239) | |
| FKBP-221 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCg | EDGGEFDSSLPRNQPFVFSL |
| | A117W | ggGAGTTCGATAGCAGCCTGCCTAGAAACCAGCCT | GTGERIKGWDQGLLGMCEGE |
| | V85R | TTCGTGTTTAGCCTCGGCACCGGCGAAcggATCAA | KRKLVIPSELGYGERGWPPK |
| | DRD | AGGCTGGGATCAAGGCCTGCTGGGAATGTGCGAAG | IPGGATLVFEVELLKIER |
| | M54A | GCGAGAAGAGAAAGCTGGTCATCCCTAGCGAACTC | (SEQ ID NO: 242) |
| | T25G | GGCTACGGCGAAAGAGGCTGGCCTCCAAAGATTCC | |
| | | TGGCGGAGCCACACTGGTGTTCGAAGTGGAACTGC | |
| | | TGAAGATCGAGCGA (SEQ ID NO: 241) | |
| FKBP-222 | Q72R, | CACTGCtggATCAAGAGCAGAAAAGGCGACGTGCT | HCWIKSRKGDVLHMHYTGKL |
| | Q84E, | GCACATGCACTACACCGGCAAGCTGGAAGATGGCc | EDGREFDSSLPRNQPFVFSL |
| | A117W | ggGAGTTCGATAGCAGCCTGCCTAGAAACCAGCCT | GTGEVIKGWDQGLLGMCEGE |
| | DRD | TTCGTGTTTAGCCTCGGCACCGGCGAAGTGATCAA | KRKLVIPSELGYGERGWPPK |
| | P4W | AGGCTGGGATCAAGGCCTGCTGGGAATGTGCGAAG | IPGGATLVFEVELLKIER |
| | T25R | GCGAGAAGAGAAAGCTGGTCATCCCTAGCGAACTC | (SEQ ID NO: 244) |
| | | GGCTACGGCGAAAGAGGCTGGCCTCCAAAGATTCC | |
| | | TGGCGGAGCCACACTGGTGTTCGAAGTGGAACTGC | |
| | | TGAAGATCGAGCGA (SEQ ID NO: 243) | |
| FKBP-223 | Q72R, | CACTGCtggATCAAGAGCAGAAAAGGCGACGTGCT | HCWIKSRKGDVLHAHYTGKL |
| | Q84E, | GCACgctCACTACACCGGCAAGCTGGAAGATGGCc | EDGREFDSSLPRNQPFVFSL |
| | A117W | ggGAGTTCGATAGCAGCCTGCCTAGAAACCAGCCT | GTGERIKGWDQGLLGMCEGE |
| | V85R | TTCGTGTTTAGCCTCGGCACCGGCGAAcggATCAA | KRKLVIPSELGYGERGWPPK |
| | DRD | AGGCTGGGATCAAGGCCTGCTGGGAATGTGCGAAG | IPGGATLVFEVELLKIER |
| | M54A | GCGAGAAGAGAAAGCTGGTCATCCCTAGCGAACTC | (SEQ ID NO: 246) |
| | P4W | GGCTACGGCGAAAGAGGCTGGCCTCCAAAGATTCC | |
| | T25R | TGGCGGAGCCACACTGGTGTTCGAAGTGGAACTGC | |
| | | TGAAGATCGAGCGA (SEQ ID NO: 245) | |
| FKBP-224 | Q72R, | CACTGCagaATCAAGAGCAGAAAAGGCGACGTGCT | HCRIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGEVIKGWDQGLLGMCEGE |
| | DRD | TTCGTGTTTAGCCTCGGCACCGGCgaaGTGATCAA | KRKLVIPSELGYGERGWPPK |
| | P43R | AGGCTGGGATCAAGGCCTGCTGGGAATGTGCGAAG | IPGGATLVFEVELLKIER |
| | | GCGAGAAGAGAAAGCTGGTCATCCCTAGCGAACTC | (SEQ ID NO: 248) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 247) | |
| FKBP-225 | Q72R, | CACTGCagaATCAAGAGCAGAAAAGGCGACGTGCT | HCRIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIER |
| | P43R | GCGAGAAGAGAAAGCTGGTCATCCCTAGCGAACTC | (SEQ ID NO: 250) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 249) | |
| FKBP-226 | Q72R, | CACTGCagaATCAAGAGCAGAAAAGGCGACGTGCT | HCRIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIER |
| | P43R | GCGAGAAGAGAAAGCTGGTCATCCCTAGCGAACTC | (SEQ ID NO: 252) |
| | M54A | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 251) | |
| FKBP-227 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGEVIKGWDQGLLGMCEGE |
| | DRD | TTCGTGTTTAGCCTCGGCACCGGCgaaGTGATCAA | KRKLVIPSELGYGERGWPPK |
| | E137R | AGGCTGGGATCAAGGCCTGCTGGGAATGTGCGAAG | IPGGATLVFEVELLKIRR |
| | | GCGAGAAGAGAAAGCTGGTCATCCCTAGCGAACTC | (SEQ ID NO: 254) |
| | | GGCTACGGCGAAAGAGGCtggCCTCCAAAGATTCC | |
| | | TGGCGGAGCCACACTGGTGTTCGAAGTGGAACTGC | |
| | | TGAAGATCaggCGA (SEQ ID NO: 253) | |
| FKBP-228 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIRR |
| | E137R | GCGAGAAGAGAAAGCTGGTCATCCCTAGCGAACTC | (SEQ ID NO: 256) |
| | | GGCTACGGCGAAAGAGGCtggCCTCCAAAGATTCC | |
| | | TGGCGGAGCCACACTGGTGTTCGAAGTGGAACTGC | |
| | | TGAAGATCaggCGA (SEQ ID NO: 255) | |
| FKBP-229 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIRR |
| | E137R | GCGAGAAGAGAAAGCTGGTCATCCCTAGCGAACTC | (SEQ ID NO: 258) |
| | M54A | GGCTACGGCGAAAGAGGCtggCCTCCAAAGATTCC | |
| | | TGGCGGAGCCACACTGGTGTTCGAAGTGGAACTGC | |
| | | TGAAGATCaggCGA (SEQ ID NO: 257) | |
| FKBP-230 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATATGA | EDMTEFDSSLPRNQPFVFSL |
| | A117W | CCGAGTTCGATAGCAGCCTGCCTagaAACCAGCCT | GTGEVIKGWDQGLLGMCEGE |
| | DRD | TTCGTGTTTAGCCTCGGCACCGGCgaaGTGATCAA | KRKLVIPSELGYGERGWPPK |
| | G63M | AGGCTGGGATCAAGGCCTGCTGGGAATGTGCGAAG | IPGGATLVFEVELLKIER |
| | | GCGAGAAGAGAAAGCTGGTCATCCCTAGCGAACTC | (SEQ ID NO: 260) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 259) | |
| FKBP-231 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATATGA | EDMTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIER |
| | G63M | GCGAGAAGAGAAAGCTGGTCATCCCTAGCGAACTC | (SEQ ID NO: 262) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 261) | |
| FKBP-232 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATATGA | EDMTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIER |
| | G63M | GCGAGAAGAGAAAGCTGGTCATCCCTAGCGAACTC | (SEQ ID NO: 264) |
| | M54A | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 263) | |
| FKBP-233 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGEVIKGWTQGLLGMCEGE |
| | DRD | TTCGTGTTTAGCCTCGGCACCGGCgaaGTGATCAA | KRKLVIPSELGYGERGWPPK |
| | D90T | AGGCTGGacgCAAGGCCTGCTGGGAATGTGCGAAG | IPGGATLVFEVELLKIER |
| | | GCGAGAAGAGAAAGCTGGTCATCCCTAGCGAACTC | (SEQ ID NO: 266) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 265) | |
| FKBP-234 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWTQGLLGMCEGE |
| | V85R | TTCGTGTTTAGCCTCGGCACCGGCgaacggATCAA | KRKLVIPSELGYGERGWPPK |
| | DRD | AGGCTGGacgCAAGGCCTGCTGGGAATGTGCGAAG | IPGGATLVFEVELLKIER |
| | D90T | GCGAGAAGAGAAAGCTGGTCATCCCTAGCGAACTC | (SEQ ID NO: 268) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 267) | |
| FKBP-235 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWTQGLLGMCEGE |
| | V85R | TTCGTGTTTAGCCTCGGCACCGGCgaacggATCAA | KRKLVIPSELGYGERGWPPK |
| | DRD | AGGCTGGacgCAAGGCCTGCTGGGAATGTGCGAAG | IPGGATLVFEVELLKIER |
| | D90T | GCGAGAAGAGAAAGCTGGTCATCCCTAGCGAACTC | (SEQ ID NO: 270) |
| | M54A | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 269) | |
| FKBP-236 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGEVIKGWDQGLLGMCEGE |
| | DRD | TTCGTGTTTAGCCTCGGCACCGGCgaaGTGATCAA | KRKLVIPSELGYNERGWPPK |
| | G113N | AGGCTGGGATCAAGGCCTGCTGGGAATGTGCGAAG | IPGGATLVFEVELLKIER |
| | | GCGAGAAGAGAAAGCTGGTCATCCCTAGCGAACTC | (SEQ ID NO: 272) |
| | | GGCTACaatGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 271) | |
| FKBP-237 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYNERGWPPK |
| | DRD | AGGCTGGGATCAAGGCCTGCTGGGAATGTGCGAAG | IPGGATLVFEVELLKIER |
| | G113N | GCGAGAAGAGAAAGCTGGTCATCCCTAGCGAACTC | (SEQ ID NO: 274) |
| | | GGCTACaatGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 273) | |
| FKBP-238 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYNERGWPPK |
| | DRD | AGGCTGGGATCAAGGCCTGCTGGGAATGTGCGAAG | IPGGATLVFEVELLKIER |
| | G113N | GCGAGAAGAGAAAGCTGGTCATCCCTAGCGAACTC | (SEQ ID NO: 276) |
| | M54A | GGCTACaatGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 275) | |
| FKBP-239 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGtatGATGGCA | YDGTEFDSSLPRNQPFVFSL |
| | A117W | CCGAGTTCGATAGCAGCCTGCCTagaAACCAGCCT | GTGEVIKGWDQGLLGMCEGE |
| | DRD | TTCGTGTTTAGCCTCGGCACCGGCgaaGTGATCAA | KRKLVIPSELGYGERGWPPK |
| | E61Y | AGGCTGGGATCAAGGCCTGCTGGGAATGTGCGAAG | IPGGATLVFEVELLKIER |
| | | GCGAGAAGAGAAAGCTGGTCATCCCTAGCGAACTC | (SEQ ID NO: 278) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 277) | |
| FKBP-240 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGtatGATGGCA | YDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIER |
| | E61Y | GCGAGAAGAGAAAGCTGGTCATCCCTAGCGAACTC | (SEQ ID NO: 280) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 279) | |
| FKBP-241 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGtatGATGGCA | YDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIER |
| | E61Y | GCGAGAAGAGAAAGCTGGTCATCCCTAGCGAACTC | (SEQ ID NO: 282) |
| | M54A | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 281) | |
| FKBP-242 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGEVIKGWDQGLLGMCEGE |
| | DRD | TTCGTGTTTAGCCTCGGCACCGGCgaaGTGATCAA | KRKLVIPSELGYGERGWSPK |
| | P118S | AGGCTGGGATCAAGGCCTGCTGGGAATGTGCGAAG | IPGGATLVFEVELLKIER |
| | | GCGAGAAGAGAAAGCTGGTCATCCCTAGCGAACTC | (SEQ ID NO: 284) |
| | | GGCTACGGCGAAAGAGGCtggtcgCCAAAGATTCC | |
| | | TGGCGGAGCCACACTGGTGTTCGAAGTGGAACTGC | |
| | | TGAAGATCGAGCGA (SEQ ID NO: 283) | |
| FKBP-243 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWSPK |
| | DRD | AGGCTGGGATCAAGGCCTGCTGGGAATGTGCGAAG | IPGGATLVFEVELLKIER |
| | P118S | GCGAGAAGAGAAAGCTGGTCATCCCTAGCGAACTC | (SEQ ID NO: 286) |
| | | GGCTACGGCGAAAGAGGCtggtcgCCAAAGATTCC | |
| | | TGGCGGAGCCACACTGGTGTTCGAAGTGGAACTGC | |
| | | TGAAGATCGAGCGA (SEQ ID NO: 285) | |
| FKBP-244 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWSPK |
| | DRD | AGGCTGGGATCAAGGCCTGCTGGGAATGTGCGAAG | IPGGATLVFEVELLKIER |
| | P118S | GCGAGAAGAGAAAGCTGGTCATCCCTAGCGAACTC | (SEQ ID NO: 288) |
| | M54A | GGCTACGGCGAAAGAGGCtggtcgCCAAAGATTCC | |
| | | TGGCGGAGCCACACTGGTGTTCGAAGTGGAACTGC | |
| | | TGAAGATCGAGCGA (SEQ ID NO: 287) | |
| FKBP-245 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSHLPRNQPFVFSL |
| | A117W | CCGAGTTCGATAGCcatCTGCCTagaAACCAGCCT | GTGEVIKGWDQGLLGMCEGE |
| | DRD | TTCGTGTTTAGCCTCGGCACCGGCgaaGTGATCAA | KRKLVIPSELGYGERGWPPK |
| | S69H | AGGCTGGGATCAAGGCCTGCTGGGAATGTGCGAAG | IPGGATLVFEVELLKIER |
| | | GCGAGAAGAGAAAGCTGGTCATCCCTAGCGAACTC | (SEQ ID NO: 290) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 289) | |
| FKBP-246 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSHLPRNQPFVFSL |
| | A117W | CCGAGTTCGATAGCcatCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIER |
| | S69H | GCGAGAAGAGAAAGCTGGTCATCCCTAGCGAACTC | (SEQ ID NO: 292) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 291) | |
| FKBP-247 | Q72R, | CACTGCCCAATCAAGAGCAGAAAAGGCGACGTGCT | HCPIKSRKGDVLHAHYTGKL |
| | Q84E, | GCACgctCACTACACCGGCAAGCTGGAAGATGGCA | EDGTEFDSHLPRNQPFVFSL |
| | A117W | CCGAGTTCGATAGCcatCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIER |
| | S69H | GCGAGAAGAGAAAGCTGGTCATCCCTAGCGAACTC | (SEQ ID NO: 294) |
| | M54A | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 293) | |
| FKBP-248 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDDSLPRNQPFVFSL |
| | A117W | CCGAGTTCGATgacAGCCTGCCTagaAACCAGCCT | GTGEVIKGWDQGLLGMCEGE |
| | DRD | TTCGTGTTTAGCCTCGGCACCGGCgaaGTGATCAA | KRKLVIPSELGYGERGWPPK |
| | S68D | AGGCTGGGATCAAGGCCTGCTGGGAATGTGCGAAG | IPGGATLVFEVELLKIER |
| | | GCGAGAAGAGAAAGCTGGTCATCCCTAGCGAACTC | (SEQ ID NO: 296) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 295) | |
| FKBP-249 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDDSLPRNQPFVFSL |
| | A117W | CCGAGTTCGATgacAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIER |
| | S68D | GCGAGAAGAGAAAGCTGGTCATCCCTAGCGAACTC | (SEQ ID NO: 298) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 297) | |
| FKBP-250 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGEVIKGWDQGLLGMCEGE |
| | DRD | TTCGTGTTTAGCCTCGGCACCGGCgaaGTGATCAA | KRKLVIPSELGYGERGWPPK |
| | E130F | AGGCTGGGATCAAGGCCTGCTGGGAATGTGCGAAG | IPGGATLVFFVELLKIER |
| | | GCGAGAAGAGAAAGCTGGTCATCCCTAGCGAACTC | (SEQ ID NO: 300) |
| | | GGCTACGGCGAAAGAGGCtggCCTCCAAAGATTCC | |
| | | TGGCGGAGCCACACTGGTGTTCtttGTGGAACTGC | |
| | | TGAAGATCGAGCGA (SEQ ID NO: 299) | |
| FKBP-251 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFFVELLKIER |
| | E130F | GCGAGAAGAGAAAGCTGGTCATCCCTAGCGAACTC | (SEQ ID NO: 302) |
| | | GGCTACGGCGAAAGAGGCtggCCTCCAAAGATTCC | |
| | | TGGCGGAGCCACACTGGTGTTCtttGTGGAACTGC | |
| | | TGAAGATCGAGCGA (SEQ ID NO: 301) | |
| FKBP-252 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGEVIKGWDQGLLGMCEGE |
| | DRD | TTCGTGTTTAGCCTCGGCACCGGCgaaGTGATCAA | KRKLVIPSELGYGERGWPPK |
| | I136P | AGGCTGGGATCAAGGCCTGCTGGGAATGTGCGAAG | IPGGATLVFEVELLKPER |
| | | GCGAGAAGAGAAAGCTGGTCATCCCTAGCGAACTC | (SEQ ID NO: 304) |
| | | GGCTACGGCGAAAGAGGCtggCCTCCAAAGATTCC | |
| | | TGGCGGAGCCACACTGGTGTTCGAAGTGGAACTGC | |
| | | TGAAGccaGAGCGA (SEQ ID NO: 303) | |
| FKBP-253 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKPER |
| | I136P | GCGAGAAGAGAAAGCTGGTCATCCCTAGCGAACTC | (SEQ ID NO: 306) |
| | | GGCTACGGCGAAAGAGGCtggCCTCCAAAGATTCC | |
| | | TGGCGGAGCCACACTGGTGTTCGAAGTGGAACTGC | |
| | | TGAAGccaGAGCGA (SEQ ID NO: 305) | |
| FKBP-254 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGEVIKGWDQGLLGMCERE |
| | DRD | TTCGTGTTTAGCCTCGGCACCGGCgaaGTGATCAA | KRKLVIPSELGYGERGWPPK |
| | G99R | AGGCTGGGATCAAGGCCTGCTGGGAATGTGCGAAc | IPGGATLVFEVELLKIER |
| | | ggGAGAAGAGAAAGCTGGTCATCCCTAGCGAACTC | (SEQ ID NO: 308) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 307) | |
| FKBP-255 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCERE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAc | IPGGATLVFEVELLKIER |
| | G99R | ggGAGAAGAGAAAGCTGGTCATCCCTAGCGAACTC | (SEQ ID NO: 310) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 309) | |
| FKBP-256 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIMR |
| | E137M | GCGAGAAGAGAAAGCTGGTCATCCCTAGCGAACTC | (SEQ ID NO: 312) |
| | | GGCTACGGCGAAAGAGGCtggCCTCCAAAGATTCC | |
| | | TGGCGGAGCCACACTGGTGTTCGAAGTGGAACTGC | |
| | | TGAAGATCatgCGA (SEQ ID NO: 311) | |
| FKBP-257 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIFR |
| | E137F | GCGAGAAGAGAAAGCTGGTCATCCCTAGCGAACTC | (SEQ ID NO: 314) |
| | | GGCTACGGCGAAAGAGGCtggCCTCCAAAGATTCC | |
| | | TGGCGGAGCCACACTGGTGTTCGAAGTGGAACTGC | |
| | | TGAAGATCtttCGA (SEQ ID NO: 313) | |
| FKBP-258 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFFVELLKIER |
| | E130F | GCGAGAAGAGAAAGCTGGTCATCCCTAGCGAACTC | (SEQ ID NO: 316) |
| | | GGCTACGGCGAAAGAGGCtggCCTCCAAAGATTCC | |
| | | TGGCGGAGCCACACTGGTGTTCtttGTGGAACTGC | |
| | | TGAAGATCGAGCGA (SEQ ID NO: 315) | |
| FKBP-259 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFKVELLKIER |
| | E130K | GCGAGAAGAGAAAGCTGGTCATCCCTAGCGAACTC | (SEQ ID NO: 318) |
| | | GGCTACGGCGAAAGAGGCtggCCTCCAAAGATTCC | |
| | | TGGCGGAGCCACACTGGTGTTCaaaGTGGAACTGC | |
| | | TGAAGATCGAGCGA (SEQ ID NO: 317) | |
| FKBP-260 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGERIKGWDQGLLGMCEGE |
| | V85R | TTCGTGTTTAGCCTCGGCACCGGCGAAcggATCAA | NRKLVIPSELGYGERGWPPK |
| | DRD | AGGCTGGGATCAAGGCCTGCTGGGAATGTGCGAAG | IPGGATLVFEVELLKIER |
| | K101N | GCGAGaatAGAAAGCTGGTCATCCCTAGCGAACTC | (SEQ ID NO: 320) |
| | | GGCTACGGCGAAAGAGGCTGGCCTCCAAAGATTCC | |
| | | TGGCGGAGCCACACTGGTGTTCGAAGTGGAACTGC | |
| | | TGAAGATCGAGCGA (SEQ ID NO: 319) | |
| FKBP-261 | Q72R, | CACTGCagaATCAAGAGCAGAAAAGGCGACGTGCT | HCRIKSRKGDVLHAHYTGKL |
| | Q84E, | GCACgctCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLYIER |
| | P43R | GCGAGAAGAGAAAGCTGGTCATCCCTAGCGAACTC | (SEQ ID NO: 322) |
| | M54A | GGCTACGGCGAAAGAGGCtggCCTCCAAAGATTCC | |
| | K135Y | TGGCGGAGCCACACTGGTGTTCGAAGTGGAACTGC | |
| | | TGtacATCGAGCGA (SEQ ID NO: 321) | |
| FKBP-262 | Q72R, | CACTGCagaATCAAGAGCAGAAAAGGCGACGTGCT | HCRIKSRKGDVLHMHYTGKL |
| | Q84E, | GCACatgCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLYIER |
| | P43R | GCGAGAAGAGAAAGCTGGTCATCCCTAGCGAACTC | (SEQ ID NO: 324) |
| | K135Y | GGCTACGGCGAAAGAGGCtggCCTCCAAAGATTCC | |
| | | TGGCGGAGCCACACTGGTGTTCGAAGTGGAACTGC | |
| | | TGtacATCGAGCGA (SEQ ID NO: 323) | |
| FKBP-263 | Q72R, | CACTGCagaATCAAGAGCAGAAAAGGCGACGTGCT | HCRIKSRKGDVLHMHYTGKL |
| | Q84G, | GCACatgCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGGRIKGWDQGLLGMCEGE |
| | V85R | TTCGTGTTTAGCCTCGGCACCGGCggacggATCAA | KRKLVIPSELGYGERGWPPK |
| | DRD | AGGCTGGGATCAAGGCCTGCTGGGAATGTGCGAAG | IPGGATLVFEVELLYIER |
| | P43R | GCGAGAAGAGAAAGCTGGTCATCCCTAGCGAACTC | (SEQ ID NO: 326) |
| | K135Y | GGCTACGGCGAAAGAGGCtggCCTCCAAAGATTCC | |
| | | TGGCGGAGCCACACTGGTGTTCGAAGTGGAACTGC | |
| | | TGtacATCGAGCGA (SEQ ID NO: 325) | |
| FKBP-264 | Q72R, | CACTGCCCAATCAAGAGCAGAAAAGGCGACGTGCT | HCPIKSRKGDVLHMHYTGKL |
| | S79W, | GCACatgCACTACACCGGCAAGCTGGAAGATGGCA | EDGTEFDSSLPRNQPFVFWL |
| | Q84R, | CCGAGTTCGATAGCAGCCTGCCTagaAACCAGCCT | GTGRRIKGWDQGLLGMCEGE |
| | A117W | TTCGTGTTTtggCTCGGCACCGGCagacggATCAA | KRKLVIPSELGYGERGWPPK |
| | DRD | AGGCTGGGATCAAGGCCTGCTGGGAATGTGCGAAG | IPGGATLVFEVELLKIER |
| | V85R | GCGAGAAGAGAAAGCTGGTCATCCCTAGCGAACTC | (SEQ ID NO: 328) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 327) | |
| FKBP-265 | Q72R, | CACTGCCCAATCAAGAGCAGAAAAGGCGACGTGCT | HCPIKSRKGDVLHMHYTGKL |
| | T82W, | GCACatgCACTACACCGGCAAGCTGGAAGATGGCA | EDGTEFDSSLPRNQPFVFSL |
| | Q84R, | CCGAGTTCGATAGCAGCCTGCCTagaAACCAGCCT | GWGRRIKGWDQGLLGMCEGE |
| | A117W | TTCGTGTTTAGCCTCGGCtggGGCagacggATCAA | KRKLVIPSELGYGERGWPPK |
| | DRD | AGGCTGGGATCAAGGCCTGCTGGGAATGTGCGAAG | IPGGATLVFEVELLKIER |
| | V85R | GCGAGAAGAGAAAGCTGGTCATCCCTAGCGAACTC | (SEQ ID NO: 330) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 329) | |
| FKBP-266 | Q72R, | CACTGCCCAATCAAGAGCAGAAAAGGCcttGTGCT | HCPIKSRKGLVLHMHYTGKL |
| | Q84E, | GCACatgCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIER |
| | D50L | GCGAGAAGAGAAAGCTGGTCATCCCTAGCGAACTC | (SEQ ID NO: 332) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 331) | |
| FKBP-267 | Q72R, | CACTGCCCAATCAAGAGCAGAAAAGGCGACGTGCT | HCPIKSRKGDVLHMHYTGKL |
| | Q84E, | GCACatgCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGWCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAtggTGCGAAG | IPGGATLVFEVELLKIER |
| | M96W | GCGAGAAGAGAAAGCTGGTCATCCCTAGCGAACTC | (SEQ ID NO: 334) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 333) | |
| FKBP-268 | Q72R, | CACcgtCCAATCAAGAGCAGAAAAGGCGACGTGCT | HRPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | GIPGATLVFEVELLKIER |
| | C42R | GCGAGAAGAGAAAGCTGGTCATCCCTAGCGAACTC | (SEQ ID NO: 336) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 335) | |
| FKBP-269 | Q72R, | CACTGCCCAATCAAGAGCAGAAAAGGCGACGTGCT | HCPIKSRKGDVLQMHYTGKL |
| | Q84E, | GcagATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIER |
| | H53Q | GCGAGAAGAGAAAGCTGGTCATCCCTAGCGAACTC | (SEQ ID NO: 338) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 337) | |
| FKBP-270 | Q72R, | CACTGCCCAATCAAGAGCAGAAAAGGCGACGTGat | HCPIKSRKGDVMHMHYTGKL |
| | Q84E, | gCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIER |
| | L52M | GCGAGAAGAGAAAGCTGGTCATCCCTAGCGAACTC | (SEQ ID NO: 340) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 339) | |
| FKBP-271 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRRLVIPSELGYGERGWPPK |
| | DRD | AGGCTGGGATCAAGGCCTGCTGGGAATGTGCGAAG | IPGGATLVFEVELLKIER |
| | K103R | GCGAGAAGAGAcgtCTGGTCATCCCTAGCGAACTC | (SEQ ID NO: 342) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 341) | |
| FKBP-272 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTAGAAACCAGCCT | GTGERIKGWDQGLLGMCEGE |
| | V85R | TTCGTGTTTAGCCTCGGCACCGGCGAAcggATCAA | KRKLVIPSELGYGERGWPPK |
| | DRD | AGGCTGGGATCAAGGCCTGCTGGGAATGTGCGAAG | IPGGATLVFEVELLLIER |
| | K135L | GCGAGAAGAGAAAGCTGGTCATCCCTAGCGAACTC | (SEQ ID NO: 344) |
| | | GGCTACGGCGAAAGAGGCTGGCCTCCAAAGATTCC | |
| | | TGGCGGAGCCACACTGGTGTTCGAAGTGGAACTGC | |
| | | TGttgATCGAGCGA (SEQ ID NO: 343) | |
| FKBP-273 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGGRGWPPK |
| | DRD | AGGCTGGGATCAAGGCCTGCTGGGAATGTGCGAAG | IPGGATLVFEVELLKIER |
| | E114G | GCGAGAAGAGAAAGCTGGTCATCCCTAGCGAACTC | (SEQ ID NO: 346) |
| | | GGCTACGGCggtAGAGGCtggCCTCCAAAGATTCC | |
| | | TGGCGGAGCCACACTGGTGTTCGAAGTGGAACTGC | |
| | | TGAAGATCGAGCGA (SEQ ID NO: 345) | |
| FKBP-274 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSKLGYGERGWPPK |
| | DRD | AGGCTGGGATCAAGGCCTGCTGGGAATGTGCGAAG | IPGGATLVFEVELLKIER |
| | E109K | GCGAGAAGAGAAAGCTGGTCATCCCTAGCaaaCTC | (SEQ ID NO: 348) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 347) | |
| FKBP-275 | Q72R, | CACTGCCCAATCAAGAGCAGAAAAgacGACGTGCT | HCPIKSRKDDVLHMHYTGKL |
| | Q84E, | GCACATGCACTACACCGGCAAGCTGGAAGATGGCA | EDGTEFDSSLPRNQPFVFSL |
| | A117W | CCGAGTTCGATAGCAGCCTGCCTagaAACCAGCCT | GTGERIKGWDQGLLGMCEGE |
| | V85R | TTCGTGTTTAGCCTCGGCACCGGCgaacggATCAA | KRKLVIPSELGYGERGWPPK |
| | DRD | AGGCTGGGATCAAGGCCTGCTGGGAATGTGCGAAG | IPGGATLVFEVELLKIER |
| | G49D | GCGAGAAGAGAAAGCTGGTCATCCCTAGCGAACTC | (SEQ ID NO: 350) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 349) | |
| FKBP-276 | Q72R, | CACTGCCCAATCAAGAGCAGAAAAGGCGACGTGCT | HCPIKSRKGDVLHMHYTGKL |
| | Q74R, | GCACATGCACTACACCGGCAAGCTGGAAGATGGCA | EDGTEFDSSLPRNRPFVFSL |
| | Q84E, | CCGAGTTCGATAGCAGCCTGCCTagaAACagaCCT | GTGERIKGWDQGLLGMCEGE |
| | A117W | TTCGTGTTTAGCCTCGGCACCGGCgaacggATCAA | KRKLVIPSELGYGERGWPPK |
| | V85R | AGGCTGGGATCAAGGCCTGCTGGGAATGTGCGAAG | IPGGATLVFEVELLKIER |
| | | GCGAGAAGAGAAAGCTGGTCATCCCTAGCGAACTC | (SEQ ID NO: 352) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 351) | |
| FKBP-277 | Q72R, | CACTGCCCAATCAAGAGCAGAAAAGGCGACGTGCT | HCPIKSRKGDVLHMHYTGKL |
| | Q74R, | GCACATGCACTACACCGGCAAGCTGGAAGATGGCA | EDGTEFDSSLPRNRPFVFSL |
| | Q84E, | CCGAGTTCGATAGCAGCCTGCCTagaAACagaCCT | GTGERIKGWDQGLLGMCEGE |
| | A117W | TTCGTGTTTAGCCTCGGCACCGGCGAAcggATCAA | KRKLVIPSELGYGERGWPPK |
| | V85R, | AGGCTGGGATCAAGGCCTGCTGGGAATGTGCGAAG | IPGGATLVFEVELLYIER |
| | K135Y | GCGAGAAGAGAAAGCTGGTCATCCCTAGCGAACTC | (SEQ ID NO: 354) |
| | | GGCTACGGCGAAAGAGGCTGGCCTCCAAAGATTCC | |
| | | TGGCGGAGCCACACTGGTGTTCGAAGTGGAACTGC | |
| | | TGtacATCGAGCGA (SEQ ID NO: 353) | |
| FKBP-278 | Q72R, | CACTGCCCAATCAAGAGCAGAAAAGGCGACGTGCT | HCPIKSRKGDVLHMHYTGKL |
| | Q74R, | GCACATGCACTACACCGGCAAGCTGGAAGATGGCA | EDGTEFDSSLPRNRPFVFSL |
| | Q84E, | CCGAGTTCGATAGCAGCCTGCCTagaAACagaCCT | GTGERIKGWDQGLLGMCEGE |
| | A117W | TTCGTGTTTAGCCTCGGCACCGGCgaacggATCAA | KRKLVIPSELGYGERGWPPK |
| | V85R, | AGGCTGGGATCAAGGCCTGCTGGGAATGTGCGAAG | IPGGATLVFFVELLKIER |
| | E130F | GCGAGAAGAGAAAGCTGGTCATCCCTAGCGAACTC | (SEQ ID NO: 356) |
| | | GGCTACGGCGAAAGAGGCtggCCTCCAAAGATTCC | |
| | | TGGCGGAGCCACACTGGTGTTCtttGTGGAACTGC | |
| | | TGAAGATCGAGCGA (SEQ ID NO: 355) | |
| FKBP-279 | Q72R, | CACTGCCCAATCAAGAGCAGAAAAGGCGACGTGCT | HCPIKSRKGDVLHMHYTGKL |
| | Q74R, | GCACATGCACTACACCGGCAAGCTGGAAGATGGCA | EDGTEFDSSLPRNRPFVFSL |
| | Q84E, | CCGAGTTCGATAGCAGCCTGCCTagaAACagaCCT | GTGERIKGWDQGLLGMCEGE |
| | A117W | TTCGTGTTTAGCCTCGGCACCGGCgaacggATCAA | KRKLVIPSELGYGERGWPPI |
| | V85R, | AGGCTGGGATCAAGGCCTGCTGGGAATGTGCGAAG | IPGGATLVFEVELLKIER |
| | K120I | GCGAGAAGAGAAAGCTGGTCATCCCTAGCGAACTC | (SEQ ID NO: 358) |
| | | GGCTACGGCGAAAGAGGCtggCCTCCAatcATTCC | |
| | | TGGCGGAGCCACACTGGTGTTCGAAGTGGAACTGC | |
| | | TGAAGATCGAGCGA (SEQ ID NO: 357) | |
| FKBP-280 | Q72R, | CACTGCCCAATCAAGAGCAGAAAAGGCGACGTGCT | HCPIKSRKGDVLHMHYTGKL |
| | Q74R, | GCACATGCACTACACCGGCAAGCTGGAAGATGGCA | EDGTEFDSSLPRNRPFVFSL |
| | Q84E, | CCGAGTTCGATAGCAGCCTGCCTagaAACagaCCT | GTGERIKGWDQGLLGMCEGE |
| | A117W | TTCGTGTTTAGCCTCGGCACCGGCgaacggATCAA | KRKLVIPSELGYGERGWPPI |
| | V85R, | AGGCTGGGATCAAGGCCTGCTGGGAATGTGCGAAG | IPGGATLVFEVELLYIER |
| | K135Y, | GCGAGAAGAGAAAGCTGGTCATCCCTAGCGAACTC | (SEQ ID NO: 360) |
| | K120I | GGCTACGGCGAAAGAGGCtggCCTCCAatcATTCC | |
| | | TGGCGGAGCCACACTGGTGTTCGAAGTGGAACTGC | |
| | | TGtacATCGAGCGA (SEQ ID NO: 359) | |
| FKBP-281 | Q72R, | CACTGCCCAATCAAGAGCAGAAAAGGCGACGTGCT | HCPIKSRKGDVLHMHYTGKL |
| | Q74R, | GCACATGCACTACACCGGCAAGCTGGAAGATGGCA | EDGTEFDSSLPRNRPFVFSL |
| | Q84E, | CCGAGTTCGATAGCAGCCTGCCTagaAACagaCCT | GTGERIKGWDQGLLGMCEGE |
| | A117W | TTCGTGTTTAGCCTCGGCACCGGCgaacggATCAA | KRKLVIPSELGYGERGWPPI |
| | V85R, | AGGCTGGGATCAAGGCCTGCTGGGAATGTGCGAAG | IPGGATLVFFVELLKIER |
| | E130F, | GCGAGAAGAGAAAGCTGGTCATCCCTAGCGAACTC | (SEQ ID NO: 362) |
| | K120I | GGCTACGGCGAAAGAGGCtggCCTCCAatcATTCC | |
| | | TGGCGGAGCCACACTGGTGTTCtttGTGGAACTGC | |
| | | TGAAGATCGAGCGA (SEQ ID NO: 361) | |
| FKBP-282 | Q72R, | CACTGCCCAATCAAGAGCAGAAAAGGCGACGTGCT | HCPIKSRKGDVLHMHYTGKL |
| | Q74W, | GCACATGCACTACACCGGCAAGCTGGAAGATGGCA | EDGTEFDSSLPRNWPFVFSL |
| | Q84E, | CCGAGTTCGATAGCAGCCTGCCTagaAACtggCCT | GTGERIKGWDQGLLGMCEGE |
| | A117W | TTCGTGTTTAGCCTCGGCACCGGCgaacggATCAA | KRKLVIPSELGYGERGWPPK |
| | V85R | AGGCTGGGATCAAGGCCTGCTGGGAATGTGCGAAG | IPGGATLVFEVELLKIER |
| | | GCGAGAAGAGAAAGCTGGTCATCCCTAGCGAACTC | (SEQ ID NO: 364) |
| | | GGCTACGGCGAAAGAGGCtggCCTCCAAAGATTCC | |
| | | TGGCGGAGCCACACTGGTGTTCGAAGTGGAACTGC | |
| | | TGAAGATCGAGCGA (SEQ ID NO: 363) | |
| FKBP-283 | Q72R, | CACTGCCCAATCAAGAGCAGAAAAGGCGACGTGCT | HCPIKSRKGDVLHMHYTGKL |
| | Q74W, | GCACATGCACTACACCGGCAAGCTGGAAGATGGCA | EDGTEFDSSLPRNWPFVFSL |
| | Q84E, | CCGAGTTCGATAGCAGCCTGCCTagaAACtggCCT | GTGERIKGWDQGLLGMCEGE |
| | A117W | TTCGTGTTTAGCCTCGGCACCGGCGAAcggATCAA | KRKLVIPSELGYGERGWPPK |
| | V85R, | AGGCTGGGATCAAGGCCTGCTGGGAATGTGCGAAG | IPGGATLVFEVELLYIER |
| | K135Y | GCGAGAAGAGAAAGCTGGTCATCCCTAGCGAACTC | (SEQ ID NO: 366) |
| | | GGCTACGGCGAAAGAGGCTGGCCTCCAAAGATTCC | |
| | | TGGCGGAGCCACACTGGTGTTCGAAGTGGAACTGC | |
| | | TGtacATCGAGCGA (SEQ ID NO: 365) | |
| FKBP-284 | Q72R, | CACTGCCCAATCAAGAGCAGAAAAGGCGACGTGCT | HCPIKSRKGDVLHMHYTGKL |
| | Q74W, | GCACATGCACTACACCGGCAAGCTGGAAGATGGCA | EDGTEFDSSLPRNWPFVFSL |
| | Q84E, | CCGAGTTCGATAGCAGCCTGCCTagaAACtggCCT | GTGERIKGWDQGLLGMCEGE |
| | A117W | TTCGTGTTTAGCCTCGGCACCGGCgaacggATCAA | KRKLVIPSELGYGERGWPPK |
| | V85R, | AGGCTGGGATCAAGGCCTGCTGGGAATGTGCGAAG | IPGGATLVFFVELLKIER |
| | E130F | GCGAGAAGAGAAAGCTGGTCATCCCTAGCGAACTC | (SEQ ID NO: 368) |
| | | GGCTACGGCGAAAGAGGCtggCCTCCAAAGATTCC | |
| | | TGGCGGAGCCACACTGGTGTTCtttGTGGAACTGC | |
| | | TGAAGATCGAGCGA (SEQ ID NO: 367) | |
| FKBP-285 | Q72R, | CACTGCCCAATCAAGAGCAGAAAAGGCGACGTGCT | HCPIKSRKGDVLHMHYTGKL |
| | Q74W, | GCACATGCACTACACCGGCAAGCTGGAAGATGGCA | EDGTEFDSSLPRNWPFVFSL |
| | Q84E, | CCGAGTTCGATAGCAGCCTGCCTagaAACtggCCT | GTGERIKGWDQGLLGMCEGE |
| | A117W | TTCGTGTTTAGCCTCGGCACCGGCgaacggATCAA | KRKLVIPSELGYGERGWPPI |
| | V85R, | AGGCTGGGATCAAGGCCTGCTGGGAATGTGCGAAG | IPGGATLVFEVELLKIER |
| | K120I | GCGAGAAGAGAAAGCTGGTCATCCCTAGCGAACTC | (SEQ ID NO: 370) |
| | | GGCTACGGCGAAAGAGGCtggCCTCCAatcATTCC | |
| | | TGGCGGAGCCACACTGGTGTTCGAAGTGGAACTGC | |
| | | TGAAGATCGAGCGA (SEQ ID NO: 369) | |
| FKBP-286 | Q72R, | CACTGCCCAATCAAGAGCAGAAAAGGCGACGTGCT | HCPIKSRKGDVLHMHYTGKL |
| | Q74W, | GCACATGCACTACACCGGCAAGCTGGAAGATGGCA | EDGTEFDSSLPRNWPFVFSL |
| | Q84E, | CCGAGTTCGATAGCAGCCTGCCTagaAACtggCCT | GTGERIKGWDQGLLGMCEGE |
| | A117W | TTCGTGTTTAGCCTCGGCACCGGCgaacggATCAA | KRKLVIPSELGYGERGWPPI |
| | V85R, | AGGCTGGGATCAAGGCCTGCTGGGAATGTGCGAAG | IPGGATLVFEVELLYIER |
| | K135Y, | GCGAGAAGAGAAAGCTGGTCATCCCTAGCGAACTC | (SEQ ID NO: 372) |
| | K120I | GGCTACGGCGAAAGAGGCtggCCTCCAatcATTCC | |
| | | TGGCGGAGCCACACTGGTGTTCGAAGTGGAACTGC | |
| | | TGtacATCGAGCGA (SEQ ID NO: 371) | |
| FKBP-287 | Q72R, | CACTGCCCAATCAAGAGCAGAAAAGGCGACGTGCT | HCPIKSRKGDVLHMHYTGKL |
| | Q74W, | GCACATGCACTACACCGGCAAGCTGGAAGATGGCA | EDGTEFDSSLPRNWPFVFSL |
| | Q84E, | CCGAGTTCGATAGCAGCCTGCCTagaAACtggCCT | GTGERIKGWDQGLLGMCEGE |
| | A117W | TTCGTGTTTAGCCTCGGCACCGGCgaacggATCAA | KRKLVIPSELGYGERGWPPI |
| | V85R, | AGGCTGGGATCAAGGCCTGCTGGGAATGTGCGAAG | IPGGATLVFFVELLKIER |
| | E130F, | GCGAGAAGAGAAAGCTGGTCATCCCTAGCGAACTC | (SEQ ID NO: 374) |
| | K120I | GGCTACGGCGAAAGAGGCtggCCTCCAatcATTCC | |
| | | TGGCGGAGCCACACTGGTGTTCtttGTGGAACTGC | |
| | | TGAAGATCGAGCGA (SEQ ID NO: 373) | |
| FKBP-288 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W, | CCGAGTTCGATAGCAGCCTGCCTagaAACCAGCCT | GTGERIKGWDQGLLGMCEGE |
| | V85R, | TTCGTGTTTAGCCTCGGCACCGGCgaacggATCAA | KRKLVIPSELGYGERGWPPI |
| | K120I | AGGCTGGGATCAAGGCCTGCTGGGAATGTGCGAAG | IPGGATLVFEVELLKIER |
| | | GCGAGAAGAGAAAGCTGGTCATCCCTAGCGAACTC | (SEQ ID NO: 376) |
| | | GGCTACGGCGAAAGAGGCtggCCTCCAatcATTCC | |
| | | TGGCGGAGCCACACTGGTGTTCGAAGTGGAACTGC | |
| | | TGAAGATCGAGCGA (SEQ ID NO: 375) | |
| FKBP-289 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W, | CCGAGTTCGATAGCAGCCTGCCTagaAACCAGCCT | GTGERIKGWDQGLLGMCEGE |
| | V85R, | TTCGTGTTTAGCCTCGGCACCGGCgaacggATCAA | KRKLVIPSELGYGERGWPPI |
| | K120I, | AGGCTGGGATCAAGGCCTGCTGGGAATGTGCGAAG | IPGGATLVFEVELLYIER |
| | K135Y | GCGAGAAGAGAAAGCTGGTCATCCCTAGCGAACTC | (SEQ ID NO: 378) |
| | | GGCTACGGCGAAAGAGGCtggCCTCCAatcATTCC | |
| | | TGGCGGAGCCACACTGGTGTTCGAAGTGGAACTGC | |
| | | TGtacATCGAGCGA (SEQ ID NO: 377) | |
| FKBP-290 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W, | CCGAGTTCGATAGCAGCCTGCCTagaAACCAGCCT | GTGERIKGWDQGLLGMCEGE |
| | V85R, | TTCGTGTTTAGCCTCGGCACCGGCgaacggATCAA | KRKLVIPSELGYGERGWPPI |
| | K120I, | AGGCTGGGATCAAGGCCTGCTGGGAATGTGCGAAG | IPGGATLVFFVELLKIER |
| | E130F | GCGAGAAGAGAAAGCTGGTCATCCCTAGCGAACTC | (SEQ ID NO: 380) |
| | | GGCTACGGCGAAAGAGGCtggCCTCCAatcATTCC | |
| | | TGGCGGAGCCACACTGGTGTTCtttGTGGAACTGC | |
| | | TGAAGATCGAGCGA (SEQ ID NO: 379) | |
| FKBP-291 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W, | CCGAGTTCGATAGCAGCCTGCCTagaAACCAGCCT | GTGERIKGWDQGLLGMCEGE |
| | V85R, | TTCGTGTTTAGCCTCGGCACCGGCgaacggATCAA | KRKLVIPSELGYGERGWPPK |
| | E130N | AGGCTGGGATCAAGGCCTGCTGGGAATGTGCGAAG | IPGGATLVFNVELLKIER |
| | | GCGAGAAGAGAAAGCTGGTCATCCCTAGCGAACTC | (SEQ ID NO: 382) |
| | | GGCTACGGCGAAAGAGGCtggCCTCCAAAGATTCC | |
| | | TGGCGGAGCCACACTGGTGTTCaatGTGGAACTGC | |
| | | TGAAGATCGAGCGA (SEQ ID NO: 381) | |
| FKBP-292 | Q72R, | CACTGCCCAATCAAGAGCAGAAAAGGCGACGTGCT | HCPIKSRKGDVLHMHYTGKL |
| | Q84E, | GCACATGCACTACACCGGCAAGCTGGAAGATGGCA | EDGTEFDSSLPRNQPFVFSL |
| | A117W, | CCGAGTTCGATAGCAGCCTGCCTagaAACCAGCCT | GTGERIKGWDQGLLGMCEGE |
| | V85R, | TTCGTGTTTAGCCTCGGCACCGGCGAAcggATCAA | KRKLVIPSELGYGERGWPPK |
| | K135Q | AGGCTGGGATCAAGGCCTGCTGGGAATGTGCGAAG | IPGGATLVFEVELLQIER |
| | | GCGAGAAGAGAAAGCTGGTCATCCCTAGCGAACTC | (SEQ ID NO: 384) |
| | | GGCTACGGCGAAAGAGGCTGGCCTCCAAAGATTCC | |
| | | TGGCGGAGCCACACTGGTGTTCGAAGTGGAACTGC | |
| | | TGcagATCGAGCGA (SEQ ID NO: 383) | |

TABLE 11
Top 30 Drug Responsive Domains (DRDs) for Cas9 Screen

| Construct | | | |
|---|---|---|---|
| # | Mutation | Nucleic Acid Sequence | Amino Acid Sequence |
| FKBP-166 | Q72R, | CACTGCCCAATCAAGAGCAGAAAAG | HCPIKSRKGDVLHAHYTGKLEDGTE |
| | S79W, | GCGACGTGCTGCACgctCACTACAC | FDSSLPRNQPFVFWLGTGERIKGWD |
| | Q84E, | CGGCAAGCTGGAAGATGGCACCGAG | QGLLGMCEGEKRKLVIPSELGYGER |
| | A117W | TTCGATAGCAGCCTGCCTagaAACC | GWPPKIPGGATLVFEVELLKIER |
| | DRD | AGCCTTTCGTGTTTtggCTCGGCAC | (SEQ ID NO: 132) |
| | M54A | CGGCgaacggATCAAAGGCTGGGAT | |
| | V85R | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCtggCCTCCAAAGATTCCTGGCG | |
| | | GAGCCACACTGGTGTTCGAAGTGGA | |
| | | ACTGCTGAAGATCGAGCGA | |
| | | (SEQ ID NO: 131) | |
| FKBP-167 | Q72R, | CACTGCCCAATCAAGAGCAGAAAAG | HCPIKSRKGDVLHAHYTGKLEDGTE |
| | S79W, | GCGACGTGCTGCACgctCACTACAC | FDSSLPRNQPFVFWLGTGRRIKGWD |
| | Q84R, | CGGCAAGCTGGAAGATGGCACCGAG | QGLLGMCEGEKRKLVIPSELGYGER |
| | A117W | TTCGATAGCAGCCTGCCTagaAACC | GWPPKIPGGATLVFEVELLKIER |
| | DRD | AGCCTTTCGTGTTTtggCTCGGCAC | (SEQ ID NO: 134) |
| | M54A | CGGCagacggATCAAAGGCTGGGAT | |
| | V85R | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCtggCCTCCAAAGATTCCTGGCG | |
| | | GAGCCACACTGGTGTTCGAAGTGGA | |
| | | ACTGCTGAAGATCGAGCGA | |
| | | (SEQ ID NO: 133) | |
| FKBP-168 | Q72R, | CACTGCCCAATCAAGAGCAGAAAAG | HCPIKSRKGDVLHAHYTGKLEDGTE |
| | T82W, | GCGACGTGCTGCACgctCACTACAC | FDSSLPRNQPFVFSLGWGRRIKGWD |
| | Q84R, | CGGCAAGCTGGAAGATGGCACCGAG | QGLLGMCEGEKRKLVIPSELGYGER |
| | A117W | TTCGATAGCAGCCTGCCTagaAACC | GWPPKIPGGATLVFEVELLKIER |
| | DRD | AGCCTTTCGTGTTTAGCCTCGGCtg | (SEQ ID NO: 136) |
| | M54A | gGGCagacggATCAAAGGCTGGGAT | |
| | V85R | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCtggCCTCCAAAGATTCCTGGCG | |
| | | GAGCCACACTGGTGTTCGAAGTGGA | |
| | | ACTGCTGAAGATCGAGCGA | |
| | | (SEQ ID NO: 135) | |
| FKBP-170 | Q72R, | CACTGCCCAATCAAGAGCAGAAAAG | HCPIKSRKGDVLHAHYTGKLEDGTE |
| | T82W, | GCGACGTGCTGCACgctCACTACAC | FDSSLPRNQPFVFSLGWGERIKGWD |
| | Q84E, | CGGCAAGCTGGAAGATGGCACCGAG | QGLLGMCEGEKRKLVIPSELGYGER |
| | A117W | TTCGATAGCAGCCTGCCTagaAACC | GWPPKIPGGATLVFEVELLKIER |
| | DRD | AGCCTTTCGTGTTTAGCCTCGGCtg | (SEQ ID NO: 140) |
| | M54A | gGGCgaacggATCAAAGGCTGGGAT | |
| | V85R | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCtggCCTCCAAAGATTCCTGGCG | |
| | | GAGCCACACTGGTGTTCGAAGTGGA | |
| | | ACTGCTGAAGATCGAGCGA | |
| | | (SEQ ID NO: 139) | |
| FKBP-181 | Q72R, | CACTGCCCAATCAAGAGCAGAAAAG | HCPIKSRKGLVLHAHYTGKLEDGTE |
| | Q84E, | GCcttGTGCTGCACgctCACTACAC | FDSSLPRNQPFVFSLGTGERIKGWD |
| | A117W | CGGCAAGCTGGAAGATGGCACCGAG | QGLLGMCEGEKRKLVIPSELGYGER |
| | V85R | TTCGATAGCAGCCTGCCTagaAACC | GWPPKIPGGATLVFEVELLKIER |
| | DRD | AGCCTTTCGTGTTTAGCCTCGGCAC | (SEQ ID NO: 162) |
| | M54A | CGGCgaacggATCAAAGGCTGGGAT | |
| | D50L | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCtggCCTCCAAAGATTCCTGGCG | |
| | | GAGCCACACTGGTGTTCGAAGTGGA | |
| | | ACTGCTGAAGATCGAGCGA | |
| | | (SEQ ID NO: 161) | |
| FKBP-182 | Q72R, | CACTGCCCAATCAAGAGCAGAAAAG | HCPIKSRKGDVLHAHYTGKLEDGTE |
| | Q84E, | GCGACGTGCTGCACgctCACTACAC | FDSSLPRNQPFVFSLGTGERIKGWD |
| | A117W | CGGCAAGCTGGAAGATGGCACCGAG | QGLLGWCEGEKRKLVIPSELGYGER |
| | V85R | TTCGATAGCAGCCTGCCTagaAACC | GWPPKIPGGATLVFEVELLKIER |
| | DRD | AGCCTTTCGTGTTTAGCCTCGGCAC | (SEQ ID NO: 164) |
| | M54A | CGGCgaacggATCAAAGGCTGGGAT | |
| | M96W | CAAGGCCTGCTGGGAtggTGCGAAG | |
| | | GCGAGAAGAGAAAGCTGGTCATCCC | |
| | | TAGCGAACTCGGCTACGGCGAAAGA | |
| | | GGCtggCCTCCAAAGATTCCTGGCG | |
| | | GAGCCACACTGGTGTTCGAAGTGGA | |
| | | ACTGCTGAAGATCGAGCGA | |
| | | (SEQ ID NO: 163) | |
| FKBP-191 | Q72R, | CACTGCCCAATCAAGAGCAGAAAAt | HCPIKSRKSDVLHMHYTGKLEDGTE |
| | Q84E, | ccGACGTGCTGCACATGCACTACAC | FDSSLPRNQPFVFSLGTGERIKGWD |
| | A117W | CGGCAAGCTGGAAGATGGCACCGAG | QGLLGMCEGEKRKLVIPSELGYGER |
| | V85R | TTCGATAGCAGCCTGCCTAGAAACC | GWPPKIPGGATLVFEVELLKIER |
| | DRD | AGCCTTTCGTGTTTAGCCTCGGCAC | (SEQ ID NO: 182) |
| | G49S | CGGCGAAcggATCAAAGGCTGGGAT | |
| | | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCTGGCCTCCAAAGATTCCTGGCG | |
| | | GAGCCACACTGGTGTTCGAAGTGGA | |
| | | ACTGCTGAAGATCGAGCGA | |
| | | (SEQ ID NO: 181) | |
| FKBP-192 | Q72R, | CACTGCCCAATCAAGAGCAGAAAAG | HCPIKSRKGDVLHMHYTGKLEDGTE |
| | Q84E, | GCGACGTGCTGCACATGCACTACAC | FDSSLPRNQLFVFSLGTGERIKGWD |
| | A117W | CGGCAAGCTGGAAGATGGCACCGAG | QGLLGMCEGEKRKLVIPSELGYGER |
| | V85R | TTCGATAGCAGCCTGCCTAGAAACC | GWPPKIPGGATLVFEVELLKIER |
| | DRD | AGctgTTCGTGTTTAGCCTCGGCAC | (SEQ ID NO: 184) |
| | P75L | CGGCGAAcggATCAAAGGCTGGGAT | |
| | | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCTGGCCTCCAAAGATTCCTGGCG | |
| | | GAGCCACACTGGTGTTCGAAGTGGA | |
| | | ACTGCTGAAGATCGAGCGA | |
| | | (SEQ ID NO: 183) | |
| FKBP-195 | Q72R, | CACTGCCCAATCAAGAGCAGAAAAG | HCPIKSRKGDVLHMHYTGKLEDGTE |
| | Q84E, | GCGACGTGCTGCACATGCACTACAC | FDSSLPRNQPFVFSLGTGERIKGWD |
| | A117W | CGGCAAGCTGGAAGATGGCACCGAG | QGLLGMCEGEKRKLVIPSELGYGER |
| | V85R | TTCGATAGCAGCCTGCCTAGAAACC | GWPPKIPGGATLVFEVELLYIER |
| | DRD | AGCCTTTCGTGTTTAGCCTCGGCAC | (SEQ ID NO: 190) |
| | K135Y | CGGCGAAcggATCAAAGGCTGGGAT | |
| | | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCTGGCCTCCAAAGATTCCTGGCG | |
| | | GAGCCACACTGGTGTTCGAAGTGGA | |
| | | ACTGCTGtacATCGAGCGA | |
| | | (SEQ ID NO: 189) | |
| FKBP-197 | Q72R, | CACTGCCCAATCAAGAGCAGAAAAG | HCPIKSRKGDVLHMHYTGKLEDGTE |
| | Q84E, | GCGACGTGCTGCACATGCACTACAC | FDSSLPRNQPFVFSLGTGERIKGWD |
| | A117W | CGGCAAGCTGGAAGATGGCACCGAG | QGLLGMCEGEKRKLVIPSELGYGER |
| | V85R | TTCGATAGCAGCCTGCCTAGAAACC | GWPPKIPGGATLVFRVELLKIER |
| | DRD | AGCCTTTCGTGTTTAGCCTCGGCAC | (SEQ ID NO: 194) |
| | E130R | CGGCGAAcggATCAAAGGCTGGGAT | |
| | | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCTGGCCTCCAAAGATTCCTGGCG | |
| | | GAGCCACACTGGTGTTCagaGTGGA | |
| | | ACTGCTGAAGATCGAGCGA | |
| | | (SEQ ID NO: 193) | |
| FKBP-198 | Q72R, | CACTGCCCAATCAAGAGCAGAAAAG | HCPIKSRKGDVLHMHYTGKLEDGTE |
| | Q84E, | GCGACGTGCTGCACATGCACTACAC | FDSSLPRNQPFVFSLGTGERIKGWD |
| | A117W | CGGCAAGCTGGAAGATGGCACCGAG | QGLLGMCEGEWRKLVIPSELGYGER |
| | V85R | TTCGATAGCAGCCTGCCTAGAAACC | GWPPKIPGGATLVFEVELLKIER |
| | DRD | AGCCTTTCGTGTTTAGCCTCGGCAC | (SEQ ID NO: 196) |
| | K101W | CGGCGAACggATCAAAGGCTGGGAT | |
| | | CAAGGCCTGCTGGGAATGTGCGAAG | |
| | | GCGAGtggAGAAAGCTGGTCATCCC | |
| | | TAGCGAACTCGGCTACGGCGAAAGA | |
| | | GGCTGGCCTCCAAAGATTCCTGGCG | |
| | | GAGCCACACTGGTGTTCGAAGTGGA | |
| | | ACTGCTGAAGATCGAGCGA | |
| | | (SEQ ID NO: 195) | |
| FKBP-199 | Q72R, | CACTGCCCAATCAAGAGCAGAAAAG | HCPIKSRKGDVLHMHYTGKLEDGTE |
| | Q84E, | GCGACGTGCTGCACATGCACTACAC | FDSSLPRNQPFVFSLGTGERIKGWD |
| | A117W | CGGCAAGCTGGAAGATGGCACCGAG | QGLLGMCLGEKRKLVIPSELGYGER |
| | V85R | TTCGATAGCAGCCTGCCTAGAAACC | GWPPKIPGGATLVFEVELLKIER |
| | DRD | AGCCTTTCGTGTTTAGCCTCGGCAC | (SEQ ID NO: 198) |
| | E98L | CGGCGAAcggATCAAAGGCTGGGAT | |
| | | CAAGGCCTGCTGGGAATGTGCttgG | |
| | | GCGAGAAGAGAAAGCTGGTCATCCC | |
| | | TAGCGAACTCGGCTACGGCGAAAGA | |
| | | GGCTGGCCTCCAAAGATTCCTGGCG | |
| | | GAGCCACACTGGTGTTCGAAGTGGA | |
| | | ACTGCTGAAGATCGAGCGA | |
| | | (SEQ ID NO: 197) | |
| FKBP-200 | Q72R, | CACTGCCCAATCAAGAGCAGAAAAG | HCPIKSRKGDVLHMHYTGKLESGTE |
| | Q84E, | GCGACGTGCTGCACATGCACTACAC | FDSSLPRNQPFVFSLGTGERIKGWD |
| | A117W | CGGCAAGCTGGAAtctGGCACCGAG | QGLLGMCEGEKRKLVIPSELGYGER |
| | V85R | TTCGATAGCAGCCTGCCTAGAAACC | GWPPKIPGGATLVFEVELLKIER |
| | DRD | AGCCTTTCGTGTTTAGCCTCGGCAC | (SEQ ID NO: 200) |
| | D62S | CGGCGAAcggATCAAAGGCTGGGAT | |
| | | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCTGGCCTCCAAAGATTCCTGGCG | |
| | | GAGCCACACTGGTGTTCGAAGTGGA | |
| | | ACTGCTGAAGATCGAGCGA | |
| | | (SEQ ID NO: 199) | |
| FKBP-201 | Q72R, | CACTGCCCAATCAAGAGCAGAAAAG | HCPIKSRKGDVLLMHYTGKLEDGTE |
| | Q84E, | GCGACGTGCTGttgATGCACTACAC | FDSSLPRNQPFVFSLGTGERIKGWD |
| | A117W | CGGCAAGCTGGAAGATGGCACCGAG | QGLLGMCEGEKRKLVIPSELGYGER |
| | V85R | TTCGATAGCAGCCTGCCTAGAAACC | GWPPKIPGGATLVFEVELLKIER |
| | DRD | AGCCTTTCGTGTTTAGCCTCGGCAC | (SEQ ID NO: 202) |
| | H53L | CGGCGAAcggATCAAAGGCTGGGAT | |
| | | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCTGGCCTCCAAAGATTCCTGGCG | |
| | | GAGCCACACTGGTGTTCGAAGTGGA | |
| | | ACTGCTGAAGATCGAGCGA | |
| | | (SEQ ID NO: 201) | |
| FKBP-203 | Q72R, | CACTGCCCAATCAAGAGCAGAAAAG | HCPIKSRKGDVLHAHYTGKLEDGTE |
| | Q84E, | GCGACGTGCTGCACgctCACTACAC | FDSSLPRNQLFVFSLGTGERIKGWD |
| | A117W | CGGCAAGCTGGAAGATGGCACCGAG | QGLLGMCEGEKRKLVIPSELGYGER |
| | V85R | TTCGATAGCAGCCTGCCTAGAAACC | GWPPKIPGGATLVFEVELLKIER |
| | DRD | AGctgTTCGTGTTTAGCCTCGGCAC | (SEQ ID NO: 206) |
| | M54A | CGGCGAAcggATCAAAGGCTGGGAT | |
| | P75L | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCTGGCCTCCAAAGATTCCTGGCG | |
| | | GAGCCACACTGGTGTTCGAAGTGGA | |
| | | ACTGCTGAAGATCGAGCGA | |
| | | (SEQ ID NO: 205) | |
| FKBP-209 | Q72R, | CACTGCCCAATCAAGAGCAGAAAAG | HCPIKSRKGDVLHAHYTGKLEDGTE |
| | Q84E, | GCGACGTGCTGCACgctCACTACAC | FDSSLPRNQPFVFSLGTGERIKGWD |
| | A117W | CGGCAAGCTGGAAGATGGCACCGAG | QGLLGMCEGEWRKLVIPSELGYGER |
| | V85R | TTCGATAGCAGCCTGCCTAGAAACC | GWPPKIPGGATLVFEVELLKIER |
| | DRD | AGCCTTTCGTGTTTAGCCTCGGCAC | (SEQ ID NO: 218) |
| | M54A | CGGCGAAcggATCAAAGGCTGGGAT | |
| | K101W | CAAGGCCTGCTGGGAATGTGCGAAG | |
| | | GCGAGtggAGAAAGCTGGTCATCCC | |
| | | TAGCGAACTCGGCTACGGCGAAAGA | |
| | | GGCTGGCCTCCAAAGATTCCTGGCG | |
| | | GAGCCACACTGGTGTTCGAAGTGGA | |
| | | ACTGCTGAAGATCGAGCGA | |
| | | (SEQ ID NO: 217) | |
| FKBP-214 | Q72R, | CACTGCagaATCAAGAGCAGAAAAG | HCRIKSRKGDVLHAHYTGKLEDGTE |
| | Q84G, | GCGACGTGCTGCACgctCACTACAC | FDSSLPRNQPFVFSLGTGGRIKGWD |
| | A117W | CGGCAAGCTGGAAGATGGCACCGAG | QGLLGMCEGEKRKLVIPSELGYGER |
| | V85R | TTCGATAGCAGCCTGCCTAGAAACC | GWPPKIPGGATLVFEVELLKIER |
| | DRD | AGCCTTTCGTGTTTAGCCTCGGCAC | (SEQ ID NO: 228) |
| | P43R | CGGCggacggATCAAAGGCTGGGAT | |
| | M54A | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCtggCCTCCAAAGATTCCTGGCG | |
| | | GAGCCACACTGGTGTTCGAAGTGGA | |
| | | ACTGCTGAAGATCGAGCGA | |
| | | (SEQ ID NO: 227) | |
| FKBP-215 | Q72R, | CACTGCagaATCAAGAGCAGAAAAG | HCRIKSRKGDVLHAHYTGKLEDGTE |
| | Q84G, | GCGACGTGCTGCACgctCACTACAC | FDSSLPRNQPFVFSLGTGGRIKGWD |
| | A117W | CGGCAAGCTGGAAGATGGCACCGAG | QGLLGMCEGEKRKLVIPSELGYGER |
| | V85R | TTCGATAGCAGCCTGCCTAGAAACC | GWPPKIPGGATLVFEVELLYIER |
| | DRD | AGCCTTTCGTGTTTAGCCTCGGCAC | (SEQ ID NO: 230) |
| | P43R | CGGCggacggATCAAAGGCTGGGAT | |
| | M54A | CAAGGCCTGCTGGGAATGTGCGAAG | |
| | K135Y | GCGAGAAGAGAAAGCTGGTCATCCC | |
| | | TAGCGAACTCGGCTACGGCGAAAGA | |
| | | GGCtggCCTCCAAAGATTCCTGGCG | |
| | | GAGCCACACTGGTGTTCGAAGTGGA | |
| | | ACTGCTGtacATCGAGCGA | |
| | | (SEQ ID NO: 229) | |
| FKBP-220 | Q72R, | CACTGCCCAATCAAGAGCAGAAAAG | HCPIKSRKGDVLHMHYTGKLEDGGE |
| | Q84E, | GCGACGTGCTGCACATGCACTACAC | FDSSLPRNQPFVFSLGTGERIKGWD |
| | A117W | CGGCAAGCTGGAAGATGGCgggGAG | QGLLGMCEGEKRKLVIPSELGYGER |
| | V85R | TTCGATAGCAGCCTGCCTAGAAACC | GWPPKIPGGATLVFEVELLKIER |
| | DRD | AGCCTTTCGTGTTTAGCCTCGGCAC | (SEQ ID NO: 240) |
| | T25G | CGGCGAAcggATCAAAGGCTGGGAT | |
| | | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCTGGCCTCCAAAGATTCCTGGCG | |
| | | GAGCCACACTGGTGTTCGAAGTGGA | |
| | | ACTGCTGAAGATCGAGCGA | |
| | | (SEQ ID NO: 239) | |
| FKBP-223 | Q72R, | CACTGCtggATCAAGAGCAGAAAAG | HCWIKSRKGDVLHAHYTGKLEDGRE |
| | Q84E, | GCGACGTGCTGCACgctCACTACAC | FDSSLPRNQPFVFSLGTGERIKGWD |
| | A117W | CGGCAAGCTGGAAGATGGCcggGAG | QGLLGMCEGEKRKLVIPSELGYGER |
| | V85R | TTCGATAGCAGCCTGCCTAGAAACC | GWPPKIPGGATLVFEVELLKIER |
| | DRD | AGCCTTTCGTGTTTAGCCTCGGCAC | (SEQ ID NO: 246) |
| | M54A | CGGCGAAcggATCAAAGGCTGGGAT | |
| | P4W | CAAGGCCTGCTGGGAATGTGCGAAG | |
| | T25R | GCGAGAAGAGAAAGCTGGTCATCCC | |
| | | TAGCGAACTCGGCTACGGCGAAAGA | |
| | | GGCTGGCCTCCAAAGATTCCTGGCG | |
| | | GAGCCACACTGGTGTTCGAAGTGGA | |
| | | ACTGCTGAAGATCGAGCGA | |
| | | (SEQ ID NO: 245) | |
| FKBP-226 | Q72R, | CACTGCagaATCAAGAGCAGAAAAG | HCRIKSRKGDVLHAHYTGKLEDGTE |
| | Q84E, | GCGACGTGCTGCACgctCACTACAC | FDSSLPRNQPFVFSLGTGERIKGWD |
| | A117W | CGGCAAGCTGGAAGATGGCACCGAG | QGLLGMCEGEKRKLVIPSELGYGER |
| | V85R | TTCGATAGCAGCCTGCCTAGAAACC | GWPPKIPGGATLVFEVELLKIER |
| | DRD | AGCCTTTCGTGTTTAGCCTCGGCAC | (SEQ ID NO: 252) |
| | P43R | CGGCgaacggATCAAAGGCTGGGAT | |
| | M54A | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCtggCCTCCAAAGATTCCTGGCG | |
| | | GAGCCACACTGGTGTTCGAAGTGGA | |
| | | ACTGCTGAAGATCGAGCGA | |
| | | (SEQ ID NO: 251) | |
| FKBP-228 | Q72R, | CACTGCCCAATCAAGAGCAGAAAAG | HCPIKSRKGDVLHMHYTGKLEDGTE |
| | Q84E, | GCGACGTGCTGCACATGCACTACAC | FDSSLPRNQPFVFSLGTGERIKGWD |
| | A117W | CGGCAAGCTGGAAGATGGCACCGAG | QGLLGMCEGEKRKLVIPSELGYGER |
| | V85R | TTCGATAGCAGCCTGCCTagaAACC | GWPPKIPGGATLVFEVELLKIRR |
| | DRD | AGCCTTTCGTGTTTAGCCTCGGCAC | (SEQ ID NO: 256) |
| | E137R | CGGCgaacggATCAAAGGCTGGGAT | |
| | | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCtggCCTCCAAAGATTCCTGGCG | |
| | | GAGCCACACTGGTGTTCGAAGTGGA | |
| | | ACTGCTGAAGATCaggCGA | |
| | | (SEQ ID NO: 255) | |
| FKBP-229 | Q72R, | CACTGCCCAATCAAGAGCAGAAAAG | HCPIKSRKGDVLHAHYTGKLEDGTE |
| | Q84E, | GCGACGTGCTGCACgctCACTACAC | FDSSLPRNQPFVFSLGTGERIKGWD |
| | A117W | CGGCAAGCTGGAAGATGGCACCGAG | QGLLGMCEGEKRKLVIPSELGYGER |
| | V85R | TTCGATAGCAGCCTGCCTagaAACC | GWPPKIPGGATLVFEVELLKIRR |
| | DRD | AGCCTTTCGTGTTTAGCCTCGGCAC | (SEQ ID NO: 258) |
| | E137R | CGGCgaacggATCAAAGGCTGGGAT | |
| | M54A | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCtggCCTCCAAAGATTCCTGGCG | |
| | | GAGCCACACTGGTGTTCGAAGTGGA | |
| | | ACTGCTGAAGATCaggCGA | |
| | | (SEQ ID NO: 257) | |
| FKBP-230 | Q72R, | CACTGCCCAATCAAGAGCAGAAAAG | HCPIKSRKGDVLHMHYTGKLEDMTE |
| | Q84E, | GCGACGTGCTGCACATGCACTACAC | FDSSLPRNQPFVFSLGTGEVIKGWD |
| | A117W | CGGCAAGCTGGAAGATATGACCGAG | QGLLGMCEGEKRKLVIPSELGYGER |
| | DRD | TTCGATAGCAGCCTGCCTagaAACC | GWPPKIPGGATLVFEVELLKIER |
| | G63M | AGCCTTTCGTGTTTAGCCTCGGCAC | (SEQ ID NO: 260) |
| | | CGGCgaaGTGATCAAAGGCTGGGAT | |
| | | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCtggCCTCCAAAGATTCCTGGCG | |
| | | GAGCCACACTGGTGTTCGAAGTGGA | |
| | | ACTGCTGAAGATCGAGCGA | |
| | | (SEQ ID NO: 259) | |
| FKBP-233 | Q72R, | CACTGCCCAATCAAGAGCAGAAAAG | HCPIKSRKGDVLHMHYTGKLEDGTE |
| | Q84E, | GCGACGTGCTGCACATGCACTACAC | FDSSLPRNQPFVFSLGTGEVIKGWT |
| | A117W | CGGCAAGCTGGAAGATGGCACCGAG | QGLLGMCEGEKRKLVIPSELGYGER |
| | DRD | TTCGATAGCAGCCTGCCTagaAACC | GWPPKIPGGATLVFEVELLKIER |
| | D90T | AGCCTTTCGTGTTTAGCCTCGGCAC | (SEQ ID NO: 266) |
| | | CGGCgaaGTGATCAAAGGCTGGacg | |
| | | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCtggCCTCCAAAGATTCCTGGCG | |
| | | GAGCCACACTGGTGTTCGAAGTGGA | |
| | | ACTGCTGAAGATCGAGCGA | |
| | | (SEQ ID NO: 265) | |
| FKBP-240 | Q72R, | CACTGCCCAATCAAGAGCAGAAAAG | HCPIKSRKGDVLHMHYTGKLYDGTE |
| | Q84E, | GCGACGTGCTGCACATGCACTACAC | FDSSLPRNQPFVFSLGTGERIKGWD |
| | A117W | CGGCAAGCTGtatGATGGCACCGAG | QGLLGMCEGEKRKLVIPSELGYGER |
| | V85R | TTCGATAGCAGCCTGCCTagaAACC | GWPPKIPGGATLVFEVELLKIER |
| | DRD | AGCCTTTCGTGTTTAGCCTCGGCAC | (SEQ ID NO: 280) |
| | E61Y | CGGCgaacggATCAAAGGCTGGGAT | |
| | | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCtggCCTCCAAAGATTCCTGGCG | |
| | | GAGCCACACTGGTGTTCGAAGTGGA | |
| | | ACTGCTGAAGATCGAGCGA | |
| | | (SEQ ID NO: 279) | |
| FKBP-243 | Q72R, | CACTGCCCAATCAAGAGCAGAAAAG | HCPIKSRKGDVLHMHYTGKLEDGTE |
| | Q84E, | GCGACGTGCTGCACATGCACTACAC | FDSSLPRNQPFVFSLGTGERIKGWD |
| | A117W | CGGCAAGCTGGAAGATGGCACCGAG | QGLLGMCEGEKRKLVIPSELGYGER |
| | V85R | TTCGATAGCAGCCTGCCTagaAACC | GWSPKIPGGATLVFEVELLKIER |
| | DRD | AGCCTTTCGTGTTTAGCCTCGGCAC | (SEQ ID NO: 286) |
| | P118S | CGGCgaacggATCAAAGGCTGGGAT | |
| | | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCtggtcgCCAAAGATTCCTGGCG | |
| | | GAGCCACACTGGTGTTCGAAGTGGA | |
| | | ACTGCTGAAGATCGAGCGA | |
| | | (SEQ ID NO: 285) | |
| FKBP-244 | Q72R, | CACTGCCCAATCAAGAGCAGAAAAG | HCPIKSRKGDVLHAHYTGKLEDGTE |
| | Q84E, | GCGACGTGCTGCACgctCACTACAC | FDSSLPRNQPFVFSLGTGERIKGWD |
| | A117W | CGGCAAGCTGGAAGATGGCACCGAG | QGLLGMCEGEKRKLVIPSELGYGER |
| | V85R | TTCGATAGCAGCCTGCCTagaAACC | GWSPKIPGGATLVFEVELLKIER |
| | DRD | AGCCTTTCGTGTTTAGCCTCGGCAC | (SEQ ID NO: 288) |
| | P118S | CGGCgaacggATCAAAGGCTGGGAT | |
| | M54A | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCtggtcgCCAAAGATTCCTGGCG | |
| | | GAGCCACACTGGTGTTCGAAGTGGA | |
| | | ACTGCTGAAGATCGAGCGA | |
| | | (SEQ ID NO: 287) | |
| FKBP-251 | Q72R, | CACTGCCCAATCAAGAGCAGAAAAG | HCPIKSRKGDVLHMHYTGKLEDGTE |
| | Q84E, | GCGACGTGCTGCACATGCACTACAC | FDSSLPRNQPFVFSLGTGERIKGWD |
| | A117W | CGGCAAGCTGGAAGATGGCACCGAG | QGLLGMCEGEKRKLVIPSELGYGER |
| | V85R | TTCGATAGCAGCCTGCCTagaAACC | GWPPKIPGGATLVFFVELLKIER |
| | DRD | AGCCTTTCGTGTTTAGCCTCGGCAC | (SEQ ID NO: 302) |
| | E130F | CGGCgaacggATCAAAGGCTGGGAT | |
| | | CAAGGCCTGCTGGGAATGTGCGAAG | |
| | | GCGAGAAGAGAAAGCTGGTCATCCC | |
| | | TAGCGAACTCGGCTACGGCGAAAGA | |
| | | GGCtggCCTCCAAAGATTCCTGGCG | |
| | | GAGCCACACTGGTGTTCtttGTGGA | |
| | | ACTGCTGAAGATCGAGCGA | |
| | | (SEQ ID NO: 301) | |
| FKBP-255 | Q72R, | CACTGCCCAATCAAGAGCAGAAAAG | HCPIKSRKGDVLHMHYTGKLEDGTE |
| | Q84E, | GCGACGTGCTGCACATGCACTACAC | FDSSLPRNQPFVFSLGTGERIKGWD |
| | A117W | CGGCAAGCTGGAAGATGGCACCGAG | QGLLGMCEREKRKLVIPSELGYGER |
| | V85R | TTCGATAGCAGCCTGCCTagaAACC | GWPPKIPGGATLVFEVELLKIER |
| | DRD | AGCCTTTCGTGTTTAGCCTCGGCAC | (SEQ ID NO: 310) |
| | G99R | CGGCgaacggATCAAAGGCTGGGAT | |
| | | CAAGGCCTGCTGGGAATGTGCGAAc | |
| | | ggGAGAAGAGAAAGCTGGTCATCCC | |
| | | TAGCGAACTCGGCTACGGCGAAAGA | |
| | | GGCtggCCTCCAAAGATTCCTGGCG | |
| | | GAGCCACACTGGTGTTCGAAGTGGA | |
| | | ACTGCTGAAGATCGAGCGA | |
| | | (SEQ ID NO: 309) | |

TABLE 12
Ec50 and Ec90 Values for FKBP Constructs 160-255

| | | | EC50 | | EC90 | |
|---|---|---|---|---|---|---|
| Construct | Mutation | EC50 | (Norm) | EC90 | (Norm) | FC |
| FKBP-160 | Q72R, Q84E, A117W | 0.0108 | 0.0111 | 0.04223 | 0.04824 | 25.7 |
| | V85R DRD M54A | | | | | |
| FKBP-161 | Q72R, Q84E, A117W | 0.0688 | 0.0694 | 0.512 | 0.5411 | 32.3 |
| | DRD M54S V85R | | | | | |
| FKBP-162 | Q72R, Q84E, A117W | 0.2282 | 0.2293 | 1.796 | 1.874 | 46.6 |
| | DRD M54A, del c-term R | | | | | |
| | V85R | | | | | |
| FKBP-163 | Q72R, Q84E, A117W | 0.1345 | 0.1459 | 0.7563 | 1.008 | 29.4 |
| | DRD M54A, M96V V85R | | | | | |
| FKBP-164 | Q72R, Q84E, A117W | 0.1370 | 0.1377 | 0.5804 | 0.621 | 42.7 |
| | DRD M54A, P118V V85R | | | | | |
| FKBP-165 | Q72R, Q84E, A117W | 0.2582 | 0.2606 | 0.9695 | 0.9835 | 47.6 |
| | DRD M54A, P119V V85R | | | | | |
| FKBP-166 | Q72R, S79W, Q84E, | 0.0434 | 0.0439 | 0.2197 | 0.2226 | 46.0 |
| | A117W DRD M54A | | | | | |
| | V85R | | | | | |
| FKBP-167 | Q72R, S79W, Q84R, | 0.0706 | 0.0694 | 0.3478 | 0.3345 | 46.8 |
| | A117W DRD M54A | | | | | |
| | V85R | | | | | |
| FKBP-168 | Q72R, T82W, Q84R, | 0.0637 | 0.0646 | 0.3419 | 0.3511 | 60.2 |
| | A117W DRD M54A | | | | | |
| | V85R | | | | | |
| FKBP-169 | Q72R, T82W, Q84E, | 0.0990 | 0.1031 | 0.4484 | 0.4929 | 50.1 |
| | A117W DRD M54A | | | | | |
| FKBP-170 | Q72R, T82W, Q84E, | 0.0304 | 0.0307 | 0.1333 | 0.1384 | 47.6 |
| | A117W DRD M54A | | | | | |
| | V85R | | | | | |
| FKBP-171 | Q72R, Q84E, A117W | 1.7340 | 1.9160 | 8.161 | 10.14 | 60.3 |
| | DRD M54A D50L | | | | | |
| FKBP-172 | Q72R, Q84E, A117W | 0.1556 | 0.1633 | 1.046 | 1.306 | 63.1 |
| | DRD M54A M96W | | | | | |
| FKBP-173 | Q72R, Q84E, A117W | ND | ND | ND | ND | 4 |
| | DRD M54A L133G | | | | | |
| FKBP-174 | Q72R, Q84E, A117W | ND | ND | ND | ND | 0.9 |
| | DRD M54A S36G, V131G | | | | | |
| FKBP-175 | Q72R, Q84E, A117W | ND | ND | ND | ND | 7.25 |
| | DRD M54A D50L, T57I, | | | | | |
| | I136L | | | | | |
| FKBP-176 | Q72R, Q84E, A117W | ND | ND | ND | ND | 6.8 |
| | DRD M54A G116E | | | | | |
| FKBP-177 | Q72R, Q84E, A117W | 4.3770 | 5.0700 | 18.68 | 23.86 | 22.3 |
| | DRD M54A L80G | | | | | |
| FKBP-178 | Q72R, Q84E, A117W | 4.2870 | 4.9830 | 22.34 | 28.45 | 36.4 |
| | DRD M54A F78S | | | | | |
| FKBP-179 | Q72R, Q84E, A117W | ND | ND | ND | ND | 3.1 |
| | DRD M54A H53G, R72L | | | | | |
| FKBP-180 | Q72R, Q84E, A117W | ND | ND | ND | ND | 1.2 |
| | DRD M54A F129T | | | | | |
| FKBP-181 | Q72R, Q84E, A117W | 0.5021 | 0.7223 | 14.08 | 5.057 | 52.0 |
| | V85R DRD M54A D50L | | | | | |
| FKBP-182 | Q72R, Q84E, A117W | 0.1341 | 0.1386 | 0.8185 | 0.9284 | 64.9 |
| | V85R DRD M54A M96W | | | | | |
| FKBP-183 | Q72R, Q84E, A117W | ND | ND | ND | ND | 4 |
| | V85R DRD M54A L133G | | | | | |
| FKBP-184 | Q72R, Q84E, A117W | ND | ND | ND | ND | 1 |
| | V85R DRD M54A S36G, | | | | | |
| | V131G | | | | | |
| FKBP-185 | Q72R, Q84E, A117W | 4.9530 | 5.1490 | 22.09 | 23.43 | 40.2 |
| | V85R DRD M54A D50L, | | | | | |
| | T57I, I136L | | | | | |
| FKBP-186 | Q72R, Q84E, A117W | 4.3280 | 4.8370 | 17.92 | 20.96 | 19.0 |
| | V85R DRD M54A G116E | | | | | |
| FKBP-187 | Q72R, Q84E, A117W | 3.2900 | 4.1920 | 14.51 | 21.3 | 24.0 |
| | V85R DRD M54A L80G | | | | | |
| FKBP-188 | Q72R, Q84E, A117W | 3.6930 | 4.2970 | 16.58 | 20.82 | 20.5 |
| | V85R DRD M54A F78S | | | | | |
| FKBP-189 | Q72R, Q84E, A117W | 6.0780 | 6.7900 | 25.83 | 30.11 | 23.0 |
| | V85R DRD M54A H53G, | | | | | |
| | R72L | | | | | |
| FKBP-190 | Q72R, Q84E, A117W | ND | ND | ND | ND | 0.9 |
| | V85R DRD M54A F129T | | | | | |
| FKBP-191 | Q72R, Q84E, A117W | 0.0624 | 0.0637 | 0.3666 | 0.3909 | 50.6 |
| | V85R DRD G49S | | | | | |
| FKBP-192 | Q72R, Q84E, A117W | 0.0430 | 0.0421 | 0.2295 | 0.2278 | 55.0 |
| | V85R DRD P75L | | | | | |
| FKBP-193 | Q72R, Q84E, A117W | 2.7020 | 2.9430 | 13.03 | 14.95 | 37.8 |
| | V85R DRD D90A | | | | | |
| FKBP-194 | Q72R, Q84E, A117W | 0.0200 | 0.0218 | 0.1021 | 0.1216 | 24.8 |
| | V85R DRD L104V | | | | | |
| FKBP-195 | Q72R, Q84E, A117W | 0.0342 | 0.0325 | 0.1648 | 0.1397 | 50.1 |
| | V85R DRD K135Y | | | | | |
| FKBP-196 | Q72R, Q84E, A117W | 0.0239 | 0.0236 | 0.1147 | 0.113 | 34.7 |
| | V85R DRD I136V | | | | | |
| FKBP-197 | Q72R, Q84E, A117W | 0.2129 | 0.2174 | 1.089 | 1.192 | 101.1 |
| | V85R DRD E130R | | | | | |
| FKBP-198 | Q72R, Q84E, A117W | 0.0441 | 0.0445 | 0.2529 | 0.2739 | 54.3 |
| | V85R DRD K101W | | | | | |
| FKBP-199 | Q72R, Q84E, A117W | 0.0187 | 0.0189 | 0.07171 | 0.06753 | 27.1 |
| | V85R DRD E98L | | | | | |
| FKBP-200 | Q72R, Q84E, A117W | 0.0313 | 0.0304 | 0.1605 | 0.1502 | 33.9 |
| | V85R DRD D62S | | | | | |
| FKBP-201 | Q72R, Q84E, A117W | 0.0382 | 0.0386 | 0.1438 | 0.1534 | 33.1 |
| | V85R DRD H53L | | | | | |
| FKBP-202 | Q72R, Q84E, A117W | 0.1723 | 0.1715 | 1.01 | 1.064 | 46.2 |
| | V85R DRD M54A G49S | | | | | |
| FKBP-203 | Q72R, Q84E, A117W | 0.1195 | 0.1237 | 0.6571 | 0.7471 | 66.0 |
| | V85R DRD M54A P75L | | | | | |
| FKBP-204 | Q72R, Q84E, A117W | 4.1050 | 4.1760 | 15.17 | 15.46 | 37.9 |
| | V85R DRD M54A D90A | | | | | |
| FKBP-205 | Q72R, Q84E, A117W | 0.0360 | 0.0363 | 0.1829 | 0.1837 | 30.1 |
| | V85R DRD M54A L104V | | | | | |
| FKBP-206 | Q72R, Q84E, A117W | 0.1123 | 0.1066 | 0.5731 | 0.55 | 50.1 |
| | V85R DRD M54A K135Y | | | | | |
| FKBP-207 | Q72R, Q84E, A117W | 0.0520 | 0.0513 | 0.3107 | 0.3019 | 35.4 |
| | V85R DRD M54A I136V | | | | | |
| FKBP-208 | Q72R, Q84E, A117W | 0.6309 | 0.6251 | 4.225 | 4.118 | 87.2 |
| | V85R DRD M54A E130R | | | | | |
| FKBP-209 | Q72R, Q84E, A117W | 0.0904 | 0.0923 | 0.6816 | 0.7774 | 56.0 |
| | V85R DRD M54A | | | | | |
| | K101W | | | | | |
| FKBP-210 | Q72R, Q84E, A117W | 0.0449 | 0.0466 | 0.1908 | 0.2076 | 32.8 |
| | V85R DRD M54A E98L | | | | | |
| FKBP-211 | Q72R, Q84E, A117W | 0.0781 | 0.0799 | 0.4518 | 0.4813 | 35.7 |
| | V85R DRD M54A D62S | | | | | |
| FKBP-212 | Q72R, Q84E, A117W | 0.0704 | 0.0726 | 0.2987 | 0.317 | 40.2 |
| | V85R DRD M54A H53L | | | | | |
| FKBP-213 | Q72R, Q84G, A117W | 0.0124 | 0.0123 | 0.0474 | 0.04697 | 28.8 |
| | V85R DRD P43R M54A | | | | | |
| FKBP-214 | Q72R, Q84G, A117W | 0.0908 | 0.0900 | 0.3885 | 0.3896 | 51.0 |
| | V85R DRD P43R M54A | | | | | |
| | K135Y | | | | | |
| FKBP-215 | Q72R, Q84G, A117W | 0.0908 | 0.0900 | 0.3885 | 0.3896 | 51.0 |
| | V85R DRD P43R M54A | | | | | |
| | K135Y | | | | | |
| FKBP-216 | Q72R, Q84E, A117W | 3.0360 | 3.0740 | 14.1 | 14.51 | 27.6 |
| | DRD M54A E61Y, | | | | | |
| | K101W, K135Y | | | | | |
| FKBP-217 | Q72R, Q84E, A117W | 3.0730 | 3.2400 | 16.13 | 17.46 | 36.8 |
| | V85R DRD M54A E61Y, | | | | | |
| | K101W, K135Y | | | | | |
| FKBP-218 | Q72R, Q84E, A117W | ND | ND | ND | ND | 4.46 |
| | DRD M54A, E61G, | | | | | |
| | K101G, K135E | | | | | |
| FKBP-219 | Q72R, Q84E, A117W | ND | ND | ND | ND | 5.4 |
| | V85R DRD M54A E61G, | | | | | |
| | K101G, K135E | | | | | |
| FKBP-220 | Q72R, Q84E, A117W | 0.0309 | 0.0312 | 0.1564 | 0.1652 | 34.2 |
| | V85R DRD T25G | | | | | |
| FKBP-221 | Q72R, Q84E, A117W | 0.0854 | 0.0890 | 0.4948 | 0.5667 | 41.8 |
| | V85R DRD M54A T25G | | | | | |
| FKBP-222 | Q72R, Q84E, A117W | 0.1279 | 0.1260 | 0.6124 | 0.6017 | 40.6 |
| | DRD P4W T25R | | | | | |
| FKBP-223 | Q72R, Q84E, A117W | 0.1103 | 0.1129 | 0.6741 | 0.7131 | 52.5 |
| | V85R DRD M54A P4W | | | | | |
| | T25R | | | | | |
| FKBP-224 | Q72R, Q84E, A117W | 0.0184 | 0.0192 | 0.07859 | 0.08874 | 5.2 |
| | DRD P43R | | | | | |
| FKBP-225 | Q72R, Q84E, A117W | 0.0078 | 0.0078 | 0.02535 | 0.02601 | 20.6 |
| | V85R DRD P43R | | | | | |
| FKBP-226 | Q72R, Q84E, A117W | 0.0132 | 0.0133 | 0.04459 | 0.04697 | 31.5 |
| | V85R DRD P43R M54A | | | | | |
| FKBP-227 | Q72R, Q84E, A117W | 0.0563 | 0.0553 | 0.2928 | 0.2782 | 29.0 |
| | DRD E137R | | | | | |
| FKBP-228 | Q72R, Q84E, A117W | 0.0186 | 0.0188 | 0.1163 | 0.1242 | 55.1 |
| | V85R DRD E137R | | | | | |
| FKBP-229 | Q72R, Q84E, A117W | 0.0525 | 0.0510 | 0.4842 | 0.4551 | 54.0 |
| | V85R DRD E137R M54A | | | | | |
| FKBP-230 | Q72R, Q84E, A117W | 0.1664 | 0.1571 | 0.9966 | 0.8896 | 49.0 |
| | DRD G63M | | | | | |
| FKBP-231 | Q72R, Q84E, A117W | 0.0582 | 0.0570 | 0.3964 | 0.3988 | 39.9 |
| | V85R DRD G63M | | | | | |
| FKBP-232 | Q72R, Q84E, A117W | 0.1425 | 0.1514 | 0.9383 | 1.094 | 35.0 |
| | V85R DRD G63M M54A | | | | | |
| FKBP-233 | Q72R, Q84E, A117W | 0.0608 | 0.0649 | 0.2724 | 0.335 | 47.2 |
| | DRD D90T | | | | | |
| FKBP-234 | Q72R, Q84E, A117W | 1.7010 | 1.9900 | 8.756 | 12.08 | 23.5 |
| | V85R DRD D90T | | | | | |
| FKBP-235 | Q72R, Q84E, A117W | 2.3440 | 2.4820 | 9.975 | 11.1 | 23.5 |
| | V85R DRD D90T M54A | | | | | |
| FKBP-236 | Q72R, Q84E, A117W | 0.2931 | 0.3021 | 1.809 | 1.965 | 39.1 |
| | DRD G113N | | | | | |
| FKBP-237 | Q72R, Q84E, A117W | 0.0829 | 0.0798 | 0.5498 | 0.4842 | 24.1 |
| | V85R DRD G113N | | | | | |
| FKBP-238 | Q72R, Q84E, A117W | 0.1534 | 0.1533 | 0.8465 | 0.8626 | 26.8 |
| | V85R DRD G113N M54A | | | | | |
| FKBP-239 | Q72R, Q84E, A117W | 0.0497 | 0.0513 | 0.2234 | 0.243 | 31.1 |
| | DRD E61Y | | | | | |
| FKBP-240 | Q72R, Q84E, A117W | 0.0221 | 0.0219 | 0.1106 | 0.1107 | 43.7 |
| | V85R DRD E61Y | | | | | |
| FKBP-241 | Q72R, Q84E, A117W | 0.0382 | 0.0385 | 0.2213 | 0.2224 | 43.9 |
| | V85R DRD E61Y M54A | | | | | |
| FKBP-242 | Q72R, Q84E, A117W | 0.0962 | 0.1024 | 0.4658 | 0.5388 | 24.4 |
| | DRD P118S | | | | | |
| FKBP-243 | Q72R, Q84E, A117W | 0.0336 | 0.0337 | 0.125 | 0.1272 | 37.1 |
| | V85R DRD P118S | | | | | |
| FKBP-244 | Q72R, Q84E, A117W | 0.0731 | 0.0744 | 0.3066 | 0.3284 | 51.2 |
| | V85R DRD P118S M54A | | | | | |
| FKBP-245 | Q72R, Q84E, A117W | 1.2220 | 1.2320 | 5.832 | 5.869 | 40.6 |
| | DRD S69H | | | | | |
| FKBP-246 | Q72R, Q84E, A117W | 0.3351 | 0.3284 | 1.529 | 1.515 | 30.4 |
| | V85R DRD S69H | | | | | |
| FKBP-247 | Q72R, Q84E, A117W | 0.6194 | 0.6235 | 2.806 | 2.954 | 35.0 |
| | V85R DRD S69H M54A | | | | | |
| FKBP-248 | Q72R, Q84E, A117W | 0.3378 | 0.3417 | 1.805 | 1.884 | 50.1 |
| | DRD S68D | | | | | |
| FKBP-249 | Q72R, Q84E, A117W | 0.0825 | 0.0835 | 0.4721 | 0.4937 | 33.7 |
| | V85R DRD S68D | | | | | |
| FKBP-250 | Q72R, Q84E, A117W | 0.0305 | 0.0320 | 0.1009 | 0.1139 | 22.0 |
| | DRD E130F | | | | | |
| FKBP-251 | Q72R, Q84E, A117W | 0.0302 | 0.0311 | 0.1037 | 0.1132 | 97.5 |
| | V85R DRD E130F | | | | | |
| FKBP-252 | Q72R, Q84E, A117W | ND | ND | ND | ND | 1.3 |
| | DRD I136P | | | | | |
| FKBP-253 | Q72R, Q84E, A117W | ND | ND | ND | ND | 2.3 |
| | V85R DRD I136P | | | | | |
| FKBP-254 | Q72R, Q84E, A117W | 0.0735 | 0.0726 | 0.3693 | 0.3729 | 35.6 |
| | DRD G99R | | | | | |
| FKBP-255 | Q72R, Q84E, A117W | 0.0212 | 0.0211 | 0.1007 | 0.1011 | 33.0 |
| | V85R DRD G99R | | | | | |

Norm is normalized to mCherry; Ec50 and Ec90 are in uM; ND = Not Determined; Only Constructs that had >10 fold change during initial analysis were assayed for Ec50

TABLE 13
Ec50 and Ec90 Values for FKBP Constructs 160-255

| | | | Fold |
|---|---|---|---|
| Construct | Mutation | EC50 | Change |
| FKBP-256 | Q72R, Q84E, A117W V85R DRD E137M | 0.02029 | 78 |
| FKBP-257 | Q72R, Q84E, A117W V85R DRD E137F | 0.02213 | 65 |
| FKBP-258 | Q72R, Q84E, A117W V85R DRD E130F | 0.02514 | 104 |
| FKBP-259 | Q72R, Q84E, A117W V85R DRD E130K | 0.1275 | 103 |
| FKBP-260 | Q72R, Q84E, A117W V85R DRD K101N | 0.1581 | 51 |
| FKBP-261 | Q72R, Q84E, A117W V85R DRD P43R M54A | ND | ND |
| | K135Y | | |
| FKBP-262 | Q72R, Q84E, A117W V85R DRD P43R K135Y | 0.03231 | 73 |
| FKBP-263 | Q72R, Q84G, A117W V85R DRD P43R | 0.0306 | 56 |
| | K135Y | | |
| FKBP-264 | Q72R, S79W, Q84R, A117W DRD V85R | 0.03089 | 37.08 |
| FKBP-265 | Q72R, T82W, Q84R, A117W DRD V85R | 0.01861 | 53.72 |
| FKBP-266 | Q72R, Q84E, A117W V85R DRD D50L | 0.3732 | 60 |
| FKBP-267 | Q72R, Q84E, A117W V85R DRD M96W | 0.1545 | 67 |
| FKBP-268 | Q72R, Q84E, A117W V85R DRD C42R | 0.007083 | 18 |
| FKBP-269 | Q72R, Q84E, A117W V85R DRD H53Q | 0.032 | 44 |
| FKBP-270 | Q72R, Q84E, A117W V85R DRD L52M | 0.01543 | 38 |
| FKBP-271 | Q72R, Q84E, A117W V85R DRD K103R | 0.006167 | 19 |
| FKBP-272 | Q72R, Q84E, A117W V85R DRD K135L | 0.03532 | 105.86 |
| FKBP-273 | Q72R, Q84E, A117W V85R DRD E114G | 0.02087 | 36 |
| FKBP-274 | Q72R, Q84E, A117W V85R DRD E109K | 0.0124 | 34 |
| FKBP-275 | Q72R, Q84E, A117W V85R DRD G49D | 0.0393 | 56 |
| FKBP-276 | Q72R, Q74R, Q84E, A117W V85R | 0.00788 | 7.54 |
| FKBP-277 | Q72R, Q74R, Q84E, A117W V85R, K135Y | 0.05523 | 61.04 |
| FKBP-278 | Q72R, Q74R, Q84E, A117W V85R, E130F | 0.036 | 92.73 |
| FKBP-279 | Q72R, Q74R, Q84E, A117W V85R, K120I | 0.01345 | 31.33 |
| FKBP-280 | Q72R, Q74R, Q84E, A117W V85R, K135Y, | 0.04953 | 68.34 |
| | K120I | | |
| FKBP-281 | Q72R, Q74R, Q84E, A117W V85R, E130F, | 0.03657 | 77.66 |
| | K120I | | |
| FKBP-282 | Q72R, Q74W, Q84E, A117W V85R | 0.04489 | 39.54 |
| FKBP-283 | Q72R, Q74W, Q84E, A117W V85R, K135Y | 0.4043 | 39.79 |
| FKBP-284 | Q72R, Q74W, Q84E, A117W V85R, E130F | 0.3045 | 58.23 |
| FKBP-285 | Q72R, Q74W, Q84E, A117W V85R, K120I | 0.04238 | 36.71 |
| FKBP-286 | Q72R, Q74W, Q84E, A117W V85R, K135Y, | 0.3815 | 25.96 |
| | K120I | | |
| FKBP-287 | Q72R, Q74W, Q84E, A117W V85R, E130F, | 0.311 | 47.09 |
| | K120I | | |
| FKBP-288 | Q72R, Q84E, A117W, V85R, K120I | 0.0074 | 30.1 |
| FKBP-289 | Q72R, Q84E, A117W, V85R, K120I, K135Y | 0.04788 | 73.38 |
| FKBP-290 | Q72R, Q84E, A117W, V85R, K120I, E130F | 0.03021 | 92.89 |
| FKBP-291 | Q72R, Q84E, A117W, V85R, E130N | 0.03457 | 53.37 |
| FKBP-292 | Q72R, Q84E, A117W, V85R, K135Q | 0.02232 | 50.68 |

Ec50 and Ec90 are in μM.
ND: Not determined.

TABLE 14
Rapamycin and Tacrolimus Response Values for FKBP Constructs

| | | Tacrolimus | | | | | Rapamycin | | | | |
|---|---|---|---|---|---|---|---|---|---|---|---|
| | | | EC50 | | EC90 | | | EC50 | | EC90 | |
| Construct | Mutation | EC50 | (Norm) | EC90 | (Norm) | FC | EC50 | (Norm) | EC90 | (Norm) | FC |
| FKBP-91 | Q72R, Q84E, | 0.0260 | 0.0268 | 0.111 | 0.1173 | 12.4 | 0.1009 | 0.0935 | 0.398 | 0.4141 | 3.2 |
| | A117W DRD | | | | | | | | | | |
| | M54A | | | | | | | | | | |
| FKBP-117 | Q72R, Q84E, | 0.0075 | 0.0077 | 0.025 | 0.02716 | 21.2 | 0.0455 | 0.0440 | 0.17 | 0.1712 | 7.6 |
| | A117W DRD | | | | | | | | | | |
| | V85R | | | | | | | | | | |
| FKBP-153 | Q72R, S79W, | 0.0908 | 0.0951 | 0.341 | 0.3829 | 39.7 | 0.4874 | 0.4857 | 1.518 | 1.538 | 6.1 |
| | Q84E, A117W | | | | | | | | | | |
| | DRD M54A | | | | | | | | | | |
| FKBP-155 | Q72R, S79W, | 0.2137 | 0.2264 | 0.808 | 0.947 | 61.3 | 0.3377 | 0.3289 | 1.145 | 1.181 | 7.5 |
| | Q84R, A117W | | | | | | | | | | |
| | DRD M54A | | | | | | | | | | |
| FKBP-157 | Q72R, T82W, | 0.2948 | 0.3130 | 1.145 | 1.321 | 63.5 | 0.2823 | 0.2877 | 1.012 | 1.077 | 11.2 |
| | Q84E, A117W | | | | | | | | | | |
| | DRD M54A | | | | | | | | | | |
| FKBP-166 | Q72R, S79W, | 0.043 | 0.0439 | 0.22 | 0.2226 | 46 | 0.508 | 0.4997 | 1.635 | 1.582 | 15.7 |
| | Q84E, A117W | | | | | | | | | | |
| | DRD M54A | | | | | | | | | | |
| | V85R | | | | | | | | | | |
| FKBP-167 | Q72R, S79W, | 0.071 | 0.0694 | 0.348 | 0.3345 | 46.8 | 0.597 | 0.5891 | 1.72 | 1.711 | 11.3 |
| | Q84R, A117W | | | | | | | | | | |
| | DRD M54A | | | | | | | | | | |
| | V85R | | | | | | | | | | |
| FKBP-168 | Q72R, T82W, | 0.064 | 0.0646 | 0.342 | 0.3511 | 60.2 | 0.336 | 0.3221 | 1.385 | 1.333 | 18 |
| | Q84R, A117W | | | | | | | | | | |
| | DRD M54A | | | | | | | | | | |
| | V85R | | | | | | | | | | |
| FKBP-169 | Q72R, T82W, | 0.099 | 0.1031 | 0.448 | 0.4929 | 50.1 | 0.405 | 0.4007 | 1.391 | 1.416 | 10.8 |
| | Q84E, A117W | | | | | | | | | | |
| | DRD M54A | | | | | | | | | | |
| FKBP-170 | Q72R, T82W, | 0.03 | 0.0307 | 0.133 | 0.1384 | 47.6 | 0.224 | 0.2226 | 0.785 | 0.818 | 16.1 |
| | Q84E, A117W | | | | | | | | | | |
| | DRD M54A | | | | | | | | | | |
| | V85R | | | | | | | | | | |
| FKBP-171 | Q72R, Q84E, | 1.734 | 1.916 | 8.161 | 10.14 | 60.3 | 6.116 | 6.508 | 15.98 | 18.01 | 14.1 |
| | A117W DRD | | | | | | | | | | |
| | M54A D50L | | | | | | | | | | |
| FKBP-172 | Q72R Q84E, | 0.156 | 0.1633 | 1.046 | 1.306 | 63.1 | 0.572 | 0.5717 | 2.136 | 2.293 | 21.3 |
| | A117W DRD | | | | | | | | | | |
| | M54A M96W | | | | | | | | | | |
| FKBP-177 | Q72R, Q84E, | 4.377 | 5.07 | 18.68 | 23.86 | 22.3 | 4.138 | 4.223 | 5.281 | 6.331 | 2.3 |
| | A117W DRD | | | | | | | | | | |
| | M54A L80G | | | | | | | | | | |
| FKBP-178 | Q72R, Q84E, | 4.287 | 4.983 | 22.34 | 28.45 | 36.4 | 7.477 | 46.4 | 16.82 | 151 | 2.9 |
| | A117W DRD | | | | | | | | | | |
| | M54A F78S | | | | | | | | | | |
| FKBP-181 | Q72R, Q84E, | 0.502 | 0.7223 | 14.08 | 5.057 | 52 | 4.413 | 4.235 | 11.84 | 11.48 | 15.5 |
| | A117W V85R | | | | | | | | | | |
| | DRD M54A | | | | | | | | | | |
| | D50L | | | | | | | | | | |
| FKBP-182 | Q72R, Q84E, | 0.134 | 0.1386 | 0.819 | 0.9284 | 64.9 | 1.187 | 1.217 | 3.84 | 4.112 | 21.5 |
| | A117W V85R | | | | | | | | | | |
| | DRD M54A | | | | | | | | | | |
| | M96W | | | | | | | | | | |
| FKBP-185 | Q72R, Q84E, | 4.953 | 5.149 | 22.09 | 23.43 | 40.2 | 4.105 | 8.368 | 5.338 | 22.03 | 2 |
| | A117W V85R | | | | | | | | | | |
| | DRD M54A | | | | | | | | | | |
| | D50L, T57I, | | | | | | | | | | |
| | I136L | | | | | | | | | | |
| FKBP-186 | Q72R, Q84E, | 4.328 | 4.837 | 17.92 | 20.96 | 19 | 4.265 | 4.14 | 5.868 | 5.224 | 1.7 |
| | A117W V85R | | | | | | | | | | |
| | DRD M54A | | | | | | | | | | |
| | G116E | | | | | | | | | | |
| FKBP-187 | Q72R, Q84E, | 3.29 | 4.192 | 14.51 | 21.3 | 24 | 4.173 | 4.086 | 5.311 | 5.661 | 2.3 |
| | A117W V85R | | | | | | | | | | |
| | DRD M54A | | | | | | | | | | |
| | L80G | | | | | | | | | | |
| FKBP-188 | Q72R, Q84E, | 3.693 | 4.297 | 16.58 | 20.82 | 20.5 | 4.228 | 4.124 | 5.41 | 5.831 | 2.6 |
| | A117W V85R | | | | | | | | | | |
| | DRD M54A | | | | | | | | | | |
| | F78S | | | | | | | | | | |
| FKBP-189 | Q72R, Q84E, | 6.078 | 6.79 | 25.83 | 30.11 | 23 | 4.384 | 4.157 | 5.467 | 5.526 | 2.5 |
| | A117W V85R | | | | | | | | | | |
| | DRD M54A | | | | | | | | | | |
| | H53G, R72L | | | | | | | | | | |

Constructs engineered to be less responsive to Rapamycin more specific for Tacrolimus.
FKBP-91 and FKBP-117 are controls in this experiment. Ec50 and Ec90 are in μM.

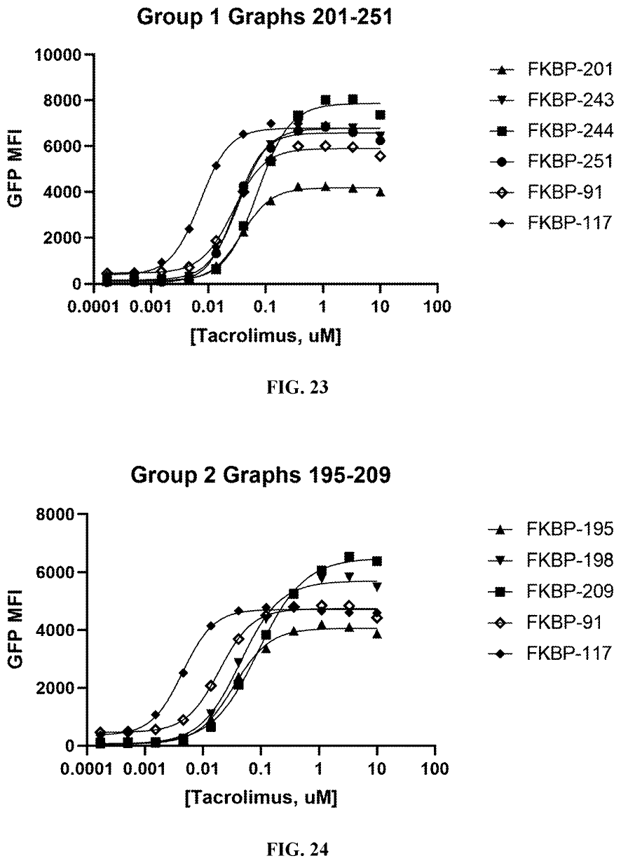

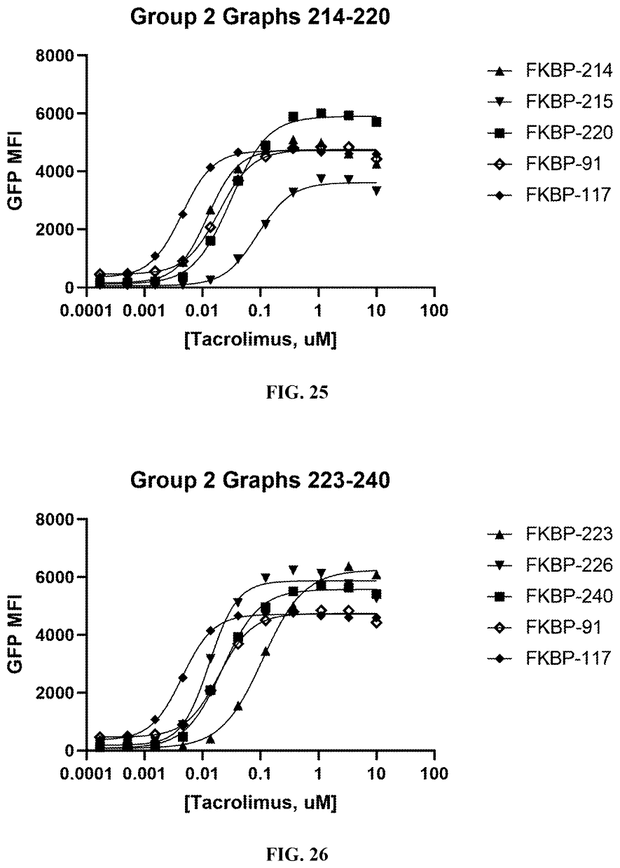

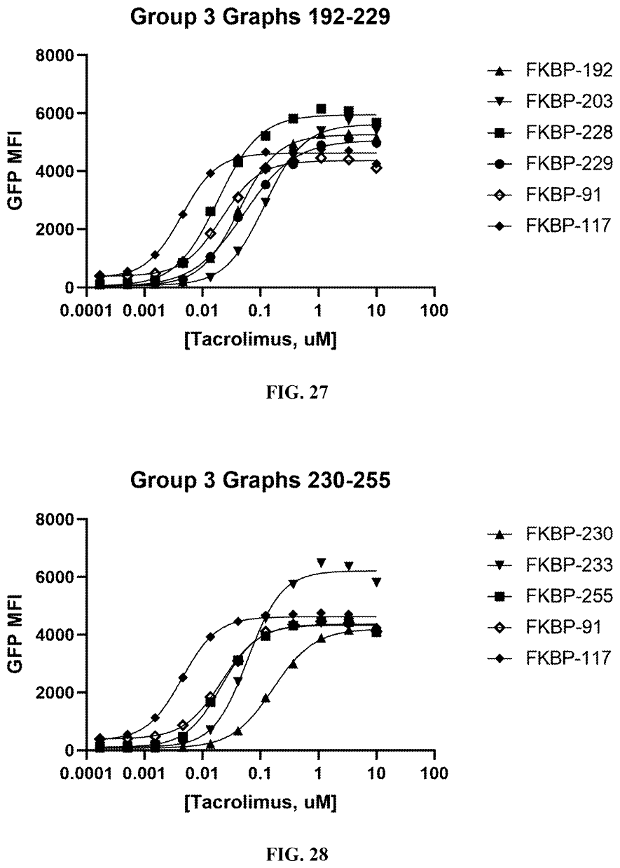

FIGS. 22-38 show the results of characterization of FKBP-160-292. Constructs were stably transduced into Jurkat cells and their response to tacrolimus was analyzed using flow cytometry. FKBP-091 and FKBP-117 was used as a comparative control for FKBP-160-FKBP-255. FKBP-195 was used as a comparative control for FKBP-256-FKBP-275. FKBP-117, FPBP-195 and FKBP-251 were used as a comparative control for FKBP-276-FKBP-292.

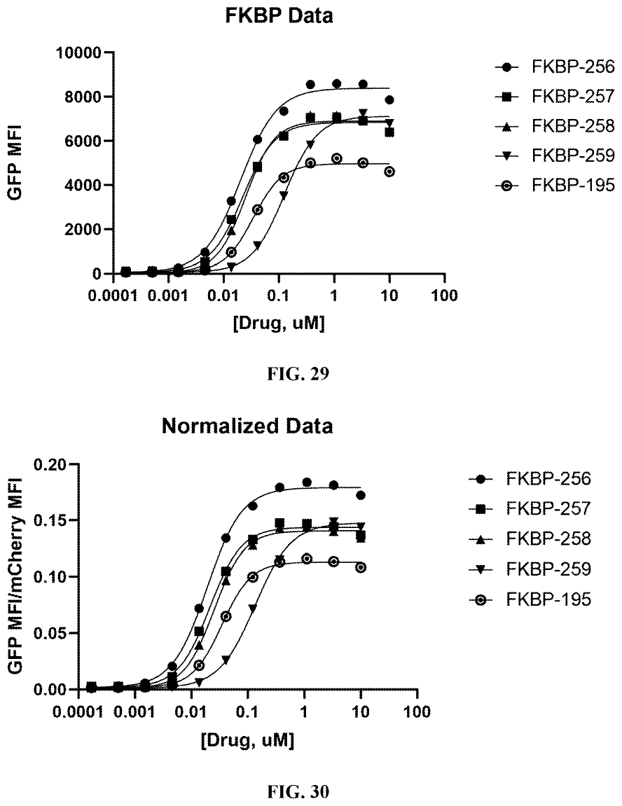

TABLE 15
Ec50 values for the graphs in FIGS. 29 and 30

| | | Ec50 uM | FC |
|---|---|---|---|
| FKBP-256 | Q72R, Q84E, | 0.020 | 78 |
| | A117W, V85R, | | |
| | DRD E137M | | |
| FKBP-257 | Q72R, Q84E, | 0.022 | 65 |
| | A117W, V85R, | | |
| | DRD E137F | | |
| FKBP-258 | Q72R, Q84E, | 0.025 | 104 |
| | A117W, V85R, | | |
| | DRD E130F | | |
| FKBP-259 | Q72R, Q84E, | 0.128 | 103 |
| | A117W, V85R, | | |
| | DRD E130K | | |
| FKBP-195 | Q72R, Q84E, | 0.034 | 66 |
| | A117W, V85R, | | |
| | DRD K135Y | | |

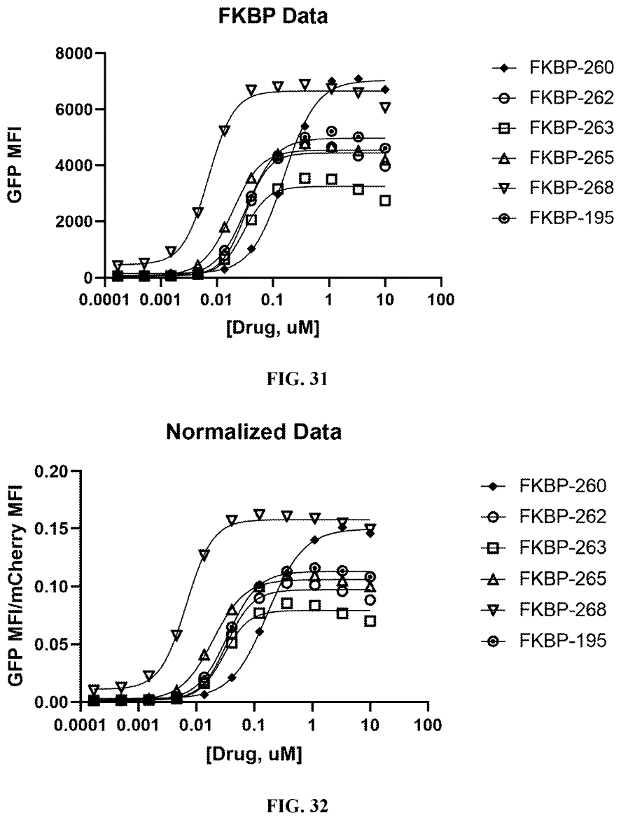

TABLE 16
Ec50 values for the graphs in FIGS. 31 and 32

| | | Ec50 uM | FC |
|---|---|---|---|
| FKBP-260 | Q72R, Q84E, | 0.1581 | 51 |
| | A117W, V85R, | | |
| | DRD K101N | | |
| FKBP-262 | Q72R, Q84E, | 0.03231 | 73 |
| | A117W, V85R, | | |
| | DRD P43R, K135Y | | |
| FKBP-263 | Q72R, Q84G, | 0.0306 | 56 |
| | A117W, V85R, | | |
| | DRD P43R, K135Y | | |
| FKBP-265 | Q72R, T82W, | 0.01861 | 58 |
| | Q84R, A117W, | | |
| | DRD V85R | | |
| FKBP-268 | Q72R, Q84E, | 0.007083 | 18 |
| | A117W, V85R, | | |
| | DRD C42R | | |
| FKBP-195 | Q72R, Q84E, | 0.034 | 66 |
| | A117W, V85R, | | |
| | DRD K135Y | | |

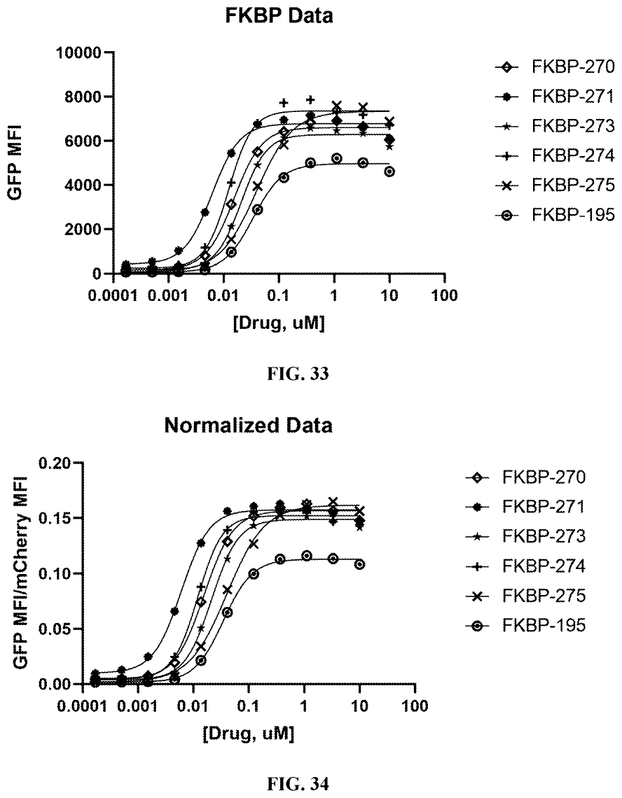

TABLE 17
Ec50 values for the graphs in FIGS. 33 and 34

| | | Ec50 uM | FC |
|---|---|---|---|
| FKBP-270 | Q72R, Q84E, | 0.01543 | 38 |
| | A117W, V85R, | | |
| | DRD L52M | | |
| FKBP-271 | Q72R, Q84E, | 0.006167 | 19 |
| | A117W, V85R, | | |
| | DRD K103R | | |
| FKBP-273 | Q72R, Q84E, | 0.02087 | 36 |
| | A117W, V85R, | | |
| | DRD E114G | | |
| FKBP-274 | Q72R, Q84E, | 0.0124 | 34 |
| | A117W, V85R, | | |
| | DRD E109K | | |
| FKBP-275 | Q72R, Q84E, | 0.0393 | 56 |
| | A117W, V85R, | | |
| | DRD G49D | | |
| FKBP-195 | Q72R, Q84E, | 0.034 | 66 |
| | A117W, V85R, | | |
| | DRD K135Y | | |

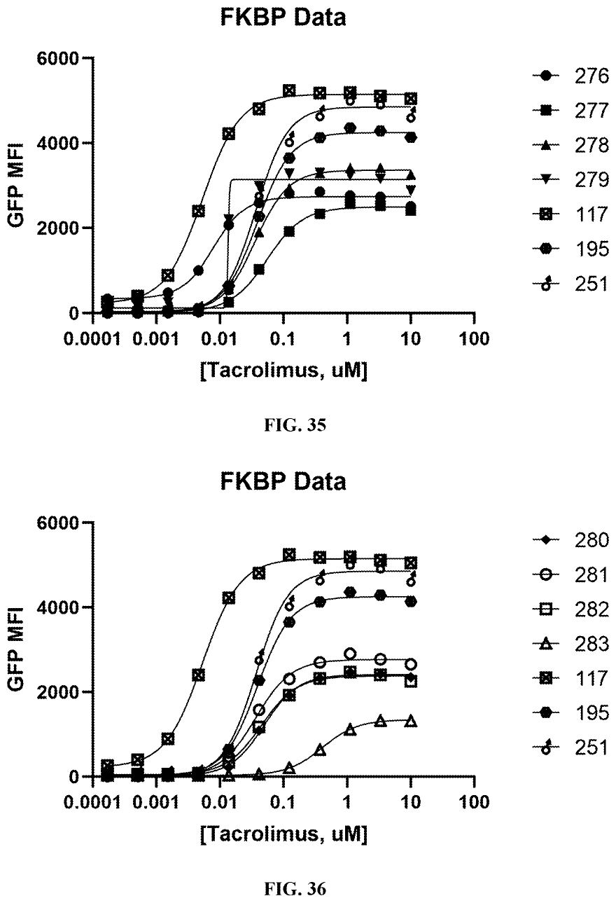

TABLE 18
Ec50, FC and Peak values for the graph in FIG. 35

| Construct | Mutation | Ec50 uM | FC | Peak |
|---|---|---|---|---|
| FKBP-276 | Q72R, Q74R, | 0.00288 | 7.54 | 2859 |
| | Q84E, A117W, | | | |
| | V85R | | | |
| FKBP-277 | Q72R, Q74R, | 0.05523 | 61.04 | 2581 |
| | Q84E, A117W, | | | |
| | V85R, K135Y | | | |
| FKBP-278 | Q72R, Q74R, | 0.03600 | 92.73 | 3425 |
| | Q84E, A117W, | | | |
| | V85R, E130F | | | |
| FKBP-279 | Q72R, Q74R, | 0.01345 | 31.33 | 3302 |
| | Q84E, A117W, | | | |
| | V85R, K120I | | | |
| FKBP-117 | Q72R, Q84E, | 0.00532 | 24.07 | 5193 |
| | A117W, V85R | | | |
| FKBP-195 | Q72R, Q84E, | 0.03903 | 89.61 | 4362 |
| | A117W, V85R, | | | |
| | K135Y | | | |
| FKBP-251 | Q72R, Q84E, | 0.03706 | 92.03 | 5078 |
| | A117W, V85R, | | | |
| | E130F | | | |

TABLE 19
Ec50, FC and Peak values for the graph in FIG. 36

| Construct | Mutation | Ec50 uM | FC | Peak |
|---|---|---|---|---|
| FKBP-280 | Q72R, Q74R, | 0.04953 | 68.34 | 2468 |
| | Q84E, A117W, | | | |
| | V85R, K135Y, | | | |
| | K120I | | | |
| FKBP-281 | Q72R, Q74R, | 0.03657 | 77.66 | 2905 |
| | Q84E, A117W, | | | |
| | V85R, E130F, | | | |
| | K120I | | | |
| FKBP-282 | Q72R, Q74R, | 0.04489 | 39.54 | 2473 |
| | Q84E, A117W, | | | |
| | V85R | | | |
| FKBP-283 | Q72R, Q74R, | 0.40430 | 39.79 | 1328 |
| | Q84E, A117W, | | | |
| | V85R, K135Y | | | |
| FKBP-117 | Q72R, Q84E, | 0.00532 | 24.07 | 5193 |
| | A117W, V85R | | | |
| FKBP-195 | Q72R, Q84E, | 0.03903 | 89.61 | 4362 |
| | A117W, V85R, | | | |
| | K135Y | | | |
| FKBP-251 | Q72R, Q84E, | 0.03706 | 92.03 | 5078 |
| | A117W, V85R, | | | |
| | E130F | | | |

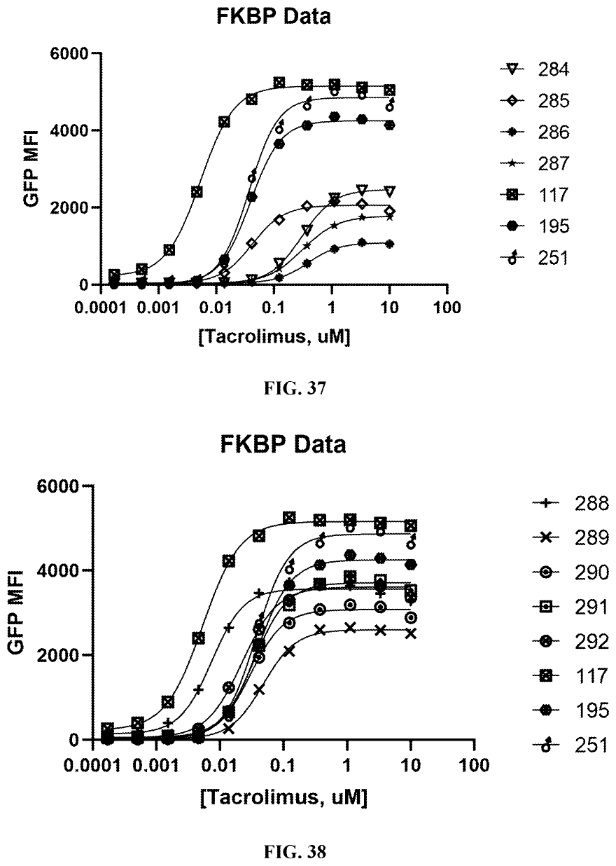

TABLE 20
Ec50, FC and Peak values for the graph in FIG. 37

| Construct | Mutation | Ec50 uM | FC | Peak |
|---|---|---|---|---|
| FKBP-284 | Q72R, Q74R, | 0.30450 | 58.23 | 2441 |
| | Q84E, A117W, | | | |
| | V85R, E130F | | | |
| FKBP-285 | Q72R, Q74R, | 0.04238 | 36.71 | 2161 |
| | Q84E, A117W, | | | |
| | V85R, K120I | | | |
| FKBP-286 | Q72R, Q74R, | 0.38150 | 25.96 | 1089 |
| | Q84E, A117W, | | | |
| | V85R, K135Y, | | | |
| | K120I | | | |
| FKBP-287 | Q72R, Q74R, | 0.3110 | 47.09 | 1747 |
| | Q84E, A117W, | | | |
| | V85R, E130F, | | | |
| | K120I | | | |
| FKBP-117 | Q72R, Q84E, | 0.00532 | 24.07 | 5193 |
| | A117W, V85R | | | |
| FKBP-195 | Q72R, Q84E, | 0.03903 | 89.61 | 4362 |
| | A117W, V85R, | | | |
| | K135Y | | | |
| FKBP-251 | Q72R, Q84E, | 0.03706 | 92.03 | 5078 |
| | A117W, V85R, | | | |
| | E130F | | | |

TABLE 21
Ec50, FC and Peak values for the graph in FIG. 38

| Construct | Mutation | Ec50 uM | FC | Peak |
|---|---|---|---|---|
| FKBP-288 | Q72R, Q84E, | 0.00740 | 30.10 | 3718 |
| | A117W, V85R, | | | |
| | K120I | | | |
| FKBP-289 | Q72R, Q84E, | 0.04788 | 73.38 | 2659 |
| | A117W, V85R, | | | |
| | K120I, K135Y | | | |
| FKBP-290 | Q72R, Q84E, | 0.03021 | 92.89 | 3191 |
| | A117W, V85R, | | | |
| | K120I, E130F | | | |
| FKBP-291 | Q72R, Q84E, | 0.03457 | 53.37 | 3859 |
| | A117W, V85R, | | | |
| | E130N | | | |
| FKBP-292 | Q72R, Q84E, | 0.02232 | 50.68 | 3797 |
| | A117W, V85R, | | | |
| | K135Q | | | |
| FKBP-117 | Q72R, Q84E, | 0.00532 | 24.07 | 5193 |
| | A117W, V85R | | | |
| FKBP-195 | Q72R, Q84E, | 0.03903 | 89.61 | 4362 |
| | A117W, V85R, | | | |
| | K135Y | | | |
| FKBP-251 | Q72R, Q84E, | 0.03706 | 92.03 | 5078 |
| | A117W, V85R, | | | |
| | E130F | | | |

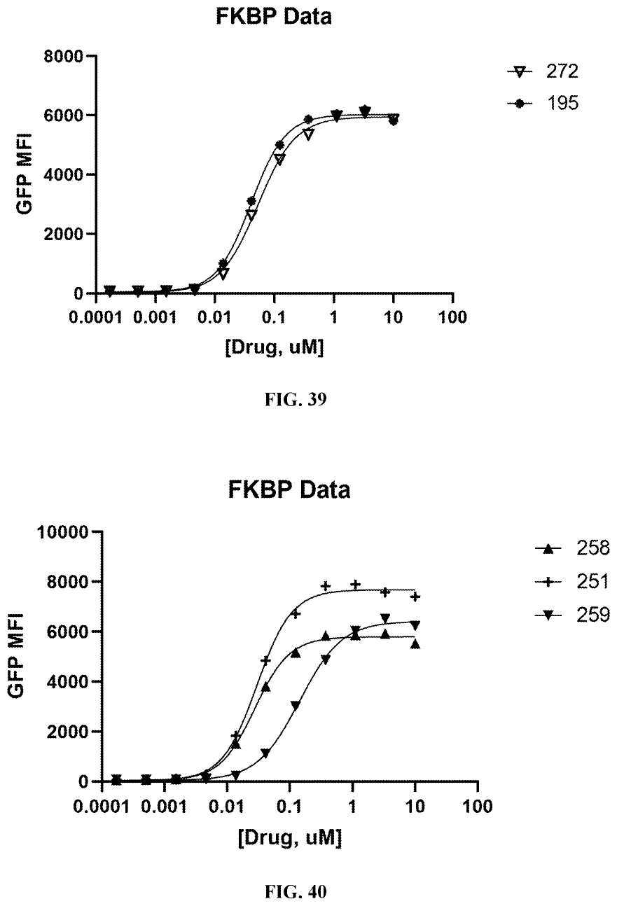

FIGS. 39-50 show the results from further engineering of Top DRDs from the Cas9 screen, particularly, providing a direct comparison of constructs either mutating to other amino acids or subtracting the M54A mutation.

TABLE 22
Ec50, FC and Peak values for the graph in FIG. 39

| Construct | Mutation | Ec50 uM | FC | Basal | Peak |
|---|---|---|---|---|---|
| FKBP-195 | Q72R, Q84E, | 0.0407 | 90 | 68.4 | 5808 |
| | A117W, | | | | |
| | V85R, | | | | |
| | K135Y | | | | |
| FKBP-272 | Q72R, Q84E, | 0.0531 | 85 | 71.5 | 5857 |
| | A117W, | | | | |
| | V85R, K135L | | | | |

TABLE 23
Ec50, FC and Peak values for the graph in FIG. 40

| Construct | Mutation | Ec50 uM | FC | Basal | Peak |
|---|---|---|---|---|---|
| FKBP-258 | Q72R, Q84E, | 0.0276 | 99 | 60.2 | 5533 |
| | A117W, | | | | |
| | V85R, DRD | | | | |
| | E130F | | | | |
| FKBP-259 | Q72R, Q84E, | 0.1428 | 90 | 72.3 | 6232 |
| | A117W, | | | | |
| | V85R, DRD | | | | |
| | E130K | | | | |
| FKBP-251 | Q72R, Q84E, | 0.0300 | 100 | 76 | 7406 |
| | A117W, | | | | |
| | V85R, DRD | | | | |
| | E130F | | | | |

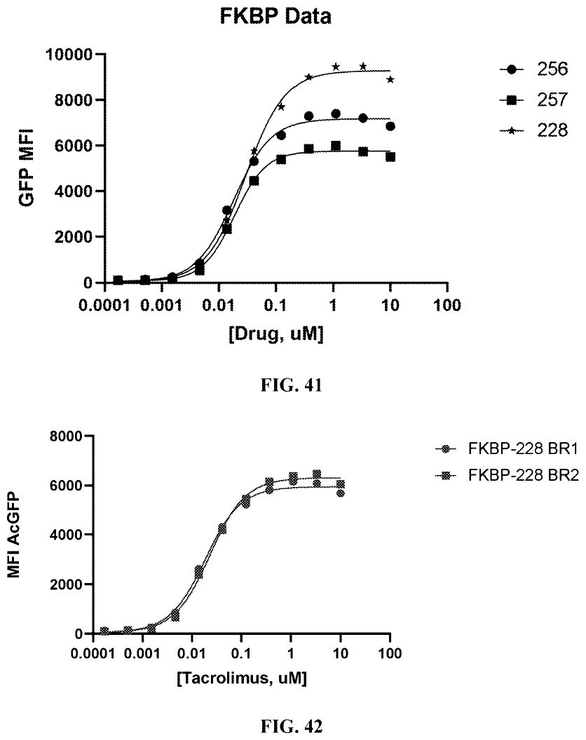

TABLE 24
Ec50, FC and Peak values for the graph in FIG. 41

| Construct | Mutation | Ec50 uM | FC | Basal | Peak |
|---|---|---|---|---|---|
| FKBP-256 | Q72R, Q84E, | 0.0181 | 65 | 111 | 6851 |
| | A117W, | | | | |
| | V85R, DRD | | | | |
| | E137M | | | | |
| FKBP-257 | Q72R, Q84E, | 0.0186 | 59 | 98 | 5501 |
| | A117W, | | | | |
| | V85R, DRD | | | | |
| | E137F | | | | |
| FKBP-228 | Q72R, Q84E, | 0.0293 | 85 | 112 | 8896 |
| | A117W, | | | | |
| | V85R, DRD | | | | |
| | E137R | | | | |

TABLE 25
Ec50, FC and Peak values for the graph in FIG. 42

| | | | | | | At |
|---|---|---|---|---|---|---|
| | Construct | Mutation | Ec50 uM | FC | Basal | 10 uM |
| BR1 | FKBP-228 | Q72R, | 0.01856 | 55.15 | 103 | 5680 |
| (Biorep 1) | | Q84E, | | | | |
| | | A117W, | | | | |
| | | V85R, DRD | | | | |
| | | E137R | | | | |
| BR2 | FKBP-228 | Q72R, | 0.02313 | 60.71 | 99.6 | 6047 |
| (Biorep 2) | | Q84E, | | | | |
| | | A117W, | | | | |
| | | V85R, DRD | | | | |
| | | E137R | | | | |

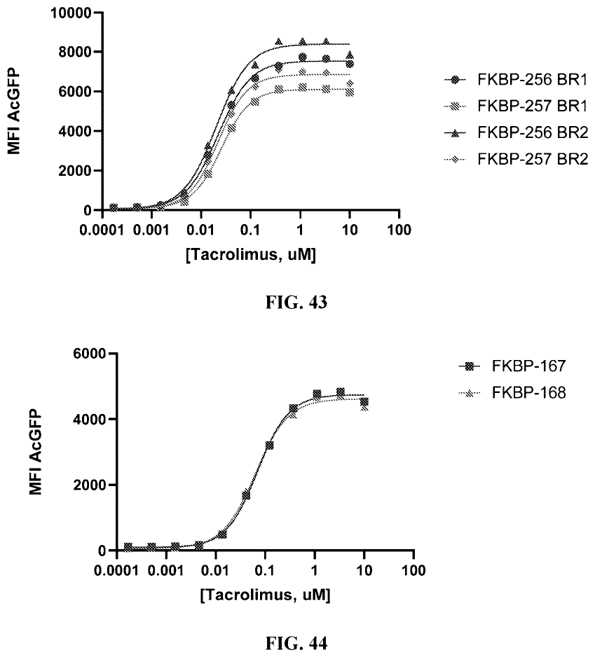

TABLE 26
Ec50, FC and Peak values for the graph in FIG. 43

| | Construct | Mutation | Ec50 uM | FC | Basal | At 10 uM |
|---|---|---|---|---|---|---|
| BR1 | FKBP-256 | Q72R, Q84E, | 0.02029 | 71.46 | 110 | 7861 |
| (Biorep 1) | | A117W, V85R, | | | | |
| | | DRD E137M | | | | |
| BR2 | FKBP-256 | Q72R, Q84E, | 0.02207 | 59.04 | 125 | 7380 |
| (Biorep 2) | | A117W, V85R, | | | | |
| | | DRD E137M | | | | |
| BR1 | FKBP-257 | Q72R, Q84E, | 0.02213 | 59.79 | 107 | 6397 |
| (Biorep 1) | | A117W, V85R, | | | | |
| | | DRD E137F | | | | |
| BR2 | FKBP-257 | Q72R, Q84E, | 0.02557 | 50.39 | 118 | 5946 |
| (Biorep 2) | | A117W, V85R, | | | | |
| | | DRD E137F | | | | |

TABLE 27
Ec50, FC and Peak values for the graph in FIG. 44

| Construct | Mutation | Ec50 uM | FC | Basal | At 10 uM |
|---|---|---|---|---|---|
| FKBP-167 | Q72R, S79W, | 0.0706 | 47.22 | 96 | 4533 |
| | Q84R, | | | | |
| | A117W, | | | | |
| | V85R, DRD | | | | |
| | M54A, V85R | | | | |
| FKBP-168 | Q72R, T82W, | 0.0637 | 60.19 | 72.7 | 4376 |
| | Q84R, | | | | |
| | A117W, | | | | |
| | V85R, DRD | | | | |
| | M54A, V85R | | | | |

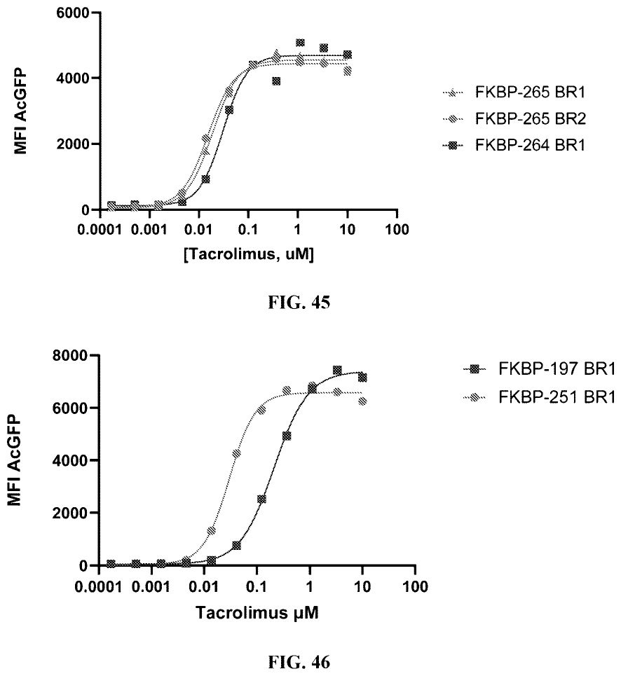

TABLE 28
Ec50, FC and Peak values for the graph in FIG. 45

| | Construct | Mutation | Ec50 uM | FC | Basal | At 10 uM |
|---|---|---|---|---|---|---|
| BR1 | FKBP-265 | Q72R, T82W, | 0.01861 | 53.72 | 78 | 4190 |
| (Biorep 1) | | Q84R, A117W, | | | | |
| | | DRD V85R | | | | |
| BR2 | FKBP-265 | Q72R, T82W, | 0.01514 | 41.58 | 102 | 4241 |
| (Biorep 2) | | Q84R, A117W, | | | | |
| | | DRD V85R | | | | |
| BR1 | FKBP-264 | Q72R, S79W, | 0.03089 | 37.06 | 127 | 4706 |
| (Biorep 1) | | Q84R, A117W, | | | | |
| | | DRD V85R | | | | |

TABLE 29
Ec50, FC and Peak values for the graph in FIG. 46

| | Construct | Mutation | Ec50 uM | FC | Basal | At 10 uM |
|---|---|---|---|---|---|---|
| BR1 | FKBP-251 | Q72R, Q84E, | 0.03015 | 97.61 | 64 | 6247 |
| (Biorep 1) | | A117W, V85R, | | | | |
| | | DRD E130F | | | | |
| BR2 | FKBP-251 | Q72R, Q84E, | 0.023 | 76.79 | 46.1 | 3540 |
| (Biorep 2) | | A117W, V85R, | | | | |
| | | DRD E130F | | | | |
| BR1 | FKBP-197 | Q72R, Q84E, | 0.2129 | 101.12 | 70.7 | 7149 |
| (Biorep 1) | | A117W, V85R, | | | | |
| | | DRD E130R | | | | |
| BR2 | FKBP-197 | Q72R, Q84E, | 0.2447 | 72.21 | 55.2 | 3986 |
| (Biorep 2) | | A117W, V85R, | | | | |
| | | DRD E130R | | | | |

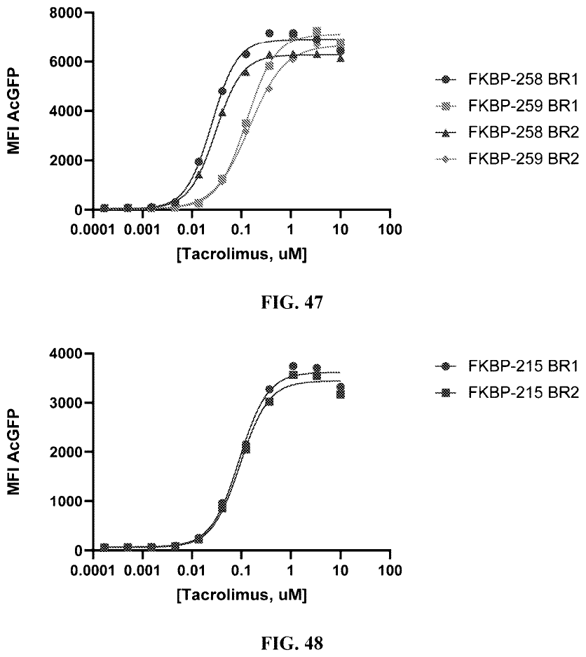

TABLE 30
Ec50, FC and Peak values for the graph in FIG. 47

| | Construct | Mutation | Ec50 uM | FC | Basal | At 10 uM |
|---|---|---|---|---|---|---|
| BR1 | FKBP-258 | Q72R, Q84E, | 0.02514 | 96.50 | 66.6 | 6427 |
| (Biorep 1) | | A117W, V85R, | | | | |
| | | DRD E130F | | | | |
| BR2 | FKBP-258 | Q72R, Q84E, | 0.03022 | 88.49 | 69.4 | 6141 |
| (Biorep 2) | | A117W, V85R, | | | | |
| | | DRD E130F | | | | |
| BR1 | FKBP-259 | Q72R, Q84E, | 0.1275 | 95.97 | 70.7 | 6785 |
| (Biorep 1) | | A117W, V85R, | | | | |
| | | DRD E130K | | | | |
| BR2 | FKBP-259 | Q72R, Q84E, | 0.1458 | 84.76 | 77.3 | 6552 |
| (Biorep 2) | | A117W, V85R, | | | | |
| | | DRD E130K | | | | |

TABLE 31
Ec50, FC and Peak values for the graph in FIG. 48

| | Construct | Mutation | Ec50 uM | FC | Basal | At 10 uM |
|---|---|---|---|---|---|---|
| BR1 | FKBP-215 | Q72R, Q84G, | 0.09082 | 51.05 | 65 | 3318 |
| (Biorep 1) | | A117W, V85R, | | | | |
| | | DRD P43R, | | | | |
| | | M54A, K135Y | | | | |
| BR2 | FKBP-215 | Q72R, Q84G, | 0.09453 | 53.30 | 59.4 | 3166 |
| (Biorep 2) | | A117W, V85R, | | | | |
| | | DRD P43R, | | | | |
| | | M54A, K135Y | | | | |

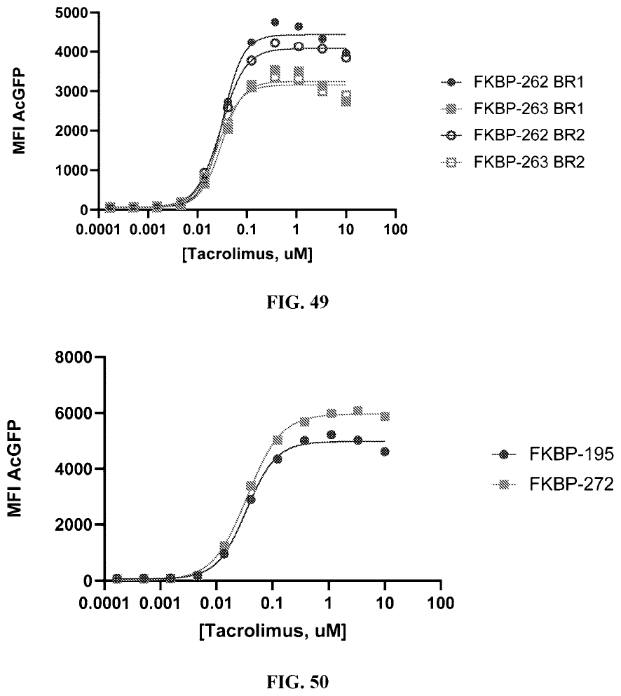

TABLE 32
Ec50, FC and Peak values for the graph in FIG. 49

| | Construct | Mutation | Ec50 uM | FC | Basal | At 10 uM |
|---|---|---|---|---|---|---|
| BR1 | FKBP-262 | Q72R, Q84E, | 0.03231 | 66.64 | 59.5 | 3965 |
| (Biorep 1) | | A117W, V85R, | | | | |
| | | DRD P43R, | | | | |
| | | K135Y | | | | |
| BR2 | FKBP-262 | Q72R, Q84E, | 0.0298 | 52.21 | 73.7 | 3848 |
| (Biorep 2) | | A117W, V85R, | | | | |
| | | DRD P43R, | | | | |
| | | K135Y | | | | |
| BR1 | FKBP-263 | Q72R, Q84G, | 0.0306 | 49.02 | 56 | 2745 |
| (Biorep 1) | | A117W, V85R, | | | | |
| | | DRD P43R, | | | | |
| | | K135Y | | | | |
| BR2 | FKBP-263 | Q72R, Q84G, | 0.02582 | 45.75 | 63.3 | 2896 |
| (Biorep 2) | | A117W, V85R, | | | | |
| | | DRD P43R, | | | | |
| | | K135Y | | | | |

TABLE 33
Ec50, FC and Peak values for the graph in FIG. 50

| Construct | Mutation | Ec50 uM | FC | Basal | At 10 uM |
|---|---|---|---|---|---|
| FKBP-195 | Q72R, Q84E, | 0.03448 | 61.07 | 75.5 | 4611 |
| | A117W, | | | | |
| | V85R, DRD | | | | |
| | K135Y | | | | |
| FKBP-272 | Q72R, Q84E, | 0.03532 | 105.86 | 55.6 | 5886 |
| | A117W, | | | | |
| | V85R, DRD | | | | |
| | K135L | | | | |

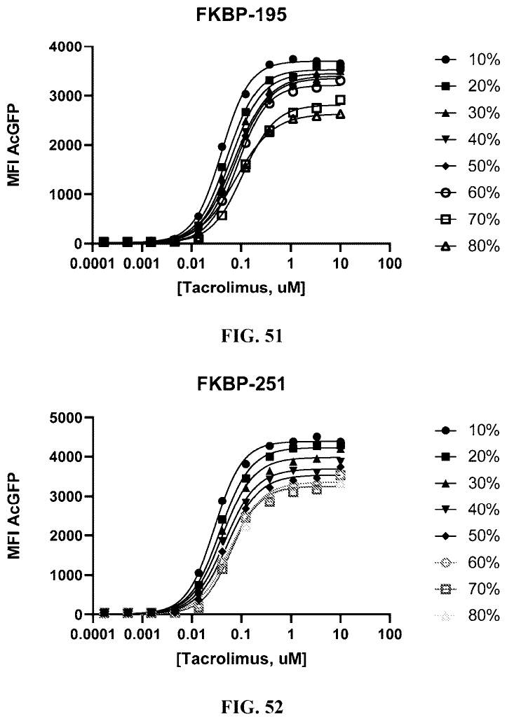

FIGS. 51-54 show the results of Tacrolimus serum experiments. Tacrolimus dilutions were made in each % FBS as shown in FIGS. 51-54. Incubation with increasing FBS results in 2 fold increase in Ec50 with 80% FBS.

TABLE 34
Ec50 values for FIG. 51, construct FKBP-195

| % Serum | Ec50 uM |
|---|---|
| 10% | 0.04047 |
| 20% | 0.05282 |
| 30% | 0.06241 |
| 40% | 0.07271 |
| 50% | 0.07901 |
| 60% | 0.08572 |
| 70% | 0.1098 |
| 80% | 0.07797 |

TABLE 35
Ec50 values for FIG. 52, construct FKBP-251

| % Serum | Ec50 uM |
|---|---|
| 10% | 0.02879 |
| 20% | 0.03741 |
| 30% | 0.04127 |
| 40% | 0.04616 |
| 50% | 0.05231 |
| 60% | 0.06087 |
| 70% | 0.0633 |
| 80% | 0.05531 |

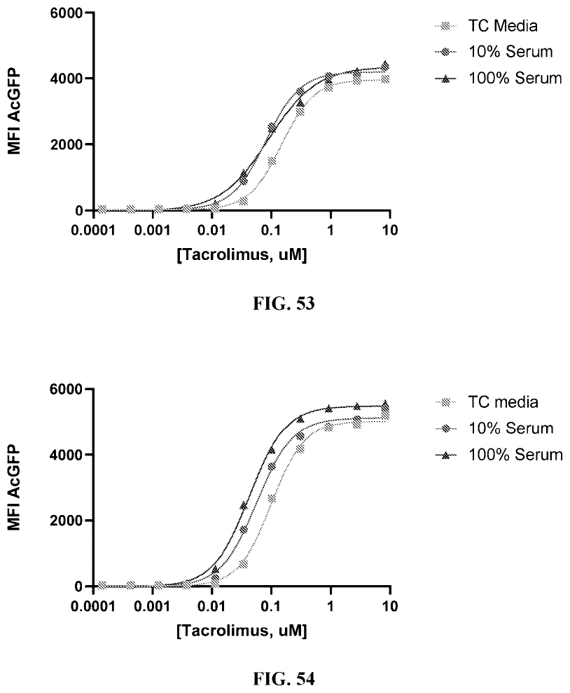

TABLE 36
Ec50 values for FIG. 53, construct FKBP-195

| | Ec50 uM |
|---|---|
| TC Media | 0.135 |
| 10% Serum | 0.073 |
| 100% Serum | 0.082 |

TABLE 37
Ec50 values for FIG. 54, construct FKBP-251

| | Ec50 uM |
|---|---|
| TC Media | 0.091 |
| 10% Serum | 0.052 |
| 100% Serum | 0.038 |

To summarize the results of the examples, initial characterization of FKBP mutants discovered from a pooled saturation mutagenesis library screen identified the V85R mutation (FKBP-117) which when combined with the original binding site mutations (Q72R, Q84E, A117W) significantly decreased the FKBP/tacrolimus Ec50 to 5 nM. To further improve the properties yielded from construct FKBP-117, a series of combinatorial mutants (FKBP-160-FKBP-255) were made to identify FKBP constructs that yielded favorable DRD properties for protein regulation-<50 nM Ec50 and a low basal off state in the absence of tacrolimus. FKBP mutants were cloned in a manner to make a fusion protein with AcGFP in order to assess FKBP mutant regulation by using AcGFP expression as a readout. Constructs were stably transduced into Jurkat cells and their response to tacrolimus was analyzed using flow cytometry. FKBP mutants that had a >10 fold change in AcGFP expression in response to dosing with 10 uM Tacrolimus were then further screened with a Tacrolimus dose response to determine the Ec50 for those constructs. FKBP-195, FKBP-228, and FKBP-251 emerged as the top FKBP constructs yielding the lowest basal expression with Ec50 values 35 nm, 19 nM and 30 nm, respectively. FKBP-215 did exhibit the lowest basal AcGFP expression with an Ec50 of 90 nM. Further iterative FKBP mutants (FKBP-256-FKBP-292) were constructed from these initial combinatorial mutants to see if we could further improve FKBP DRD properties. FKBP-272 was constructed from FKBP-195 by changing the K135Y mutation to K135L which resulted in a similar Ec50 but lower basal AcGFP expression. FKBP-263 was based on FKBP-215 removing the M54A mutation which retained the low basal AcGFP expression while improving the Ec50 to 30 nM.

1. A nucleic acid encoding
(a) an FKBP13 or a variant thereof responsive to a ligand and
(b) a payload having a biological activity,
wherein, upon expression of the nucleic acid, the FKBP13 is operably linked to the payload.

2. The nucleic acid of claim 1, wherein, upon expression of the nucleic acid, the FKBP13 interacts with an effective amount of the ligand to modulate the biological activity of the payload.

3. The nucleic acid of claim 1, wherein the encoded FKBP13 is a variant of SEQ ID NO: 1.

4. The nucleic acid of claim 1, wherein the encoded FKBP13 variant comprises one or more mutations in a ligand binding site.

5. The nucleic acid of claim 1, wherein the ligand is FK506, rapamycin, or both FK506 and rapamycin.

6. The nucleic acid of claim 5, wherein the encoded FKBP13 variant modulates the biological activity of the payload in response to FK506 more than in response to rapamycin.

7. The nucleic acid of claim 3, wherein the encoded FKBP13 variant is a fragment of FKBP13.

8. The nucleic acid of claim 7, wherein the encoded FKBP13 variant is a C-terminal fragment of FKBP13.

9. The nucleic acid of claim 1, further encoding a promoter sequence.

10. The nucleic acid of claim 1, wherein the payload comprises an RNA-guided endonuclease.

11. The nucleic acid of claim 10, wherein the endonuclease is a Cas9 endonuclease.

12. The nucleic acid of claim 11, wherein the Cas9 endonuclease is a Staphylococcus aureus Cas9 (SaCas9) or Staphylococcus lugdunensis Cas9 (SluCas9).

13. The nucleic acid of claim 10, further encoding at least one a guide RNA.

14. The nucleic acid of claim 13, wherein the nucleic acid is configured for packaging as a single nucleotide sequence in an AAV vector with a promoter sequence, a guide RNA, and a sequence that encodes a Cas9 endonuclease payload, and wherein the promoter sequence, the guide RNA, and the sequence encoding the Cas9 comprise at least 3000 base pairs.

15. The nucleic acid of claim 1, wherein the encoded FKBP13 fragment is less than 100 amino acids.

16. The nucleic acid of claim 15, wherein the encoded FKBP fragment is 90-99 amino acids.

17. The nucleic acid of claim 16, wherein the encoded FKBP13 fragment has an amino acid sequence with at least 85% identity to SEQ ID NO: 2.

18. The nucleic acid of claim 17, wherein the encoded FKBP13 comprises an amino acid sequence selected from SEQ ID NO: 3-109.

19. A genetically modified FKBP13, comprising one or more mutations in a binding site for FK506.

20. The genetically modified FKBP13 of claim 19, wherein the FKBP13 is a C-terminal fragment of SEQ ID NO:1.

21. The genetically modified FKBP13 of claim 20, comprising an amino acid sequence selected from SEQ ID NO:3-66.

22. The genetically modified FKBP13 of claim 19, wherein the FKBP13 fragment has a lower binding affinity for rapamycin than a control FKBP13 fragment without mutations in the ligand binding site.

23. The genetically modified FKBP13 of claim 19, wherein the FKBP13 has a higher binding affinity for FK506 than a control FKBP13 fragment without mutations in the ligand binding site.

24. A recombinant polypeptide encoded by the nucleic acid of claim 1, wherein the payload is operably linked to the FKBP13 or variant thereof and wherein activity of the payload in a biological system is regulatable by the concentration of the ligand contacting the FKBP13.

25. The recombinant polypeptide of claim 24, wherein the EC₅₀ of FK506 for the FKBP13 or variant thereof in the biological system is about 2-fold to about 20-fold lower than the Cₘₐₓ of FK506 in the biological system.

26. The recombinant polypeptide of claim 24, wherein the biological system is a tissue or cell.

27. The recombinant polypeptide of claim 26, wherein the biological system is mammalian blood.

28. A vector comprising the nucleic acid of claim 1.

29. The vector of claim 28, wherein the vector is a viral vector.

30. The vector of claim 28, wherein the encoded payload is an RNA-guided endonuclease and wherein the vector further comprises one or more guide RNA sequences comprising a first nucleotide sequence that hybridizes to a target DNA in the genome of a cell and a second nucleotide sequence configured to interact with the RNA-guided endonuclease.

31. The vector of claim 30, wherein the vector further comprises one or more inverted terminal repeat sequences.

32. A cell comprising the vector of claim 28.

33. The cell of claim 32, wherein the cell is a mammalian cell.

34. The cell of claim 33, wherein the mammalian cell is a human cell.

35. The cell of claim 32, wherein the cell is a muscle cell, a stem cell, or a lymphocyte.

36. A method of producing a recombinant cell, comprising introducing the vector of claim 28 into a cell.

37. The method of claim 36, wherein the cell is a mammalian cell.

38. The method of claim 37, wherein the mammalian cell is a human cell.

39. The method of claim 36, wherein the cell is a muscle cell, a stem cell, or a lymphocyte.

40. A cell produced by the method of claim 36.

41. A method of regulating a payload in a cell, comprising
(a) introducing into a cell a vector comprising a nucleic acid encoding an FKBP13 or variant thereof responsive to a ligand, wherein the nucleic acid further comprises a promoter sequence and a sequence that encodes the payload, and
(b) contacting the cell with an effective amount of the ligand.

42. A method of modifying target DNA in a cell, comprising
(a) introducing into the cell a vector comprising
(1) an RNA-guided endonuclease-encoding nucleic acid comprising a sequence that encodes an FKBP13 or variant thereof responsive to a ligand, a promoter sequence, and a sequence that encodes an RNA-guided endonuclease, and
(2) one or more guide RNAs comprising a nucleotide sequence that hybridizes to a target DNA in the genome of a cell and a nucleotide sequence configured to interact with the RNA-guided endonuclease;
(b) contacting the vector-containing cell with an effective amount of ligand to induce activity of the RNA-guided endonuclease, wherein the RNA-guided endonuclease interacts with the guide RNA and wherein the RNA-guided endonuclease specifically binds and cleaves the target DNA in the cell.

43. The method of claim 41, wherein the vector is an AAV vector

44. The method of claim 41, wherein the cell is a mammalian cell.

45. The method of claim 44, wherein the mammalian cell is a human cell.

46. The method of claim 41, wherein the cell is a muscle cell, a stem cell, or a lymphocyte.

47. A genetically modified cell produced by the method of claim 42.

48. A method of treating a disease or disorder responsive to genetic modification in a subject in need thereof, comprising
(a) introducing into one or more cells of the subject a vector comprising
(1) an RNA-guided endonuclease encoding nucleic acid comprising a sequence that encodes an FKBP13 or variant thereof responsive to a ligand, a promoter sequence and a sequence that encodes an RNA-guided endonuclease, and
(2) at least one guide RNA comprising a nucleotide sequence that hybridizes to a target DNA in the genome of a cell and a nucleotide sequence configured to interact with the RNA-guided endonuclease;
(b) contacting the vector-containing one or more cells with an effective amount of ligand to induce activity of the RNA-guided endonuclease, wherein the RNA-guided endonuclease interacts with the guide RNA and wherein the RNA-guided endonuclease specifically binds and cleaves the target DNA one or more cells of the subject.

49. A method of treating a disease or disorder responsive to genetic modification in a subject in need thereof, comprising
(a) administering to the subject one or more cells comprising a vector, wherein the vector comprises
(1) an RNA-guided endonuclease encoding nucleic acid comprising a sequence that encodes an FKBP13 or variant thereof responsive to a ligand, a promoter sequence and a sequence that encodes an RNA-guided endonuclease, and
(2) a guide RNA comprising a nucleotide sequence that hybridizes to a target DNA in the genome of the one or more cells and a nucleotide sequence configured to interact with the RNA-guided endonuclease;
(b) administering to the subject an effective amount of ligand to induce activity of the RNA-guided endonuclease, wherein the RNA-guided endonuclease interacts with the guide RNA and wherein the RNA-guided endonuclease specifically modifies the genome of one or more cells.

50. A method of treating a disease or disorder in a subject, comprising administering to the subject one or more genetically modified cells of claim 47.

51. The method of claim 48, wherein the disease or disorder is Duchenne muscular dystrophy, myotonic dystrophy, cystic fibrosis, sickle cell, beta thalassemia, alpha-1 antitrypsin deficiency, APOL1-mediated kidney disease, or Type 1 diabetes.
